(19) United States
(12) Patent Application Publication
(10) Pub. No.: US 2022/0213225 A1
(43) Pub. Date: Jul. 7, 2022

(54) MULTISPECIFIC TREG BINDING MOLECULES

(71) Applicant: INVENRA INC., Madison, WI (US)

(72) Inventors: Lucas BAILEY, Suzhou (CN); Bryan GLASER, Fitchburg, WI (US); Qufei LI, Suzhou (CN); Roland GREEN, Madison, WI (US); Dileep Kumar PULUKKUNAT, Middleton, WI (US); Bonnie HAMMER, Madison, WI (US); Daniel Justin GERHARDT, Madison, WI (US)

(21) Appl. No.: 17/264,735

(22) PCT Filed: Jul. 29, 2019
(86) PCT No.: PCT/US2019/043958

§ 371 Date: Jan. 29, 2021

Related U.S. Application Data
(60) Provisional application No. 62/712,077, filed on Jul. 30, 2018.

(51) Int. Cl. C07K 16/46; C07K 16/00; C07K 16/24; C07K 16/28

(52) U.S. Cl. CPC C07K 16/468; C07K 16/005; C07K 16/241; C07K 16/2818; C07K 16/2866; C07K 16/2878; C07K 2317/66; C07K 2317/31; C07K 2317/32; C07K 2317/524; C07K 2317/526; C07K 2317/565; C07K 2317/21

(57) ABSTRACT

Multispecific Treg-binding molecules, constructs, pharmaceutical compositions comprising the constructs, and methods of use thereof are presented.

### 1. CROSS-REFERENCE TO RELATED APPLICATIONS

This application claims priority under 35 U.S.C. § 119(e) to U.S. Provisional Application No. 62/712,077, filed Jul. 30, 2018, which is incorporated by reference in its entirety.

### 2. SEQUENCE LISTING

The instant application contains a Sequence Listing with 304 sequences which has been submitted via EFS-Web and is hereby incorporated herein by reference in its entirety. Said ASCII copy, created on Aug. 20, 2019, is named 40868WO_CRF_sequencelisting.txt, and is 549,933 bytes in size.

### 3. BACKGROUND

Regulatory T cells (Tregs) play an important role in immune homeostasis and self-tolerance. They also play key roles in controlling autoimmunity, inflammation, infection, and tumor immunity. The central role of Tregs in immune homeostasis has been demonstrated by Treg ablation studies. For example, ablation of Tregs leads to the development of fatal autoimmune disorders in mice. J Immunol Dec. 15, 2009, 183 (12) 7631-7634. Therefore, non-specific depletion of Tregs could negatively impact immune homeostasis and lead to undesirable autoimmune phenotypes.

The tumor microenvironment includes various cell types such as CD8+ T-cells, CD4+ T-cells, Tregs, macrophages, natural killer (NK) cells, dendritic cells (DCs), B cells, mast cells, and other cell types. The immune cells in the tumor microenvironment contribute to its immunosuppressive nature, promoting immune evasion, and cancer progression.

Tregs are known to infiltrate tumors. Accumulation of tumor-associated FoxP3+ Tregs and high Treg/T effector ratios in the tumor microenvironment is associated with worse prognosis in many cancers.

Tumor-associated Tregs exhibit distinct phenotypes, for example by upregulating markers associated with activation and immunosuppressive activity. For example, tumor-infiltrating Tregs exhibit higher expression of, e.g., CTLA4, LAG-3, TIM-3, PD-1, ICOS, GITR, CD25, CD44, NRP-1 and CD69, among others. Chaudhary and Elkord, Vaccines (Basel). 2016 September; 4(3): 28; Liu et al, FEBS Journal 283 (2016) 2731-2748.

Several studies have demonstrated an important role for Tregs in tumor immune self-tolerance. Tumor-associated Tregs can also promote cancer progression in other ways, e.g., by promoting tumor angiogenesis. Giatromanolaki et al. (2008) Gynecol Oncol 110, 216-221, which is incorporated by reference in its entirety. Furthermore, many studies have shown that Treg ablation reduces tumor growth, and in some cases have resulted in tumor clearance. Other studies have shown that Treg ablation can enhance cancer immunotherapy.

There has been considerable interest in selectively targeting Tregs in the tumor microenvironment. However, the selective inactivation/depletion of tumor-infiltrating Tregs present several challenges, as tumor Tregs often share the same cell surface markers as other conventional T-cells or peripheral Tregs. For example, antibody-based approaches generally target both tumor-infiltrating Tregs and activated effector T cells. De Simone et al (2016) Immunity 45, 1135-1147.

Therefore, there exists a need for improved platforms and systems for selective targeting of tumor-associated Tregs.

### 4. SUMMARY

Described herein is a multispecific Treg-binding molecule, comprising a first antigen binding site (ABS) specific for a first Treg cell surface antigen; and a second antigen binding site (ABS) specific for a second Treg cell surface antigen; wherein the first ABS binds the first Treg cell surface antigen with a Kd that is greater than 10 nM, wherein the second ABS binds the second Treg cell surface antigen with a Kd that is greater than 10 nm, wherein the second Treg cell surface antigen is not the first Treg cell surface antigen, and wherein the multispecific Treg-binding molecule binds to a target Treg with a Kd that is less than 100 nM.

In some embodiments, the target Treg is a tumor-associated Treg. In some embodiments, the target Treg expresses the first and second Treg cell surface antigens. In some embodiments, the target Treg overexpresses the first and second Treg cell surface antigens as compared to a non-target cell.

In some embodiments, the first and second Treg cell surface antigens are CTLA4 and CD25.

In some embodiments, the first ABS binds to the first Treg cell surface antigen with a Kd that is greater than 100 nM, the second ABS binds to the second Treg cell surface antigen with a Kd that is greater than 100 nM, and the multispecific Treg-binding molecule binds to a target Treg with a Kd that is less than 10 nM.

In some embodiments, the multispecific Treg-binding molecule comprises a first, second, third, and fourth polypeptide chain, wherein: (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (0 the third and the fourth polypeptides are associated through an interaction between the H and the L domains and an interaction between the I and the M domains; and (g) the first and the third polypeptides are associated through an interaction between the D and the J domains and an interaction between the E and the K domains to form the multispecific Treg-binding molecule, wherein the interaction between the A domain and the F domain form the first ABS, and wherein the interaction between the H domain and the L domain form the second ABS.

In some embodiments, wherein the first and second Treg cell surface antigens comprise antigens are each independently selected from CTLA4, CD25, OX40, GITR, TNFRII, NRP1, CD30, CD27, ICOS, TIGIT, 4-1BB, LAG-3, and PDL-2.

In some embodiments, the first and second Treg cell surface antigens are each independently selected from CTLA4, CD25, OX40, and NRP1.

In some embodiments, the first Treg cell surface antigen is CTLA4 and the second Treg cell surface antigen is CD25.

In some embodiments, the first Treg cell surface antigen is CTLA4 and the second Treg cell surface antigen is OX40.

In some embodiments, the first ABS comprises a first VL CDR1 amino acid sequence, a first VL CDR2 amino acid sequence, and a first VL CDR3 amino acid sequence of a light chain variable region (VL), wherein the first VL CDR3 sequences are selected from the VL CDR3 sequences from Table 20.

In some embodiments, the first ABS further comprises a first VH CDR1 amino acid sequence, a first VH CDR2 amino acid sequence, and a first VH CDR3 amino acid sequence of a heavy chain variable region (VH), wherein the first VH CDR1, CDR2, and CDR3 sequences are selected from the VH CDR1, CDR2, and CDR3 sequences from Table 20.

In some embodiments, the second ABS comprises a second VL CDR1 amino acid sequence, a second VL CDR2 amino acid sequence, and a second VL CDR3 amino acid sequence of a light chain variable region (VL), wherein the second VL CDR1, CDR2, and CDR3 sequences are selected from Table 20.

In some embodiments, the second ABS further comprises a second VH CDR1 amino acid sequence, a second VH CDR2 amino acid sequence, and a second VH CDR3 amino acid sequence of a heavy chain variable region (VH), wherein the second VH CDR1, CDR2, and CDR3 sequences are selected from Table 20.

In some embodiments, the multispecific Treg-binding molecule is conjugated to a therapeutic agent.

In some embodiments, the multispecific Treg-binding molecule further comprises a third ABS specific for a cytotoxic lymphocyte.

In some embodiments, the cytotoxic lymphocyte is a natural killer (NK) cell.

In some embodiments, the multispecific Treg-binding molecule binds to the target Treg with 10-fold higher avidity than a T killer cell, T helper cell, memory T cell, or peripheral non-tumor associated Treg.

In some embodiments, the target Treg is a primate Treg.

In some embodiments, the primate Treg is a human Treg or cyno Treg.

Also provided herein is a pharmaceutical composition comprising an effective amount of a multispecific Treg-binding molecule described herein and a pharmaceutically acceptable excipient.

Also described herein is a method of treating a proliferative disease in a human subject, comprising administering to the human subject a pharmaceutical composition described herein.

In some embodiments, the proliferative disease is cancer.

Also described herein is a method of suppressing activity or reducing the number of tumor-associated Tregs in a subject, comprising administering to the subject a pharmaceutical composition described herein.

Also described herein is a method of screening a set of candidate multispecific Treg-binding molecules for a multispecific Treg-binding molecule that selectively binds a tumor-associated Treg, comprising assessing binding avidity of a candidate to (i) a first population of cells comprising the first Treg cell surface antigen but not the second Treg cell surface antigen, (ii) a second population of cells comprising the second Treg cell surface antigen but not the first Treg cell surface antigen, and (iii) a third population of cells comprising the first and second Treg cell surface antigens; and selecting the candidate as a Treg-binding molecule if the binding avidity to the third population of cells is at least two-fold greater than avidity to the first or second cell. In some embodiments, the method comprises selecting the candidate as a Treg-binding molecule if the binding avidity to the third population of cells is at least ten-fold greater than avidity to the first or second population of cells. In some embodiments, the method comprises the assessing comprises contacting the first, second, and third populations of cells with a dilution series of library member concentrations. In some embodiments, the dilution series comprises library member concentrations ranging from 1-2000 nM. In some embodiments, the method comprises selecting the library member as a Treg-binding molecule if the library member exhibits less than 15% binding to the first and second populations of cells at 100 nM, but more than 50% binding to the third population of cells at 100 nM. In some embodiments, the method comprises selecting the library member as a Treg-binding molecule if the library member exhibits less than 10% binding to the first and second populations of cells at 500 nM, but more than 90% binding to the third population of cells at 500 nM.

Also provided herein is an isolated polynucleotide encoding an amino acid sequence that is at least 97% identical to any one of the sequences in Tables 16, 21, or 22. Also provided herein is a vector comprising any one or more of the isolated polynucleotides described herein. Also provided herein is a host cell comprising any one or more than one of the vectors described herein.

### 5. BRIEF DESCRIPTION OF THE DRAWINGS

FIG. 1 shows an alignment of the CH3-CH3 IgG1 dimer pair with CH1-CL. The quatemary structures align with an RMSD of ˜1.6 A2.

FIG. 2 presents schematic architectures, with respective naming conventions, for various binding molecules (also called antibody constructs) described herein.

FIG. 3 presents a higher resolution schematic of polypeptide chains and their domains, with respective naming conventions, for the bivalent 1×1 antibody constructs described herein.

FIG. 4 shows the architecture of an exemplary bivalent, monospecific, construct.

FIG. 5 shows data from a biolayer interferometry (BLI) experiment, described in Example 1, in which a bivalent monospecific binding molecule having the architecture illustrated in FIG. 4 [polypeptide 1: VL-CH3(Knob)-CH2-CH3/polypeptide 2: VH-CH3(Hole)] was assayed. The antigen binding site was specific for TNFα. The BLI response from binding molecule immobilization and TNFα binding to the immobilized construct demonstrates robust, specific, bivalent binding to the antigen. The data are consistent with a molecule having a high percentage of intended pairing of polypeptide 1 and polypeptide 2.

FIG. 6 illustrates features of an exemplary bivalent 1×1 bispecific binding molecule, “BC1”.

FIG. 7A shows size exclusion chromatography (SEC) analysis of “BC1”, demonstrating that a single-step CH1 affinity purification step (CaptureSelect™ CH1 affinity resin) yields a single, monodisperse peak via gel filtration in which >98% is unaggregated bivalent protein. FIG. 7B shows comparative literature data of SEC analysis of a CrossMab bivalent antibody construct [data from Schaefer et al. (Proc Natl Acad Sci USA. 2011 Jul. 5; 108(27):11187-92)].

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification.

FIG. 9 shows nonreducing SDS-PAGE gels of “BC1” at various stages of purification.

FIGS. 10A and 10B compare SDS-PAGE gels of “BC1” after single-step CH1-affinity purification under both non-reducing and reducing conditions (FIG. 10A) with SDS-PAGE gels of a CrossMab bispecific antibody under non-reducing and reducing conditions as published in the referenced literature (FIG. 10B).

FIGS. 11A and 11B show mass spec analysis of “BC1”, demonstrating two distinct heavy chains (FIG. 11A) and two distinct light chains (FIG. 11B) under reducing conditions.

FIG. 12 presents a mass spectrometry analysis of purified “BC1” under non-reducing conditions, confirming the absence of incomplete pairing after purification.

FIG. 13 presents accelerated stability testing data demonstrating stability of “BC1” over 8 weeks at 40° C., compared to two IgG control antibodies.

FIG. 14 illustrates features of an exemplary bivalent 1×1 bispecific binding molecule, “BC6”, further described in Example 3.

FIG. 15A presents size exclusion chromatography (SEC) analysis of “BC6” following one-step purification using the CaptureSelect™ CH1 affinity resin, demonstrating that the single step CH1 affinity purification yields a single monodisperse peak and the absence of non-covalent aggregates. FIG. 15B shows a SDS-PAGE gel of “BC6” under non-reducing conditions.

FIG. 16 illustrates features of an exemplary bivalent bispecific binding molecule, “BC28”, further described in Example 4.

FIG. 17 shows SDS-PAGE analysis under non-reducing conditions following single-step CH1 affinity purification of “BC28”, “BC29”, “BC30”, “BC31”, and “BC32”.

FIG. 18 shows SEC analysis of “BC28” and “BC30”, each following one-step purification using the CaptureSelect™ CH1 affinity resin.

FIG. 19 illustrates features of an exemplary bivalent bispecific binding molecule, “BC44”, further described in Example 5.

FIGS. 20A and 20B show size exclusion chromatography data of two bivalent binding molecules, “BC15” and “BC16”, respectively, under accelerated stability testing conditions. “BC15” and “BC16” have different variable region-CH3 junctions.

FIG. 21 presents a schematic of polypeptide chains and their domains, with respective naming conventions, for the trivalent 2×1 antibody constructs described herein.

FIG. 22 illustrates features of an exemplary trivalent 2×1 bispecific binding molecule, “BC1-2×1”, further described in Example 7.

FIG. 23 shows non-reducing SDS-PAGE of “BC1” and “BC1-2×1” protein expressed using the ThermoFisher Expi293 transient transfection system, at various stages of purification.

FIG. 24 compares the avidity of the bivalent 1×1 construct “BC1” to the avidity of the trivalent 2×1 construct “BC1-2×1” using an Octet (Pall ForteBio) biolayer interferometry analysis.

FIG. 25 illustrates salient features of a trivalent 2×1 construct, “TB111.”

FIG. 26 presents a schematic of polypeptide chains and their domains, with respective naming conventions, for the trivalent 1×2 antibody constructs described herein.

FIG. 27 illustrates features of an exemplary trivalent 1×2 construct “CTLA4-4 x Nivo x CTLA4-4”, further described in Example 10.

FIG. 28 is a SDS-PAGE gel in which the lanes showing the trivalent 1×2 construct “CTLA4-4 x Nivo x CTLA4-4” construct under non-reducing (“−DTT”) and reducing (“+DTT”) conditions have been boxed.

FIG. 29 shows a comparison of antigen binding between two antibodies: bivalent 1×1 construct “CTLA4-4x OX40-8” and the trivalent 1×2 construct “CTLA4-4 x Nivo x CTLA4-4.” “CTLA4-4x OX40-8” binds to CTLA4 monovalently, while “CTLA4-4 x Nivo x CTLA4-4” binds to CTLA4 bivalently.

FIG. 30 illustrates features of an exemplary trivalent 1×2 trispecific construct, “BC28-1×1×1a”, further described in Example 11.

FIG. 31 shows size exclusion chromatography of “BC28-1×1×1a” following transient expression and single step CH1 affinity resin purification, demonstrating a single well-defined peak.

FIG. 32 shows SDS-PAGE results with bivalent and trivalent constructs, each after transient expression and one-step purification using the CaptureSelect™ CH1 affinity resin, under non-reducing and reducing conditions, as further described in Example 12.

FIGS. 33A-33C show Octet binding analyses to 3 antigens: PD1, Antigen “A”, and CTLA4. As further described in Example 13, FIG. 33A shows binding of “BC1” to PD1 and Antigen “A”; FIG. 33B shows binding of a bivalent bispecific construct “CTLA4-4x OX40-8” to CTLA4, Antigen “A”, and PD1; FIG. 33C shows binding of trivalent trispecific “BC28-1×1×1a” to PD1, Antigen “A”, and CTLA4.

FIG. 34 presents a schematic of polypeptide chains and their domains, with respective naming conventions, for certain tetravalent 2×2 constructs described herein.

FIG. 35 illustrates certain salient features of the exemplary tetravalent 2×2 construct, “BC22-2×2” further described in Example 14.

FIG. 36 is a non-reducing SDS-PAGE gel comparing the 2×2 tetravalent “BC22-2×2” construct to a 1×2 trivalent construct “BC12-1×2” and a 2×1 trivalent construct “BC21-2×1” at different stages of purification.

FIG. 37 provides architecture for an exemplary tetravalent 2×2 construct.

FIG. 38 presents a schematic of polypeptide chains and their domains, with respective naming conventions, for certain tetravalent constructs described herein.

FIG. 39 provides exemplary architecture of a bispecific tetravalent construct.

FIG. 40 provides exemplary architecture for a trispecific tetravalent construct utilizing a common light chain strategy.

FIG. 41 shows bispecific antigen engagement by the tetravalent construct schematized in FIG. 39, demonstrating that this construct was capable of simultaneous engagement. The biolayer interferometry (BLI) response from B-Body immobilization and TNFα binding to the immobilized construct are consistent with a molecule with a high percentage of intended chain pairing.

FIG. 42 provides flow cytometry analysis of B-Body binding to cell-surface antigen. Cross-hatched signal indicates cells without antigen; dotted signal indicates transiently transfected cells with surface antigen.

FIG. 43 provides exemplary architecture of a trivalent construct.

FIG. 44 provides exemplary architecture of a trivalent construct.

FIG. 45 shows SDS-PAGE results with bivalent and trivalent constructs, each after transient expression and one-step purification using the CaptureSelect™ CH1 affinity resin, under non-reducing and reducing conditions, as further described in Example 17.

FIG. 46 shows differences in the thermal transitions for “BC24jv”, “BC26jv”, and “BC28jv” measured to assess pairing stability of junctional variants.

FIG. 47 shows SDS-PAGE analysis of bispecific antibodies comprising standard knob-hole orthogonal mutations introduced into CH3 domains found in their native positions within the Fc portion of the bispecific antibody that have been purified using a single-step CH1 affinity purification step (CaptureSelect™ CH1 affinity resin).

FIG. 48 depicts a three-dimensional model of a human IgA CH3 dimer. The white spheres denote residues that differ from a CH3 domain from human IgG.

FIG. 49 depicts an exemplary structure of a trivalent binding molecule.

FIG. 50 depicts an exemplary structure of a binding molecule comprising one or more CH1/CL orthogonal modifications.

FIG. 51 depicts an exemplary structure of a binding molecule comprising one or more CH1/CL orthogonal modifications.

FIG. 52 depicts an exemplary structure of a binding molecule comprising one or more CH1/CL orthogonal modifications.

FIG. 53 depicts an exemplary structure of a binding molecule comprising one or more CH1/CL orthogonal modifications.

FIG. 54 depicts an exemplary structure of a binding molecule comprising one or more CH1/CL orthogonal modifications.

FIG. 55 depicts an exemplary structure of a binding molecule comprising one or more CH1/CL orthogonal modifications.

FIG. 56 depicts an exemplary structure of a binding molecule comprising one or more CH1/CL orthogonal modifications.

FIG. 57 depicts an exemplary structure of a binding molecule comprising one or more CH1/CL orthogonal modifications.

FIG. 58 depicts an exemplary structure of a binding molecule comprising one or more CH1/CL orthogonal modifications.

FIG. 59 depicts an exemplary structure of a binding molecule comprising one or more CH1/CL orthogonal modifications.

FIG. 60 depicts Octet analysis of an exemplary binding molecule comprising a modification that reduces effector function.

FIG. 61 depicts results from an ADCC assay of various exemplary binding molecules comprising modifications that reduce effector function.

FIG. 62 depicts results from a Clq binding assay of various exemplary binding molecules comprising modifications that reduce effector function.

FIG. 63 depicts a schematic of the architecture of binding molecule MR-15.

FIG. 64 depicts SDS-PAGE analysis of binding molecule MR-15.

FIG. 65 depicts mass spectrogram results from an analysis of MR-15.

FIG. 66 depicts SDS-PAGE analysis of Variant 5.

FIG. 67 depicts SDS-PAGE analysis of various configurations of Variant 6.

FIG. 68 depicts results from an Octet assay assessing binding properties of a bispecific binding molecule comprising an IgA-CH3 domain swap.

FIG. 69 depicts SDS-PAGE analysis of BC1 and bispecific binding molecules comprising an IgA-CH3 domain swap and various CH3 linker sequences. Figure discloses “AGKC,” “PGKC,” “AGKGC,” and “AGKGSC” as SEQ ID NOS 96-99, respectively.

FIG. 70 depicts an exemplary model of a multispecific Treg binding molecule which preferentially binds to a target Treg expressing two particular antigens as compared to non-target cells.

FIG. 71 depicts results from candidate multispecific Treg SNIPER molecule discovery.

FIG. 72 depicts more results from candidate multispecific Treg SNIPER molecule discovery.

FIG. 73 depicts results from size exclusion chromatography of parent monoclonal antibodies CTLA4-19 and CD25-8.

FIG. 74 depicts results of a binding assay for bispecific SNIPER candidate 19×8.

FIG. 75 depicts results from a flow cytometry experiment assessing binding of SNIPER candidate 19×8 to various cell types in tumor and PBMC samples.

FIG. 76 depicts results from a flow cytometry experiment assessing binding of SNIPER candidate 19×8 to Cd8+ cells in tumor and PBMC samples.

FIG. 77 depicts results from a BLI experiment assessing binding kinetics of SNIPER candidate 19×8 to CTLA4.

FIG. 78 depicts results from a BLI experiment assessing binding kinetics of SNIPER candidate 19×8 to CD25.

FIG. 79 depicts results from a BLI experiment assessing binding kinetics of variants to remove potential aspartate isomerization sites.

FIG. 80 depicts further results from a BLI experiment assessing binding kinetics of variants to remove potential aspartate isomerization sites.

FIG. 81 depicts results of a binding assay for bispecific SNIPER-67 and SNIPER-95 variants.

FIG. 82 depicts flow cytometry results from an OX40/CD25 staining experiment.

FIG. 83 depicts flow cytometry results from a CTLA4/CD25 staining experiment.

FIG. 84 depicts steady state analysis of BLI sensorgram data for SNIPER 67.

FIG. 85 depicts steady state analysis of BLI sensorgram data for SNIPER 95.

FIG. 86 depicts presents a schematic of polypeptide chains and their domains, with respective naming conventions, described herein.

FIG. 87 shows SDS-PAGE analysis of the BA variants in Table 25.

FIG. 88 depicts architectures of the various trivalent molecules (“T26,” “T27,” “T28,” “T33,” “T34,” “T35,” “T36”, “T37,” and “T38”). Figure discloses “AGKGSC” as SEQ ID NO: 99.

FIGS. 89A and 89B shows SDS-PAGE results of Example 39. FIG. 89A shows SDS-PAGE results with the trivalent molecules T26, T27, T28, T33, T34, T35, and T37. FIG. 89B shows SDS-PAGE results with the trivalent molecules T27, T28, T33, T34, T35, and T36.

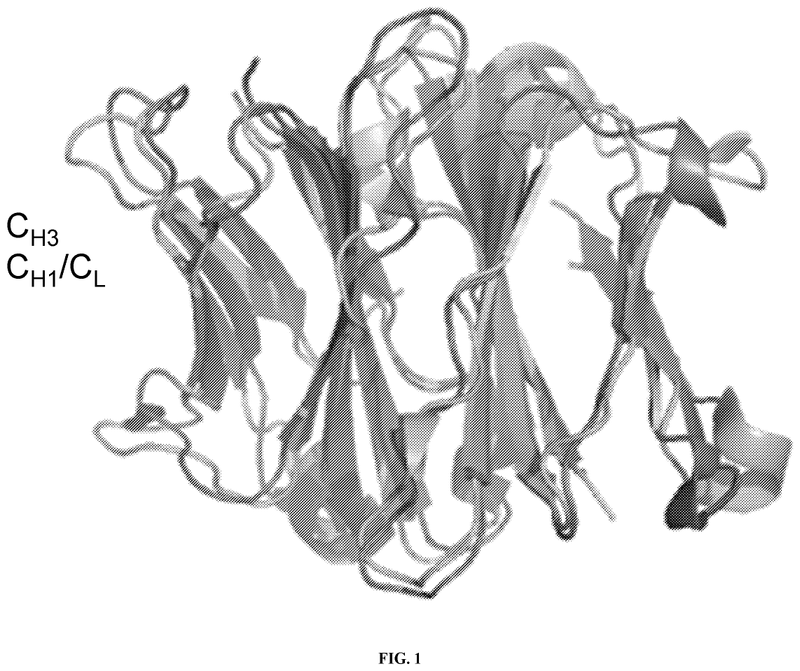

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

By “antigen binding site” or “ABS” is meant a region of a binding molecule that specifically recognizes or binds to a given antigen or epitope.

An ABS, and the binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules and stronger affinity. Likewise, a higher K_{D} value refers to a weaker affinity. K_{D} values of antibody constructs may be measured by methods known in the art including, but not limited to, bio-layer interferometry (e.g. Octet/FORTEBIO®), surface plasmon resonance (SPR) technology (e.g. Biacore), and cell binding assays. For purposes herein, affinities are dissociation equilibrium constants measured by bio-layer interferometry using Octet/FORTEBIO®.

“Specific binding,” or “selective binding,” as used interchangeably herein, generally refers to an affinity between an ABS and its cognate antigen or epitope in which the K_{D} value is below 10⁻⁶ M, 10⁻⁷ M, 10⁻⁸ M, 10⁻⁹ M, or 10⁻¹⁰ M. In some embodiments, an ABS that specifically binds a particular antigen binds to that antigen with stronger affinity than to another antigen.

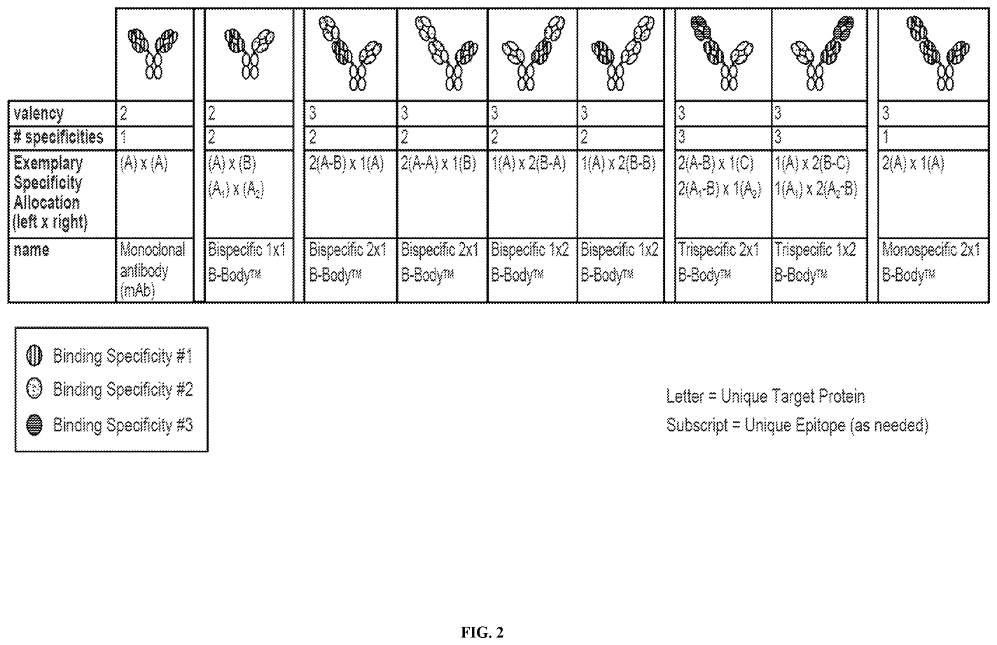

The number of ABSs in a binding molecule as described herein defines the “valency” of the binding molecule, as schematized in FIG. 2. A binding molecule having a single ABS is “monovalent”. A binding molecule having a plurality of ABSs is said to be “multivalent”. A multivalent binding molecule having two ABSs is “bivalent.” A multivalent binding molecule having three ABSs is “trivalent.” A multivalent binding molecule having four ABSs is “tetravalent.”

In various multivalent embodiments, all of the plurality of ABSs have the same recognition specificity. As schematized in FIG. 2, such a binding molecule is a “monospecific” “multivalent” binding construct. In other multivalent embodiments, at least two of the plurality of ABSs have different recognition specificities. Such binding molecules are multivalent and “multispecific”. In multivalent embodiments in which the ABSs collectively have two recognition specificities, the binding molecule is “bispecific.” In multivalent embodiments in which the ABSs collectively have three recognition specificities, the binding molecule is “trispecific.”

In multivalent embodiments in which the ABSs collectively have a plurality of recognition specificities for different epitopes present on the same antigen, the binding molecule is “multiparatopic.” Multivalent embodiments in which the ABSs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g. a multivalent binding molecule for a specific target, wherein the avidity is the cumulative or synergistic strength of interaction provided by the affinities of multiple ABSs. Avidity can be measured by the same methods as those used to determine affinity, as described above. In certain embodiments, the avidity of a binding molecule for a specific target, e.g., a target cell, is such that the interaction is a specific binding interaction, wherein the avidity between two molecules has a K_{D} value below 10⁻⁶M, 10⁻⁷M, 10⁻⁸ M, 10⁻⁹M, or 10⁻¹⁰ M. In certain embodiments, the avidity of a binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

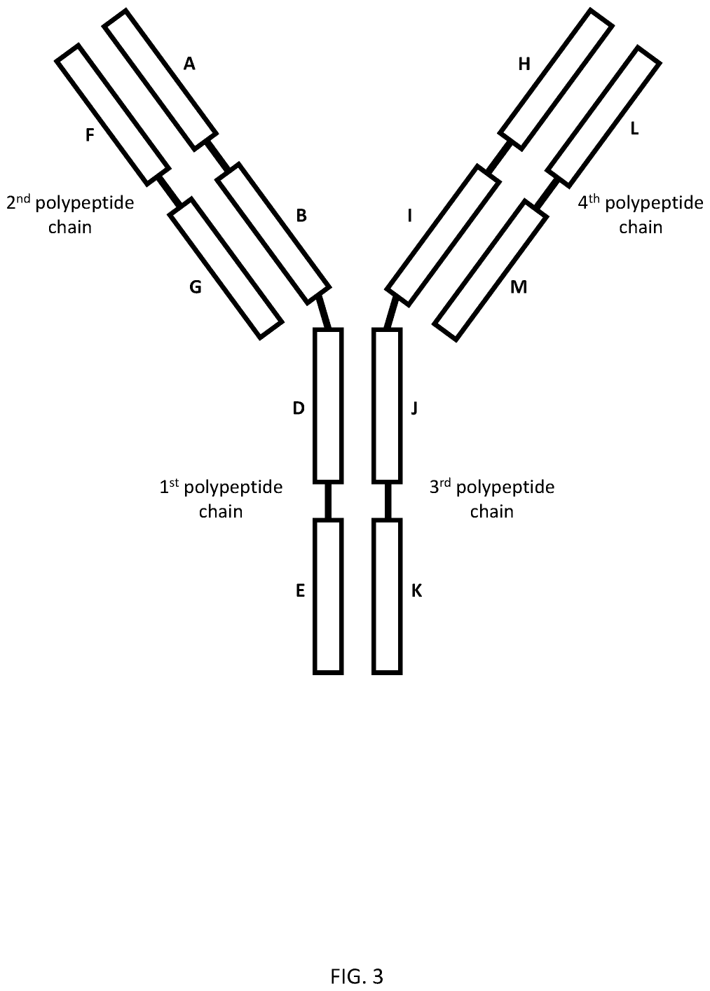

“B-Body,” as used herein and with reference to FIG. 3, refers to binding molecules comprising the features of a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the binding molecule. B-bodies are described in more detail in International Patent Application No. PCT/US2017/057268, herein incorporated by reference in its entirety.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, arthritis, or cancer. Beneficial or desired clinical results include, but are not limited to, reduction or alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

### 6.2. Other Interpretational Conventions

Unless otherwise specified, all references to sequences herein are to amino acid sequences.

Unless otherwise specified, antibody constant region residue numbering is according to the Eu index as described at www.imgt.org/IMGTScientificChart/Numbering/Hu_IGHGnber.html#refs (accessed Aug. 22, 2017) and in Edelman et al., Proc. Natl. Acad. USA, 63:78-85 (1969), which are hereby incorporated by reference in their entireties, and identifies the residue according to its location in an endogenous constant region sequence regardless of the residue's physical location within a chain of the binding molecules described herein. By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or apparent from context, as used herein the term “or” is understood to be inclusive. Unless specifically stated or apparent from context, as used herein, the terms “a”, “an”, and “the” are understood to be singular or plural. As used herein, the singular forms “a,” “an,” and “the” include the plural referents unless the context clearly indicates otherwise. The terms “include,” “such as,” and the like are intended to convey inclusion without limitation, unless otherwise specifically indicated

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### 6.3. Multispecific Treg Binding Molecules

Disclosed herein are multispecific Treg binding molecules that selectively bind to target Tregs. In some embodiments, the multispecific Treg-binding molecules bind to target Tregs with greater avidity than to non-target cells. Preferably, the multispecific Treg-binding molecules selectively bind target Tregs over non-target cells, including, e.g., peripheral non-target Tregs, CD8+ cells, CD4+ effector T cells, or other cells.

In some embodiments, the multispecific Treg-binding molecule comprises a first antigen binding site (ABS) specific for a first Treg cell surface antigen; and a second antigen binding site (ABS) specific for a second Treg cell surface antigen. In some embodiments, the first Treg cell surface antigen is not the second Treg cell surface antigen. In some embodiments, the first ABS exhibits a low binding affinity for the first Treg cell surface antigen. In some embodiments, the second ABS exhibits a low binding affinity for the second Treg cell surface antigen. In some embodiments, both the first and second ABSs exhibit low binding affinity for the first and second Treg cell surface antigens, respectively. Low binding affinity can refer to a Kd that is higher than 10 nM, higher than 20 nM, preferably higher than 50 nM, more preferably higher than 100 nM, yet more preferably higher than 200 nM.

In some embodiments, the first ABS specifically binds the first Treg cell surface antigen with a Kd that is higher than 10 nM, higher than 20 nM, preferably higher than 50 nM, more preferably higher than 100 nM, yet more preferably higher than 200 nM. In some embodiments, the first ABS specifically binds the first Treg cell surface antigen with a Kd that is between about 10-1000 nM, preferably between about 50-900 nM, more preferably between about 100-800 nM, or yet even more preferably between about 200-500 nM.

In some embodiments, the second ABS specifically binds the second Treg cell surface antigen with a Kd that is higher than 10 nM, higher than 20 nM, preferably higher than 50 nM, more preferably higher than 100 nM, yet more preferably higher than 200 nM. In some embodiments, the second ABS specifically binds the second Treg cell surface antigen with a Kd that is between about 10-1000 nM, preferably between about 50-900 nM, more preferably between about 100-800 nM, or yet even more preferably between about 200-500 nM.

In some embodiments, the first and second ABS's do not exhibit appreciable binding affinity for any other antigen. In some embodiments, the first and second ABS's exhibit a Kd to a non-target antigen that is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more than 500× higher than their Kd for the first or second Treg cell surface antigens, respectively.

The multispecific Treg-binding molecule may specifically bind to a target Treg, preferably a tumor-associated Treg, with a higher avidity than a non-target cell. In some embodiments, the multispecific Treg-binding molecule binds to the target Treg with a Kd that is less than about 500 nM, less than about 400 nM, less than about 300 nM, less than about 200 nM, less than about 100 nM, less than about 90 nM, less than about 80 nM, less than about 70 nM, less than about 60 nM, preferably less than about 50 nM, more preferably less than about 25 nM, or even more preferably less than about 10 nM. For example, the multispecific Treg-binding molecule may specifically bind to a target Treg with a Kd that is less than about 9 nM, less than about 8 nM, less than about 7 nM, less than about 6 nM, less than about 5 nM, less than about 4 nM, less than about 3 nM, less than about 2 nM, or less than about 1 nM.

The multispecific Treg-binding molecule may specifically bind to a target Treg, preferably a tumor-associated Treg with a higher avidity than the individual binding affinities of its ABS's for the first and second Treg cell surface antigens. For example, the multispecific Treg-binding molecule may exhibit a Kd for the first or second Treg cell surface marker that is higher than the binding molecule's Kd for the Treg, preferably the tumor-associated Treg. In some embodiments, the multispecific Treg-binding molecule exhibits a Kd for the first and second Treg cell surface antigens that is at least 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or more than 100× higher than the Kd of the binding molecule to the target Treg, preferably a tumor-associated Treg.

In some embodiments, the multispecific Treg-binding molecule specifically binds to a target Treg with a greater avidity than to any other non-target cell. For example, the multispecific Treg-binding molecule may bind to a target Treg with an avidity that is at least 0.1×, 0.2×, 0.3×, 0.4×, 0.5×, 0.6×, 0.7×, 0.8×, 0.9×, lx, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 750×, or 1000× greater than its avidity for a non-target cell. Preferably, the avidity of the multispecific Treg-binding molecule for a target Treg is 10× higher or greater than its avidity for a non-target cell.

The multispecific Treg-binding molecule may selectively bind to a target Treg over non-target cells. A skilled artisan may assess selective binding to the target Treg over non-target cells using any methods known in the art. An exemplary method for assessing selective binding may comprise comparing a percentage of target Tregs which are detectably labeled with the multispecific Treg-binding molecule under non-saturating assay conditions to a percentage of non-target cells which are detectably labeled with the multispecific Treg-binding molecule under the same assay conditions. For example, a ratio of the percent target Tregs bound/percent non-target cells bound by the multispecific Treg binding molecule may be used as an indication of selective binding to the target Treg. In some embodiments, a multispecific Treg binding molecule that detectably binds over 70% of target Tregs under non-saturating assay conditions binds less than 30%, less than 25%, less than 20%, or less than 15% of non-target cells under the same assay conditions. In some embodiments, a multispecific Treg binding molecule that detectably binds over 80% of target Tregs under non-saturating assay conditions binds less than 20% of non-target cells under the same assay conditions. In some embodiments, a multispecific Treg binding molecule that detectably binds over 90% of target Tregs under non-saturating assay conditions binds less than 10% of non-target cells under the same assay conditions. In some embodiments, the ratio of bound target Tregs/bound non-target cells under non-saturating assay conditions is greater than 1.5, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 110, greater than 120, greater than 130, greater than 140, greater than 150, greater than 160, greater than 170, greater than 180, greater than 190, greater than 200, greater than 210, greater than 220, greater than 230, greater than 240, greater than 250, greater than 260, greater than 270, greater than 280, greater than 290, greater than 300, greater than 310, greater than 320, greater than 330, greater than 340, greater than 350, greater than 360, greater than 370, greater than 380, greater than 390, greater than 400, greater than 410, greater than 420, greater than 430, greater than 440, greater than 450, greater than 460, greater than 470, greater than 480, greater than 490, or greater than 500.

### 6.4. Target Tregs and Non-Target Cells

Tregs generally, including target and non-target Tregs, can be distinguished from other non-target cell types, including other non-target immune cells such as T effector cells, T helper cells, and T-killer cells, based on expression of one or more markers or combinations of markers. For example, target and non-target Tregs may be distinguished from other cell types based on coexpression of CD4 and CD25. Accordingly, target and non-target Tregs may be distinguished from other cell types by virtue of being CD4+/CD25+. In some cases, target and non-target Tregs may further be distinguished from other cell types based on low or undetectable expression levels of CD127. For example, target and non-target Tregs may be distinguished from other cell types by coexpression of CD4 and CD25, and low or undetectable expression of CD127. In one embodiment, target and non-target Tregs are distinguished from other cell types by virtue of being CD4+/CD25^{hi}/CD127^{lo}. In some cases, target and non-target Tregs may be distinguished from other cell types based on expression of FoxP3. For example, target and non-target Tregs may be distinguished from other cell types by virtue of expressing FoxP3 and exhibiting low or undetectable levels of CD127. In one embodiment, target and non-target Tregs are distinguished from other cell types by virtue of being FoxP3+/CD127^{lo}. Methods of distinguishing Tregs, including target and non-target Tregs, from other immune cell types are described in, e.g., D'Arena G, Vitale C, Coscia M, et al. Regulatory T Cells and Their Prognostic Relevance in Hematologic Malignancies. Journal of Immunology Research. 2017; 2017:1832968. doi:10.1155/2017/1832968; J Exp Med. 2006 Jul. 10; 203(7):1701-11.

Non-target cells can include immune cells other than Tregs, such as, e.g., non-target lymphocytes, effector T cells, T killer cells, memory T cells, neutrophils, macrophages, eosinophils, dendritic cells, B cells. Methods of distinguishing other cell types from Tregs are described herein.

By way of example only, non-target lymphocytes may be distinguished from Tregs, including target and non-target Tregs, by virtue of being CD45+, but not exhibiting the expression profile of Tregs (e.g., not being CD4+/CD25^{hi}/CD127^{lo}, or not being)FoxP3+/CD127^{lo}.

By way of example only, T-cells generally express CD3. Accordingly, non-target T-cells may be distinguished from Tregs, including target and non-target Tregs, by virtue of being CD3+ but not exhibiting the expression profile of Tregs (e.g., not being CD4+/CD25^{hi}/CD127^{lo}, or not being)FoxP3+/CD127^{lo}.

T effector cells can include T helper, T killer, regulatory T cells (Tregs), and potentially other T cell types.

T helper cells and target and non-target Tregs generally express CD4.

Accordingly, non-target T helper cells may be distinguished from Tregs, including target and non-target Tregs, by virtue of being CD4+ but not exhibiting the expression profile of Tregs (e.g., not being CD4+/CD25/CD127^{lo}, or not being)FoxP3+/CD127^{lo}. Other methods of distinguishing T helper cells, including subsets of T helper cells, are described in, e.g., Blood. 2008 Sep. 1; 112(5):1557-69; Curr Opin Immunol. 2012 June; 24(3):297-302.

T killer cells generally express CD8. Accordingly, T killer cells may be distinguished from Tregs, including target and non-target Tregs, by virtue of being CD8+ but not exhibiting the expression profile of Tregs (e.g., not being CD4+/CD25+/CD127^{lo}, or not being)FoxP3+/CD127^{lo}.

Memory T cells generally are either CD4+ or CD8+ T cells and also express CD45RO. Accordingly, memory T cells may be distinguished from other cell types, including Tregs, by virtue of being CD4+/CD45RO+ or CD8+/CD45RO+. See, e.g., J Immunol. 1988 Apr. 1; 140(7):2171-8. Other methods of distinguishing memory T cells, including subsets of memory T cells, are described in J Immunol. 2005 Nov. 1; 175(9):5895-903; Immun Ageing. 2008 Jul. 25; 5:6. doi: 10.1186/1742-4933-5-6; Immunol Rev. 2013 September; 255(1):165-81. doi: 10.1111/imr.12087; Trends Immunol. 2011 February; 32(2):50-6; J Autoimmun. 2017 February; 77:76-88; and J Exp Med. 2007 Jul. 9; 204(7):1625-36.

In some embodiments, a target Treg is distinguished from non-target Tregs or other non-target cells based on coexpression of the first and second Treg cell surface antigens. Exemplary first and second Treg cell surface antigens are described herein. In some embodiments, the target Treg expresses one or both of the first and second Treg cell surface antigens at a higher level than non-target Tregs or other non-target cells. For example, the target Treg may express a level of such cell surface antigens that is at least 0.1×, 0.5×, lx, 1.5×, preferably at least 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or more than 100× higher than a level expressed by a non-target Treg or other non-target cell. In some embodiments, the target Treg expresses the first and second cell surface antigens at a level that is at least 1× higher than the expression level of the first and second cell surface antigens in a non-target cell. Expression levels of the first and second Treg cell surface antigens can be determined using techniques known to those of skill in the art, such as, e.g., immunologic detection, mRNA detection, and the like. In some embodiments, non-target Tregs do not coexpress both the first and second Treg cell surface antigens. In some embodiments, other non-target cells do not co-express both the first and second Treg cell surface antigens. In some embodiments, non-target cells, such as non-target Tregs express both first and second Treg cell surface antigens at a lower level, e.g., less than 50% of a level of the first and second Treg cell surface antigens as compared to a target Treg.

In some embodiments, the target Treg is a tumor-associated Treg. Tumor-associated Tregs can be, e.g., tumor-infiltrating Tregs. Tumor-infiltrating Tregs are generally localized to a tumor, e.g., in the tumor microenvironment. Accordingly, tumor-infiltrating Tregs can be obtained from a tumor sample. In some embodiments, tumor-associated Tregs exhibit an expression profile as described in De Simone et al (2016), Immunity Vol. 45, pp. 1135-1147.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of tumor-infiltrating Tregs. Preferably, the multispecific Treg-binding molecule binds at least about 60% of tumor-infiltrating Tregs. Yet more preferably, the multispecific Treg-binding molecule binds at least about 70% of tumor-infiltrating Tregs. In one embodiment, the multispecific Treg-binding molecule binds at least about 90% of tumor-infiltrating Tregs.

Tumor-associated Tregs may also be found in peripheral blood, yet exhibit an expression profile similar to that of a tumor-infiltrating Treg. For example, the peripheral, tumor-associated Treg may exhibit an expression profile as described in De Simone et al (2016), Immunity Vol. 45, pp. 1135-1147.

The tumor-associated Treg may express at least one, two, three, four, more than four, or all of the following cell surface antigens: CTLA4, CD25, OX40, GITR, TNFRII, NRP1, TIGIT, CCR8, LAYN, MAGEH1, CD27, ICOS, LAG-3, TIM-3, CD30, IL-1R2, IL-21R, 4-1BB, PDL-1, and PDL-2. In some embodiments, the tumor-associated Treg expresses at least two of CTLA4, CD25, OX40, GITR, TNFRII, NRP1, CD30, CD27, ICOS, TIGIT, 4-1BB, LAG-3, and PDL-2. In particular embodiments, the tumor-associated Treg expresses at least two of CTLA4, CD25, OX40, and NRP1. For example, the tumor-associated Treg may express CTLA4 and CD25, CTLA4 and NRP1, CTLA4 and OX40, OX40 and CD25, OX40 and NRP1, CD25 and NRP1.

In some embodiments, the tumor-associated Treg overexpresses at least one, two, three, four, more than four, or all of the following cell surface antigens as compared to a non-target cell: CTLA4, CD25, OX40, GITR, TNFRII, NRP1, TIGIT, CCR8, LAYN, MAGEH1, CD27, ICOS, LAG-3, TIM-3, CD30, IL-1R2, IL-21R, 4-1BB, PDL-1, and PDL-2. Non-target cells are described herein. In preferred embodiments, the tumor-associated Treg expresses any one of CTLA4, CD25, OX40, GITR, TNFRII, NRP1, TIGIT, CCR8, LAYN, MAGEH1, CD27, ICOS, LAG-3, TIM-3, CD30, IL-1R2, IL-21R, 4-1BB, PDL-1, and PDL-2 at a level that is at least 1× higher than the expression level of the gene or protein in a non-target cell.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of Tregs which coexpress CTLA4 and CD25. In preferred embodiments, the multispecific Treg-binding molecule binds at least about 80% of Tregs which coexpress CTLA4 and CD25. In more preferred embodiments, the multispecific Treg-binding molecule binds at least about 90% of Tregs which coexpress CTLA4 and CD25. In one embodiment, the multispecific Treg-binding molecule binds at least about 95% of Tregs which coexpress CTLA4 and CD25. In preferred embodiments, the multispecific Treg-binding molecule binds at least about 80% of Tregs which coexpress CTLA4 and CD25 and binds not more than 20% of Tregs which do not detectably coexpress CTLA4 and CD25.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of Tregs which coexpress OX40 and CD25. In preferred embodiments, the multispecific Treg-binding molecule binds at least about 80% of Tregs which coexpress OX40 and CD25. In more preferred embodiments, the multispecific Treg-binding molecule binds at least about 90% of Tregs which coexpress OX40 and CD25. In one embodiment, the multispecific Treg-binding molecule binds at least about 95% of Tregs which coexpress OX40 and CD25. In preferred embodiments, the multispecific Treg-binding molecule binds at least about 80% of Tregs which coexpress OX40 and CD25 and binds not more than 20% of Tregs which do not detectably coexpress OX40 and CD25.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of Tregs which coexpress CTLA4 and NRP1. In preferred embodiments, the multispecific Treg-binding molecule binds at least about 80% of Tregs which coexpress CTLA4 and NRP1. In more preferred embodiments, the multispecific Treg-binding molecule binds at least about 90% of Tregs which coexpress CTLA4 and NRP1. In one embodiment, the multispecific Treg-binding molecule binds at least about 95% of Tregs which coexpress CTLA4 and NRP1. In preferred embodiments, the multispecific Treg-binding molecule binds at least about 80% of Tregs which coexpress CTLA4 and NRP1 and binds not more than 20% of Tregs which do not detectably coexpress CTLA4 and NRP1.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of Tregs which coexpress CTLA4 and OX40. In preferred embodiments, the multispecific Treg-binding molecule binds at least about 80% of Tregs which coexpress CTLA4 and OX40. In more preferred embodiments, the multispecific Treg-binding molecule binds at least about 90% of Tregs which coexpress CTLA4 and OX40. In one embodiment, the multispecific Treg-binding molecule binds at least about 95% of Tregs which coexpress CTLA4 and OX40. In preferred embodiments, the multispecific Treg-binding molecule binds at least about 80% of Tregs which coexpress CTLA4 and OX40 and binds not more than 20% of Tregs which do not detectably coexpress CTLA4 and OX40.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of Tregs which coexpress OX40 and NRP1. In preferred embodiments, the multispecific Treg-binding molecule binds at least about 80% of Tregs which coexpress OX40 and NRP1. In more preferred embodiments, the multispecific Treg-binding molecule binds at least about 90% of Tregs which coexpress OX40 and NRP1. In one embodiment, the multispecific Treg-binding molecule binds at least about 95% of Tregs which coexpress OX40 and NRP1. In preferred embodiments, the multispecific Treg-binding molecule binds at least about 80% of Tregs which coexpress OX40 and NRP1 and binds not more than 20% of Tregs which do not detectably coexpress OX40 and NRP1.

In some embodiments, the multispecific Treg-binding molecule binds at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of Tregs which coexpress CD25 and NRP1. In preferred embodiments, the multispecific Treg-binding molecule binds at least about 80% of Tregs which coexpress CD25 and NRP1. In more preferred embodiments, the multispecific Treg-binding molecule binds at least about 90% of Tregs which coexpress CD25 and NRP1. In one embodiment, the multispecific Treg-binding molecule binds at least about 95% of Tregs which coexpress CD25 and NRP1. In preferred embodiments, the multispecific Treg-binding molecule binds at least about 80% of Tregs which coexpress CD25 and NRP1 and binds not more than 20% of Tregs which do not detectably coexpress CD25 and NRP1.

Non-target cells can include peripheral, non-tumor-associated Tregs. Peripheral Tregs, which may circulate in a subject's blood system in vivo, can be obtained from peripheral blood samples. Non-target Tregs may be distinguished from other cell types using methods described herein. The non-target Treg may be a peripheral Treg that is CD4+/CD25+. In some cases, the non-target Treg is a peripheral Treg that expresses low or undetectable levels of CD127. For example, the non-target Treg may be a peripheral Treg which expresses CD4 and CD25, and which exhibits low or undetectable expression of CD127. In some cases, the non-target Treg is a peripheral Treg which expresses FoxP3. The non-target Treg may be a peripheral Treg which expresses FoxP3 and expresses low or undetectable levels of CD127. The non-target Treg may be a peripheral Treg which is CD4+/CD25+/CD127^{lo}. The non-target Treg may be a peripheral Treg which is FoxP3+/CD127^{lo}.

In preferred embodiments, the non-target Treg does not exhibit an expression profile similar to that of a tumor-infiltrating Treg. For example, the non-target cell may include a peripheral Treg that does not exhibit an expression profile as described in De Simone et al. (2016), Immunity Vol. 45, pp. 1135-1147, which is incorporated by reference.

In some cases, the non-target Treg exhibits reduced or no detectable expression of one, two, three, four, more than four, or any of the following genes, as compared to tumor-associated Tregs: CTLA4, CD25, OX40, GITR, TNFRII, NRP1, TIGIT, CCR8, LAYN, MAGEH1, CD27, ICOS, LAG-3, TIM-3, CD30, IL-1R2, IL-21R, 4-1BB, PDL-1, PDL-2, CD73, CD39. In preferred embodiments, the non-target Treg exhibits reduced expression of, or does not express detectable levels of two or more of CTLA4, CD25, OX40, and NRP1. For example, the non-target Treg expresses less than about 50%, less than about 49%, less than about 48%, less than about 47%, less than about 46%, less than about 45%, less than about 44%, less than about 43%, less than about 42%, less than about 41%, less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1% of CD25, OX40, and NRP1 as compared to a tumor-associated Treg.

For example, the multispecific Treg-binding molecule may bind to less than about 10%, less than about 9%, less than about 8%, less than about 7%, less than about 6%, less than about 5%, less than about 4%, less than about 3%, less than about 2%, or less than about 1% of Tregs that do not doubly express CTLA4 and CD25. In some embodiments, the multispecific Treg-binding molecule binds to less than about 10%, less than about 9%, less than about 8%, less than about 7%, less than about 6%, less than about 5%, less than about 4%, less than about 3%, less than about 2%, or less than about 1% of Tregs that do not doubly express OX40 and CD25. In some embodiments, the multispecific Treg-binding molecule binds to less than about 10%, less than about 9%, less than about 8%, less than about 7%, less than about 6%, less than about 5%, less than about 4%, less than about 3%, less than about 2%, or less than about 1% of Tregs that do not doubly express CTLA4 and NRP1. In some embodiments, the multispecific Treg-binding molecule binds to less than about 10%, less than about 9%, less than about 8%, less than about 7%, less than about 6%, less than about 5%, less than about 4%, less than about 3%, less than about 2%, or less than about 1% of Tregs that do not doubly express CTLA4 and OX40. In some embodiments, the multispecific Treg-binding molecule binds to less than about 10%, less than about 9%, less than about 8%, less than about 7%, less than about 6%, less than about 5%, less than about 4%, less than about 3%, less than about 2%, or less than about 1% of Tregs that do not doubly express OX40 and NRP1. In some embodiments, the multispecific Treg-binding molecule binds to less than about 10%, less than about 9%, less than about 8%, less than about 7%, less than about 6%, less than about 5%, less than about 4%, less than about 3%, less than about 2%, or less than about 1% of Tregs that do not doubly express CD25 and NRP1.

In some embodiments, the multispecific Treg-binding molecule binds less than about 50%, less than about 49%, less than about 48%, less than about 47%, less than about 46%, less than about 45%, less than about 44%, less than about 43%, less than about 42%, less than about 41%, less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% of peripheral Tregs. Preferably, the multispecific Treg-binding molecule binds less than about 20% of peripheral Tregs. Yet more preferably, the multispecific Treg-binding molecule binds less than about 15% of peripheral Tregs. In one embodiment, the multispecific Treg-binding molecule binds less than about 10% of peripheral Tregs.

The multispecific Treg-binding molecule may bind more than 50% of tumor-infiltrating Tregs and less than 50% of peripheral CD4+ T-cells. In preferred embodiments, the multispecific Treg-binding molecule binds more than 65% of tumor-infiltrating Tregs and less than 10% of peripheral CD4+ T-cells. In yet more preferred embodiments, the multispecific Treg-binding molecule binds more than 70% of tumor-infiltrating Tregs and less than 5% of peripheral CD4+ T-cells. In one embodiment, the multispecific Treg-binding molecule binds more than 70% of tumor-infiltrating Tregs and less than 2% of peripheral CD4+ T-cells.

The multispecific Treg-binding molecule may bind more than 50% of tumor-infiltrating Tregs and less than 50% of peripheral CD8+ T-cells. In preferred embodiments, the multispecific Treg-binding molecule binds more than 65% of tumor-infiltrating Tregs and less than 30% of peripheral CD8+ T-cells. In yet more preferred embodiments, the multispecific Treg-binding molecule binds more than 70% of tumor-infiltrating Tregs and less than 20% of peripheral CD8+ T-cells. In one embodiment, the multispecific Treg-binding molecule binds more than 70% of tumor-infiltrating Tregs and less than 10% of peripheral CD8+ T-cells.

The multispecific Treg binding molecules can target a majority of tumor-infiltrating Tregs while sparing a majority of other peripheral lymphocytes, thereby reducing a key mechanism of tumor immune tolerance while maintaining immune homeostasis and self-tolerance. For example, the multispecific Treg binding molecules can target a majority of tumor-infiltrating Tregs while sparing a majority of peripheral Tregs. The multispecific Treg-binding molecule may bind more than 50% of tumor-infiltrating Tregs and less than 50% of peripheral Tregs. In preferred embodiments, the multispecific Treg-binding molecule binds more than 65% of tumor-infiltrating Tregs and less than 25% of peripheral Tregs. In yet more preferred embodiments, the multispecific Treg-binding molecule binds more than 70% of tumor-infiltrating Tregs and less than 15% of peripheral Tregs. In one embodiment, the multispecific Treg-binding molecule binds more than 70% of tumor-infiltrating Tregs and less than 10% of peripheral Tregs.

The multispecific Treg binding molecules can target a majority of tumor-infiltrating Tregs while sparing a majority of other tumor-infiltrating lymphocytes, thereby reducing a key mechanism of tumor immune tolerance while maintaining immune attack on tumor cells. For example, the multispecific Treg binding molecules can target a majority of tumor-infiltrating Tregs while sparing a majority of CD4+ tumor-infiltrating T-cells. The multispecific Treg-binding molecule may bind more than 50% of tumor-infiltrating Tregs and less than 50% of total tumor-infiltrating CD4+ T-cells. In preferred embodiments, the multispecific Treg-binding molecule binds more than 65% of tumor-infiltrating Tregs and less than 30% of total tumor-infiltrating CD4+ T-cells. In yet more preferred embodiments, the multispecific Treg-binding molecule binds more than 70% of tumor-infiltrating Tregs and less than 20% of total tumor-infiltrating CD4+ T-cells. In one embodiment, the multispecific Treg-binding molecule binds more than 70% of tumor-infiltrating Tregs and less than 10% of total tumor-infiltrating CD4+ T-cells.

For other example, the multispecific Treg binding molecules can target a majority of tumor-infiltrating Tregs while sparing a majority of tumor-infiltrating CD8+ cells, thereby reducing a key mechanism of tumor immune tolerance while maintaining immune attack on tumor cells. The multispecific Treg-binding molecule may bind more than 50% of tumor-infiltrating Tregs and less than 50% of total tumor-infiltrating CD8+ T-cells. In preferred embodiments, the multispecific Treg-binding molecule binds more than 65% of tumor-infiltrating Tregs and less than 30% of total tumor-infiltrating CD8+ T-cells. In yet more preferred embodiments, the multispecific Treg-binding molecule binds more than 70% of tumor-infiltrating Tregs and less than 20% of total tumor-infiltrating CD8+ T-cells. In one embodiment, the multispecific Treg-binding molecule binds more than 70% of tumor-infiltrating Tregs and less than 10% of total tumor-infiltrating CD8+ T-cells.

### 6.4.1. Exemplary Treg Cell Surface Antigens

The first and second Treg cell surface antigens bound by the first and second ABSs may include at least one cell surface protein that is overexpressed in tumor-infiltrating Tregs as compared to other lymphocytes, such as peripheral Tregs. Such overexpressed proteins are described in, e.g., De Simone et al (2016) Immunity 45, 1135-1147. In some embodiments, the first and second Treg cell surface antigens are each independently selected from CTLA4, CD25, CD73, CD39, OX40, GITR, TNFRII, NRP1, TIGIT, CCR8, LAYN, MAGEH1, CD27, ICOS, LAG-3, TIM-3, CD30, IL-1R2, IL-21R, 4-1BB, CCR4, CXCR4, CCR5, PDL-1, and PDL-2. In specific embodiments, the first and second Treg cell surface antigens are each independently selected from CTLA4, CD25, OX40, GITR, TNFRII, NRP1, CD30, CD27, ICOS, TIGIT, 4-1BB, LAG-3, and PDL-2. In yet more specific embodiments, the first and second Treg cell surface antigens are each independently selected from CTLA4, CD25, OX40, GITR, TNFRII, and NRP1. In more specific embodiments, the first and second Treg cell surface antigens are each independently selected from CD25, CTLA4, NRP1, and OX40.

In preferred embodiments, the first and second Treg cell surface antigens are CD25 and OX40, CD25 and CTLA-4, CD25 and NRP1, OX40 and CTLA-4, OX40 and NRP1, or CTLA-4 and NRP1. In one embodiment, the first and second Treg cell surface antigens are CD25 and CTLA-4.

Other Antigens

The multispecific Treg binding molecule can further comprise one or more additional ABSs. The additional ABS may be chosen to specifically bind a wide variety of molecular targets. Preferably, the additional ABSs does not specifically bind to the first or second Treg cell surface antigen. For example, an additional ABS may specifically bind E-Cad, CLDN7, FGFR2b, N-Cad, Cad-11, FGFR2c, ERBB2, ERBB3, FGFR1, FOLR1, IGF-Ira, GLP1R, PDGFRa, PDGFRb, EPHB6, ABCG2, CXCR4, CXCR7, Integrin-avb3, SPARC, VCAM, ICAM, Annexin, TNFα, CD137, angiopoietin 2, angiopoietin 3, BAFF, beta amyloid, C5, CA-125, CD147, CD125, CD147, CD152, CD19, CD20, CD22, CD23, CD24, CD25, CD274, CD28, CD3, CD30, CD33, CD37, CD4, CD40, CD44, CD44v4, CD44v6, CD44v7, CD50, CD51, CD52, CEA, CSF1R, CTLA-2, DLL4, EGFR, EPCAM, HER3, GD2 ganglioside, GDF-8, Her2/neu, CD2221, IL-17A, IL-12, IL-23, IL-13, IL-6, IL-23, an integrin, CD11a, MUC1, Notch, TAG-72, TGFβ, TRAIL-R2, VEGF-A, VEGFR-1, VEGFR2, VEGFc, hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, Fas, CD27, CD30, and 4-1BBL); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)); in embodiments relating to bispecific antibodies, the antibody may for example bind two of these targets.

Furthermore, the Fc portion of the heavy chain of an antibody may be used to target Fc receptor-expressing cells such as the use of the Fc portion of an IgE antibody to target mast cells and basophils.

The additional ABS may specifically bind a TNF receptor. Exemplary TNF receptors include, but are not limited to, TNFR1 (also known as CD120a and TNFRSF1A), TNFR2 (also known as CD120b and TNFRSF1B), TNFRSF3 (also known as LTβR), TNFRSF4 (also known as OX40 and CD134), TNFRSF5 (also known as CD40), TNFRSF6 (also known as FAS and CD95), TNFRSF6B (also known as DCR3), TNFRSF7 (also known as CD27), TNFRSF8 (also known as CD30), TNFRSF9 (also known as 4-1BB), TNFRSF10A (also known as TRAILR1, DR4, and CD26), TNFRSF10B (also known as TRAILR2, DR5, and CD262), TNFRSF10C (also known as TRAILR3, DCR1, CD263), TNFRSF10D (also known as TRAILR4, DCR2, and CD264), TNFRSF11A (also known as RANK and CD265), TNFRSF11B (also known as OPG), TNFRSF12A (also known as FN14, TWEAKR, and CD266), TNFRSF13B (also known as TACI and CD267), TNFRSF13C (also known as BAFFR, BR3, and CD268), TNFRSF14 (also known as HVEM and CD270), TNFRSF16 (also known as NGFR, p75NTR, and CD271), or TNFRSF17 (also known as BCMA and CD269), TNFRSF18 (also known as GITR and CD357), TNFRSF19 (also known as TROY, TAJ, and TRADE), TNFRSF21 (also known as CD358), TNFRSF25 (also known as Apo-3, TRAMP, LARD, or WS-1), EDA2R (also known as XEDAR).

The additional ABS may specifically bind an immune-oncology target, e.g., a checkpoint inhibitor. Exemplary checkpoint inhibitors include, but are not limited to, checkpoint inhibitor targets such as PD1, PDL1, CTLA-4, PDL2, B7-H3, B7-H4, BTLA, TIM3, GALS, LAG3, VISTA, KIR, 2B4, BY55, and CGEN-15049.

In preferred embodiments, the additional ABS specifically binds a surface molecule expressed by another cell type. The other cell type may be a cytotoxic lymphocyte, such as, e.g., a natural killer (NK) cell or macrophage. Exemplary cell surface molecules expressed on NK cells include, e.g., CD16, NKG2A, NKp46, and CD56. Exemplary molecules expressed by macrophages include, e.g., CD47, CD14, CD40, CD11b, CD64, EMR1 (human), lysozyme M, MAC-1/MAC-3, and CD68.

In particular embodiments, the multispecific Treg binding molecule is a trivalent trispecific binding molecule comprising two different ABS's that specifically bind two different antigens associated with target Tregs, and an additional ABS that specifically binds a cell surface antigen on a cytotoxic immune cell, such as a natural killer cell.

In some embodiments, the one or more affinities of individual ABSs for the two antigens associated with target Tregs have a high K_{D} value that qualifies as weakly binding their respective antigens or epitopes on their own, but the avidity of the trivalent trispecific binding molecule for the target Treg has a K_{D} value such that the interaction is a specific binding interaction.

In a series of embodiments, an additional antigen binding site or sites may be chosen that specifically target tumor-associated cells.

Exemplary structural features of the multispecific Treg binding molecules

Further aspects of the multispecific Treg-binding molecules useful for the invention are provided.

With reference to FIG. 3, in a series of embodiments, the multispecific Treg-binding molecules comprise a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the multispecific Treg-binding molecule.

In a series of embodiments, (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein the third polypeptide chain comprises the CH1 domain and domain I is the CH1 domain, or portion thereof, domain H has a variable region domain amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and wherein domain M has a CL amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the multispecific Treg-binding molecule.

In some embodiments, the multispecific Treg-binding molecule comprises a native antibody architecture, wherein domains A and H comprise VH amino acid sequences, domains F and L comprise VL amino acid sequences, domains B and I comprise CH1, domains G and M comprise CL, domains D and J comprise CH2, and domains E and K comprise CH3.

In preferred embodiments, the multispecific Treg-binding molecule is a B-Body™. B-Body™ binding molecules are described in International Patent Application No. PCT/US2017/057268. In some embodiments, the multispecific Treg-binding molecule is structured as described in FIG. 3, wherein domains A and H comprise VL, domains B and G comprise CH3, domain I comprises CL or CH1, domain M comprises CH1 or CL, domains D and J comprise CH2, and domains E and K comprise CH3. In some embodiments, domain I comprises CL and domain M comprises CH1. In some embodiments, domain I is CH1 and domain M is CL.

In some embodiments, the multispecific Treg-binding molecule is a CrossMab™. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230, each of which is incorporated herein in its entirety. In some embodiments, the multispecific Treg-binding molecule is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other. In some embodiments, the multispecific Treg-binding molecule is structured as described in FIG. 3, wherein A is VH, B is CH1, D is CH2, E is CH3, F is VL, G is CL, H is VL or VH, I is CL, J is CH2, K is CH3, L is VH or VL, and M is CH1.

In some embodiments, the multispecific Treg-binding molecule is an antibody having a general architecture described in U.S. Pat. No. 8,871,912 and WO2016087650, each of which is incorporated herein in its entirety. In some embodiments, the multispecific Treg-binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair. In some embodiments, the multispecific Treg-binding molecule is structured as described in FIG. 3, wherein A is VH, B is CH3, D is CH2, E is CH3, F is VL, G is CH3, H is VH, I is CH1, J is CH2, K is CH3, L is VL, and M is CL.

In some embodiments, the multispecific Treg-binding molecule is as described in WO2017011342, which is incorporated herein in its entirety. In some embodiments, the multispecific Treg-binding molecule is structured as described in FIG. 3, wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL.

In some embodiments, the multispecific Treg-binding molecule is as described in WO2006093794, which is incorporated by reference. In some embodiments, the multispecific Treg-binding molecule is structured as described in FIG. 3, wherein A is VH, B is CH1, D is CH2, E is CH3, F is VL, G is CL, H is VL, I is CL or CH1, J is CH2, K is CH3, L is VH, and M is CH1 or CL.

Domain A (Variable Region)

In the multispecific Treg-binding molecules, domain A has a variable region domain amino acid sequence. Variable region domain amino acid sequences, as described herein, are variable region domain amino acid sequences of an antibody including VL and VH antibody domain sequences. VL and VH sequences are described in greater detail herein. In a preferred embodiment, domain A has a VL antibody domain sequence and domain F has a VH antibody domain sequence. In some embodiments, domain A has a VH antibody domain sequence and domain F has a VL antibody domain sequence.

### 6.4.1.1. VL Regions

The VL amino acid sequences useful in the multispecific Treg-binding molecules described herein are antibody light chain variable domain sequences. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail herein.

In various embodiments, VL amino acid sequences are mutated sequences of naturally occurring sequences. In certain embodiments, the VL amino acid sequences are lambda (λ) light chain variable domain sequences. In certain embodiments, the VL amino acid sequences are kappa (κ) light chain variable domain sequences. In a preferred embodiment, the VL amino acid sequences are kappa (κ) light chain variable domain sequences.

In the multispecific Treg-binding molecules described herein, the C-terminus of domain A is connected to the N-terminus of domain B. In certain embodiments, domain A has a VL amino acid sequence that is mutated at its C-terminus at the junction between domain A and domain B, as described in greater detail herein.

### 6.4.1.2. Complementarity Determining Regions

VH and VL amino acid sequences may comprise highly variable sequences termed “complementarity determining regions” (CDRs), typically three CDRs (CDR1, CD2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries.

### 6.4.1.3. Framework Regions and CDR Grafting

VH and VL amino acid sequences may comprise “framework region” (FR) sequences. FRs are generally conserved sequence regions that act as a scaffold for interspersed CDRs (see Section 6.4.1.2.), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.4.1.4. VH Regions

The VH amino acid sequences in the multispecific Treg-binding molecules described herein are antibody heavy chain variable domain sequences. In a typical antibody arrangement in both nature and in the multispecific Treg-binding molecules described herein, a specific VH amino acid sequence associates with a specific VL amino acid sequence to form an antigen-binding site. In various embodiments, VH amino acid sequences are mammalian sequences, including human sequences, synthesized sequences, or combinations of non-human mammalian, mammalian, and/or synthesized sequences, as described in further detail herein. In various embodiments, VH amino acid sequences are mutated sequences of naturally occurring sequences.

### 6.4.2. Domain B (Constant Region)

In the multispecific Treg-binding molecules, Domain B has a constant region domain sequence. Constant region domain amino acid sequences, as described herein, are sequences of a constant region domain of an antibody.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype. In preferred specific embodiments, the constant region sequence is a CH3 sequence. CH3 sequences are described in greater detail herein. In other preferred embodiments, the constant region sequence is an orthologous CH2 sequence. Orthologous CH2 sequences are described in greater detail herein.

In some embodiments, domain B has a CH1 sequence. In some embodiments, domain B has a CH2 sequence from IgE. In some embodiments, domain B has a CH2 sequence from IgM.

In particular embodiments, for example wherein the valency of the binding molecule is three or greater than three, the constant region sequence is a CH1 or Cl sequence. CH1 and Cl sequences are described herein. In some embodiments, the constant region sequence is a Cl sequence. In some embodiments, the CH1 or Cl sequence comprises one or more CH1 or Cl orthogonal modifications described herein.

In particular embodiments, the constant region sequence has been mutated to include one or more orthogonal mutations. In a preferred embodiment, domain B has a constant region sequence that is a CH3 sequence comprising knob-hole (synonymously, “knob-in-hole,” “KIH”) orthogonal mutations, as described in greater detail herein, and either a S354C or a Y349C mutation that forms an engineered disulfide bridge with a CH3 domain containing an orthogonal mutation, as described in in greater detail herein. In some preferred embodiments, the knob-hole orthogonal mutation is a T366W mutation.

### 6.4.2.1. CH3 Regions

CH3 amino acid sequences, as described herein, are sequences of the C-terminal domain of an antibody heavy chain.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype or CH4 sequences from an IgE or IgM isotype. In a specific embodiment, the CH3 sequences are from an IgG isotype. In a preferred embodiment, the CH3 sequences are from an IgG1 isotype. In some embodiments, the CH3 sequence is from an IgA isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a multispecific Treg-binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail herein.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1m1 allotype are replaced. In a preferred embodiment, isoallotype mutations D356E and L358M are made in the CH3 sequence.

In a preferred embodiment, domain B has a human IgG1 CH3 amino acid sequence with the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In certain embodiments, domain B has a human IgG1 CH3 sequence with a 447C mutation incorporated into an otherwise endogenous CH3 sequence.

In the multispecific Treg-binding molecules described herein, the N-terminus of domain B is connected to the C-terminus of domain A. In certain embodiments, domain B has a CH3 amino acid sequence that is mutated at its N-terminus at the junction between domain A and domain B, as described in greater detail herein and Example 6.

In the multispecific Treg-binding molecules, the C-terminus of domain B is connected to the N-terminus of domain D. In certain embodiments, domain B has a CH3 amino acid sequence that is extended at the C-terminus at the junction between domain B and domain D, as described in greater detail herein.

In some embodiments, domain B comprises a human IgA CH3 sequence. An exemplary human IgA CH3 sequence is

(SEQ ID NO: 184)
TFRPEVHLLPPPSEELALNELVTLTCLARGFSPKDVLVRWLQGS
QELPREKYLTWASRQEPSQGTTTFAVTSILRVAAEDWKKGDTFS
CMVGHEALPLAFTQKTIDRL

In some embodiments, the IgA-CH3 sequence comprises a CH3 linker sequence described herein.

### 6.4.2.2. Orthologous CH2 Regions

CH2 amino acid sequences, as described herein, are sequences of the third domain of an antibody heavy chain, with reference from the N-terminus to C-terminus. CH2 amino acid sequences, in general, are discussed in more detail herein. In a series of embodiments, a multispecific Treg-binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the multispecific Treg-binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the multispecific Treg-binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the multispecific Treg-binding molecules are described in more detail in international PCT applications WO2017/011342 and WO2017/106462, herein incorporated by reference in their entirety.

### 6.4.2.3. CH1 and CL Regions

CH1 amino acid sequences, as described herein, are sequences of the second domain of an antibody heavy chain, with reference from the N-terminus to C-terminus. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The CL amino acid sequences useful in the multispecific Treg-binding molecules described herein are antibody light chain constant domain sequences. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (2) light chain constant domain sequences. In particular embodiments, the CL amino acid sequences are human lambda light chain constant domain sequences. In preferred embodiments, the lambda (2) light chain sequence is UniProt accession number POCG04.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

In certain embodiments, the CH1 sequence and the CL sequences are both endogenous sequences. In certain embodiments, the CH1 sequence and the CL sequences separately comprise respectively orthogonal modifications in endogenous CH1 and CL sequences, as discussed in greater detail herein. It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the multispecific Treg-binding molecule having a portion of the CH1 sequences described herein can be bound by the CH1 binding reagent.

Without wishing to be bound by theory, the CH1 domain is also unique in that it's folding is typically the rate limiting step in the secretion of IgG (Feige et al. Mol Cell. 2009 Jun. 12; 34(5):569-79; herein incorporated by reference in its entirety). Thus, purifying the multispecific Treg-binding molecules based on the rate limiting component of CH1 comprising polypeptide chains can provide a means to purify complete complexes from incomplete chains, e.g., purifying complexes having a limiting CH1 domain from complexes only having one or more non-CH1 comprising chains.

While the CH1 limiting expression may be a benefit in some aspects, as discussed, there is the potential for CH1 to limit overall expression of the complete multispecific Treg-binding molecules. Thus, in certain embodiments, the expression of the polypeptide chain comprising the CH1 sequence(s) is adjusted to improve the efficiency of the multispecific Treg-binding molecules forming complete complexes. In an illustrative example, the ratio of a plasmid vector constructed to express the polypeptide chain comprising the CH1 sequence(s) can be increased relative to the plasmid vectors constructed to express the other polypeptide chains. In another illustrative example, the polypeptide chain comprising the CH1 sequence(s) when compared to the polypeptide chain comprising the CL sequence(s) can be the smaller of the two polypeptide chains. In another specific embodiment, the expression of the polypeptide chain comprising the CH1 sequence(s) can be adjusted by controlling which polypeptide chain has the CH1 sequence(s). For example, engineering the multispecific Treg-binding molecule such that the CH1 domain is present in a two-domain polypeptide chain (e.g., the 4th polypeptide chain described herein), instead of the CH1 sequence's native position in a four-domain polypeptide chain (e.g., the 3rd polypeptide chain described herein), can be used to control the expression of the polypeptide chain comprising the CH1 sequence(s). However, in other aspects, a relative expression level of CH1 containing chains that is too high compared to the other chains can result in incomplete complexes the have the CH1 chain, but not each of the other chains. Thus, in certain embodiments, the expression of the polypeptide chain comprising the CH1 sequence(s) is adjusted to both reduce the formation incomplete complexes without the CH1 containing chain, and to reduce the formation incomplete complexes with the CH1 containing chain but without the other chains present in a complete complex.

### 6.4.3. Domain D (Constant Region)

In the multispecific Treg-binding molecules described herein, domain D has a constant region amino acid sequence. Constant region amino acid sequences are described in more detail herein.

In a preferred series of embodiments, domain D has a CH2 amino acid sequence. CH2 amino acid sequences, as described herein, are CH2 amino acid sequences of the third domain of a native antibody heavy chain, with reference from the N-terminus to C-terminus. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from an IgA1, IgA2, IgD, IgE, IgG1, IgG2, IgG3, IgG4, or IgM isotype. In a preferred embodiment, the CH2 sequences are from an IgG1 isotype.

In certain embodiments, the CH2 sequences are endogenous sequences. In particular embodiments, the sequence is UniProt accession number P01857 amino acids 111-223. In a preferred embodiment, the CH2 sequences have an N-terminal hinge region peptide that connects the N-terminal variable domain-constant domain segment to the CH2 domain, as discussed in more detail herein. In some embodiments, the CH2 sequence comprises one or more mutations that reduce effector function, as discussed in more detail herein.

In the multispecific Treg-binding molecules, the N-terminus of domain D is connected to the C-terminus of domain B. In certain embodiments, domain B has a CH3 amino acid sequence that is extended at the C-terminus at the junction between domain D and domain B, as described in greater detail herein.

### 6.4.4. Domain E (Constant Region)

In the multispecific Treg-binding molecules, domain E has a constant region domain amino acid sequence. Constant region amino acid sequences are described in more detail herein.

In certain embodiments, the constant region sequence is a CH3 sequence. CH3 sequences are described in greater detail herein. In particular embodiments, the constant region sequence has been mutated to include one or more orthogonal mutations. In a preferred embodiment, domain E has a constant region sequence that is a CH3 sequence comprising knob-hole (synonymously, “knob-in-hole,” “KIH”) orthogonal mutations, as described in greater detail herein, and either a S354C or a Y349C mutation that forms an engineered disulfide bridge with a CH3 domain containing an orthogonal mutation, as described in greater detail herein. In some preferred embodiments, the knob-hole orthogonal mutation is a T366W mutation.

In certain embodiments, the constant region domain sequence is a CH1 sequence. In particular embodiments, the CH1 amino acid sequence of domain E is the only CH1 amino acid sequence in the multispecific Treg-binding molecule. In certain embodiments, the N-terminus of the CH1 domain is connected to the C-terminus of a CH2 domain, as described in greater detail herein. In certain embodiments, the constant region sequence is a CL sequence. In certain embodiments, the N-terminus of the CL domain is connected to the C-terminus of a CH2 domain, as described in greater detail herein. CH1 and CL sequences are described in further detail herein.

### 6.4.5. Domain F (Variable Region)

In the multispecific Treg-binding molecules, domain F has a variable region domain amino acid sequence. Variable region domain amino acid sequences, as discussed in greater detail herein, are variable region domain amino acid sequences of an antibody including VL and VH antibody domain sequences. VL and VH sequences are described in greater detail herein. In a preferred embodiment, domain F has a VH antibody domain sequence. In some embodiments, domain F has a VL antibody domain sequence.

### 6.4.6. Domain G

In the multispecific Treg-binding molecules, domain G has a constant region amino acid sequence. Constant region amino acid sequences are described in more detail herein.

In preferred embodiments, domain G has a CH3 amino acid sequence. CH3 sequences are described in greater detail herein.

In certain preferred embodiments, domain G has a human IgG1 CH3 sequence with the following mutational changes: S354C; and a tripeptide insertion, 445P, 446G, 447K. In some preferred embodiments, domain G has a human IgG1 CH3 sequence with the following mutational changes: S354C; and 445P, 446G, 447K tripeptide insertion. In some preferred embodiments, domain G has a human IgG1 CH3 sequence with the following changes: L351D, and a tripeptide insertion of 445G, 446E, 447C.

In some embodiments, domain G has a human IgA CH3 sequence. An exemplary human IgA CH3 sequence is described herein.

In some embodiments, domain G has a CL sequence. In some embodiments, domain G has a CH2 sequence from IgE. In some embodiments, domain G has a CH2 sequence from IgM.

In particular embodiments, for example wherein the valency of the binding molecule is three or greater than three, the constant region sequence is a CH1 or Cl sequence. In some embodiments wherein domain B is a Cl sequence, domain G is a CH1 sequence. CH1 and Cl sequences are described herein. In some embodiments, the CH1 or Cl sequence comprises one or more CH1 or Cl orthogonal modifications described herein.

In some embodiments of the multispecific Treg-binding molecules, the C-terminus of domain G is connected to the N-terminus of domain D. In certain embodiments, domain G has a CH3 amino acid sequence that is extended at the C-terminus at the junction between domain G and domain D, as described in greater detail herein.

### 6.4.7. Domain H (Variable Region)

In the multispecific Treg-binding molecules, domain H has a variable region domain amino acid sequence. Variable region domain amino acid sequences, as discussed in greater detail herein, are variable region domain amino acid sequences of an antibody including VL and VH antibody domain sequences. VL and VH sequences are described in greater detail herein. In a preferred embodiment, domain H has a VL antibody domain sequence. In some embodiments, domain H has a VH antibody domain sequence.

### 6.4.8. Domain I (Constant Region)

In the multispecific Treg-binding molecules, domain I has a constant region domain amino acid sequence. Constant region domain amino acid sequences are described in greater detail herein. In a series of preferred embodiments of the multispecific Treg-binding molecules, domain I has a CL amino acid sequence. In another series of embodiments, domain I has a CH1 amino acid sequence. CH1 and CL amino acid sequences are described in further detail herein.

### 6.4.9. Domain J (CH2)

In the multispecific Treg-binding molecules, domain J has a CH2 amino acid sequence. CH2 amino acid sequences are described in greater detail herein. In a preferred embodiment, the CH2 amino acid sequence has an N-terminal hinge region that connects domain J to domain I, as described in more detail herein. In some embodiments, the CH2 sequence comprises one or more mutations that reduce effector function, as discussed in more detail herein.

In the multispecific Treg-binding molecules, the C-terminus of domain J is connected to the N-terminus of domain K. In particular embodiments, domain J is connected to the N-terminus of domain K that has a CH1 amino acid sequence or CL amino acid sequence, as described in further detail herein.

### 6.4.10. Domain K (Constant Region)

In the multispecific Treg-binding molecules, domain K has a constant region domain amino acid sequence. Constant region domain amino acid sequences are described in greater detail herein. In certain embodiments, the constant region sequence is a CH3 sequence. CH3 sequences are described in greater detail herein. In a preferred embodiment, domain K has a constant region sequence that is a CH3 sequence comprising knob-hole orthogonal mutations, as described in greater detail herein; isoallotype mutations, as described in more detail above; and either a S354C or a Y349C mutation that forms an engineered disulfide bridge with a CH3 domain containing an orthogonal mutation, as described in greater detail herein. In some preferred embodiments, the knob-hole orthogonal mutations combined with isoallotype mutations are the following mutational changes: D356E, L358M, T366S, L368A, and Y407V.

In certain embodiments, the constant region domain sequence is a CH1 sequence. In particular embodiments, the CH1 amino acid sequence of domain K is the only CH1 amino acid sequence in the multispecific Treg-binding molecule. In certain embodiments, the N-terminus of the CH1 domain is connected to the C-terminus of a CH2 domain, as described in greater detail herein. In certain embodiments, the constant region sequence is a CL sequence. In certain embodiments, the N-terminus of the CL domain is connected to the C-terminus of a CH2 domain, as described in greater detail herein. CH1 and CL sequences are described in further detail herein.

### 6.4.11. Domain L (Variable Region)

In the multispecific Treg-binding molecules, domain L has a variable region domain amino acid sequence. Variable region domain amino acid sequences, as discussed in greater detail herein, are variable region domain amino acid sequences of an antibody including VL and VH antibody domain sequences. VL and VH sequences are described in greater detail herein. In a preferred embodiment, domain L has a VH antibody domain sequence. In some embodiments, domain L has a VL antibody domain sequence.

### 6.4.12. Domain M (Constant Region)

In the multispecific Treg-binding molecules, domain M has a constant region domain amino acid sequence. Constant region domain amino acid sequences are described in greater detail herein. In a series of preferred embodiments of the multispecific Treg-binding molecules, domain I has a CH1 amino acid sequence and domain M has a CL amino acid sequence. In another series of preferred embodiments, domain I has a CL amino acid sequence and domain M has a CH1 amino acid sequence. CH1 and CL amino acid sequences are described in further detail herein.

### 6.4.13. Pairing of Domains A & F

In the multispecific Treg-binding molecules, a domain A VL or VH amino acid sequence and a cognate domain F VH or VL amino acid sequence are associated and form an antigen binding site (ABS). The A:F antigen binding site (ABS) is capable of specifically binding an epitope of an antigen. Antigen binding by an ABS is described in greater detail herein.

In a variety of multivalent embodiments, the ABS formed by domains A and F (A:F) is identical in sequence to one or more other ABSs within the multispecific Treg-binding molecule and therefore has the same recognition specificity as the one or more other sequence-identical ABSs within the multispecific Treg-binding molecule.

In a variety of multivalent embodiments, the A:F ABS is non-identical in sequence to one or more other ABSs within the multispecific Treg-binding molecule. In certain embodiments, the A:F ABS has a recognition specificity different from that of one or more other sequence-non-identical ABSs in the multispecific Treg-binding molecule. In particular embodiments, the A:F ABS recognizes a different antigen from that recognized by at least one other sequence-non-identical ABS in the multispecific Treg-binding molecule. In particular embodiments, the A:F ABS recognizes a different epitope of an antigen that is also recognized by at least one other sequence-non-identical ABS in the multispecific Treg-binding molecule. In these embodiments, the ABS formed by domains A and F recognizes an epitope of antigen, wherein one or more other ABSs within the multispecific Treg-binding molecule recognizes the same antigen but not the same epitope.

### 6.4.14. Pairing of Domains B & G

In the multispecific Treg-binding molecules described herein, a domain B constant region amino acid sequence and a domain G constant region amino acid sequence are associated. Constant region domain amino acid sequences are described in greater detail herein.

In a series of preferred embodiments, domain B and domain G have CH3 amino acid sequences. CH3 sequences are described in greater detail herein. In various embodiments, the amino acid sequences of the B and the G domains are identical. In certain of these embodiments, the sequence is an endogenous CH3 sequence. The sequence may be a CH3 sequence from human IgG1. The sequence may be a sequence from human IgA.

In a variety of embodiments, the amino acid sequences of the B and the G domains are different, and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

Orthogonal modifications” or synonymously “orthogonal mutations” as described herein are one or more engineered mutations in an amino acid sequence of an antibody domain that alter the affinity of binding of a first domain having orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail herein. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail herein.

### 6.4.14.1. Orthogonal Engineered Disulfide Bridges in CH3

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

In some embodiments, the orthogonal engineered disulfide bridge is between a first IgA-CH3 domain and a second IgA-CH3 domain. In some embodiments, the mutations that generate such engineered disulfide bridge is a H350C mutation in one of the first or second IgA-CH3 domains and a P355C mutation in the other IgA-CH3 domain.

For clarity, the residue designated “H350” in the IgA-CH3 domain sequence is the underlined “H” residue in the following endogenous IgA-CH3 amino acid sequence:

(SEQ ID NO: 184)
TFRPEVHLLPPPSEELALNELVTLTCLARGFSPKDVLVRWLQGS
QELPREKYLTWASRQEPSQGTTTFAVTSILRVAAEDWKKGDTFS
CMVGHEALPLAFTQKTIDRL.

By way of example, an IgA-CH3 amino acid domain sequence with a “H350C” mutation in an otherwise endogenous IgA-CH3 domain has the following sequence:

(SEQ ID NO: 303)
TFRPEVCLLPPPSEELALNELVTLTCLARGFSPKDVLVRWLQG
SQELPREKYLTWASRQEPSQGTTTFAVTSILRVAAEDWKKGDT
FSCMVGHEALPLAFTQKTIDRL.

For clarity, the residue designated “P355” in the IgA-CH3 domain sequence is the underlined “P” residue in the following endogenous IgA-CH3 amino acid sequence:

(SEQ ID NO: 184)
TFRPEVHLLPPPSEELALNELVTLTCLARGFSPKDVLVRWLQG
SQELPREKYLTWASRQEPSQGTTTFAVTSILRVAAEDWKKGDT
FSCMVGHEALPLAFTQKTIDRL.

By way of example, an IgA-CH3 amino acid domain sequence with a “P355C” mutation in an otherwise endogenous IgA-CH3 domain has the following sequence:

(SEQ ID NO: 304)
TFRPEVHLLPPCSEELALNELVTLTCLARGFSPKDVLVRWLQG
SQELPREKYLTWASRQEPSQGTTTFAVTSILRVAAEDWKKGDT
FSCMVGHEALPLAFTQKTIDRL.

### 6.4.14.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681)), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and aY407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and aY407V mutation in a second domain.

### 6.4.14.3. Orthogonal Charge-Pair Mutations

In a variety of embodiments, orthogonal modifications are charge-pair mutations. As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

### 6.4.14.4. IgA-CH3 Isotype Domain Substitution

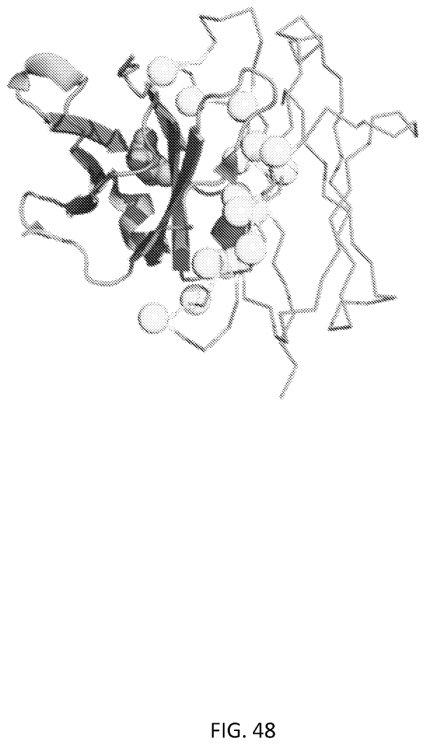

FIG. 48 depicts a rendition of a human IgA CH3 dimer. Non-identical residues in reference to human IgG CH3 are depicted as white spheres. In some embodiments, it is desirable to reduce an undesired association of a first and second domain, which may contain CH3 sequences, with a third and fourth domain, which may also contain CH3 sequences. In such cases, use of CH3 sequences from human IgA (IgA-CH3) in the first and/or second domain may improve antibody assembly and stability by reducing such undesired associations. In some embodiments of a multispecific Treg-binding molecule wherein the third and fourth domain comprise IgG-CH3 sequences, the first and/or second domain comprises IgA-CH3 sequences.

In some embodiments, at least one of the first or second domain comprise a CH3 linker sequence as described herein. In some embodiments, both the first and second domain comprise a CH3 linker sequence as described herein. In some embodiments, the first comprises a first CH3 linker sequence and the second domain comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids.

In particular embodiments, it is desirable to reduce an undesired association of domains B or G, which may contain CH3 sequences, with domains E and K, which may also contain CH3 sequences. In such cases, use of CH3 sequences from human IgA (IgA-CH3) in domains B and/or G may improve antibody assembly and stability by reducing such undesired associations. In some embodiments of a multispecific Treg-binding molecule wherein domains E and K comprise IgG-CH3 sequences, domains B and G comprises IgA-CH3 sequences.

In particular embodiments, at least one of domains B and G comprise a CH3 linker sequence as described herein. In some embodiments, both domains B and G comprise a CH3 linker sequence as described herein. In some embodiments, domain B comprises a first CH3 linker sequence and domain G comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids. In some embodiments, the first CH3 linker and second CH3 linker are 1-10, 2-8, or 3-6 amino acids in length. In some embodiments, the first CH3 linker is 3 amino acids in length and the second CH3 linker is 5 or 6 amino acids in length.

In some embodiments, the first CH3 linker and the second CH3 linker are provided in Table 9, as described herein.

In some embodiments, the first CH3 linker and second CH3 linker each comprise an amino acid cysteine substitution in the endogenous IgA-CH3 sequence. In some embodiments, the first CH3 linker and second CH3 linker each consist of an amino acid cysteine substitution in the endogenous IgA-CH3 sequence. In some embodiments, the first CH3 linker is a H350C substitution and the second CH3 linker is a P355C substitution. In some embodiments, the first CH3 linker is a P355C substitution and the second CH3 linker is a H350C substitution.

For clarity, the residue designated “H350” in the IgA-CH3 domain sequence is the underlined “H” residue in the following endogenous IgA-CH3 sequence:

(SEQ ID NO: 184)
TFRPEVHLLPPPSEELALNELVTLTCLARGFSPKDVLVRWLQ
GSQELPREKYLTWASRQEPSQGTTTFAVTSILRVAAEDWKKG
DTFSCMVGHEALPLAFTQKTIDRL.

By way of example, an IgA-CH3 amino acid domain sequence with a “H350C” mutation in an otherwise endogenous IgA-CH3 domain has the following sequence:

(SEQ ID NO: 303)
TFRPEVCLLPPPSEELALNELVTLTCLARGFSPKDVLVRWLQ
GSQELPREKYLTWASRQEPSQGTTTFAVTSILRVAAEDWKKG
DTFSCMVGHEALPLAFTQKTIDRL.

For clarity, the residue designated “P355” in the IgA-CH3 domain sequence is the underlined “P” residue in the following endogenous IgA-CH3 sequence:

(SEQ ID NO: 184)
TFRPEVHLLPPPSEELALNELVTLTCLARGFSPKDVLVRWLQ
GSQELPREKYLTWASRQEPSQGTTTFAVTSILRVAAEDWKKG
DTFSCMVGHEALPLAFTQKTIDRL.

By way of example, an IgA-CH3 amino acid domain sequence with a “P355C” mutation in an otherwise endogenous IgA-CH3 domain has the following sequence:

(SEQ ID NO: 304)
TFRPEVHLLPPCSEELALNELVTLTCLARGFSPKDVLVRWLQ
GSQELPREKYLTWASRQEPSQGTTTFAVTSILRVAAEDWKKG
DTFSCMVGHEALPLAFTQKTIDRL.

In some embodiments, the first CH3 linker and the second CH3 linker are provided in Table 9, as described herein

In some embodiments, the first CH3 linker and the second CH3 linker are provided in Table 25, as described herein.

In preferred embodiments, the first CH3 linker is AGC and the second CH3 linker is AGKGSC (SEQ ID NO: 99). In some embodiments, the first CH3 linker is AGKGC (SEQ ID NO: 98) and the second CH3 linker is AGC. In some embodiments, the first CH3 linker is AGKGSC (SEQ ID NO: 99) and the second CH3 linker is AGC. In some embodiments, the first CH3 linker is AGKC (SEQ ID NO: 96) and the second CH3 linker is AGC. In some embodiments, the first CH3 linker is a P355C amino acid substitution and the second CH3 linker is a H350C amino acid substitution.

In some embodiments, wherein the first and second domains comprise IgA-CH3 sequences and the third and fourth domains comprise IgA-CH3 sequences, unwanted associations between the first or second domains with either the third or fourth domains are reduced when the first and second domains comprise a first and second CH3 linker, respectively, and the third and fourth domains comprise a third and fourth CH3 linker, respectively. In some embodiments, the first and second CH3 linkers on the first and second domains preferentially pair with each other and do not preferentially pair with the third or fourth CH3 linkers on the third and fourth domains. In some embodiments, the third and fourth CH3 linkers on the third and fourth domains preferentially pair with each other and do not preferentially pair with the first or second CH3 linkers on the first and second domains. In some embodiments, the first and second CH3 linkers are selected from Table 9, and the third and fourth CH3 linkers each comprise an amino acid cysteine substitution in the endogenous IgA-CH3 sequence. In some embodiments, the third CH3 linker and fourth CH3 linker each consist of an amino acid cysteine substitution in the endogenous IgA-CH3 sequence. Exemplary cysteine substitutions in endogenous IgA-CH3 sequences are described herein.

### 6.4.15. Pairing of Domains E & K

In various embodiments, the E domain has a CH3 amino acid sequence.

In various embodiments, the K domain has a CH3 amino acid sequence.

In a variety of embodiments, the amino acid sequences of the E and K domains are identical, wherein the sequence is an endogenous CH3 sequence. CH3 sequences are described herein. In some embodiments, the CH3 sequences of domains E and K are IgG-CH3 sequences.

In a variety of embodiments, the sequences of the E and K domains are different. In a variety of embodiments, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In certain embodiments, the orthogonal modifications include, but are not limited to, engineered disulfide bridges, knob-in-hole mutations, and charge-pair mutations, as described in greater detail herein. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations.

### 6.4.16. Pairing of Domains I & M and Domains H & L

In a variety of embodiments, domain I has a CL sequence and domain M has a CH1 sequence. In a variety of embodiments, domain H has a VL sequence and domain L has a VH sequence. In a preferred embodiment, domain H has a VL amino acid sequence, domain I has a CL amino acid sequence, domain L has a VH amino acid sequence, and domain M has a CH1 amino acid sequence. In another preferred embodiment, domain H has a VL amino acid sequence, domain I has a CL amino acid sequence, domain L has a VH amino acid sequence, domain M has a CH1 amino acid sequence, and domain K has a CH3 amino acid sequence.

In a variety of embodiments, the amino acid sequences of the I domain and the M domain separately comprise respectively orthogonal modifications in an endogenous sequence, wherein the I domain interacts with the M domain, and wherein neither the I domain nor the M domain significantly interacts with a domain lacking the orthogonal modification. In a series of embodiments, the orthogonal mutations in the I domain are in a CL sequence and the orthogonal mutations in the M domain are in CH1 sequence. Orthogonal mutations are in CH1 and CL sequences are described in more detail herein.

In a variety of embodiments, the amino acid sequences of the H domain and the L domain separately comprise respectively orthogonal modifications in an endogenous sequence, wherein the H domain interacts with the L domain, and wherein neither the H domain nor the L domain significantly interacts with a domain lacking the orthogonal modification. In a series of embodiments, the orthogonal mutations in the H domain are in a VL sequence and the orthogonal mutations in the L domain are in VH sequence. In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

In certain embodiments, the interaction between the A domain and the F domain form a first antigen binding site specific for a first antigen, and the interaction between the H domain and the L domain form a second antigen binding site specific for a second antigen. In certain embodiments, the interaction between the A domain and the F domain form a first antigen binding site specific for a first antigen, and the interaction between the H domain and the L domain form a second antigen binding site specific for the first antigen.

### 6.4.17. Trivalent Binding Molecules

In another series of embodiments, the multispecific Treg-binding molecules have three antigen binding sites and are therefore termed “trivalent.”

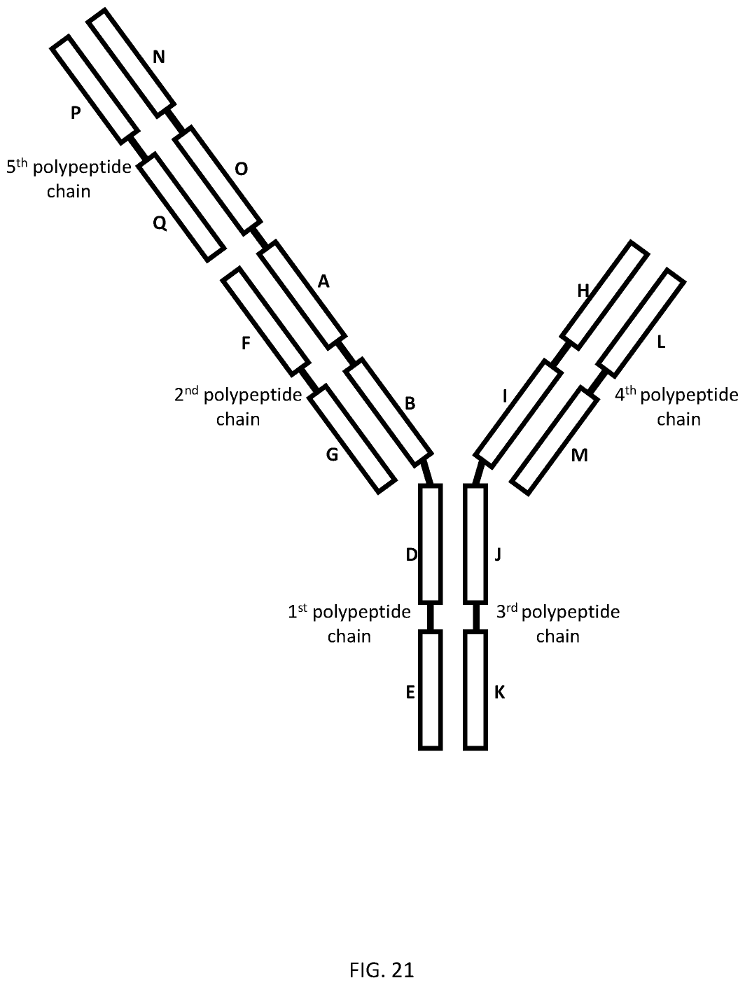

With reference to FIG. 21, in various trivalent embodiments the multispecific Treg-binding molecules further comprise a fifth polypeptide chain, wherein (a) the first polypeptide chain further comprises a domain N and a domain O, wherein the domains are arranged, from N-terminus to C-terminus, in a N-O-A-B-D-E orientation, and wherein domain N has a VL amino acid sequence, domain O has a constant region amino acid sequence; (b) the multispecific Treg-binding molecule further comprises a fifth polypeptide chain, comprising: a domain P and a domain Q, wherein the domains are arranged, from N-terminus to C-terminus, in a P-Q orientation, and wherein domain P has a VH amino acid sequence and domain Q has a constant region amino acid sequence; and (c) the first and the fifth polypeptides are associated through an interaction between the N and the P domains and an interaction between the O and the Q domains to form the multispecific Treg-binding molecule. As schematized in FIG. 2, these trivalent embodiments are termed “2×1” trivalent constructs.

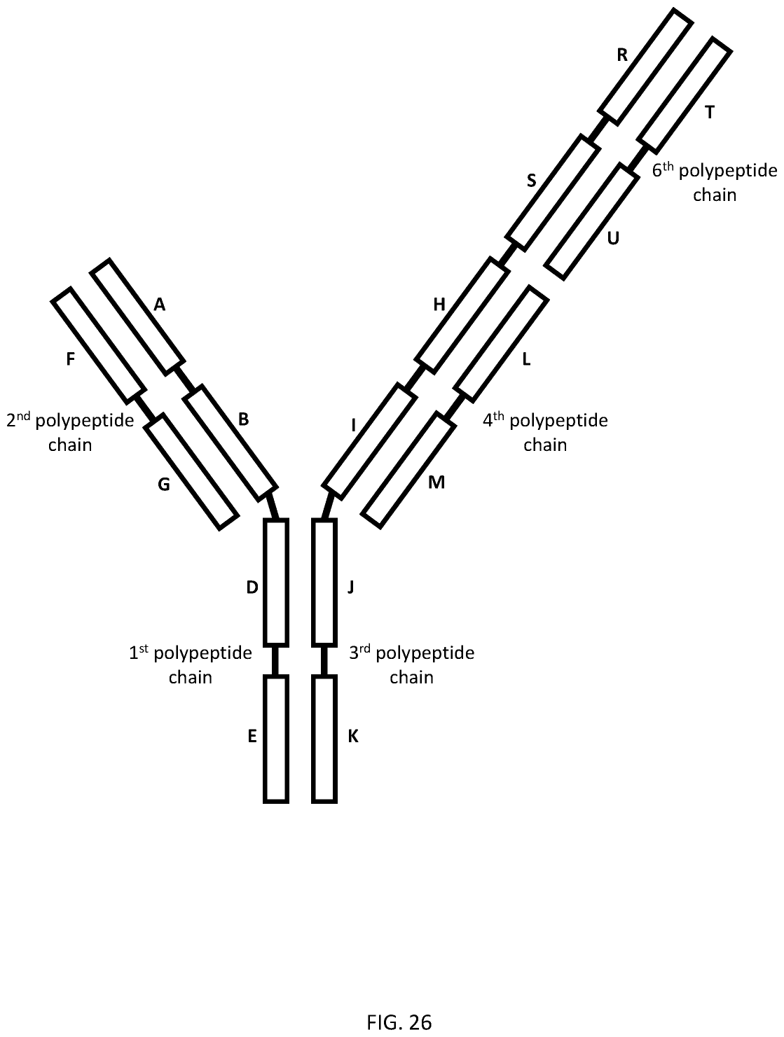

With reference to FIG. 26, in a further series of trivalent embodiments, the multispecific Treg-binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a VL amino acid sequence and domain S has a constant domain amino acid sequence; (b) the multispecific Treg-binding molecule further comprises a sixth polypeptide chain, comprising: a domain T and a domain U, wherein the domains are arranged, from N-terminus to C-terminus, in a T-U orientation, and wherein domain T has a VH amino acid sequence and domain U has a constant domain amino acid sequence; and (c) the third and the sixth polypeptides are associated through an interaction between the R and the T domains and an interaction between the S and the U domains to form the multispecific Treg-binding molecule. As schematized in FIG. 2, these trivalent embodiments are termed “1×2” trivalent constructs.

In a variety of embodiments, the domain O is connected to domain A through a peptide linker. In a variety of embodiments, the domain S is connected to domain H through a peptide linker. In a preferred embodiment, the peptide linker connecting either domain O to domain A or connecting domain S to domain H is a 6 amino acid GSGSGS peptide sequence (SEQ ID NO: 40), as described in more detail herein.

### 6.4.17.1. Trivalent 2×1 Bispecific Constructs [2(A−A)×1(B)]

With reference to FIG. 21, in a variety of embodiments the amino acid sequences of domain N and domain A are identical, the amino acid sequences of domain H is different from domains N and A, the amino acid sequences of domain O and domain B are identical, the amino acid sequences of domain I is different from domains O and B, the amino acid sequences of domain P and domain F are identical, the amino acid sequences of domain L is different from domains P and F, the amino acid sequences of domain Q and domain G are identical, the amino acid sequences of domain M is different from domains Q and G; and the interaction between the A domain and the F domain form a first antigen binding site specific for a first antigen, the interaction between the H domain and the L domain form a second antigen binding site specific for a second antigen, and the domain N and domain P form a third antigen binding site specific for the first antigen.

### 6.4.17.2. Trivalent 2×1 Bispecific Constructs [2(A−B)×1(A)]

With reference to FIG. 21, in a variety of embodiments the amino acid sequences of domain N and domain H are identical, the amino acid sequences of domain A is different from domains N and H, the amino acid sequences of domain O and domain I are identical, the amino acid sequences of domain B is different from domains O and I, the amino acid sequences of domain P and domain L are identical, the amino acid sequences of domain F is different from domains P and L, the amino acid sequences of domain Q and domain M are identical, the amino acid sequences of domain G is different from domains Q and M; and the interaction between the A domain and the F domain form a first antigen binding site specific for a first antigen, the interaction between the H domain and the L domain form a second antigen binding site specific for a second antigen, and the domain N and domain P form a third antigen binding site specific for the second antigen.

### 6.4.17.3. Trivalent 2×1 Trispecific Constructs [2(A−B)×1(C)]

With reference to FIG. 21, in a variety of embodiments, the amino acid sequences of domain N, domain A, and domain H are different, the amino acid sequences of domain O, domain B, and domain I are different, the amino acid sequences of domain P, domain F, and domain L are different, and the amino acid sequences of domain Q, domain G, and domain M are different; and the interaction between the A domain and the F domain form a first antigen binding site specific for a first antigen, the interaction between the H domain and the L domain form a second antigen binding site specific for a second antigen, and the domain N and domain P form a third antigen binding site specific for a third antigen.

In certain embodiments, domain O has a constant region sequence that is a CL from a kappa light chain and domain Q has a constant region sequence that is a CH1 from an IgG1 isotype, as discussed in more detail herein. In a preferred embodiment, domain O and domain Q have CH3 sequences such that they specifically associate with each other, as discussed in more detail herein.

### 6.4.17.4. Trivalent 1×2 Bispecific Constructs [1(A)×2(B−A)]

With reference to FIG. 26, in a variety of embodiments, the amino acid sequences of domain R and domain A are identical, the amino acid sequences of domain H is different from domain R and A, the amino acid sequences of domain S and domain B are identical, the amino acid sequences of domain I is different from domain S and B, the amino acid sequences of domain T and domain F are identical, the amino acid sequences of domain L is different from domain T and F, the amino acid sequences of domain U and domain G are identical, the amino acid sequences of domain M is different from domain U and G and the interaction between the A domain and the F domain form a first antigen binding site specific for a first antigen, the interaction between the H domain and the L domain form a second antigen binding site specific for a second antigen, and the domain R and domain T form a third antigen binding site specific for the first antigen.

### 6.4.17.5. Trivalent 1×2 Bispecific Constructs [1(A)×2(B−B)]

In a variety of embodiments, the multispecific Treg-binding molecule further comprises a second CH1 domain, or portion thereof. With reference to FIG. 26, in specific embodiments, the amino acid sequences of domain R and domain H are identical, the amino acid sequences of domain A is different from domain R and H, the amino acid sequences of domain S and domain I are identical, the amino acid sequences of domain B is different from domain S and I, the amino acid sequences of domain T and domain L are identical, the amino acid sequences of domain F is different from domain T and L, the amino acid sequences of domain U and domain M are identical, the amino acid sequences of domain G is different from domain U and M and the interaction between the A domain and the F domain form a first antigen binding site specific for a first antigen, the interaction between the H domain and the L domain form a second antigen binding site specific for a second antigen, and the domain R and domain T form a third antigen binding site specific for the second antigen.

In particular embodiments, the amino acid sequences of domain S and domain I are CH1 sequences. In particular embodiments, the amino acid sequences of domain U and domain M are CH1 sequences.

### 6.4.17.6. Trivalent 1×2 Trispecific Constructs [1(A)×2(B−C)]

With reference to FIG. 26, in a variety of embodiments, the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and the interaction between the A domain and the F domain form a first antigen binding site specific for a first antigen, the interaction between the H domain and the L domain form a second antigen binding site specific for a second antigen, and the domain R and domain T form a third antigen binding site specific for a third antigen.

In particular embodiments, domain S has a constant region sequence that is a CL from a kappa light chain and domain U has a constant region sequence that is a CH1 from an IgG1 isotype, as discussed in more detail herein. In a preferred embodiment, domain S and domain U have CH3 sequences such that they specifically associate with each other, as discussed in more detail herein.

In certain embodiments, the multispecific Treg-binding molecule further comprises a second CH1 domain, or portion thereof. In particular embodiments, the amino acid sequences of domain S and domain I are CH1 sequences. In particular embodiments, the amino acid sequences of domain U and domain M are CH1 sequences.

### 6.4.18. Tetravalent 2×2 Binding Molecules

In a variety of embodiments, the multispecific Treg-binding molecules have 4 antigen binding sites and are therefore termed “tetravalent.”

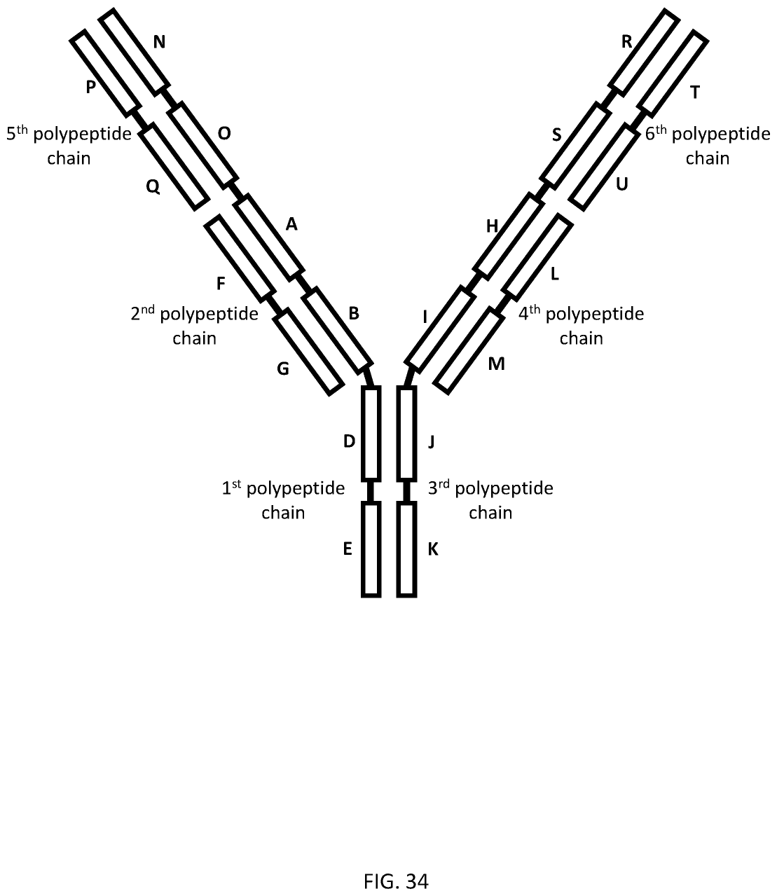

With reference to FIG. 34, in a further series of embodiments, the multispecific Treg-binding molecules further comprise a fifth and a sixth polypeptide chain, wherein (a) the first polypeptide chain further comprises a domain N and a domain O, wherein the domains are arranged, from N-terminus to C-terminus, in a N-O-A-B-D-E orientation; (b) the third polypeptide chain further comprises a domain R and a domain S, wherein the domains are arranged, from N-terminus to C-terminus, in a R-S-H-I-J-K orientation; (c) the multispecific Treg-binding molecule further comprises a fifth and a sixth polypeptide chain, wherein the fifth polypeptide chain comprises a domain P and a domain Q, wherein the domains are arranged, from N-terminus to C-terminus, in a P-Q orientation, and the sixth polypeptide chain comprises a domain T and a domain U, wherein the domains are arranged, from N-terminus to C-terminus, in a T-U orientation; and (d) the first and the fifth polypeptides are associated through an interaction between the N and the P domains and an interaction between the O and the Q domains, and the third and the sixth polypeptides are associated through an interaction between the R and the T domains and an interaction between the S and the U domains to form the multispecific Treg-binding molecule.

In a variety of embodiments, the domain O is connected to domain A through a peptide linker and the domain S is connected to domain H through a peptide linker. In a preferred embodiment, the peptide linker connecting domain O to domain A and connecting domain S to domain H is a 6 amino acid GSGSGS peptide sequence (SEQ ID NO: 40), as described in more detail herein.

### 6.4.18.1. Tetravalent 2×2 Bispecific Constructs

With reference to FIG. 34, in a series of tetravalent 2×2 bispecific binding molecules, the amino acid sequences of domain N and domain A are identical, the amino acid sequences of domain H and domain R are identical, the amino acid sequences of domain O and domain B are identical, the amino acid sequences of domain I and domain S are identical, the amino acid sequences of domain P and domain F are identical, the amino acid sequences of domain L and domain T are identical, the amino acid sequences of domain Q and domain G are identical, the amino acid sequences of domain M and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for a second antigen, and the interaction between the R domain and the T domain form a fourth antigen binding site specific for the second antigen.

With reference to FIG. 34, in another series of tetravalent 2×2 bispecific binding molecules, the amino acid sequences of domain H and domain A are identical, the amino acid sequences of domain N and domain R are identical, the amino acid sequences of domain I and domain B are identical, the amino acid sequences of domain O and domain S are identical, the amino acid sequences of domain L and domain F are identical, the amino acid sequences of domain P and domain T are identical, the amino acid sequences of domain M and domain G are identical, the amino acid sequences of domain Q and domain U are identical; and wherein the interaction between the A domain and the F domain form a first antigen binding site specific for a first antigen, the domain N and domain P form a second antigen binding site specific for a second antigen, the interaction between the H domain and the L domain form a third antigen binding site specific for the first antigen, and the interaction between the R domain and the T domain form a fourth antigen binding site specific for the second antigen.

### 6.4.19. Domain Junctions

### 6.4.19.1. Junctions Connecting VL and CH3 Domains

In a variety of embodiments, the amino acid sequence that forms a junction between the C-terminus of a VL domain and the N-terminus of a CH3 domain is an engineered sequence. In certain embodiments, one or more amino acids are deleted or added in the C-terminus of the VL domain. In certain embodiments, the junction connecting the C-terminus of a VL domain and the N-terminus of a CH3 domain is one of the sequences described in Table 2 herein. In particular embodiments, A111 is deleted in the C-terminus of the VL domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In certain embodiments, one or more amino acids are deleted or added to both the C-terminus of the VL domain and the N-terminus of the CH3 domain. In particular embodiments, A111 is deleted in the C-terminus of the VL domain and P343 is deleted in the N-terminus of the CH3 domain. In a preferred embodiment, A111 and V110 are deleted in the C-terminus of the VL domain. In another preferred embodiment, A111 and V110 are deleted in the C-terminus of the VL domain and the N-terminus of the CH3 domain has a P343V mutation.

### 6.4.19.2. Junctions Connecting VH and CH3 Domains

In a variety of embodiments, the amino acid sequence that forms a junction between the C-terminus of a VH domain and the N-terminus of a CH3 domain is an engineered sequence. In certain embodiments, one or more amino acids are deleted or added in the C-terminus of the VH domain. In certain embodiments, the junction connecting the C-terminus of a VH domain and the N-terminus of the CH3 domain is one of the sequences described in Table 3 herein. In particular embodiments, K117 and G118 are deleted in the C-terminus of the VH domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In particular embodiments, P343, R344, and E345 are deleted in the N-terminus of the CH3 domain. In certain embodiments, one or more amino acids are deleted or added to both the C-terminus of the VH domain and the N-terminus of the CH3 domain.

In a preferred embodiment, T116, K117, and G118 are deleted in the C-terminus of the VH domain.

### 6.4.19.3. Junctions Connecting CH3 C-terminus to CH2 N-terminus (Hinge)

In the multispecific Treg-binding molecules described herein, the N-terminus of the CH2 domain has a “hinge” region amino acid sequence. As used herein, hinge regions are sequences of an antibody heavy chain that link the N-terminal variable domain-constant domain segment of an antibody and a CH2 domain of an antibody. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains). As used herein, the hinge region amino acid sequence is SEQ ID NO: 56.

In a variety of embodiments, a CH3 amino acid sequence is extended at the C-terminus at the junction between the C-terminus of the CH3 domain and the N-terminus of a CH2 domain. In certain embodiments, a CH3 amino acid sequence is extended at the C-terminus at the junction between the C-terminus of the CH3 domain and a hinge region, which in turn is connected to the N-terminus of a CH2 domain. In a preferred embodiment, the CH3 amino acid sequence is extended by inserting a CH3 amino acid extension sequence (“CH3 linker sequence” or “CH3 linker”). In some embodiments, the CH3 amino acid extension sequence is followed by the DKTHT motif (SEQ ID NO: 185) of an IgG1 hinge region. In some embodiments, the CH3 amino acid extension sequence is 3-10 amino acids in length. In some embodiments, the CH3 amino acid extension sequence is 3-8 amino acids in length. In some embodiments, the CH3 amino acid extension sequence is 3-6 amino acids in length.

In some embodiments, the CH3 amino acid extension sequence is a PGK tripeptide. In some embodiments, the CH3 amino acid extension sequence is an AGC tripeptide. In some embodiments, the CH3 amino acid extension sequence is a GEC tripeptide. In some embodiments, the CH3 amino acid extension sequence is AGKC (SEQ ID NO:96). In some embodiments, the CH3 amino acid extension sequence is PGKC (SEQ ID NO:97). In some embodiments, the CH3 amino acid extension sequence is AGKGC (SEQ ID NO:98). In some embodiments, the CH3 amino acid extension sequence is AGKGSC (SEQ ID NO:99).

In a particular embodiment, the extension at the C-terminus of the CH3 domain incorporates amino acid sequences that can form a disulfide bond with orthogonal C-terminal extension of another CH3 domain. In a preferred embodiment, the extension at the C-terminus of the CH3 domain incorporates a KSC tripeptide sequence that is followed by the DKTHT motif (SEQ ID NO: 185) of an IgG1 hinge region that forms a disulfide bond with orthogonal C-terminal extension of another CH3 domain that incorporates a GEC motif of a kappa light chain.

In some embodiments of a multispecific Treg-binding molecule wherein domains B and G comprise CH3 amino acid sequences, domain B comprises a first CH3 linker sequence and domain G comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids.

In some embodiments, the first CH3 linker and the second CH3 linker are provided in Table 9, as described herein.

In preferred embodiments, the first CH3 linker is AGC and the second CH3 linker is AGKGSC (SEQ ID NO: 99). In some embodiments, the first CH3 linker is AGKGC (SEQ ID NO: 98) and the second CH3 linker is AGC. In some embodiments, the first CH3 linker is AGKGSC (SEQ ID NO: 99) and the second CH3 linker is AGC. In some embodiments, the first CH3 linker is AGKC (SEQ ID NO: 96) and the second CH3 linker is AGC.

### 6.4.19.4. Junctions Connecting CL C-Terminus and CH2 N-Terminus (Hinge)

In a variety of embodiments, a CL amino acid sequence is connected through its C-terminus to a hinge region, which in turn is connected to the N-terminus of a CH2 domain. Hinge region sequences are described in more detail herein. In a preferred embodiment, the hinge region amino acid sequence is SEQ ID NO:56.

### 6.4.19.5. Junctions Connecting CH2 C-Terminus to Constant Region Domain

In a variety of embodiments, a CH2 amino acid sequence is connected through its C-terminus to the N-terminus of a constant region domain. Constant regions are described in more detail herein. In a preferred embodiment, the CH2 sequence is connected to a CH3 sequence via its endogenous sequence. In other embodiments, the CH2 sequence is connected to a CH1 or CL sequence. Examples discussing connecting a CH2 sequence to a CH1 or CL sequence are described in more detail in U.S. Pat. No. 8,242,247, which is hereby incorporated in its entirety.

### 6.4.19.6. Junctions Connecting Domain O to Domain A or Domain S to Domain H on Trivalent and Tetravalent Molecules

In a variety of embodiments, heavy chains of antibodies (e.g. the first and third polypeptide chains) are extended at their N-terminus to include additional domains that provide additional ABSs. With reference to FIG. 21, FIG. 26, and FIG. 34, in certain embodiments, the C-terminus of the constant region domain amino acid sequence of a domain O and/or a domain S is connected to the N-terminus of the variable region domain amino acid sequence of a domain A and/or a domain H, respectively. In some preferred embodiments, the constant region domain is a CH3 amino acid sequence and the variable region domain is a VL amino acid sequence. In some preferred embodiments, the constant region domain is a CL amino acid sequence and the variable region domain is a VL amino acid sequence. In certain embodiments, the constant region domain is connected to the variable region domain through a peptide linker. In a preferred embodiment, the peptide linker is a 6 amino acid GSGSGS peptide sequence (SEQ ID NO: 40).

In a variety of embodiments, light chains of antibodies (e.g. the second and fourth polypeptide chains) are extended at their N-terminus to include additional variable domain-constant domain segments of an antibody. In certain embodiments, the constant region domain is a CH1 amino acid sequence and the variable region domain is a VH amino acid sequence.

### 6.5. Exemplary Bivalent Binding Molecules

In a further aspect, bivalent binding molecules are provided.

With reference to FIG. 3, in a series of embodiments the multispecific Treg-binding molecules comprise a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the multispecific Treg-binding molecule.

In a preferred embodiment, domain E has a CH3 amino acid sequence, domain H has a VL amino acid sequence, domain I has a CL amino acid sequence, domain K has a CH3 amino acid sequence, domain L has a VH amino acid sequence, and domain M has a CH1 amino acid sequence.

In certain embodiments, the interaction between the A domain and the F domain form a first antigen binding site specific for a first antigen, and the interaction between the H domain and the L domain form a second antigen binding site specific for a second antigen, and the multispecific Treg-binding molecule is a bispecific bivalent binding molecule. In certain embodiments, the interaction between the A domain and the F domain form a first antigen binding site specific for a first antigen, and the interaction between the H domain and the L domain form a second antigen binding site specific for the first antigen, and the multispecific Treg-binding molecule is a monospecific bivalent binding molecule.

### 6.5.1. Bivalent Bispecific B-Body “BC1”

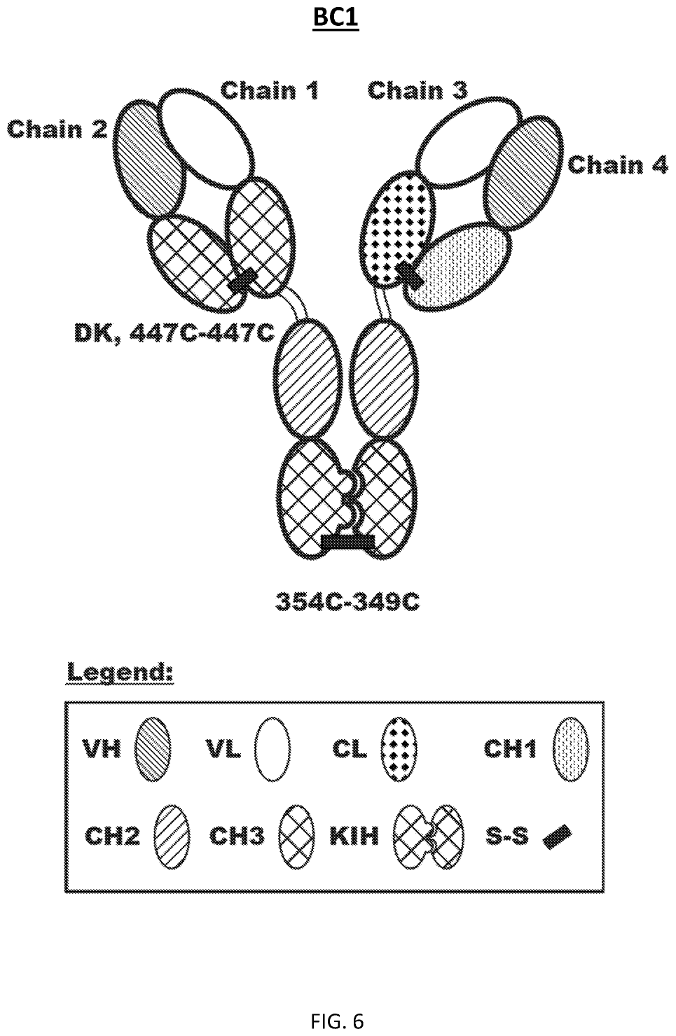

With reference to FIG. 3 and FIG. 6, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a T366K mutation and a C-terminal extension incorporating a KSC tripeptide sequence that is followed by the DKTHT motif (SEQ ID NO: 185) of an IgG1 hinge region, domain D has a human IgG1 CH2 amino acid sequence, and domain E has human IgG1 CH3 amino acid with a S354C and T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a L351D mutation and a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:8, the second polypeptide chain has the sequence SEQ ID NO:9, the third polypeptide chain has the sequence SEQ ID NO:10, and the fourth polypeptide chain has the sequence SEQ ID NO:11.

### 6.5.2. Bivalent Bispecific B-Body “BC6”

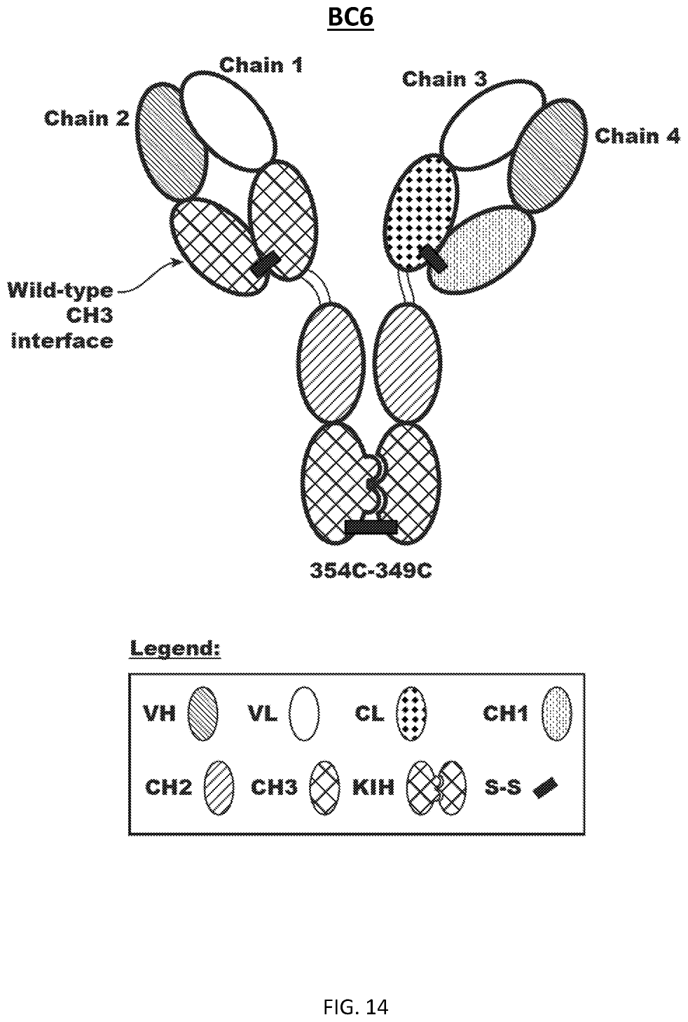

With reference to FIG. 3 and FIG. 14, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a C-terminal extension incorporating a KSC tripeptide sequence that is followed by the DKTHT motif (SEQ ID NO: 185) of an IgG1 hinge region, domain D has a human IgG1 CH2 amino acid sequence, and domain E has human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

### 6.5.3. Bivalent Bispecific B-Body “BC28”

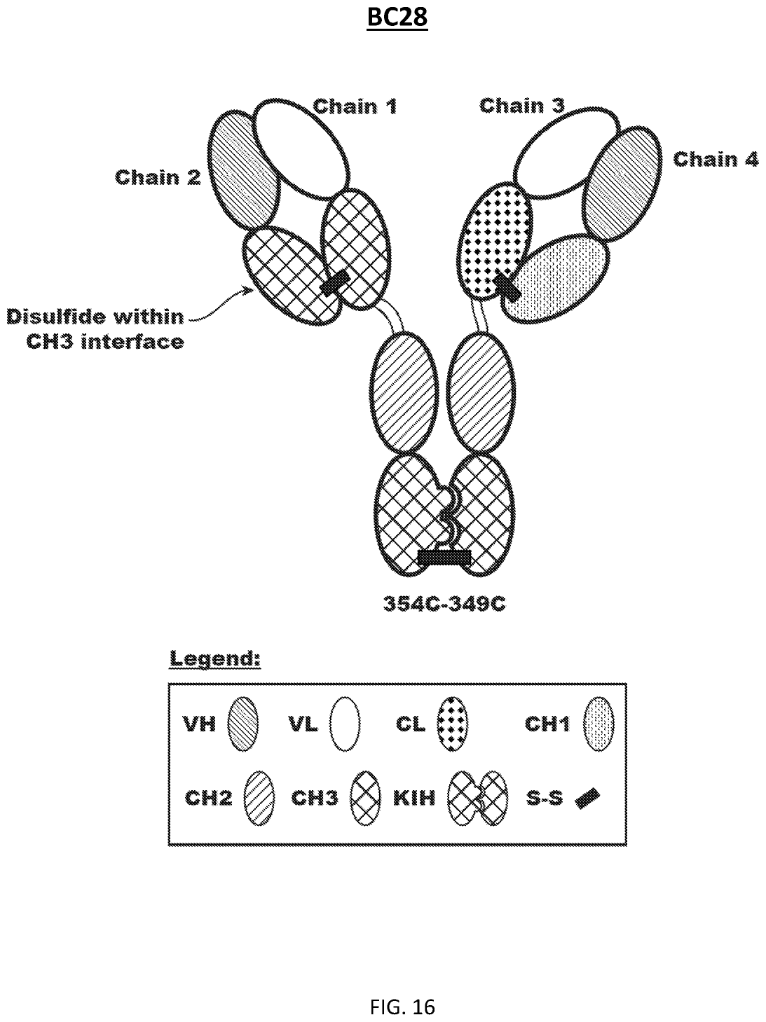

With reference to FIG. 3 and FIG. 16, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation and a C-terminal extension incorporating a PGK tripeptide sequence that is followed by the DKTHT motif (SEQ ID NO: 185) of an IgG1 hinge region, domain D has a human IgG1 CH2 amino acid sequence, and domain E has a human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:24, the second polypeptide chain has the sequence SEQ ID NO:25, the third polypeptide chain has the sequence SEQ ID NO:10, and the fourth polypeptide chain has the sequence SEQ ID NO:11.

### 6.5.4. Bivalent Bispecific B-Body “BC44”

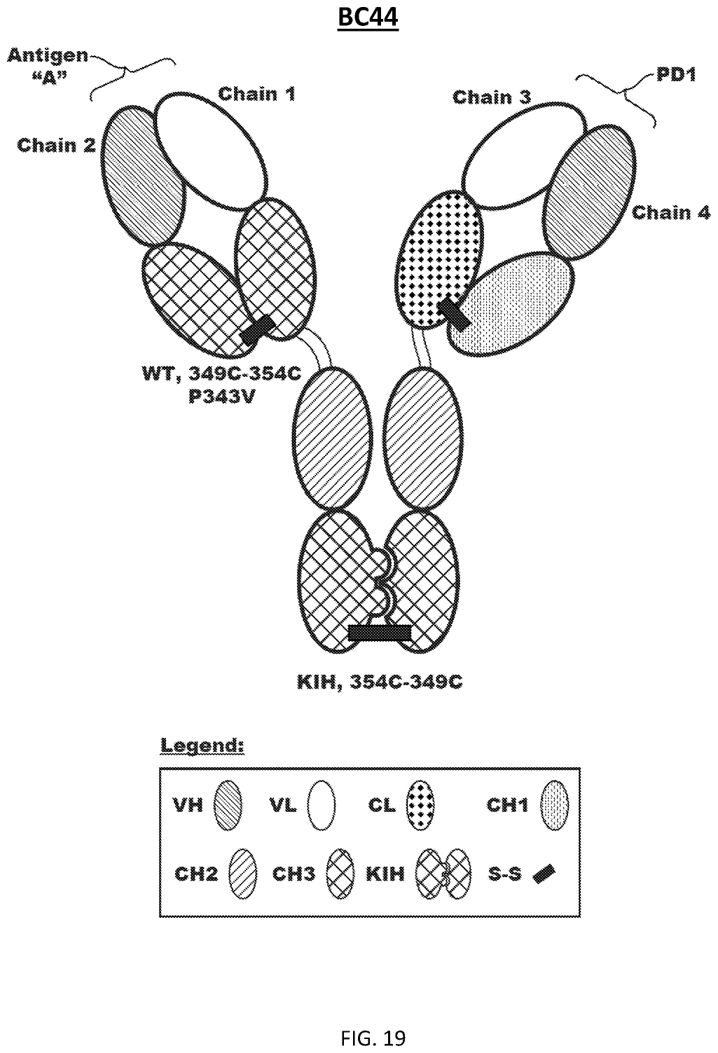

With reference to FIG. 3 and FIG. 19, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation, a P343V mutation, and a C-terminal extension incorporating a PGK tripeptide sequence that is followed by the DKTHT motif (SEQ ID NO: 185) of an IgG1 hinge region, domain D has a human IgG1 CH2 amino acid sequence, and domain E has human IgG1 CH3 amino acid with a S354C mutation and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, T366S, L368A, and aY407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:32, the second polypeptide chain has the sequence SEQ ID NO:25, the third polypeptide chain has the sequence SEQ ID NO:10, and the fourth polypeptide chain has the sequence SEQ ID NO:11.

### 6.5.5. Exemplary Bivalent Binding Molecules with IgA-CH3 Domain Pairs

With reference to FIG. 3, in a series of embodiments, the multispecific Treg-binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a variable region amino acid sequence, domain B has a human IgA CH3 amino acid sequence, domain D has a human IgG1 CH2 amino acid sequence, and domain E has human IgG1 CH3 amino acid sequence; (b) the second polypeptide chain has a domain F and a domain G, wherein the domains are arranged, from N-terminus to C-terminus, in a F-G orientation, and wherein domain F has a variable region amino acid sequence and domain G has a human IgA CH3 amino acid sequence; (c) the third polypeptide chain has a domain H, a domain I, a domain J, and a domain K, wherein the domains are arranged, from N-terminus to C-terminus, in a H-I-J-K orientation, and wherein domain H has a variable region amino acid sequence, domain I has a constant region amino acid sequence, domain J has a human IgG1 CH2 amino acid sequence, and domain K has a human IgG1 CH3 amino acid sequence; (d) the fourth polypeptide chain has a domain L and a domain M, wherein the domains are arranged, from N-terminus to C-terminus, in a L-M orientation, and wherein domain L has a variable region amino acid sequence and domain M has a constant region amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the multispecific Treg-binding molecule. In some embodiments, domain A and domain F form a first antigen binding site specific for a first antigen; and domain H and domain L form a second antigen binding site specific for a second antigen.

In some embodiments, domain A comprises a VH amino acid sequence, domain F comprises a VL amino acid sequence, domain H comprises a VH amino acid sequence, domain I comprises a CH1 amino acid sequence, domain L comprises a VL amino acid sequence, and domain M comprises a CL amino acid sequence. In some embodiments, domain A comprises a first VH amino acid sequence and domain F comprises a first VL amino acid sequence, domain H comprises a second VH amino acid sequence and domain L comprises a second VL amino acid sequence.

In preferred embodiments, domain A comprises a VL amino acid sequence, domain F comprises a VH amino acid sequence, domain H comprises a VL amino acid sequence, domain L comprises a VH amino acid sequence, domain I comprises a CL amino acid sequence, and domain M comprises a CH1 amino acid sequence. In some embodiments, the CL amino acid sequence is a CL-kappa sequence. In some embodiments, domain A comprises a first VL amino acid sequence and domain F comprises a first VH amino acid sequence, domain H comprises a second VL amino acid sequence and domain L comprises a second VH amino acid sequence.

In some embodiments, domain E further comprises a S354C and T366W mutation in the human IgG1 CH3 amino acid sequence. In some embodiments, domain K further comprises a Y349C, a D356E, a L358M, a T366S, a L368A, and a Y407V mutation in the human IgG1 CH3 amino acid sequence.

In some embodiments, domain B comprises a first CH3 linker sequence as described herein that is followed by the DKTHT motif (SEQ ID NO: 185) of an IgG1 hinge region; and domain G comprises a second CH3 linker sequence as described herein. In some embodiments, the first CH3 linker sequence associates with the second CH3 linker sequence by formation of a disulfide bridge between cysteine residues of the first and second CH3 linker sequences.

In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids. In some embodiments, the first CH3 linker is AGC and the second CH3 linker is AGKGSC (SEQ ID NO: 99). In some embodiments, the first CH3 linker is AGKGC (SEQ ID NO: 98) and the second CH3 linker is AGC. In some embodiments, the first CH3 linker is AGKGSC (SEQ ID NO: 99) and the second CH3 linker is AGC. In some embodiments, the first CH3 linker is AGKC (SEQ ID NO: 96) and the second CH3 linker is AGC.

In some embodiments, the multispecific Treg-binding molecule further comprises one or more CH1/CL modifications as described in herein

In some embodiments, the multispecific Treg-binding molecule further comprises a modification that reduces effector function as described herein.

### 6.6. Exemplary Trivalent Binding Molecules

### 6.6.1. Trivalent 1×2 Bispecific B-Body “BC28-1×2”

With reference to Section 6.5.3. and FIG. 26, in a series of embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has the first VL amino acid sequence and domain S has a human IgG1 CH3 amino acid sequence with a Y349C mutation and a C-terminal extension incorporating a PGK tripeptide sequence that is followed by GSGSGS linker peptide (SEQ ID NO: 40) connecting domain S to domain H; (b) the binding molecule further comprises a sixth polypeptide chain, comprising: a domain T and a domain U, wherein the domains are arranged, from N-terminus to C-terminus, in a T-U orientation, and wherein domain T has the first VH amino acid sequence and domain U has a human IgG1 CH3 amino acid sequence with a S354C mutation and a C-terminal extension incorporating a PGK tripeptide sequence; (c) the third and the sixth polypeptides are associated through an interaction between the R and the T domains and an interaction between the S and the U domains to form the binding molecule, and (d) domain R and domain T form a third antigen binding site specific for the first antigen.

In preferred embodiments, the first polypeptide chain has the sequence SEQ ID NO:24, the second polypeptide chain has the sequence SEQ ID NO:25, the third polypeptide chain has the sequence SEQ ID NO:37, the fourth polypeptide chain has the sequence SEQ ID NO:11, and the sixth polypeptide chain has the sequence SEQ ID NO:25.

### 6.6.2. Trivalent 1×2 Trispecific B-Body “BC28-1×1×1a”

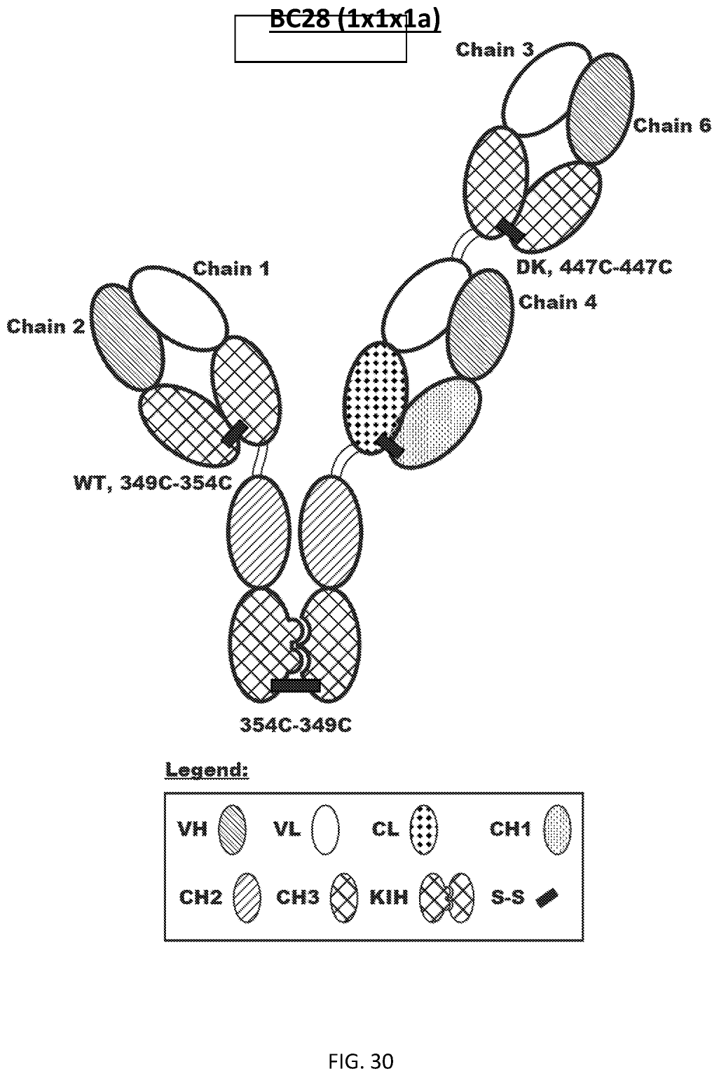

With reference to Section 6.5.3. and FIG. 26 and FIG. 30, in a series of embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a third VL amino acid sequence and domain S has a human IgG1 CH3 amino acid sequence with a T366K mutation and a C-terminal extension incorporating a KSC tripeptide sequence that is followed by GSGSGS linker peptide (SEQ ID NO: 40) connecting domain S to domain H; (b) the binding molecule further comprises a sixth polypeptide chain, comprising: a domain T and a domain U, wherein the domains are arranged, from N-terminus to C-terminus, in a T-U orientation, and wherein domain T has a third VH amino acid sequence and domain U has a human IgG1 CH3 amino acid sequence with a L351D mutation and a C-terminal extension incorporating a GEC amino acid disulfide motif; and (c) the third and the sixth polypeptides are associated through an interaction between the R and the T domains and an interaction between the S and the U domains to form the binding molecule, and (d) domain R and domain T form a third antigen binding site specific for a third antigen.

In preferred embodiments, the first polypeptide chain has the sequence SEQ ID NO:24, the second polypeptide chain has the sequence SEQ ID NO:25, the third polypeptide chain has the sequence SEQ ID NO:45, the fourth polypeptide chain has the sequence SEQ ID NO:11, and the sixth polypeptide chain has the sequence SEQ ID NO: 53.

### 6.6.3. Trivalent Molecules with CH/CL, IgA-CH3 and IgG-CH3

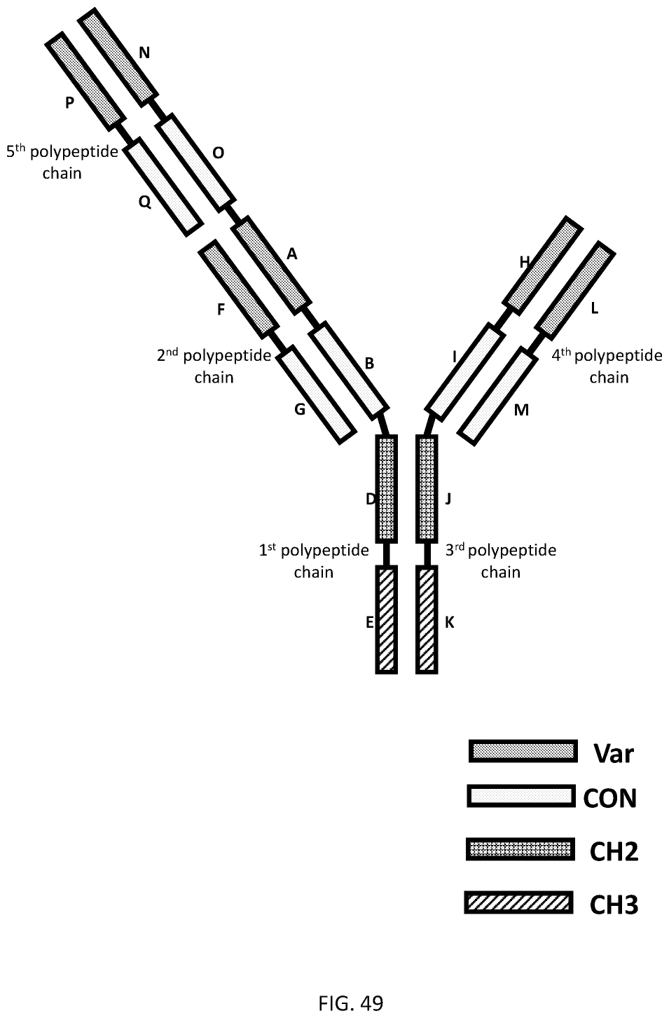

FIG. 49 depicts an exemplary structure of a trivalent binding molecule. With reference to FIG. 49, in various embodiments, the multispecific Treg-binding molecules comprises a first polypeptide chain, a second polypeptide chain, a third polypeptide chain, a fourth polypeptide chain, and a fifth polypeptide chain, wherein (a) the first polypeptide chain comprises a domain N, a domain O, a domain A, a domain B, a domain D, and a domain E, wherein the domains are arranged, from N-terminus to C-terminus, in a N-O-A-B-D-E orientation, and wherein domain N has a variable region amino acid sequence, domain O has a constant region amino acid sequence; domain A has a variable region amino acid sequence, domain B has a constant region amino acid sequence, domain D has a CH2 sequence, and domain E has a CH3 sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, domain J has a CH2 sequence, and domain K has a CH3 sequence; (d) the fourth polypeptide chain comprises a domain L and a domain M, wherein the domains are arranged, from N-terminus to C-terminus, in a L-M orientation, and wherein domain L has a variable region domain amino acid sequence and domain M comprises a constant region amino acid sequence; (e) the fifth polypeptide chain comprises a domain P and a domain Q, wherein the domains are arranged, from N-terminus to C-terminus, in a P-Q orientation, and wherein domain P comprises a variable region amino acid sequence and domain Q comprises a constant region amino acid sequence; (f) domains B and G form a first domain pair of associated constant region domains (“first domain pair”), domains I and M form a second domain pair of associated constant region domains (“second domain pair”), and domains Q and O form a third domain pair of associated constant region domains (“third domain pair”); (g) at least one of the first, second, and third domain pairs is a CH1/CL pair; (h) at least one of the first, second, and third domain pairs is an IgG-CH3/IgG-CH3 pair; and (i) at least one of the first, second, and third domain pairs is an IgA-CH3/IgA-CH3 pair. In some embodiments, the multispecific Treg-binding molecule is formed by domain interactions, including but not necessarily exclusive to interactions of the first domain pair, interactions of the second domain pair, interactions of the third domain pair, an association between domains D and J, and an association between domains E and K.

In some embodiments, domains A and F associate to form a first antigen binding site; domains H and L associate to form a second antigen binding site; and domains N and P associate to form a third antigen binding site.

In some embodiments, the first domain pair is an IgA-CH3/IgA-CH3 pair, the second domain pair is an IgG-CH3/IgG-CH3 pair, and the third domain pair is a CH1/CL pair. In some embodiments, the first domain pair is an IgA-CH3/IgA-CH3 pair, the second domain pair is a CH1/CL pair, and the third domain pair is an IgG-CH3/IgG-CH3 pair. In some embodiments, the first domain pair is an IgG-CH3/IgG-CH3 pair, the second domain pair is an IgA-CH3/IgA-CH3 pair, and the third domain pair is a CH1/CL pair. In some embodiments, the first domain pair is an IgG-CH3/IgG-CH3 pair, the second domain pair is a CH1/CL pair, and the third domain pair is an IgA-CH3/IgA-CH3 pair. In some embodiments, the first domain pair is a CH1/CL pair, the second domain pair is an IgA-CH3/IgA-CH3 pair, and the third domain pair is an IgG-CH3/IgG-CH3 pair. In some embodiments, the first domain pair is a CH1/CL pair, the second domain pair is an IgG-CH3/IgG-CH3 pair, and the third domain pair is an IgA-CH3/IgA-CH3 pair.

In some embodiments, an association between domains A and F form a first antigen binding site, an association between domains H and L form a second antigen binding site, and an association between domains N and P form a third antigen binding site. In some embodiments, the first antigen binding site, the second antigen binding site, and the third antigen binding site bind to the same antigen. In some embodiments, the first antigen binding site and second antigen binding site bind to a first antigen, and the third antigen binding site binds to a second antigen. In some embodiments, the first antigen binding site and third antigen binding site bind to a first antigen, and the second antigen binding site binds to a second antigen. In some embodiments, the first antigen binding site binds to a first antigen, and the second antigen binding site and third antigen binding site binds to a second antigen. In some embodiments, the first antigen binding site binds to a first antigen, the second antigen binding site binds to a second antigen, and the third antigen binding site binds to a third antigen.

In some embodiments, domains E and K comprise a knob-in-hole orthogonal modification, as described herein.

In some embodiments, the CH1/CL pair comprises one or more CH1/CL orthogonal modifications as described herein.

In some embodiments, the IgG-CH3/IgG-CH3 pair comprises one or more orthogonal modifications described herein.

In some embodiments, the Fc region of the multispecific Treg-binding molecule comprises one or more mutations in CH2 which reduce effector function. Such mutations are described herein.

### 6.7. Exemplary Binding Molecules with CH1/CL Modifications

In some embodiments, a multispecific Treg-binding molecule described herein comprises one or more orthogonal CH1/CL modifications described above. In some embodiments, the multispecific Treg-binding molecule, generally comprising an architecture as described in FIG. 3, 21, 26, 30 or 34, comprises one or more orthogonal modifications in one or more CH1/CL domain-associated pairs. In some cases, the one or more CH1/CL domain-associated pairs comprising the one or more orthogonal CH1/CL modifications have non-identical sets of CH1/CL orthogonal modifications as compared to the other CH1/CL domain-associated pairs. For example, the multispecific Treg-binding molecule may comprise one or more orthogonal modifications in a CH1/CL pair of one arm of the Y-shaped structure. For another example, a multispecific Treg-binding molecule having a general Y-shaped architecture as described in FIG. 3, 21, 26, 30 or 34, may comprise one or more orthogonal modifications in CH1/CL pairs in both arms of the Y-shaped structure, wherein each CH1/CL pair comprises non-identical CH1/CL orthogonal modifications as compared to the other CH1/CL pairs. In some embodiments, a multispecific Treg-binding molecule having a general architecture as described in FIG. 21, 26, 30 or 34, comprises at least a first CH1/CL pair and a second CH1/CL pair in one arm of the Y-shaped structure, wherein the first CH1/CL pair comprise non-identical CH1/CL orthogonal modifications as compared to the second CH1/CL pair.

In some embodiments, the first CH1/CL pair comprises a first charged-pair orthogonal mutation and the second CH1/CL pair comprises a second charged-pair orthogonal mutation, in the same amino acid position, wherein the second charged-pair orthogonal mutation is oppositely charged as compared to the first charged-pair orthogonal mutation. In some embodiments, the first CH1/CL pair comprises a first charged-pair orthogonal mutation that introduces a positively-charged residue in an amino acid position of CH1 and a negatively-charged residue in the orthogonal CL position, and the second CH1/CL pair comprises a second charged-pair orthogonal mutation that introduces a negatively-charged residue in the same amino acid position of CH1 and a positively-charged residue in the orthogonal CL position. In some embodiments, the first CH1/CL pair comprises a first charged-pair orthogonal mutation that introduces a negatively-charged residue in an amino acid position of CH1 and a positively-charged residue in the orthogonal CL position, and the second CH1/CL pair comprises a second charged-pair orthogonal mutation that introduces a positively-charged residue in the same amino acid position of CH1 and a negatively-charged residue in the orthogonal CL position. For example, the first CH1/CL pair may comprise a CH1 domain comprising a G166D mutation and a CL domain comprising a N138K mutation, and the second CH1/CL pair may comprise a CH1 domain comprising a G166K mutation and a CL domain comprising a N138D mutation. For other example, the first CH1/CL pair may comprise a CH1 domain comprising a G166K mutation and a CL domain comprising a N138D mutation, and the second CH1/CL pair may comprise a CH1 domain comprising a G166D mutation and a CL domain comprising a N138K mutation. In some embodiments, the first or second CH1/CL pair may further comprise an engineered disulfide bridge described in Table 12 herein. In some embodiments, the engineered disulfide bridge comprises an orthogonal L128C mutation in CH1 and F118C mutation in CL.

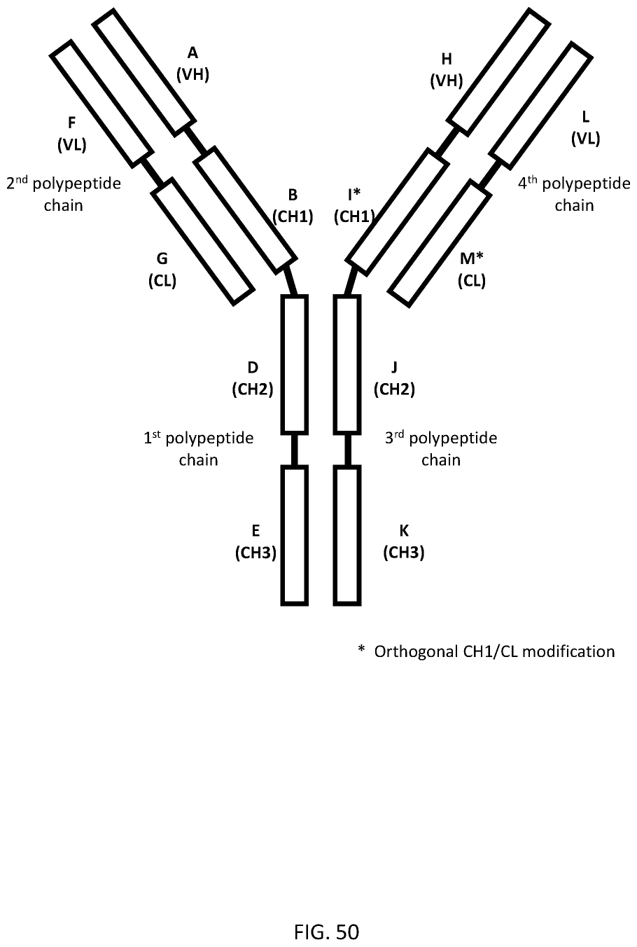

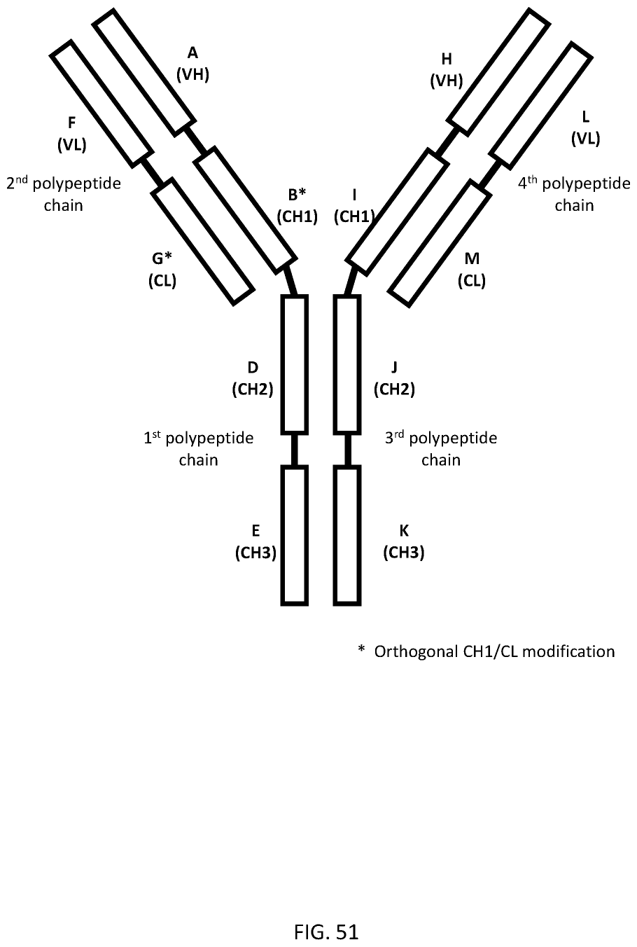

In some embodiments, the multispecific Treg-binding molecule is structured as described in FIG. 3, wherein domain B comprises CH1 and domain G comprises CL, thereby forming a first CH1/CL associated domain pair; domain I comprises CH1 and domain M comprises CL, thereby forming a second CH1/CL associated domain pair, and wherein the first and second CH1/CL pairs each comprise non-identical sets of CH1/CL orthogonal modifications. In some embodiments, the first CH1/CL pair comprises an L128C/F118C engineered disulfide bridge and a G166D/N138K orthogonal charged-pair mutation, and the second CH1/CL pair comprises a G166K/N138D orthogonal charged-pair mutation. In some embodiments, the first CH1/CL pair comprises an L128C/F118C engineered disulfide bridge and a G162K/N138D orthogonal charged-pair mutation, and the second CH1/CL pair comprises a G162D/N138K orthogonal charged-pair mutation. In some cases, the multispecific Treg-binding molecule further comprises a knob-in-hole orthogonal modification described herein. Exemplary binding molecule are depicted in FIGS. 50-52.

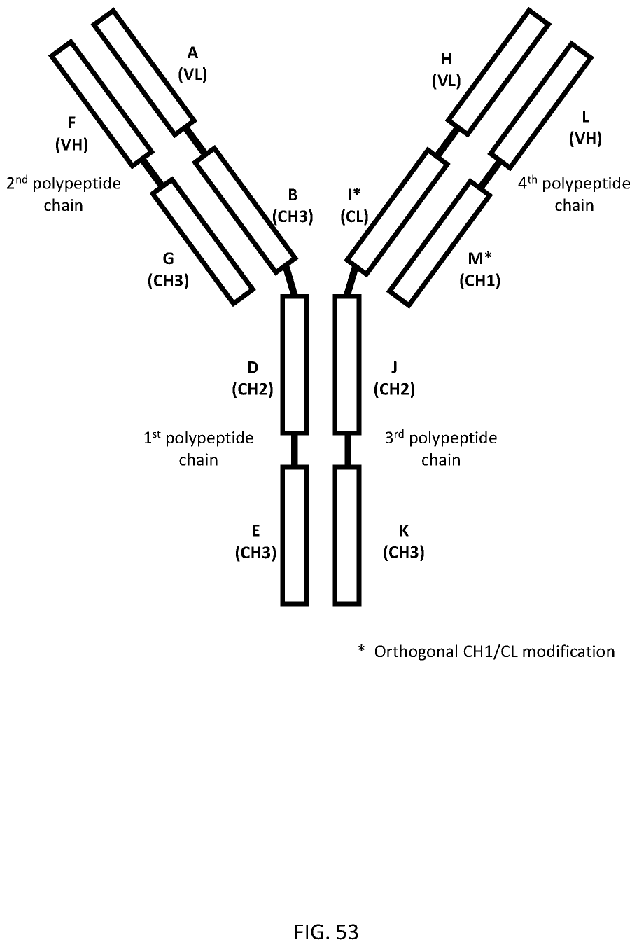

In some embodiments, the multispecific Treg-binding molecule is a B-Body™. B-Body™ binding molecules are described in International Patent Application No. PCT/US2017/057268, which is hereby incorporated by reference in its entirety. In some embodiments, the multispecific Treg-binding molecule is structured as described in FIG. 3, wherein A is VL, B is CH3, D is CH2, E is CH3, F is VH, G is CH3, H is VL, I is CL, J is CH2, K is CH3, L is VH, and M is CH1, and wherein domain pair I and M comprise one or more CH1/CL orthogonal modification as described in Tables X1 and X2. An exemplary binding molecule is depicted in FIG. 53.

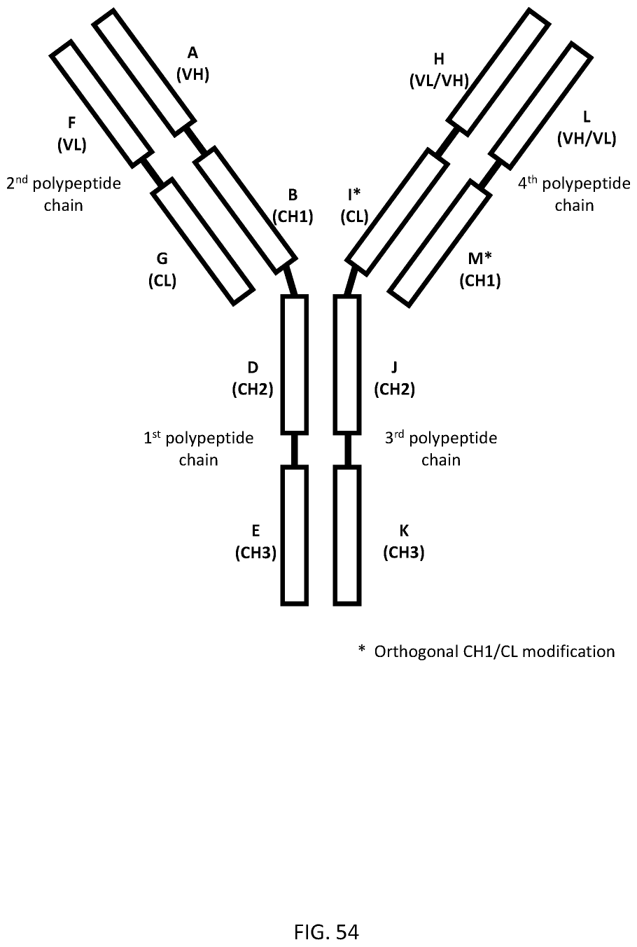

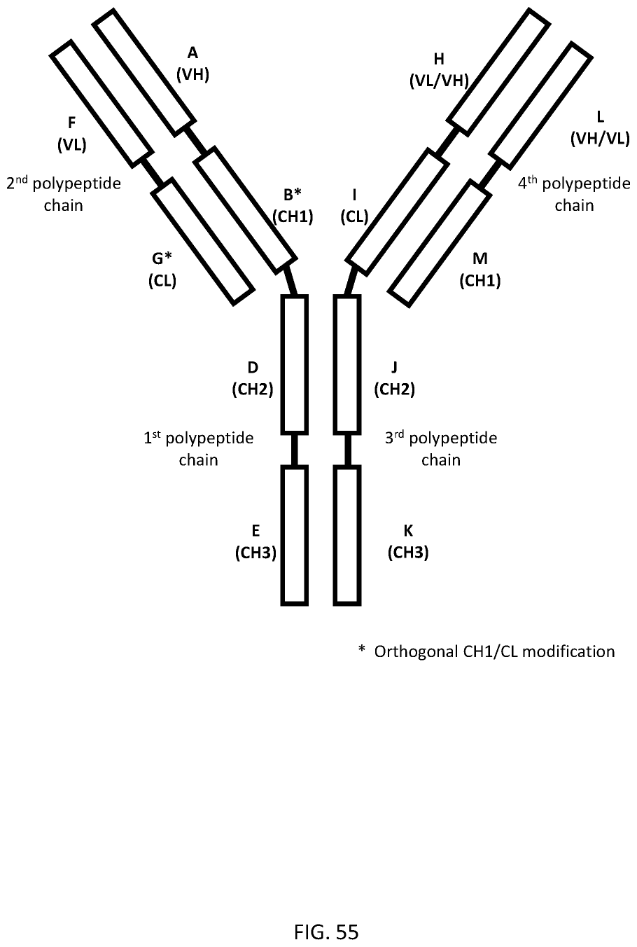

In some embodiments, the multispecific Treg-binding molecule is a CrossMab™ antibody comprising one or more CH1/CL orthogonal modifications described in Tables X1 and X2. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230, each of which is hereby incorporated by reference in its entirety for all that it teaches. In some embodiments, the multispecific Treg-binding molecule is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other; wherein constant domains CL and CH1 of) the light chain and heavy chain of an antibody specifically binding to the first or second antigen comprises one or more CH1/CL orthogonal modifications described in Tables X1 and X2. In some embodiments, the multispecific Treg-binding molecule is structured as described in FIG. 3, wherein A is VH, B is CH1, D is CH2, E is CH3, F is VL, G is CL, H is VL or VH, I is CL, J is CH2, K is CH3, L is VH or VL, and M is CH1, and wherein at least one of domain pairs B and G, and I and M, comprise one or more CH1/CL orthogonal modification as described in Tables X1 and X2. In some cases, domain pair B and G comprise one or more CH1/CL orthogonal modifications and domain pair I and M does not. In other cases, domain pair I and M comprise one or more CH1/CL orthogonal modifications and domain pair B and G does not. In yet other cases, both domain pair B and G and domain pair I and M comprise non-identical sets of one or more CH1/CL orthogonal modifications. Exemplary binding molecules are depicted in FIGS. 54 and 55.

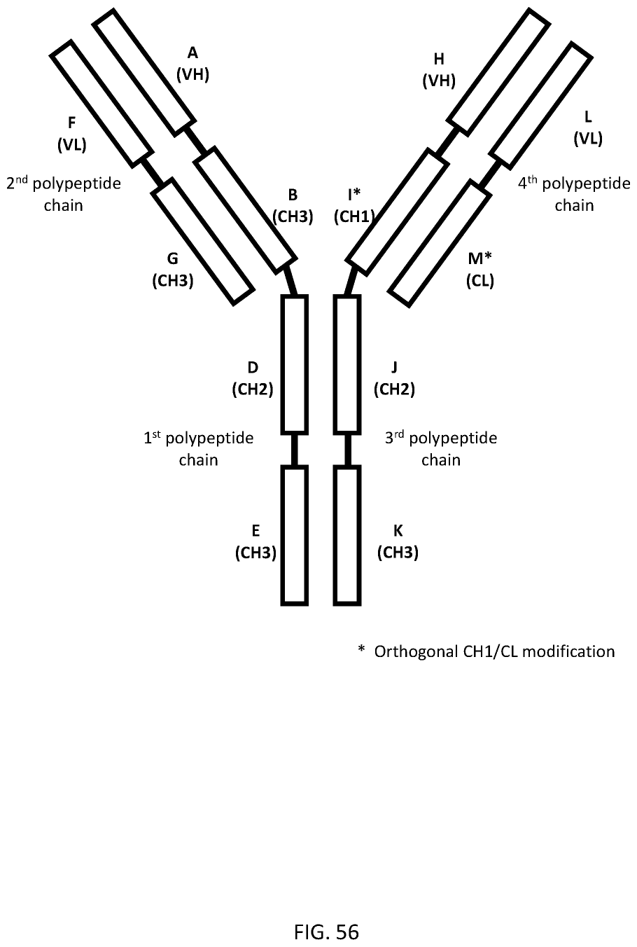

In some embodiments, the multispecific Treg-binding molecule is an antibody having a general architecture described in U.S. Pat. No. 8,871,912 and WO2016087650. In some embodiments, the multispecific Treg-binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair, wherein the antibody further comprises an additional light chain composed of VL-CL and an additional heavy chain composed of VH-CH1-CH2-CH3, and wherein the CH1 and CL comprise one or more CH1/CL orthogonal modifications described in Tables X1 and X2. In some embodiments, the multispecific Treg-binding molecule is structured as described in FIG. 3, wherein A is VH, B is CH3, D is CH2, E is CH3, F is VL, G is CH3, H is VH, I is CH1, J is CH2, K is CH3, L is VL, and M is CL, and wherein domain pair I and M comprise one or more CH1/CL orthogonal modifications as described in Tables X1 and X2. An exemplary binding molecule is depicted in FIG. 56.

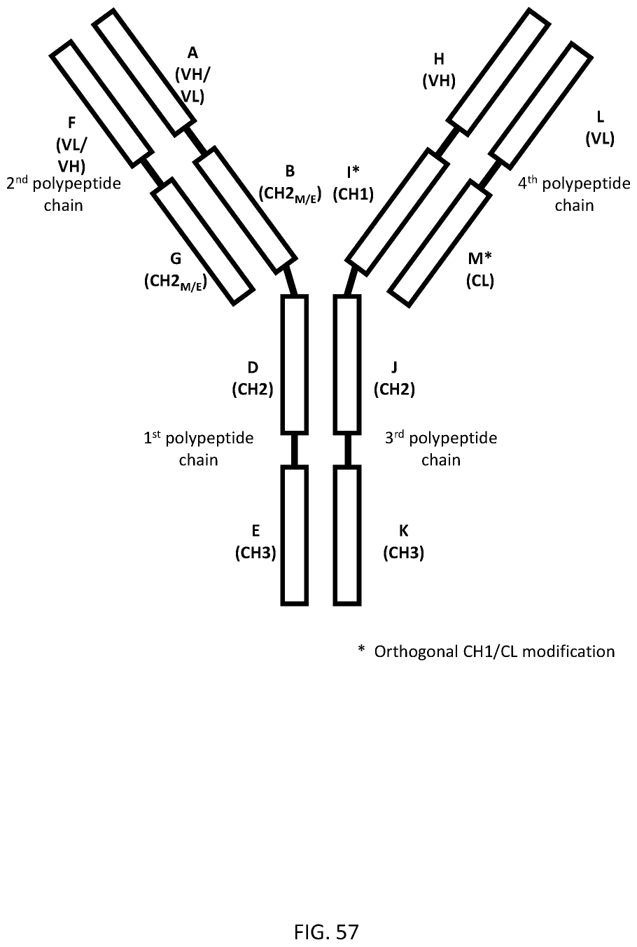

In some embodiments, the multispecific Treg-binding molecule is as described in WO2017011342. In some embodiments, the multispecific Treg-binding molecule is structured as described in FIG. 3, wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL, and wherein domain pair I and M comprise one or more CH1/CL orthogonal modification as described in Tables X1 and X2. An exemplary binding molecule is depicted in FIG. 57.

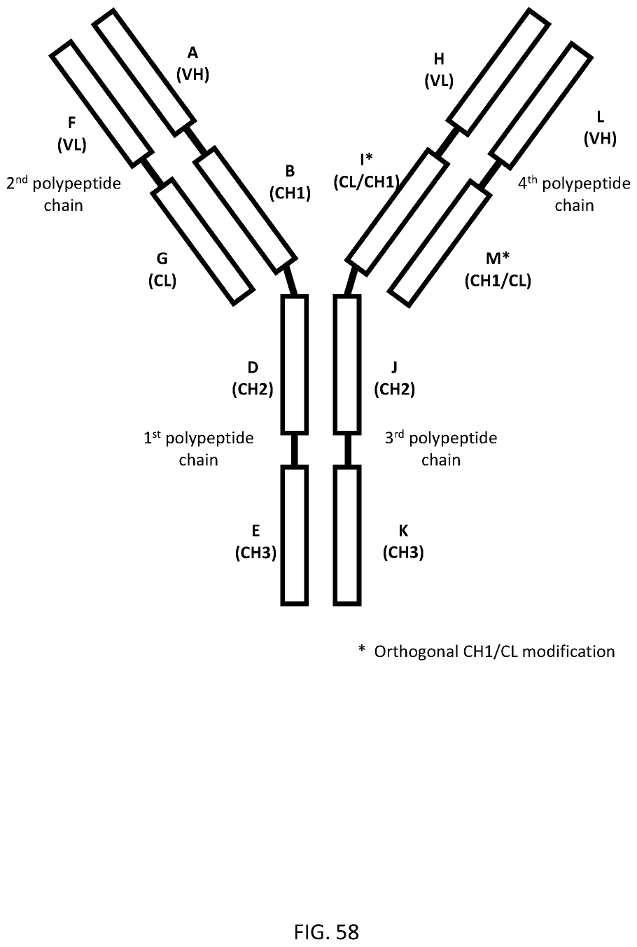

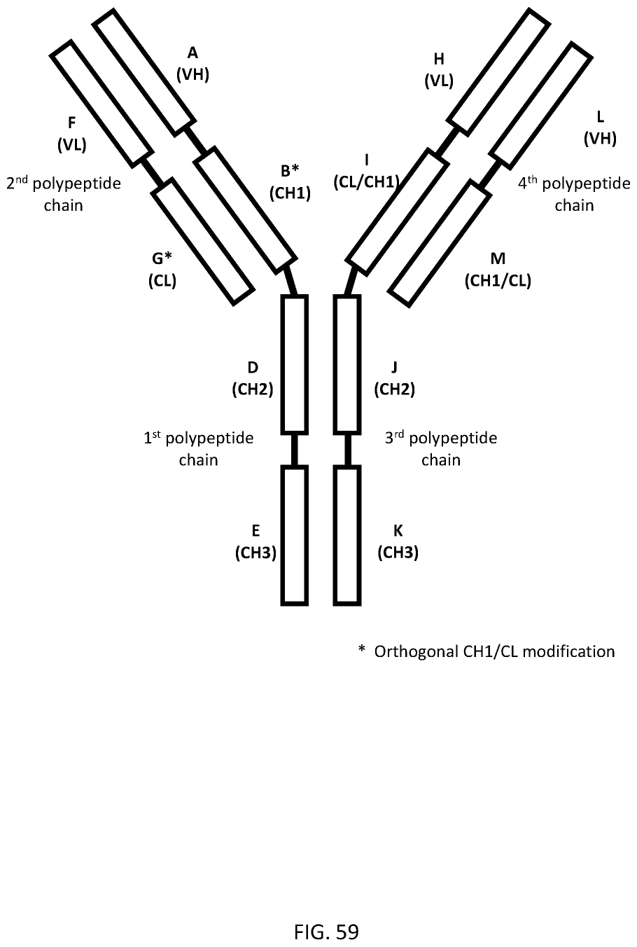

In some embodiments, the multispecific Treg-binding molecule is as described in WO2006093794. In some embodiments, the multispecific Treg-binding molecule is structured as described in FIG. 3, wherein A is VH, B is CH1, D is CH2, E is CH3, F is VL, G is CL, H is VL, I is CL or CH1, J is CH2, K is CH3, L is VH, and M is CH1 or CL, and wherein at least one of domain pairs B and G, and I and M, comprise one or more CH1/CL orthogonal modification as described in Tables X1 and X2. In some cases, domain pair B and G comprise one or more CH1/CL orthogonal modifications and domain pair I and M does not. In other cases, domain pair I and M comprise one or more CH1/CL orthogonal modifications and domain pair B and G does not. In yet other cases, both domain pair B and G and domain pair I and M comprise non-identical sets of one or more CH1/CL orthogonal modifications. Exemplary binding molecules are depicted in FIGS. 58 and 59.

‘It is contemplated that binding molecules comprising one or more CH1/CL modifications described herein may further comprise modifications of one or more other domains. For example, any of the multispecific Treg-binding molecules comprising one or more CH1/Cl modifications, described herein may further comprise knob-in-hole mutations, described herein, mutations that reduce effector function, as described herein, and/or IgA-CH3 domain paring as described herein.

### 6.8. Other Binding Molecule Platforms

The various antibody platforms described above are not limiting. The antigen binding sites described herein, including specific CDR subsets, can be formatted into any binding molecule platform including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches. Furthermore, any of the modifications and mutations described herein, can be formatted into any binding molecule platform described herein.

### 6.9. Further Modifications

In a further series of embodiments, the multispecific Treg-binding molecule has additional modifications.

### 6.9.1. CH1 and CL Orthogonal Modifications

In certain embodiments, the CH1 sequence and the CL sequences separately comprise respectively orthogonal modifications in endogenous CH1 and CL sequences.

“Orthogonal modifications” or synonymously “orthogonal mutations” as described herein are one or more engineered mutations in an amino acid sequence of an antibody domain that alter the affinity of binding of a first domain having orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail below. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail herein.

In certain embodiments, the CH1 sequence and the CL sequence of the CH1/CL pair separately comprise respectively orthogonal modifications in endogenous CH1 and CL sequences. In other embodiments, one sequence of the CH1/CL pair comprises at least one modification while the other sequence of the CH1/CL pair does not comprise a modification in the respectively orthogonal amino acid position.

A CH1/CL orthogonal modification may affect the CH1/CL domain pairing via an interaction between a modified residue in the CH1 domain and a corresponding modified or unmodified residue in the CL domain.

It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the multispecific Treg-binding molecule having a portion of the CH1 sequences described herein can be bound by the CH1 binding reagent.

Exemplary CH1/CL orthogonal modifications: engineered disulfide bridges.

Some embodiments of a CH1/CL orthogonal modification comprise an engineered disulfide bridge between engineered cysteines in CH1 and CL. Such engineered disulfide bridges may stabilize an interaction between the polypeptide comprising the modified CH1 and the polypeptide comprising the corresponding modified CL.

An orthogonal CH1/CL modification comprising an engineered disulfide bridge can comprise, by way of example only, a CH1 domain having an engineered cysteine at position 128, 129, 138, 141, 168, or 171, as numbered by the EU index. Such an orthogonal CH1/CL modification comprising an engineered disulfide bridge may further comprise, by way of example only, a CL domain having an engineered cysteine at position 116, 118, 119, 164, 162, or 210, as numbered by the EU index.

For example, a CH1/CL orthogonal modification may be selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the CH1/CL orthogonal modification comprises an engineered cysteine at position 141 of the CH1 sequence and position 118 of the CL sequence, as numbered by the EU index.

In some embodiments, the CH1/CL orthogonal modification comprises an engineered cysteine at position 168 of the CH1 sequence and position 164 of the CL sequence, as numbered by the EU index. In some embodiments, the CH1/CL orthogonal modification comprises an engineered cysteine at position 128 of the CH1 sequence and position 118 of the CL sequence, as numbered by the EU index. In some embodiments, the CH1/CL orthogonal modification comprises an engineered cysteine at position 171 of the CH1 sequence and position 162 of the CL sequence, as numbered by the EU index. In some embodiments, the CL sequence is a CL-lambda sequence. In preferred embodiments, the CL sequence is a CL-kappa sequence. In some embodiments, the engineered cysteines are at position 128 of the CH1 sequence and position 118 of the CL Kappa sequence, as numbered by the EU index.

Table 12 below provides exemplary CH1/CL orthogonal modifications comprising an engineered disulfide bridge between CH1 and CL, numbered according to the EU index.

TABLE 12
exemplary CH1/CL engineered disulfide bridges

| CH1 mutation | CL mutation |
|---|---|
| A141C | F118C |
| H168C | T164C |
| L128C | F118C |
| P171C | S162C |

In a series of preferred embodiments, the mutations that provide non-endogenous (engineered) cysteine amino acids are a F118C mutation in the CL sequence with a corresponding A141C in the CH1 sequence, or a F118C mutation in the CL sequence with a corresponding L128C in the CH1 sequence, a T164C mutation in the CL sequence with a corresponding H168C mutation in the CH1 sequence, or a S162C mutation in the CL sequence with a corresponding P171C mutation in the CH1 sequence, as numbered by the Eu index.

CH1/CL Orthogonal Modifications: Charged-Pair Mutations

In a variety of embodiments, the orthogonal modifications in the CL sequence and the CH1 sequence are charge-pair mutations. As used herein, charge-pair mutations are amino acid substitutions that affect the charge of a residue in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches.

In some cases, the CH1/CL charge-pair mutations are a N138K mutation in the CL sequence with a corresponding G166D in the CH1 sequence, or a N138D mutation in the CL sequence with a corresponding G166K in the CH1 sequence, as numbered by the Eu index. In some embodiments, the charge-pair mutations are a P127E mutation in CH1 sequence with a corresponding E123K mutation in the corresponding Cl sequence. In some embodiments, the charge-pair mutations are a P127K mutation in CH1 sequence with a corresponding E123 (not mutated) in the corresponding CL sequence.

Table 13 below provides exemplary CH1/CL orthogonal charged-pair modifications.

TABLE 13
exemplary CH1/CL orthogonal charged-pair modifications

| CH1 mutation | CL mutation |
|---|---|
| G166D | N138K |
| G166D | N138D |
| G166K | N138K |
| G166K | N138D |
| P127E | E123K |
| P127E | No mutation (E123) |
| P127K | E123K |
| P127K | No mutation (E123) |

### 6.9.1.1. Combinations of CH1/CL Orthogonal Modifications

In certain embodiments, the CH1 and CL domains of a single CH1/CL pair separately contain two or more respectively orthogonal modifications in endogenous CH1 and CL sequences. For instance, the CH1 and CL sequence may contain a first orthogonal modification and a second orthogonal modification in the endogenous CH1 and CL sequences. The two or more respectively orthogonal modifications in endogenous CH1 and CL sequences can be selected from any of the CH1/CL orthogonal modifications described herein.

In some embodiments, the first orthogonal modification is an orthogonal charge-pair mutation, and the second orthogonal modification is an orthogonal engineered disulfide bridge. In some embodiments, the first orthogonal modification is an orthogonal charge-pair mutation as described in Table 13, and the additional orthogonal modification comprise an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the first orthogonal modification is an orthogonal charge-pair mutation as described in Table 13, and the additional orthogonal modification comprise an engineered disulfide bridge as described in Table 12. In some embodiments, the first orthogonal modification comprises an L128C mutation in the CH1 sequence and an F118C mutation in the CL sequence, and the second orthogonal modification comprises a modification of residue 166 in the same CH1 sequence and a modification of residue 138 in the same CL sequence as described herein. In some embodiments, the first orthogonal modification comprises an L128C mutation in the CH1 sequence and an F118C mutation in the CL sequence, and the second orthogonal modification comprises a G166D mutation in the CH1 sequence and a N138K mutation in the CL sequence. In some embodiments, the first orthogonal modification comprises an L128C mutation in the CH1 sequence and an F118C mutation in the CL sequence, and the second orthogonal modification comprises a G166K mutation in the CH1 sequence and a N138D mutation in the CL sequence.

### 6.9.2. Antibody-Drug Conjugates

In various embodiments, the multispecific Treg-binding molecule is conjugated to a therapeutic agent (i.e. drug) to form a multispecific Treg-binding molecule-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the multispecific Treg-binding molecule through a linker peptide, as discussed in more detail herein.

Methods of preparing antibody-drug conjugates (ADCs) that can be adapted to conjugate drugs to the multispecific Treg-binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### 6.9.3. Additional Binding Moieties

In various embodiments, the multispecific Treg-binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the multispecific Treg-binding molecule. In certain embodiments, the one or more additional binding moieties are specific for the same antigen or epitope of the ABSs within the multispecific Treg-binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the multispecific Treg-binding molecule using in vitro methods including, but not limited to, reactive chemistry and affinity tagging systems, as discussed in more detail herein. In certain embodiments, the one or more additional binding moieties are attached to the multispecific Treg-binding molecule through Fc-mediated binding (e.g. Protein A/G). In certain embodiments, the one or more additional binding moieties are attached to the multispecific Treg-binding molecule using recombinant DNA techniques, such as encoding the nucleotide sequence of the fusion product between the multispecific Treg-binding molecule and the additional binding moieties on the same expression vector (e.g. plasmid).

### 6.9.4. Functional/Reactive Groups

In various embodiments, the multispecific Treg-binding molecule has modifications that comprise functional groups or chemically reactive groups that can be used in downstream processes, such as linking to additional moieties (e.g. drug conjugates and additional binding moieties, as discussed in more detail herein) and downstream purification processes.

In certain embodiments, the modifications are chemically reactive groups including, but not limited to, reactive thiols (e.g. maleimide based reactive groups), reactive amines (e.g. N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g. HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178.), the entirety of which is hereby incorporated by reference for all it teaches.

### 6.9.5. Reduced Effector Function

In certain embodiments, the multispecific Treg-binding molecule has one or more engineered mutations in an amino acid sequence of an antibody domain that reduce the effector functions generally associated with antibody binding. Effector functions include, but are not limited to, cellular functions that result from an Fc receptor binding to an Fc portion of an antibody, such as antibody dependent cellular cytotoxicity (ADCC), complement fixation (e.g. Clq binding), antibody dependent cellular-mediated phagocytosis (ADCP), opsonization. Engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each herein incorporated by reference in their entirety.

In specific embodiments, the multispecific Treg-binding molecule has one or more engineered mutations in an amino acid sequence of an antibody domain that reduce binding of an Fc portion of the binding molecule by FcR receptors. In some embodiments, the FcR receptors are FcRγ receptors. In some embodiments, the FcR receptors are FcRγR1 receptors. In some embodiments, the FcR receptors are FcγRIIa receptors. In some embodiments, the FcR receptors are FcγRIIIA receptors.

In specific embodiments, the one or more engineered mutations that reduce effector function are mutations in a CH2 domain of an antibody. In various embodiments, the one or more engineered mutations comprise a mutation at position L234 of the CH2 domain. In some embodiments, the mutation at position L234 is L234A. In some embodiments, the mutation at position L234 is L234G. In various embodiments, the one or more engineered mutations comprise a mutation at position L235 of the CH2 domain. In some embodiments, the mutation at position L235 is L235A. In some embodiments, the mutation at position L235 is L235G. In various embodiments, the one or more engineered mutations comprise mutations at positions L234 and L235 of the CH2 domain. In some embodiments, the mutations at positions L234 and L235 of the CH2 domain are L234A and L235A. In some embodiments, the mutations at positions L234 and L235 of the CH2 domain are L234G and L235G.

In various embodiments, the one or more engineered mutations comprise a mutation at position P329 of the CH2 domain. In some embodiments, the mutation at position P329 of the CH2 domain is P329A. In some embodiments, the mutation at position P329 of the CH2 domain is P329G. In some embodiments, the mutation at position P329 of the CH2 domain is P329K.

In other embodiments, the one or more engineered mutations are at positions L234, L235, and P329 of the CH2 domain. In particular embodiments, the one or more engineered mutations are L234A, L235A, and P329A of the CH2 domain. In particular embodiments, the one or more engineered mutations are L234A, L235A, and P329G of the CH2 domain. In preferred embodiments, the one or more engineered mutations are L234A, L235A, and P329K of the CH2 domain. In particular embodiments, the one or more engineered mutations are L234G, L235G, and P329A of the CH2 domain. In particular embodiments, the one or more engineered mutations are L234G, L235G, and P329G of the CH2 domain. In particular embodiments, the one or more engineered mutations are L234G, L235G, and P329K of the CH2 domain.

### 6.9.5.1. Exemplary Binding Molecules with Reduced Effector Function

In some embodiments, the multispecific Treg-binding molecule is structured as described in FIG. 3, wherein domains A and H are VH, domains B and I are CH1, domains D and J are CH2, domains E and K are CH3, domains F and L are VL, and domains G and M are CL, and wherein at least one CH2 sequence of the multispecific Treg-binding molecule comprises one or more mutations that reduce effector function as described herein. In some cases, the one or more CH2 mutations comprise L234A, L235A, and P329K. In some cases, the one or more CH2 mutations comprise L234A, L235A, and P329A. In some cases, the one or more CH2 mutations comprise L234A, L235A, and P329G. In some cases, the one or more CH2 mutations comprise L234G, L235G, and P329A. In some cases, the one or more CH2 mutations comprise L234G, L235G, and P329G. In some cases, the one or more CH2 mutations comprise L234G, L235G, and P329K. In some cases, the multispecific Treg-binding molecule further comprises one or more CH1/CL orthogonal modifications as described herein. In some cases, the multispecific Treg-binding molecule further comprises a knob-in-hole orthogonal modification described herein.

In some embodiments, the multispecific Treg-binding molecule is a B-Body™. B-Body™ binding molecules are described in International Patent Application No. PCT/US2017/057268. In some embodiments, the multispecific Treg-binding molecule is structured as described in FIG. 3, wherein A is VL, B is CH3, D is CH2, E is CH3, F is VH, G is CH3, H is VL, I is CL, J is CH2, K is CH3, L is VH, and M is CH1, and wherein at least one CH2 sequence of the multispecific Treg-binding molecule comprises one or more mutations that reduce effector function as described herein. In some embodiments, the multispecific Treg-binding molecule is a trivalent binding molecule as described herein, wherein at least one CH2 sequence of the multispecific Treg-binding molecule comprises one or more mutations that reduce effector function as described herein.

In some embodiments, the multispecific Treg-binding molecule is a CrossMab antibody comprising one or more CH1/CL orthogonal modifications described in Tables 12 and 13. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230, each of which is hereby incorporated by reference in its entirety for all that it teaches. In some embodiments, the multispecific Treg-binding molecule is a bivalent, bispecific antibody, comprising: a) the light chain and heavy chain of an antibody specifically binding to a first Treg cell surface antigen; and b) the light chain and heavy chain of an antibody specifically binding to a Treg cell surface second antigen, wherein constant domains CL and CH1 from the antibody specifically binding to a second Treg cell surface antigen are replaced by each other; and wherein the multispecific Treg-binding molecule comprises one or more mutations that reduce effector function as described herein. In some embodiments, the multispecific Treg-binding molecule is structured as described in FIG. 3, wherein A is VH, B is CH1, D is CH2, E is CH3, F is VL, G is CL, H is VL or VH, I is CL, J is CH2, K is CH3, L is VH or VL, and M is CH1, and wherein the multispecific Treg-binding molecule comprises one or more mutations that reduce effector function as described herein.

In some embodiments, the multispecific Treg-binding molecule is an antibody having a general architecture described in U.S. Pat. No. 8,871,912 and WO2016087650. In some embodiments, the multispecific Treg-binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair, wherein the antibody further comprises an additional light chain composed of VL-CL and an additional heavy chain composed of VH-CH1-CH2-CH3, and wherein the multispecific Treg-binding molecule comprises one or more mutations that reduce effector function as described herein. In some embodiments, the multispecific Treg-binding molecule is structured as described in FIG. 3, wherein A is VH, B is CH3, D is CH2, E is CH3, F is VL, G is CH3, H is VH, I is CH1, J is CH2, K is CH3, L is VL, and M is CL, and wherein the multispecific Treg-binding molecule comprises one or more mutations that reduce effector function as described herein.

In some embodiments, the multispecific Treg-binding molecule is as described in WO2017011342. In some embodiments, the multispecific Treg-binding molecule is structured as described in FIG. 3, wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL, and wherein the multispecific Treg-binding molecule comprises one or more mutations that reduce effector function as described herein.

In some embodiments, the multispecific Treg-binding molecule is as described in WO2006093794. In some embodiments, the multispecific Treg-binding molecule is structured as described in FIG. 3, wherein A is VH, B is CH1, D is CH2, E is CH3, F is VL, G is CL, H is VL, I is CL or CH1, J is CH2, K is CH3, L is VH, and M is CH1 or CL and wherein the multispecific Treg-binding molecule comprises one or more mutations that reduce effector function as described herein.

It is contemplated that binding molecules comprising one or more mutations that reduce effector function, described herein may further comprise modifications of one or more other domains. For example, any of the multispecific Treg-binding molecules in this section may further comprise knob-in-hole mutations, described herein and/or CH1/CL orthogonal modifications as described herein.

It is to be understood that any of the modifications described in this application are not limited to the exemplary embodiments listed above, but are instead applicable to any binding molecule platform, including but not limited to the binding molecule platforms described herein. In addition, it is contemplated that multispecific Treg-binding molecules may include any combination of the modifications described herein.

### 6.10. Methods of Purification

A method of purifying a multispecific Treg-binding molecule comprising a B-body platform is provided herein.

In a series of embodiments, the method comprises the steps of: i) contacting a sample comprising the multispecific Treg-binding molecule with a CH1 binding reagent, wherein the multispecific Treg-binding molecule comprises at least a first, a second, a third, and a fourth polypeptide chain associated in a complex, wherein the complex comprises at least one CH1 domain, or portion thereof, and wherein the number of CH1 domains in the complex is at least one fewer than the valency of the complex, and wherein the contacting is performed under conditions sufficient for the CH1 binding reagent to bind the CH1 domain, or portion thereof; and ii) purifying the complex from one or more incomplete complexes, wherein the incomplete complexes do not comprise the first, the second, the third, and the fourth polypeptide chain.

In a typical, naturally occurring, antibody, two heavy chains are associated, each of which has a CH1 domain as the second domain, numbering from N-terminus to C-terminus. Thus, a typical antibody has two CH1 domains. CH1 domains are described in more detail herein. In a variety of the multispecific Treg-binding molecules described herein, the CH1 domain typically found in the protein has been substituted with another domain, such that the number of CH1 domains in the protein is effectively reduced. In a non-limiting illustrative example, the CH1 domain of a typical antibody can be substituted with a CH3 domain, generating an antigen-binding protein having only a single CH1 domain.

Binding molecules can also refer to molecules based on antibody architectures that have been engineered such that they no longer possess a typical antibody architecture. For example, an antibody can be extended at its N or C terminus to increase the valency (described in more detail herein) of the antigen-binding protein, and in certain instances the number of CH1 domains is also increased beyond the typical two CH1 domains. Such molecules can also have one or more of their CH1 domains substituted, such that the number of CH1 domains in the protein is at least one less than the valency of the antigen-binding protein. In some embodiments, the number of CH1 domains that are substituted by other domains generates a multispecific Treg-binding molecule having only a single CH1 domain. In other embodiments, the number of CH1 domains substituted by another domain generates a multispecific Treg-binding molecule having two or more CH1 domains, but at least one fewer than the valency of the antigen-binding protein. In particular embodiments, where a multispecific Treg-binding molecule has two or more CH1 domains, the multiple CH1 domains can all be in the same polypeptide chain. In other particular embodiments, where a multispecific Treg-binding molecule has two or more CH1 domains, the multiple CH1 domains can be a single CH1 domain in multiple copies of the same polypeptide chain present in the complete complex.

### 6.10.1. CH1 Binding Reagents

In exemplary non-limiting methods of purifying binding molecules, a sample comprising the multispecific Treg-binding molecules is contacted with CH1 binding reagents. CH1 binding reagents, as described herein, can be any molecule that specifically binds a CH1 epitope. The various CH1 sequences that provide the CH1 epitope are described in more detail herein, and specific binding is described in more detail herein.

In some embodiments, CH1 binding reagents are derived from immunoglobulin proteins and have an antigen binding site (ABS) that specifically binds the CH1 epitope. In particular embodiments, the CH1 binding reagent is an antibody, also referred to as an “anti-CH1 antibody.” The anti-CH1 antibody can be derived from a variety of species. In particular embodiments, the anti-CH1 antibody is a mammalian antibody, including, but not limited to mouse, rat, hamster, rabbit, camel, donkey, goat, and human antibodies. In specific embodiments, the anti-CH1 antibody is a single-domain antibody. Single-domain antibodies, as described herein, have a single variable domain that forms the ABS and specifically binds the CH1 epitope. Exemplary single-domain antibodies include, but are not limited to, heavy chain antibodies derived from camels and sharks, as described in more detail in international application WO 2009/011572, herein incorporated by reference for all it teaches. In a preferred embodiment, the anti-CH1 antibody is a camel derived antibody (also referred to as a “camelid antibody”). Exemplary camelid antibodies include, but are not limited to, human IgG-CH1 CaptureSelect™ (ThermoFisher, #194320010) and human IgA-CH1 (ThermoFisher, #194311010). In some embodiments, the anti-CH1 antibody is a monoclonal antibody. Monoclonal antibodies are typically produced from cultured antibody-producing cell lines. In other embodiments, the anti-CH1 antibody is a polyclonal antibody, i.e., a collection of different anti-CH1 antibodies that each recognize the CH1 epitope. Polyclonal antibodies are typically produced by collecting the antibody containing serum of an animal immunized with the antigen of interest, or fragment thereof, here CH1.

In some embodiments, CH1 binding reagents are molecules not derived from immunoglobulin proteins. Examples of such molecules include, but are not limited to, aptamers, peptoids, and affibodies, as described in more detail in Perret and Boschetti (Biochimie, February 2018, Vol 145:98-112),which is hereby incorporated by reference in its entirety for all that it teaches.

### 6.10.2. Solid Supports

In exemplary non-limiting methods of purifying binding molecules, the CH1 binding reagent can be attached to a solid support in various embodiments of the invention. Solid supports, as described herein, refers to a material to which other entities can be attached or immobilized, e.g., the CH1 binding reagent. Solid supports, also referred to as “carriers,” are described in more detail in international application WO 2009/011572.

In specific embodiments, the solid support comprises a bead or nanoparticle. Examples of beads and nanoparticles include, but are not limited to, agarose beads, polystyrene beads, magnetic nanoparticles (e.g., Dynabeads™, ThermoFisher), polymers (e.g., dextran), synthetic polymers (e.g., Sepharose™), or any other material suitable for attaching the CH1 binding reagent. In particular embodiments, the solid support is modified to enable attachment of the CH1 binding reagent. Example of solid support modifications include, but are not limited to, chemical modifications that form covalent bonds with proteins (e.g., activated aldehyde groups) and modifications that specifically pair with a cognate modification of a CH1 binding reagent (e.g., biotin-streptavidin pairs, disulfide linkages, polyhistidine-nickel, or “click-chemistry” modifications such as azido-alkynyl pairs).

In certain embodiments, the CH1 binding reagent is attached to the solid support prior to the CH1 binding reagent contacting the multispecific Treg-binding molecules, herein also referred to as an “anti-CH1 resin.” In some embodiments, anti-CH1 resins are dispersed in a solution. In other embodiments, anti-CH1 resins are “packed” into a column. The anti-CH1 resin is then contacted with the multispecific Treg-binding molecules and the CH1 binding reagents specifically bind the multispecific Treg-binding molecules.

In other embodiments, the CH1 binding reagent is attached to the solid support after the CH1 binding reagent contacts the multispecific Treg-binding molecules. As a non-limiting illustration, a CH1 binding reagent with a biotin modification can be contacted with the multispecific Treg-binding molecules, and subsequently the CH1 binding reagent/binding molecule mixture can be contacted with streptavidin modified solid support to attach the CH1 binding reagent to the solid support, including CH1 binding reagents specifically bound to the multispecific Treg-binding molecules.

In methods wherein the CH1 binding reagents are attached to solid supports, in a variety of embodiments, the bound binding molecules are released, or “eluted,” from the solid support forming an eluate having the multispecific Treg-binding molecules. In some embodiments, the bound binding molecules are released through reversing the paired modifications (e.g., reduction of the disulfide linkage), adding a reagent to compete off the multispecific Treg-binding molecules (e.g., adding imidazole that competes with a polyhistidine for binding to nickel), cleaving off the multispecific Treg-binding molecules (e.g., a cleavable moiety can be included in the modification), or otherwise interfering with the specific binding of the CH1 binding reagent for the multispecific Treg-binding molecule. Methods that interfere with specific binding include, but are not limited to, contacting binding molecules bound to CH1 binding reagents with a low-pH solution. In preferred embodiment, the low-pH solution comprises 0.1 M acetic acid pH 4.0. In other embodiments, the bound binding molecules can be contacted with a range of low-pH solutions, i.e., a “gradient.”

### 6.10.3. Further Purification

In some embodiments of the exemplary non-limiting methods, a single iteration of the method using the steps of contacting the multispecific Treg-binding molecules with the CH1 binding reagents, followed by eluting the multispecific Treg-binding molecules, is used to purify the multispecific Treg-binding molecules from the one or more incomplete complexes. In particular embodiments, no other purifying step is performed. In other embodiments, one or more additional purification steps are performed to further purify the multispecific Treg-binding molecules from the one or more incomplete complexes. The one or more additional purification steps include, but are not limited to, purifying the multispecific Treg-binding molecules based on other protein characteristics, such as size (e.g., size exclusion chromatography), charge (e.g., ion exchange chromatography), or hydrophobicity (e.g., hydrophobicity interaction chromatography). In a preferred embodiment, an additional cation exchange chromatograph is performed. Additionally, the multispecific Treg-binding molecules can be further purified repeating contacting the multispecific Treg-binding molecules with the CH1 binding reagents as described above, as well as modifying the CH1 purification method between iterations, e.g., using a step elution for the first iteration and a gradient elution for a subsequent elution.

### 6.10.4. Assembly and Purity of Complexes

In the embodiments of the present invention, at least four distinct polypeptide chains associate together to form a complete complex, i.e., the multispecific Treg-binding molecule. However, incomplete complexes can also form that do not contain the at least four distinct polypeptide chains. For example, incomplete complexes may form that only have one, two, or three of the polypeptide chains. In other examples, an incomplete complex may contain more than three polypeptide chains, but does not contain the at least four distinct polypeptide chains, e.g., the incomplete complex inappropriately associates with more than one copy of a distinct polypeptide chain. The method of the invention purifies the complex, i.e., the completely assembled binding molecule, from incomplete complexes.

Methods to assess the efficacy and efficiency of the purification steps are well known to those skilled in the art and include, but are not limited to, SDS-PAGE analysis, ion exchange chromatography, size exclusion chromatography, and mass spectrometry. Purity can also be assessed according to a variety of criteria. Examples of criterion include, but are not limited to: 1) assessing the percentage of the total protein in an eluate that is provided by the completely assembled binding molecule, 2) assessing the fold enrichment or percent increase of the method for purifying the desired products, e.g., comparing the total protein provided by the completely assembled binding molecule in the eluate to that in a starting sample, 3) assessing the percentage of the total protein or the percent decrease of undesired products, e.g., the incomplete complexes described above, including determining the percent or the percent decrease of specific undesired products (e.g., unassociated single polypeptide chains, dimers of any combination of the polypeptide chains, or trimers of any combination of the polypeptide chains). Purity can be assessed after any combination of methods described herein. For example, purity can be assessed after a single iteration of using the anti-CH1 binding reagent, as described herein, or after additional purification steps, as described in more detail herein. The efficacy and efficiency of the purification steps may also be used to compare the methods described using the anti-CH1 binding reagent to other purification methods known to those skilled in the art, such as Protein A purification.

### 6.11. Methods of Manufacturing

The multispecific Treg-binding molecules described herein can readily be manufactured by expression using standard cell free translation, transient transfection, and stable transfection approaches currently used for antibody manufacture. In specific embodiments, Expi293 cells (ThermoFisher) can be used for production of the multispecific Treg-binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

As further described in the Examples below, the expressed proteins can be readily separated from undesired proteins and protein complexes using a CH1 affinity resin, such as the CaptureSelect CH1 resin and provided protocol from ThermoFisher. Other purification strategies include, but are not limited to, use of Protein A, Protein G, or Protein A/G reagents. Further purification can be affected using ion exchange chromatography as is routinely used in the art.

### 6.12. Pharmaceutical Compositions

In another aspect, pharmaceutical compositions are provided that comprise a multispecific Treg-binding molecule as described herein.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), which is incorporated by reference in its entirety.

The one or more pharmaceutical excipients can include an anti-foaming agent. Any suitable anti-foaming agent may be used. In some aspects, the anti-foaming agent is selected from an alcohol such as, e.g., octyl alcohol, capryl alcohol, ethyl alcohol, 2-ethyl-hexanol, or oleyl alcohol; an ether, an oil, a silicone, a surfactant, a wax, and combinations thereof. In some aspects, the anti-foaming agent is selected from ethylene bis stearamide, a mineral oil, a vegetable oil, an ester wax, a fatty alcohol wax, a paraffin wax, a long chain fatty alcohol, a fatty acid ester, a fatty acid soap, a silicon glycol, fluorosilicone, polyethylene glycol-polypropylene glycol copolymer, polydimethylsiloxane-silicon dioxide, sorbitan trioleate, dimethicone, simethicone, and combinations thereof.

The one or more pharmaceutical excipients can include a cosolvent. Illustrative examples of cosolvents include butylene glycol, ethanol, dimethylacetamide, glycerin, poly(ethylene) glycol, propylene glycol, and combinations thereof.

The one or more pharmaceutical excipients can include a buffer. Illustrative examples of buffers include acetate, borate, carbonate, guar gum, lactate, phosphate, citrate, hydroxide, diethanolamine, glycine, monoethanolamine, methionine, malate, monosodium glutamate, and combinations thereof.

The one or more pharmaceutical excipients can include a carrier or filler. Exemplary carriers or fillers include, e.g., lactose, maltodextrin, mannitol, sorbitol, chitosan, stearic acid, xanthan gum, guar gum, and combinations thereof.

The one or more pharmaceutical excipients can include a surfactant. Exemplary surfactants include -alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, vitamin E polyethylene (glycol) succinate, and combinations thereof.

The one or more pharmaceutical excipients can include an anti-caking agent. Illustrative examples of anti-caking agents include calcium phosphate (tribasic), hydroxymethyl cellulose, hydroxypropyl cellulose, magnesium oxide, and combinations thereof.

The one or more pharmaceutical excipients can include a solvent. Exemplary solvents include, e.g., saline solutions, such as sterile isotonic saline solutions, dextrose solutions, sterile water for injection, and the like.

Other excipients that may be used in the pharmaceutical composition can include, by way of example only, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, stabilizing agents, sugars, and combinations thereof.

The pharmaceutical composition can be in particulate form, such as microparticles or nanoparticles. Microparticles and nanoparticles may be formed from any suitable material, such as a polymer or a lipid. For example, the microparticle or nanoparticle can be a liposome.

The pharmaceutical composition can be in an anhydrous form. Anydrous forms can be prepared using anhydrous or low moisture containing ingredients and low moisture or low humidity conditions. An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In various embodiments, the pharmaceutical composition comprises the multispecific Treg-binding molecule at a concentration of 0.1 mg/ml-100 mg/ml. In specific embodiments, the pharmaceutical composition comprises the multispecific Treg-binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the multispecific Treg-binding molecule at a concentration of more than 10 mg/ml. In certain embodiments, the multispecific Treg-binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the multispecific Treg-binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

### 6.13. Methods

### 6.13.1. Methods of Treatment

In another aspect, methods of treatment are provided, the methods comprising administering a multispecific Treg-binding molecule as described herein to a subject in an amount effective to treat the subject. In some embodiments, the multispecific Treg-binding molecule directs a therapeutic agent to a target Treg in a subject. Exemplary therapeutic agents are described herein. In some embodiments, the therapeutic agent suppresses activity of a target Treg in a subject. The target Treg is preferably a tumor-associated Treg.

In specific embodiments, the specific targeting of the tumor-associated Tregs using a multispecific Treg-binding molecule described herein results in suppressing activity of tumor-associated Tregs. In some embodiments, the specific targeting of the tumor-associated Tregs using a multispecific Treg-binding molecule described herein results in depletion (e.g. killing) of the tumor-associated Tregs. In preferred embodiments, the depletion of the tumor-associated Tregs is mediated by an antibody-drug conjugate (ADC) modification, such as an antibody conjugated to a toxin, as discussed in more detail herein.

In some embodiments, a multispecific Treg binding molecule of the present disclosure is used to treat a proliferative disease. The proliferative disease may be, e.g., a cancer. The cancer may be a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In some embodiments, the cancer may be a neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Also contemplated herein is a method of diagnosis or theranosis, comprising detecting tumor-associated Tregs in a subject or biological sample obtained from the subject, using a multispecific Treg binding molecule disclosed herein.

A multispecific Treg binding molecule of the present disclosure may be administered to a subject for the treatment of, e.g., cancer, autoimmunity, transplantation rejection, post-traumatic immune responses, graft-versus-host disease, ischemia, stroke, and infectious diseases, for example by targeting viral antigens, such as gp120 of HIV.

The multispecific Treg binding molecule may be administered to a subject per se or as a pharmaceutical composition. Exemplary pharmaceutical compositions are described herein.

The multispecific Treg binding molecule may be administered to a subject by any route known in the art. For example, the multispecific Treg binding molecule may be administered to a human subject via, e.g., intraarterial, intramuscular, intradermal, intravenous, intraperitoneal, intranasal, parenteral, pulmonary, subcutaneous administration, topical, oral, sublingual, intratumoral, peritumoral, intralesional, intrasynovial, intrathecal, intra-cerebrospinal, or perilesional administration.

The multispecific Treg binding molecule may be administered as a bolus or by continuous infusion over a period of time. In some embodiments, the multispecific Treg binding molecule can be administered to achieve a steady-state concentration of the binding molecule in blood or serum of the subject. The steady-state concentration can be determined by measurement according to techniques available to those of skill or can be based on the physical characteristics of the subject such as height, weight and age. In certain embodiments, treatment can be initiated with one or more loading doses of the multispecific Treg binding molecule or composition provided herein followed by one or more maintenance doses. The loading dose may be a higher dose than subsequent doses.

It is understood that the route of administration and the dosing regimen can be determined and or adjusted by a clinician, based on one or more factors such as, e.g., the condition or disease to be treated, the severity of the disease, physical characteristics of the subject, e.g., height, weight, age, general health, prior medical history, and the like.

The multispecific Treg binding molecule may optionally be administered with one or more additional agents useful to prevent or treat a disease or disorder. The effective amount of such additional agents may depend on, e.g., the amount of the multispecific Treg binding molecule present in the formulation, the type of disorder or treatment, and the other factors known in the art or described herein.

### 6.13.2. Methods of Selecting a Candidate Multispecific Treg Binding Molecule

Also provided herein is a method of selecting a candidate multispecific Treg binding molecule.

A set of candidate multispecific Treg binding molecules may be generated by any methods known in the art.

In an exemplary embodiment, a phage display library is screened for a first set of variants that bind to the first Treg cell surface antigen, and is also screened for a second set of variants that bind to the second Treg cell surface antigen. In some embodiments, the first and second sets of variants are selected to bind to the first or second Treg cell surface antigens with a Kd of 100 nM or higher. Variable regions of the first and second sets of variants are then formatted into a scaffold multispecific binding molecule structure in a combinatorial fashion to create a set of candidate multispecific Treg binding molecules.

In another exemplary embodiment, variable regions of known monospecific antibodies to the first and second Treg cell surface antigen are formatted into a scaffold multispecific binding molecule structure in a combinatorial fashion to create a set of candidate multispecific Treg binding molecules.

In yet another exemplary embodiment, host animals are immunized with the first or second Treg cell surface antigen, optionally with an adjuvant. The host animal can be, e.g., a mouse, rabbit, rat, goat, guinea pig, donkey, or chicken. Candidate parent antibodies which selectively bind to the first or second Treg cell surface antigens may be isolated from the serum of the host animals. The candidate parent molecules may be further screened for parent molecules that bind to the first or second Treg cell surface antigens with a Kd of 100 nM or higher. Variable regions of these parent molecules that bind to the first or second Treg cell surface antigens are then formatted into a scaffold multispecific binding molecule structure in a combinatorial fashion to create a set of candidate multispecific Treg binding molecules.

Other methods of generating candidate molecules include hybridoma, yeast display, mammalian display, ribosome display, RNA display, and the like.

The set of candidate multispecific Treg binding molecules may be screened for a multispecific Treg-binding molecule that selectively binds a tumor-associated Treg using any methods known in the art.

In an exemplary embodiment, screening may be performed by assessing binding avidity of a candidate binding molecule to (i) a first population of cells comprising the first Treg cell surface antigen, (ii) a second population of cells comprising the second Treg cell surface antigen but not the first Treg cell surface antigen, and (iii) a third population of cells comprising the first and second Treg cell surface antigens; and selecting the candidate as a multispecific Treg-binding molecule if the binding avidity to the third population of cells is at least 2× greater than avidity to the first or second population of cells.

In some embodiments, the method comprises selecting the candidate as a multispecific Treg-binding molecule if the binding avidity to the third population of cells is at least 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or more than 100× greater than avidity to the first or second population of cells. In preferred embodiments, a candidate is selected as a multispecific Treg-binding molecule if the binding avidity to the third population of cells is at least 5× or greater than 5× than avidity to the first or second population of cells.

It is to be understood that a population of cells can include any number of cells. For instance, the first, second, or third populations of cells can include just one cell, or can include more than one cell.

The first, second, and third populations of cells can be aliquoted to one or more chambers, wells, or other compartments.

The aliquots or compartments of cells can be contacted with different concentrations of the candidate binding molecules. For example, the different concentrations of the candidate binding molecule can follow a serial dilution curve.

Binding avidity of the candidate binding molecules to the cells can be assessed by any methods known in the art. By way of example only, binding affinity may be assessed by direct or indirect immunofluorescence, surface plasmon resonance (SPR), Bio-Layer Interferometry (BLI), radioimmunoassay (RIA), flow cytometry, enzyme-linked immunosorbent assay (ELISA) or other methods.

### 6.14. Examples

The following examples are provided by way of illustration, not limitation.

### 6.14.1. Methods

Non-limiting, illustrative methods for the purification of the various antigen-binding proteins and their use in various assays are described in more detail below.

### 6.14.1.1. Expi293 Expression

The various antigen-binding proteins tested were expressed using the Expi293 transient transfection system according to manufacturer's instructions. Briefly, four plasmids coding for four individual chains were mixed at 1:1:1:1 mass ratio, unless otherwise stated, and transfected with ExpiFectamine 293 transfection kit to Expi 293 cells. Cells were cultured at 37° C. with 8% CO2, 100% humidity and shaking at 125 rpm. Transfected cells were fed once after 16-18 hours of transfections. The cells were harvested at day 5 by centrifugation at 2000 g for 10 munities. The supernatant was collected for affinity chromatography purification.

### 6.14.1.2. Protein A and Anti-CH1 Purification

Cleared supernatants containing the various antigen-binding proteins were separated using either a Protein A (ProtA) resin or an anti-CH1 resin on an AKTA Purifier FPLC. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1 M acetic acid pH 4.0. The elution was monitored by absorbance at 280 nm and the elution peaks were pooled for analysis. For anti-CH1 purification, a 1 mL CaptureSelect™ XL column (ThermoFisher) was equilibrated with PBS. The sample was loaded onto the column at 5 ml/min. The sample was eluted using 0.1 M acetic acid pH 4.0. The elution was monitored by absorbance at 280 nm and the elution peaks were pooled for analysis.

### 6.14.1.3. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 95° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

### 6.14.1.4. IEX Chromatography

Samples containing the various separated antigen-binding proteins were analyzed by cation exchange chromatography for the ratio of complete product to incomplete product and impurities. Cleared supernatants were analyzed with a 5-ml MonoS (GE Lifesciences) on an AKTA Purifier FPLC. The MonoS column was equilibrated with buffer A 10 mM MES pH 6.0. The samples were loaded onto the column at 2 ml/min. The sample was eluted using a 0-30% gradient with buffer B (10 mM MES pH 6.0, 1 M sodium chloride) over 6 CV. The elution was monitored by absorbance at 280 nm and the purity of the samples were calculated by peak integration to identify the abundance of the monomer peak and contaminants peaks. The monomer peak and contaminant peaks were separately pooled for analysis by SDS-PAGE as described above.

### 6.14.1.5. Analytical SEC Chromatography

Samples containing the various separated antigen-binding proteins were analyzed by analytical size exclusion chromatography for the ratio of monomer to high molecular weight product and impurities. Cleared supernatants were analyzed with an industry standard TSK G3000SWxl column (Tosoh Bioscience) on an Agilent 1100 HPLC. The TSK column was equilibrated with PBS. 25 μL of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

### 6.14.1.6. Mass Spectrometry

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were both tested in the reduced format to specifically identify each chain by molecular weight. Samples were all tested under non-reducing conditions to identify the molecular weights of all complexes in the samples. Mass spec analysis was used to identify the number of unique products based on molecular weight.

### 6.14.1.7. Antibody Discovery by Phage Display

Phage display of human Fab libraries are carried out using standard protocols. Phage clones are screened for the ability to bind an antigen of interest by phage ELISA using standard protocols. Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pill. The light chain and heavy chain are expressed as a separate polypeptides, and the light chain and heavy chain-pIII fusion assemble in the bacterial periplasm, where the redox potential enables disulfide bond formation, to form the antibody containing the candidate ABS.

The library was created using sequences derived from a specific human heavy chain variable domain (VH3-23) and a specific human light chain variable domain (Vk-1). Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:74) and light chain (SEQ ID NO:75) scaffolds used in the library are listed below, where a lower case “x” represents CDR amino acids that were varied to create the library, and bold italic represents the CDR sequences that were constant.

Diversity was created through Kunkel mutagenesis using primers to introduce diversity into VL CDR3 and VH CDR1 (H1), CDR2 (H2) and CDR3 (H3) to mimic the diversity found in the natural antibody repertoire, as described in more detail in Kunkel, T A (PNAS Jan. 1, 1985. 82 (2) 488-492), herein incorporated by reference in its entirety. Briefly, single-stranded DNA were prepared from isolated phage using standard procedures and Kunkel mutagenesis carried out. Chemically synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to ˜5×1012 phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD600-0.6. After 20 minutes, infected cells were sub-cultured in 25 mL 2×YT+Ampicillin and M13K07 helper phage and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for the target antigen. Target-specific enrichment was confirmed using polyclonal and monoclonal phage ELISA. DNA sequencing was used to determine isolated Fab clones containing a candidate ABS.

To measure binding affinity in discovery campaigns, the VL and VH domains are formatted into a bivalent monospecific native human full-length IgG1 architecture and immobilized to a biosensor on an Octet (Pall ForteBio) biolayer interferometer. Soluble antigens are then added to the system and binding measured.

For experiments performed using the B-Body format, VL variable regions of individual clones are formatted into Domain A and/or H, and VH region into Domain F and/or L of a bivalent 1×1 B-Body “BC1” scaffold shown below and with reference to FIG. 3.

“BC1” Scaffold:
1^{st} polypeptide chain (SEQ ID NO:78)
   Domain A=Antigen 1 B-Body Domain A/H Scaffold (SEQ ID NO:76)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:79):
   Domain F=Antigen 1 B-Body Domain F/L Scaffold (SEQ ID NO:77)
   Domain G=CH3 (L351D; 445G, 446E, 447C tripeptide insertion)
3^{rd} polypeptide chain (SEQ ID NO:80):
   Domain H=Antigen 2 B-Body Domain A/H Scaffold (SEQ ID NO:76)
   Domain I=CL (Kappa)
   Domain J=CH2
   Domain K=CH3 (Y349C, D356E, L358M, T366S, L368A, Y407V)
4^{th} polypeptide chain (SEQ ID NO:81):
   Domain L=Antigen 2 B-Body Domain F/L Scaffold (SEQ ID NO:77)
   Domain M=CH1.

For BC1 1×2 formats, the variable domains were formatted into the 1(A)×2(B−A) format described herein. Polypeptide Chain 2 and Chain 6 are identical in the 1(A)×2(B−A) format.

### 6.14.2. Example 1: Bivalent Monospecific Construct and Bivalent Bispecific Construct

A bivalent monospecific B-Body recognizing TNFα was constructed with the following architecture (VL(Certolizumab)-CH3(Knob)-CH2-CH3/VH(Certolizumab)-CH3(Hole)) using standard molecular biology procedures. In this construct,
1st polypeptide chain (SEQ ID NO:1)
   Domain A=VL (certolizumab)
   Domain B=CH3 (IgG1) (knob: S354C+T366W)
   Domain D=CH2 (IgG1)
   Domain E=CH3 (IgG1)
2nd polypeptide chain (SEQ ID NO:2)
   Domain F=VH (certolizumab)
   Domain G=CH3 (IgG1) (hole:Y349C, T366'S, L368A, Y407V)
3rd polypeptide chain:
   identical to the 1st polypeptide chain
4th polypeptide chain:
   identical to the 2nd polypeptide chain.

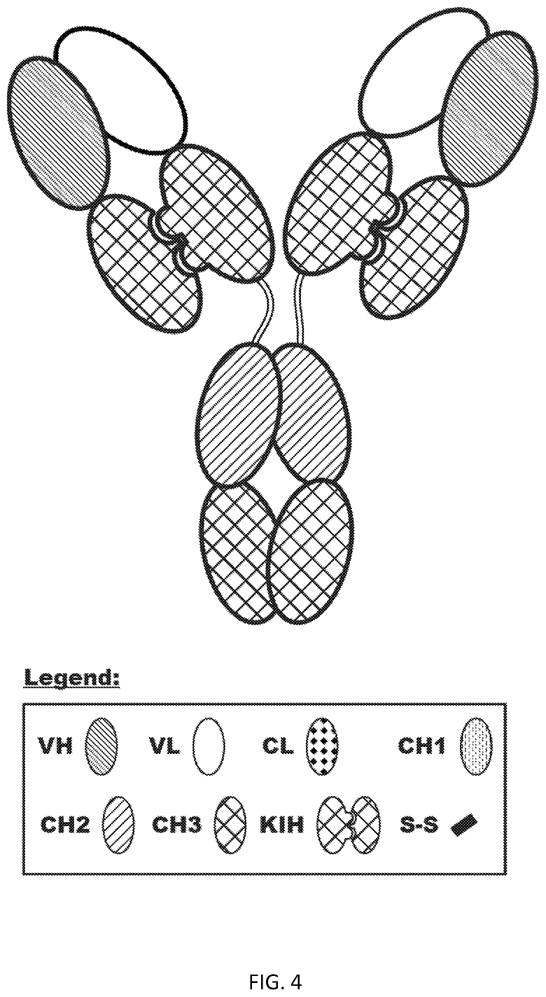

Domain and polypeptide chain references are in accordance with FIG. 3. The overall construct architecture is illustrated in FIG. 4. The sequence of the first polypeptide chain, with domain A identified in shorthand as “(VL)”, is provided in SEQ ID NO:1. The sequence of the second polypeptide chain, with domain F identified in shorthand as “(VH)”, is provided in SEQ ID NO:2.

The full-length construct was expressed in an E. coli cell free protein synthesis expression system for ˜18 hours at 26° C. with gentle agitation. Following expression, the cell-free extract was centrifuged to pellet insoluble material and the supernatant was diluted 2× with 10× Kinetic Buffer (Forte Bio) and used as the analyte for biolayer interferometry.

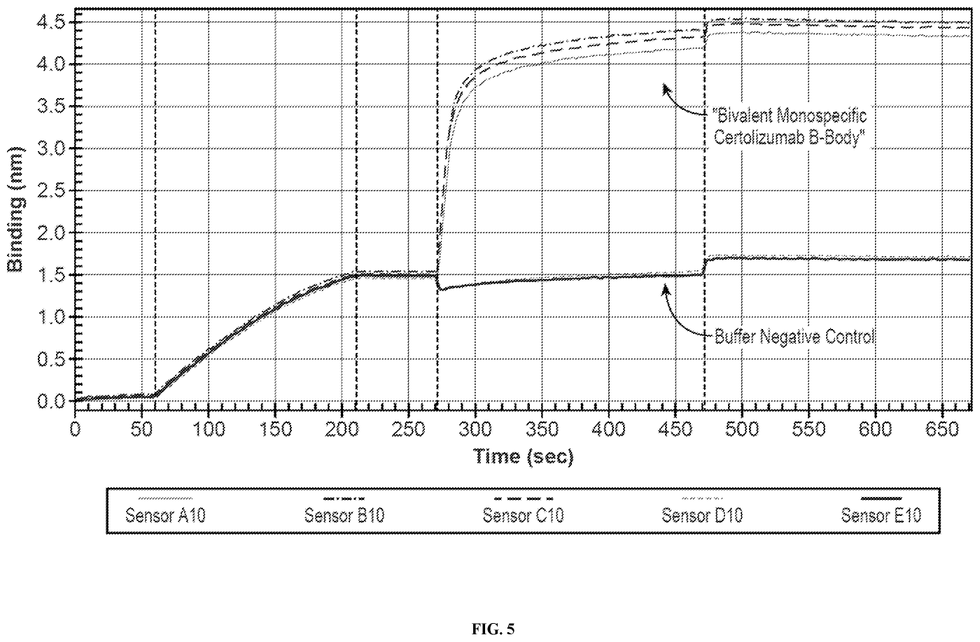

Biotinylated TNFα was immobilized on a streptavidin sensor to give a wave shift response of ˜1.5 nm. After establishing a baseline with 10× kinetic buffer, the sensor was dipped into the antibody construct analyte solution. The construct gave a response of ˜3 nm, comparable to the traditional IgG format of certolizumab, demonstrating the ability of the bivalent monospecific construct to assemble into a functional, full-length antibody. Results are shown in FIG. 5.

We also constructed a bivalent bispecific antibody with the following domain architecture:
1st polypeptide chain: VL-CH3-CH2-CH3(Knob)
2nd polypeptide chain: VH-CH3
3rd polypeptide chain: VL-CL-CH2-CH3(Hole)
4th polypeptide chain VH-CH1.

The sequences (except for the variable region sequences) are provided respectively in SEQ ID NO:3 (1st polypeptide chain), SEQ ID NO:4 (2nd polypeptide chain), SEQ ID NO:5 (3rd polypeptide chain), SEQ ID NO:6 (4th polypeptide chain).

### 6.14.3. Example 2: Bivalent Bispecific B-Body “BC1”

We constructed a bivalent bispecific construct, termed “BC1”, specific for PD1 and a second antigen, “Antigen A”). Salient features of the “BC1” architecture are illustrated in FIG. 6.

In greater detail, with domain and polypeptide chain references in accordance with FIG. 3 and modifications from native sequence indicated in parentheses, the architecture was:
1^{st} polypeptide chain (SEQ ID NO:8)
   Domain A=VL (“Antigen A”)
   Domain B=CH3 (T366K; 445K, 446S, 447C tripeptide insertion)
   Domain D=CH2
   Domain E=CH3 (T366W, S354C)
2nd polypeptide chain (SEQ ID NO:9):
   Domain F=VH (“Antigen A”)
   Domain G=CH3 (L351D; 445G, 446E, 447C tripeptide insertion)
3rd polypeptide chain (SEQ ID NO:10):
   Domain H=VL (“Nivo”)
   Domain I=CL (Kappa)
   Domain J=CH2
   Domain K=CH3 (Y349C, D356E, L358M, T366S, L368A, Y407V)
4th polypeptide chain (SEQ ID NO:11):
   Domain L=VH (“Nivo”)
   Domain M=CH1.

The A domain (SEQ ID NO: 12) and F domain (SEQ ID NO: 16) form an antigen binding site (A:F) specific for “Antigen A”. The H domain has the VH sequence from nivolumab and the L domain has the VL sequence from nivolumab; H and L associate to form an antigen binding site (H:L) specific for human PD1.

The B domain (SEQ ID NO:13) has the sequence of human IgG1 CH3 with several mutations: T366K, 445K, 446S, and 447C insertion. The T366K mutation is a charge pair cognate of the L351D residue in Domain G. The “447C” residue on domain B comes from the C-terminal KSC tripeptide insertion.

Domain D (SEQ ID NO: 14) has the sequence of human IgG1 CH2

Domain E (SEQ ID NO: 15) has the sequence of human IgG1 CH3 with the mutations T366W and S354C. The 366W is the “knob” mutation. The 354C introduces a cysteine that is able to form a disulfide bond with the cognate 349C mutation in Domain K.

Domain G (SEQ ID NO:17) has the sequence of human IgG1 CH3 with the following mutations: L351D, and 445G, 446E, 447C tripeptide insertion. The L351D mutation introduces a charge pair cognate to the Domain B T366K mutation. The “447C” residue on domain G comes from the C-terminal GEC tripeptide insertion.

Domain I (SEQ ID NO: 19) has the sequence of human C kappa light chain (CIO

Domain J [SEQ ID NO: 20] has the sequence of human IgG1 CH2 domain, and is identical to the sequence of domain D.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in Domain E. The 356E and L358M introduce isoallotype amino acids that reduce immunogenicity. The 366S, 368A, and 407V are “hole” mutations.

Domain M [SEQ ID NO: 23] has the sequence of the human IgG1 CH1 region.

“BC1” could readily be expressed at high levels using mammalian expression at concentrations greater than 100 μg/ml.

We found that the bivalent bispecific “BC1” protein could easily be purified in a single step using a CH1-specific CaptureSelect™ affinity resin from ThermoFisher.

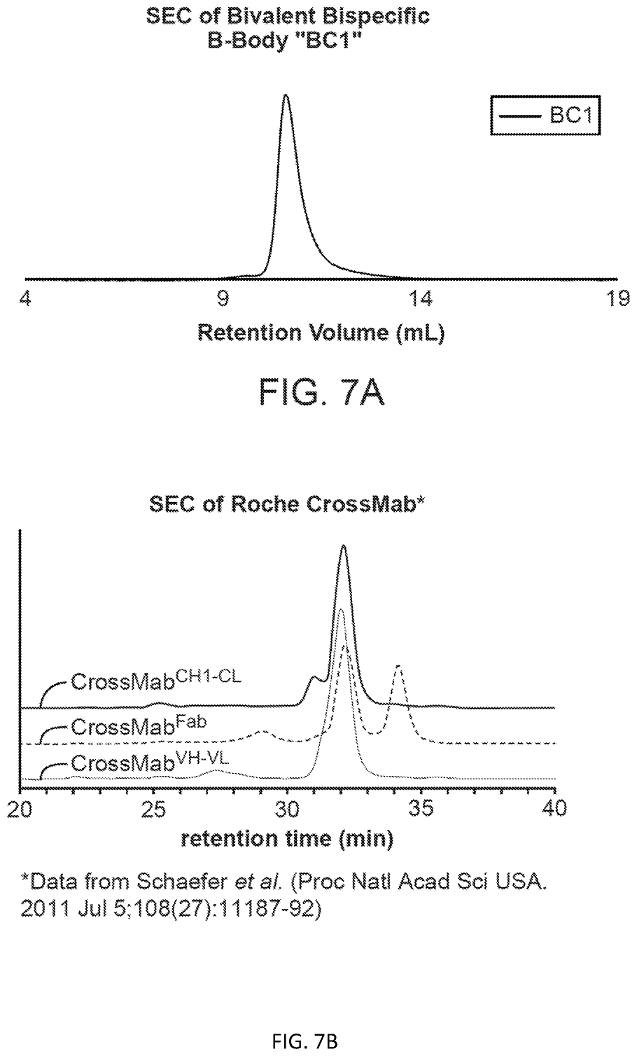

As shown in FIG. 7A, SEC analysis demonstrates that a single-step CH1 affinity purification step yields a single, monodisperse peak via gel filtration in which >98% is monomer. FIG. 7B shows comparative literature data of SEC analysis of a CrossMab bivalent antibody construct.

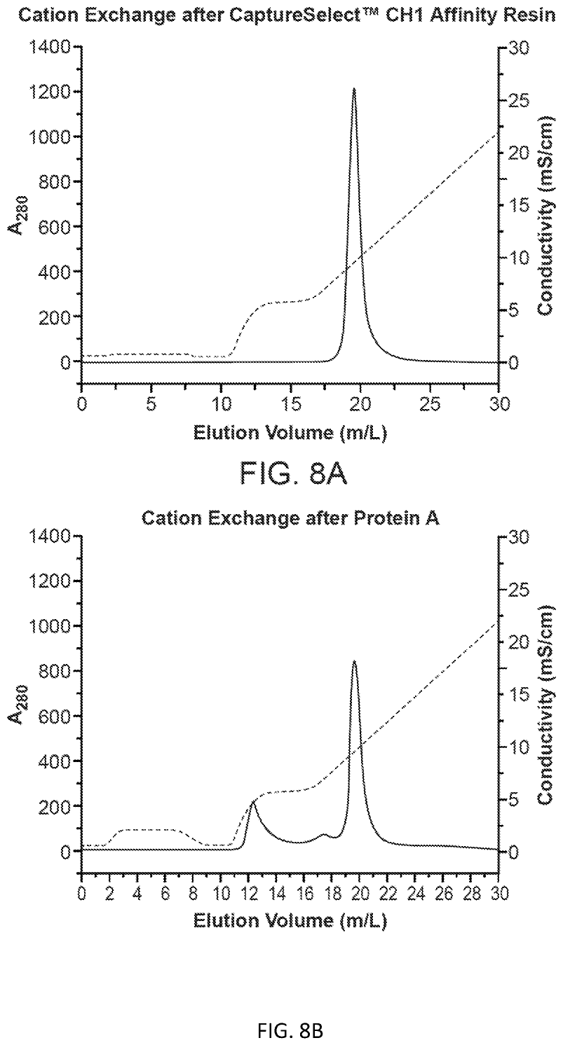

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification, showing additional elution peaks consistent with the co-purification of incomplete assembly products.

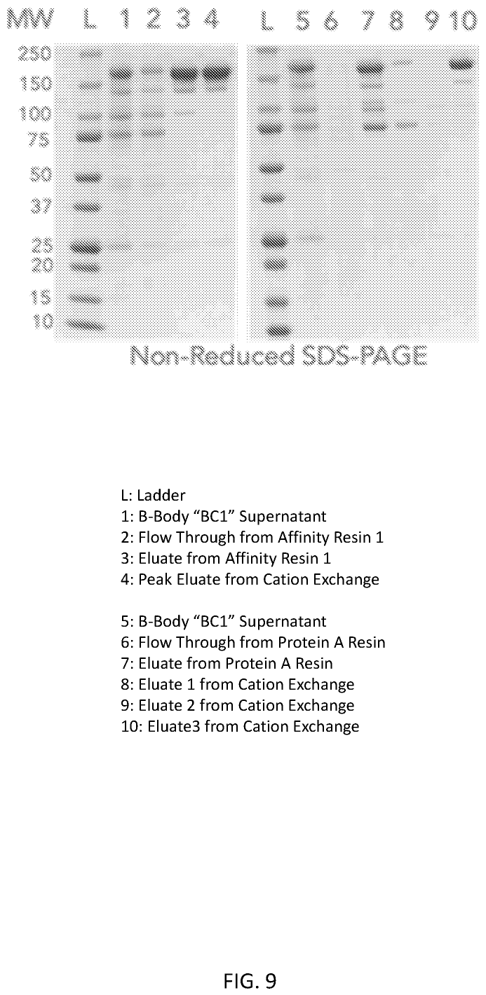

FIG. 9 shows SDS-PAGE gels under non-reducing conditions. As seen in lane 3, single-step purification of “BC1” with CH1 affinity resin provides a nearly homogeneous single band, with lane 4 showing minimal additional purification with a subsequent cation exchange polishing step. Lane 7, by comparison, shows less substantial purification using standard Protein A purification, with lanes 8-10 demonstrating further purification of the Protein A purified material using cation exchange chromatography.

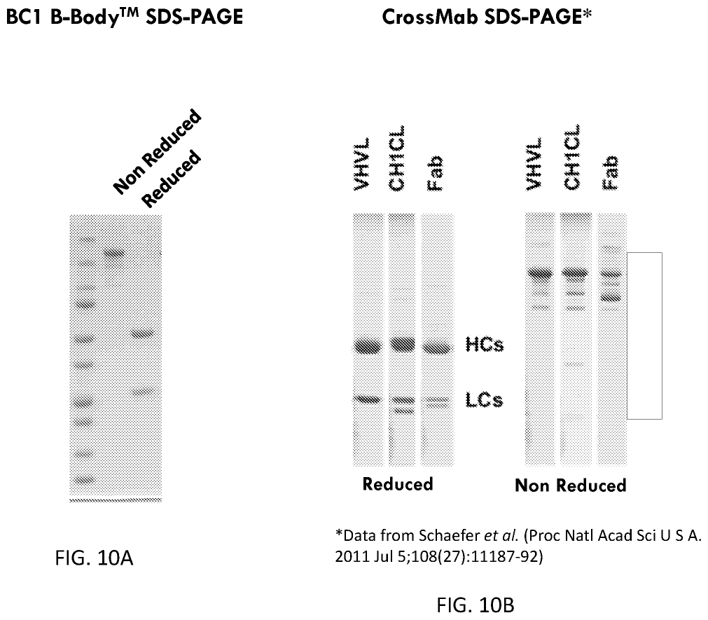

FIG. 10 compares SDS-PAGE gels of “BC1” after single-step CH1-affinity purification, under both non-reducing and reducing conditions (Panel A) with SDS-PAGE gels of a CrossMab bispecific antibody under non-reducing and reducing conditions as published in the referenced literature (Panel B).

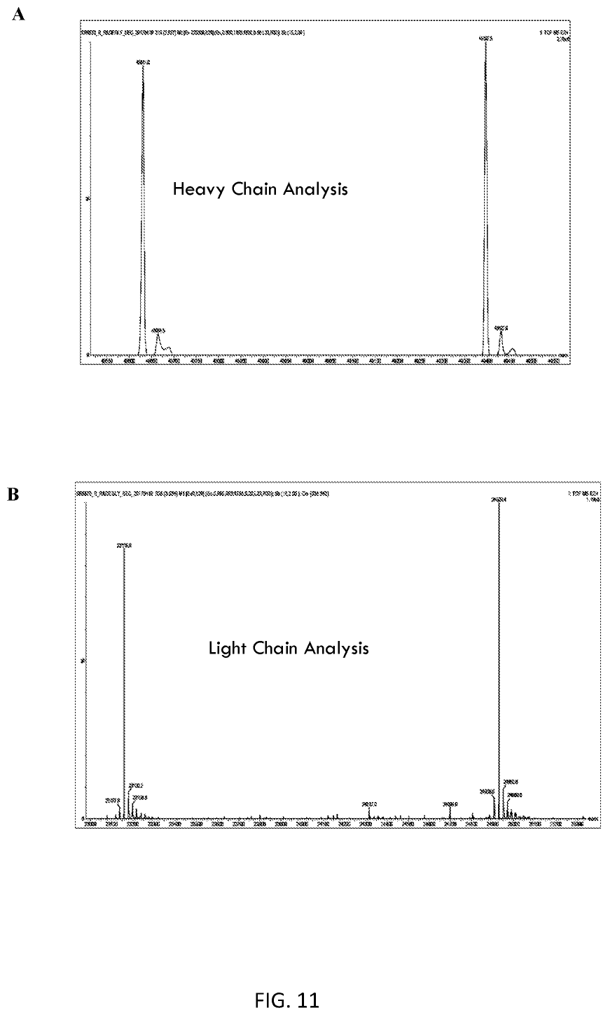

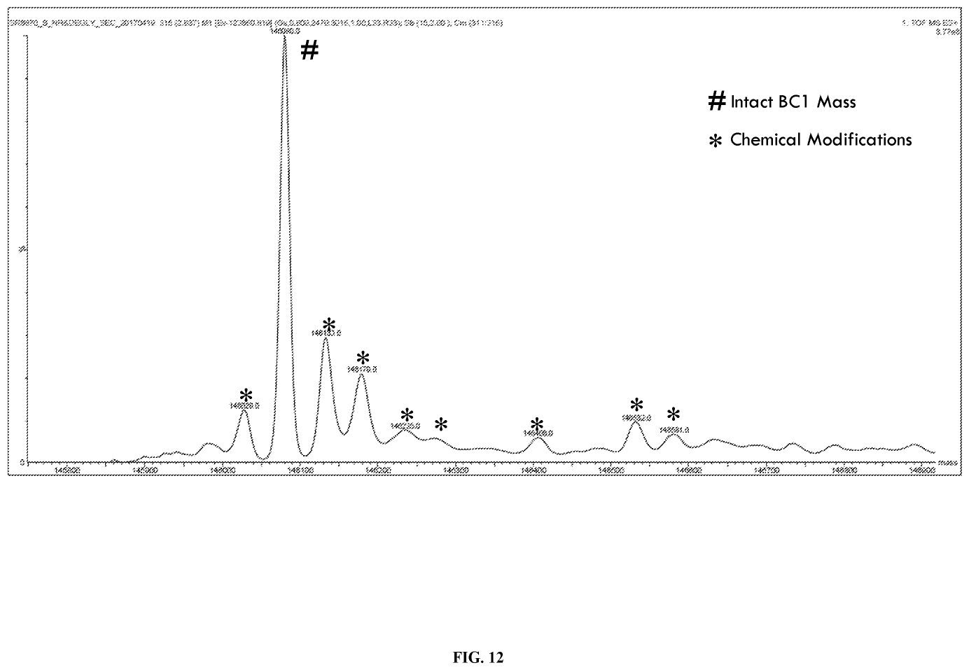

FIG. 11 shows mass spec analysis of “BC1”, demonstrating two distinct heavy chains (FIG. 11A) and two distinct light chains (FIG. 11B) under reducing conditions. The mass spectrometry data in FIG. 12 confirms the absence of incomplete pairing after purification.

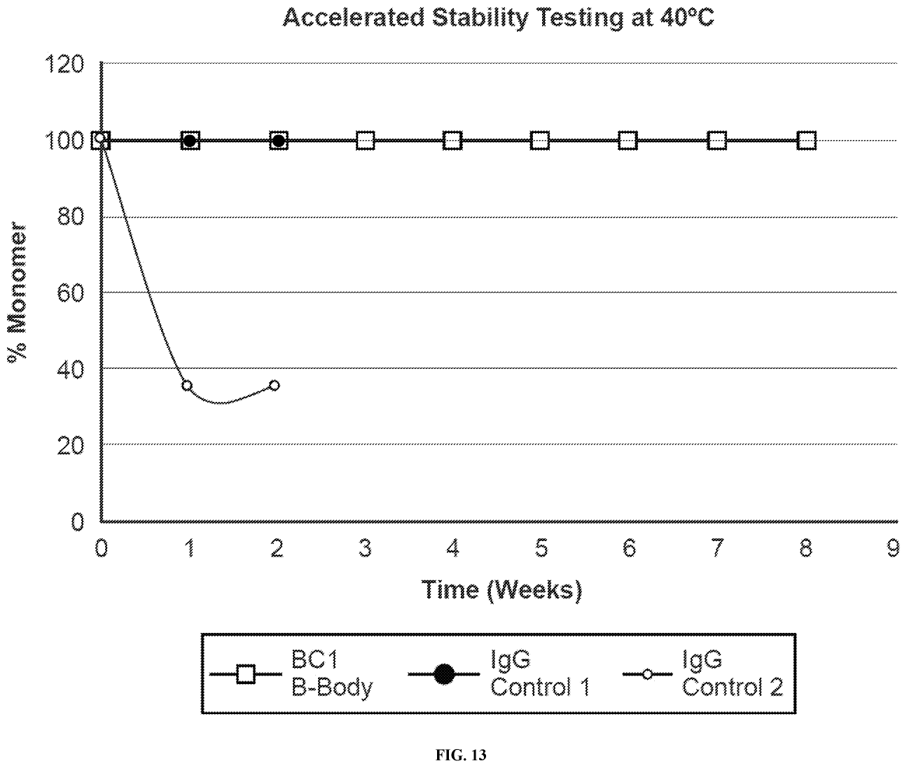

Accelerated stability testing was performed to evaluate the long-term stability of the “BC1” B-Body design. The purified B-Body was concentrated to 8.6 mg/ml in PBS buffer and incubated at 40° C. The structural integrity was measured weekly using analytical size exclusion chromatography (SEC) with a Shodex KW-803 column. The structural integrity was determined by measuring the percentage of intact monomer (% Monomer) in relation to the formation of aggregates. Data are shown in FIG. 13. The IgG Control 1 is a positive control with good stability properties. IgG Control 2 is a negative control that is known to aggregate under the incubation conditions. The “BC1” B-Body has been incubated for 8 weeks without any loss of structural integrity as determined by the analytical SEC.

We have also determined that “BC1” has high thermostability, with a TM of the bivalent construct of ˜72° C.

Table 1 compares “BC1” to CrossMab in key developability characteristics:

TABLE 1

| | | Roche | |
|---|---|---|---|
| Parameter | Unit | CrossMab* | “BC1” |
| Purification yield after | mg/L | 58.5 | 300 |
| protein A/SEC | | | |
| Homogeneity After purification | % SEC Area | 50-85 | 98 |
| Denaturation Temp (Tm) | degrees C. | 69.2 | 72 |

*Data from Schaefer et al. (Proc Natl Acad Sci USA. 2011 Jul. 5; 108(27): 11187-92)

### 6.14.4. Example 3: Bivalent Bispecific B-Body “BC6”

We constructed a bivalent bispecific B-Body, termed “BC6”, that is identical to “BC1” but for retaining wild type residues in Domain B at residue 366 and Domain G at residue 351. “BC6” thus lacks the charge-pair cognates T366K and L351D that had been designed to facilitate correct pairing of domain B and domain Gin “BC1”. Salient features of the “BC6” architecture are illustrated in FIG. 14.

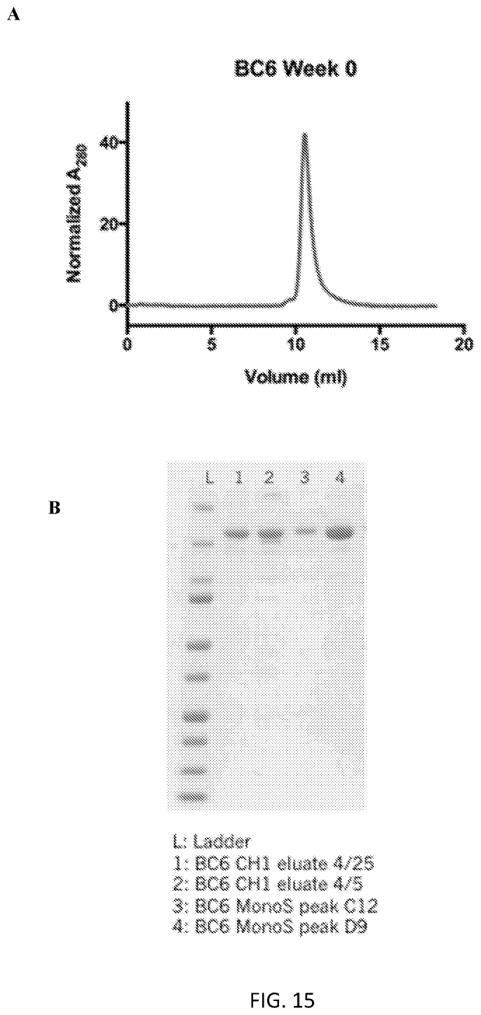

Notwithstanding the absence of the charge-pair residues present in “BC1”, we found that a single step purification of “BC6” using CH1 affinity resin resulted in a highly homogeneous sample. FIG. 15A shows SEC analysis of “BC6” following one-step purification using the CaptureSelect™ CH1 affinity resin. The data demonstrate that the single step CH1 affinity purification yields a single monodisperse peak, similar to what we observed with “BC1”, demonstrating that the disulfide bonds between polypeptide chains 1 and 2 and between polypeptide chains 3 and 4 are intact. The chromatogram also shows the absence of non-covalent aggregates.

FIG. 15B shows a SDS-PAGE gel under non-reducing conditions, with lane 1 loaded with a first lot of “BC6” after a single-step CH1 affinity purification, lane 2 loaded with a second lot of “BC6” after a single-step CH1 affinity purification. Lanes 3 and 4 demonstrate further purification can be achieved with ion exchange chromatography subsequent to CH1 affinity purification.

### 6.14.5. Example 4: Bivalent Bispecific B-Bodies “BC28”, “BC29”, “BC30”, “BC31”

We constructed bivalent 1×1 bispecific B-Bodies “BC28”, “BC29”, “BC30” and “BC31” having an engineered disulfide within the CH3 interface in Domains B and G as an alternative S-S linkage to the C-terminal disulfide present in “BC1” and “BC6”. Literature indicates that CH3 interface disulfide bonding is insufficient to enforce orthogonality in the context of Fc CH3 domains. The general architecture of these B-Body constructs is schematized in FIG. 16 with salient features of “BC28” summarized below:
Polypeptide chain 1: “BC28” chain 1 (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
Polypeptide chain 2: “BC28” chain 2 (SEQ ID NO:25)
   Domain F=VH (Antigen “A”)
   Domain G=CH3 (S354C; 445P, 446G, 447K insertion)
Polypeptide chain 3: “BC1” chain 3 (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
Polypeptide chain 4: “BC1” chain 4 (SEQ ID NO:11)
   Domain L=VH (“Nivo”)
   Domain M=CH1

The “BC28” A:F antigen binding site is specific for “Antigen A”. The “BC28” H:L antigen binding site is specific for PD1 (nivolumab sequences). “BC28” domain B has the following changes as compared to wild type CH3: Y349C; 445P, 446G, 447K insertion. “BC28” domain E has the following changes as compared to wild type CH3: S354C, T366W. “BC28” domain G has the following changes as compared to wild type: S354C; 445P, 446G, 447K insertion.

“BC28” thus has an engineered cysteine at residue 349C of Domain B and engineered cysteine at residue 354C of domain G (“349C-354C”).

“BC29” has engineered cysteines at residue 351C of Domain B and 351C of Domain G (“351C-351C”). “BC30” has an engineered cysteine at residue 354C of Domain B and 349C of Domain G (“354C-349C”). BC31 has an engineered cysteine at residue 394C and engineered cysteine at 394C of Domain G (“394C-394C”). BC32 has engineered cysteines at residue 407C of Domain B and 407C of Domain G (“407C-407C”).

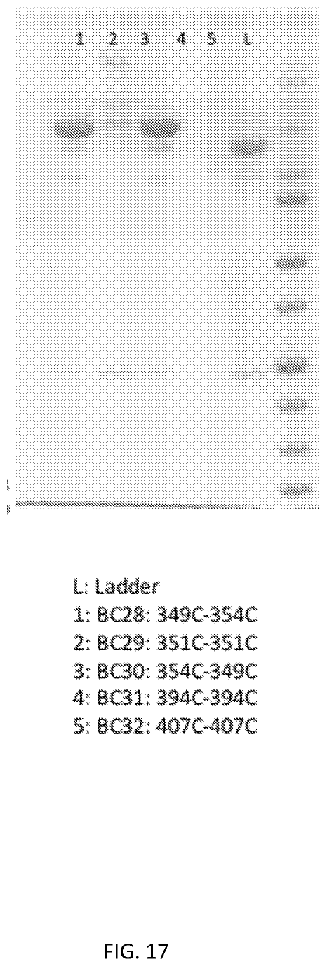

FIG. 17 shows SDS-PAGE analysis under non-reducing conditions following one-step purification using the CaptureSelect™ CH1 affinity resin. Lanes 1 and 3 show high levels of expression and substantial homogeneity of intact “BC28” (lane 1) and “BC30” (lane 3). Lane 2 shows oligomerization of BC29. Lanes 4 and 5 show poor expression of BC31 and BC32, respectively, and insufficient linkage in BC32. Another construct, BC9, which had cysteines introduced at residue 392 in domain B and 399 in Domain G (“392C-399C”), a disulfide pairing reported by Genentech, demonstrated oligomerization on SDS PAGE (data not shown).

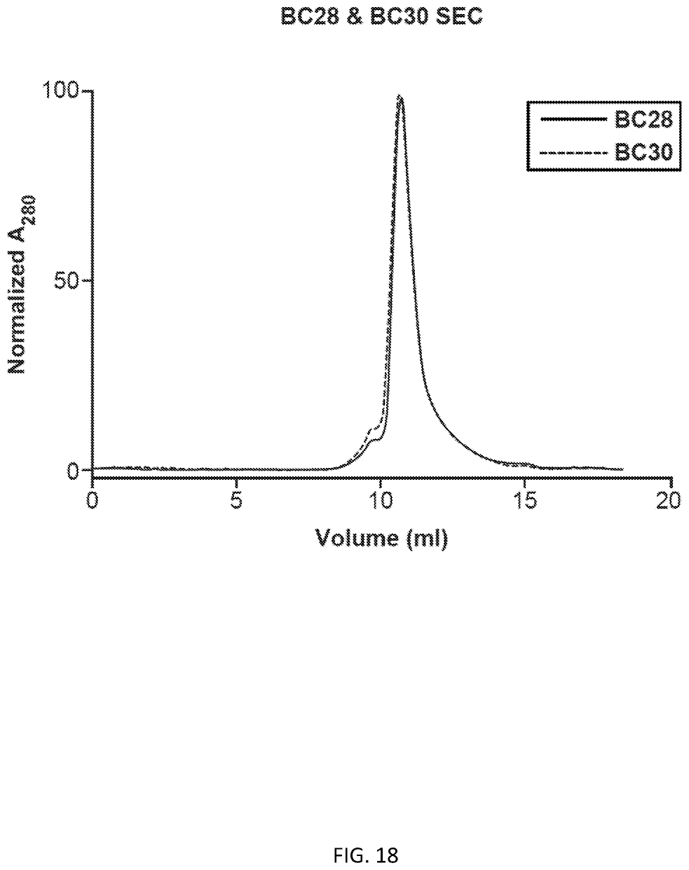

FIG. 18 shows SEC analysis of “BC28” and “BC30” following one-step purification using the CaptureSelect™ CH1 affinity resin. We have also demonstrated that “BC28” can readily be purified using a single step purification using Protein A resin (results not shown).

### 6.14.6. Example 5: Bivalent Bispecific B-Body “BC44”

FIG. 19 shows the general architecture of the bivalent bispecific 1×1 B-Body “BC44”, our currently preferred bivalent bispecific 1×1 construct.
first polypeptide chain (′BC44″ chain 1) (SEQ ID NO:32)
   Domain A=VL (Antigen “A”)
   Domain B=CH3 (P343V; Y349C; 445P, 446G, 447K insertion)
   Domain E=CH2
   Domain E=CH3 (S354C, T366W)
second polypeptide chain (=“BC28” polypeptide chain 2) (SEQ NO:25)
   Domain F=VH (Antigen “A”)
   Domain G=CH3 (S354C; 445P, 446G, 447K insertion)
third polypeptide chain (=“BC1” polypeptide chain 3) (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
fourth polypeptide chain (=“BC1” polypeptide chain 4) (SEQ ID NO:11)
   Domain L=VH (“Nivo”)
   Domain M=CH1.

### 6.14.7. Example 6: Variable-CH3 Junction Engineering

We produced a series of variants in which we mutated the VL-CH3 junction between Domains A and B and the VH-CH3 junction between domains F and G to assess the expression level, assembly and stability of bivalent 1×1 B-Body constructs. Although there are likely many solutions, to reduce introduction of T cell epitopes we chose to only use residues found naturally within the VL, VH and CH3 domains. Structural assessment of the domain architecture further limits desirable sequence combinations. Table 2 and Table 3 below show junctions for several junctional variants based on “BC1” and other bivalent constructs.

TABLE 2
Variants of Variable Domain/Constant Domain
Junctions for 1^{st} Polypeptide Chain

| | VL | | | | | | CH3 | | | | |
|---|---|---|---|---|---|---|---|---|---|---|---|
| Variant | 106 | 107 | 108 | 109 | 110 | 111 | 343 | 344 | 345 | 346 | Sequence |
| BC1 | I | K | R | T | | | P | R | E | P | IKRTPREP |
| | | | | | | | | | | | (SEQ ID |
| | | | | | | | | | | | NO: 57) |
| BC13 | I | K | R | T | | | P | R | E | P | IKRTPREP |
| | | | | | | | | | | | (SEQ ID |
| | | | | | | | | | | | NO: 57) |
| BC14 | I | K | R | T | | | P | R | E | P | IKRTPREP |
| | | | | | | | | | | | (SEQ ID |
| | | | | | | | | | | | NO: 57) |
| BC15 | I | K | R | T | V | | | R | E | P | IKRTVREP |
| | | | | | | | | | | | (SEQ ID |
| | | | | | | | | | | | NO: 58) |
| BC16 | I | K | R | T | | | | R | E | P | IKRTREP |
| | | | | | | | | | | | (SEQ ID |
| | | | | | | | | | | | NO: 59) |
| BC17 | I | K | R | T | V | | P | R | E | P | IKRTVPREP |
| | | | | | | | | | | | (SEQ ID |
| | | | | | | | | | | | NO: 60) |
| BC24 | I | K | R | T | | | P | R | E | P | IKRTPREP |
| | | | | | | | | | | | (SEQ ID |
| | | | | | | | | | | | NO: 57) |
| BC25 | I | K | R | T | | | P | R | E | P | IKRTPREP |
| | | | | | | | | | | | (SEQ ID |
| | | | | | | | | | | | NO: 57) |
| BC26 | I | K | R | T | V | A | | | E | P | IKRTVAEP |
| | | | | | | | | | | | (SEQ ID |
| | | | | | | | | | | | NO: 61) |
| BC27 | I | K | R | T | V | A | P | R | E | P | IKRTVA |
| | | | | | | | | | | | PREP |
| | | | | | | | | | | | (SEQ ID |
| | | | | | | | | | | | NO: 62) |
| BC44 | I | K | R | T | | | V | R | E | P | IKRTVREP |
| | | | | | | | | | | | (SEQ ID |
| | | | | | | | | | | | NO: 58) |
| BC45 | I | K | R | T | | | P | R | E | P | IKRTPREP |
| | | | | | | | | | | | (SEQ ID |
| | | | | | | | | | | | NO: 57) |
| BC5 | I | K | R | T | | | P | R | E | P | IKRTPREP |
| | | | | | | | | | | | (SEQ ID |
| | | | | | | | | | | | NO: 57) |
| BC6 | I | K | R | T | | | P | R | E | P | IKRTPREP |
| | | | | | | | | | | | (SEQ ID |
| | | | | | | | | | | | NO: 57) |
| BC28 | I | K | R | T | | | P | R | E | P | IKRTPREP |
| | | | | | | | | | | | (SEQ ID |
| | | | | | | | | | | | NO: 57) |
| BC30 | I | K | R | T | | | P | R | E | P | IKRTPREP |
| | | | | | | | | | | | (SEQ ID |
| | | | | | | | | | | | NO: 57) |

TABLE 3
Variants of Variable Domain/Constant Domain
Junctions for 2^{nd} Polypeptide Chain

| Vari- | VH | | | | | | | CH3 | | | | |
|---|---|---|---|---|---|---|---|---|---|---|---|---|
| ant | 112 | 113 | 114 | 115 | 116 | 117 | 118 | 343 | 344 | 345 | 346 | Sequence |
| BC1 | S | S | A | S | | | | P | R | E | P | SSASPREP |
| | | | | | | | | | | | | (SEQ ID |
| | | | | | | | | | | | | NO: 63) |
| BC13 | S | S | A | S | T | | | | R | E | P | SSASTREP |
| | | | | | | | | | | | | (SEQ ID |
| | | | | | | | | | | | | NO: 64) |
| BC14 | S | S | A | S | T | | | P | R | E | P | SSASTP |
| | | | | | | | | | | | | REP |
| | | | | | | | | | | | | (SEQ ID |
| | | | | | | | | | | | | NO: 65) |
| BC15 | S | S | A | S | | | | P | R | E | P | SSASPREP |
| | | | | | | | | | | | | (SEQ ID |
| | | | | | | | | | | | | NO: 63) |
| BC16 | S | S | A | S | | | | P | R | E | P | SSASPREP |
| | | | | | | | | | | | | (SEQ ID |
| | | | | | | | | | | | | NO: 63) |
| BC17 | S | S | A | S | | | | P | R | E | P | SSASPREP |
| | | | | | | | | | | | | (SEQ ID |
| | | | | | | | | | | | | NO: 63) |
| BC24 | S | S | A | S | T | K | G | | | E | P | SSASTK |
| | | | | | | | | | | | | GEP |
| | | | | | | | | | | | | (SEQ ID |
| | | | | | | | | | | | | NO: 66) |
| BC25 | S | S | A | S | T | K | G | | R | E | P | SSASTKGR |
| | | | | | | | | | | | | EP |
| | | | | | | | | | | | | (SEQ ID |
| | | | | | | | | | | | | NO: 67) |
| BC26 | S | S | A | S | | | | P | R | E | P | SSASPREP |
| | | | | | | | | | | | | (SEQ ID |
| | | | | | | | | | | | | NO: 63) |
| BC27 | S | S | A | S | | | | P | R | E | P | SSASPREP |
| | | | | | | | | | | | | (SEQ ID |
| | | | | | | | | | | | | NO: 63) |
| BC44 | S | S | A | S | | | | P | R | E | P | SSASPREP |
| | | | | | | | | | | | | (SEQ ID |
| | | | | | | | | | | | | NO: 63) |
| BC45 | S | S | A | S | | | | P | R | E | P | SSASPREP |
| | | | | | | | | | | | | (SEQ ID |
| | | | | | | | | | | | | NO: 63) |
| BC5 | S | S | A | S | | | | P | R | E | P | SSASPREP |
| | | | | | | | | | | | | (SEQ ID |
| | | | | | | | | | | | | NO: 63) |
| BC6 | S | S | A | S | | | | P | R | E | P | SSASPREP |
| | | | | | | | | | | | | (SEQ ID |
| | | | | | | | | | | | | NO: 63) |
| BC28 | S | S | A | S | | | | P | R | E | P | SSASPREP |
| | | | | | | | | | | | | (SEQ ID |
| | | | | | | | | | | | | NO: 63) |
| BC30 | S | S | A | S | | | | P | R | E | P | SSASPREP |
| | | | | | | | | | | | | (SEQ ID |
| | | | | | | | | | | | | NO: 63) |

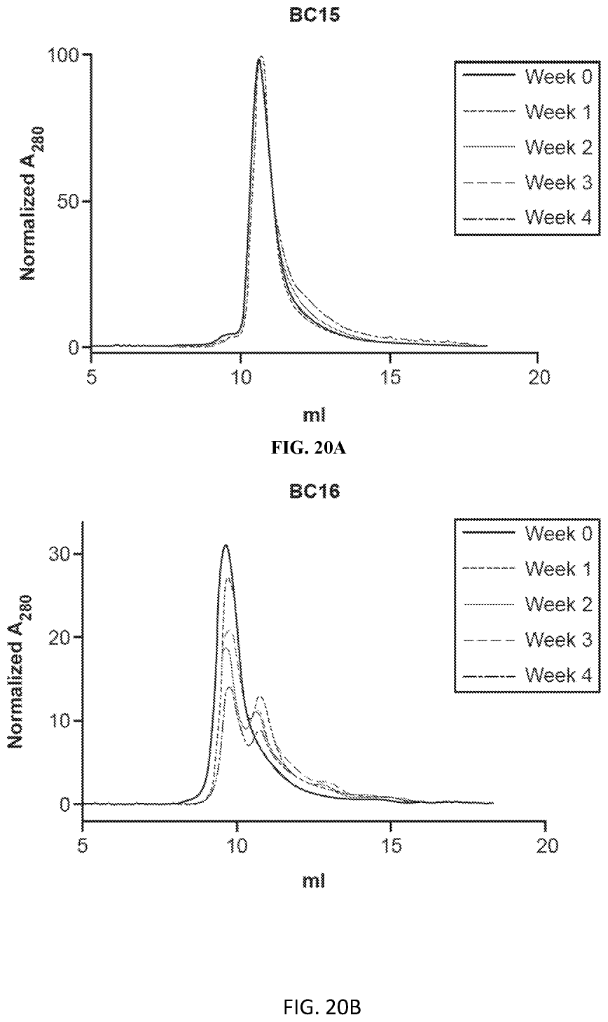

FIG. 20 shows size exclusion chromatography of “BC15” and “BC16” samples at the indicated week of an accelerated stability testing protocol at 40° C. “BC15” remained stable; “BC16” proved to be unstable over time.

### 6.14.8. Example 7: Trivalent 2×1 Bispecific B-Body Construct (“BC1-2×1”)

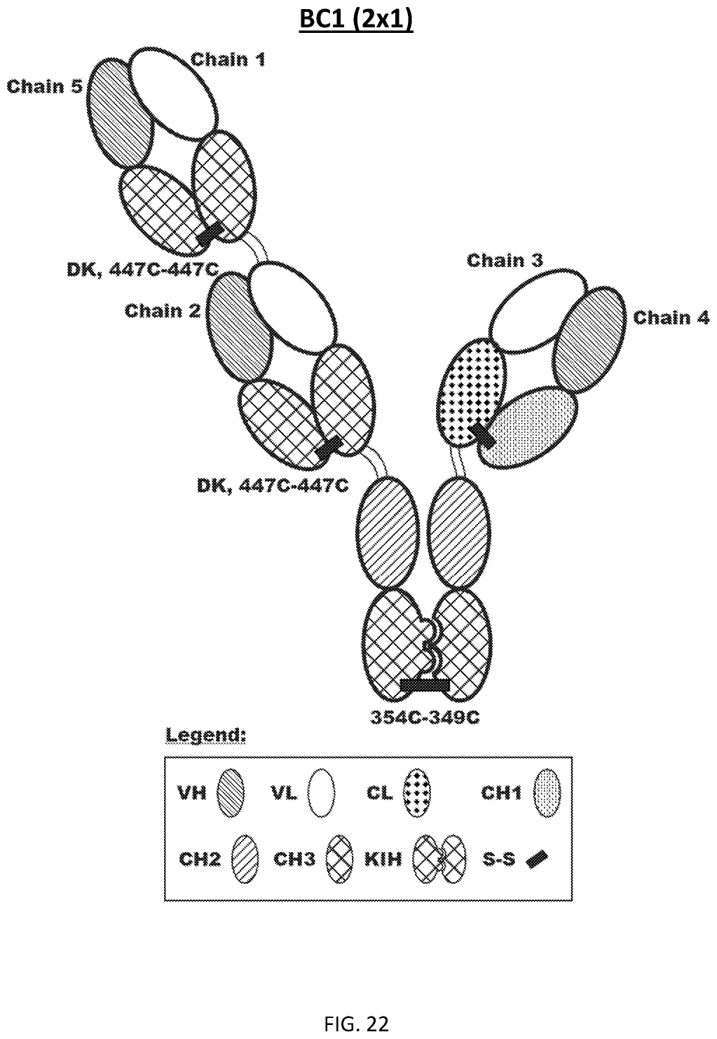

We constructed a trivalent 2×1 bispecific B-Body “BC1-2×1” based on “BC1”. Salient features of the architecture are illustrated in FIG. 22.

In greater detail, using the domain and polypeptide chain references summarized in FIG. 21,
1^{st} polypeptide chain
   Domain N=VL (“Antigen A”)
   Domain O=CH3 (T366K, 447C)
   Domain A=VL (“Antigen A”)
   Domain B=CH3 (T366K, 447C)
   Domain D=CH2
   Domain E=CH3 (Knob, 354C)
5^{th} polypeptide chain (=“BC1” chain 2)
   Domain P=VH (“Antigen A”)
   Domain Q=CH3 (L351D, 447C)
2^{nd} polypeptide chain (=“BC1” chain 2)
   Domain F=VH (“Antigen A”)
   Domain G=CH3 (L351D, 447C)
3^{rd} polypeptide chain (=“BC1” chain 3)
   Domain H=VL (“Nivo”)
   Domain I=CL (Kappa)
   Domain J=CH2
   Domain K=CH3 (Hole, 349C)
4^{th} polypeptide chain (=“BC1” chain 4)
   Domain L=VH (“Nivo”)
   Domain M=CH1.

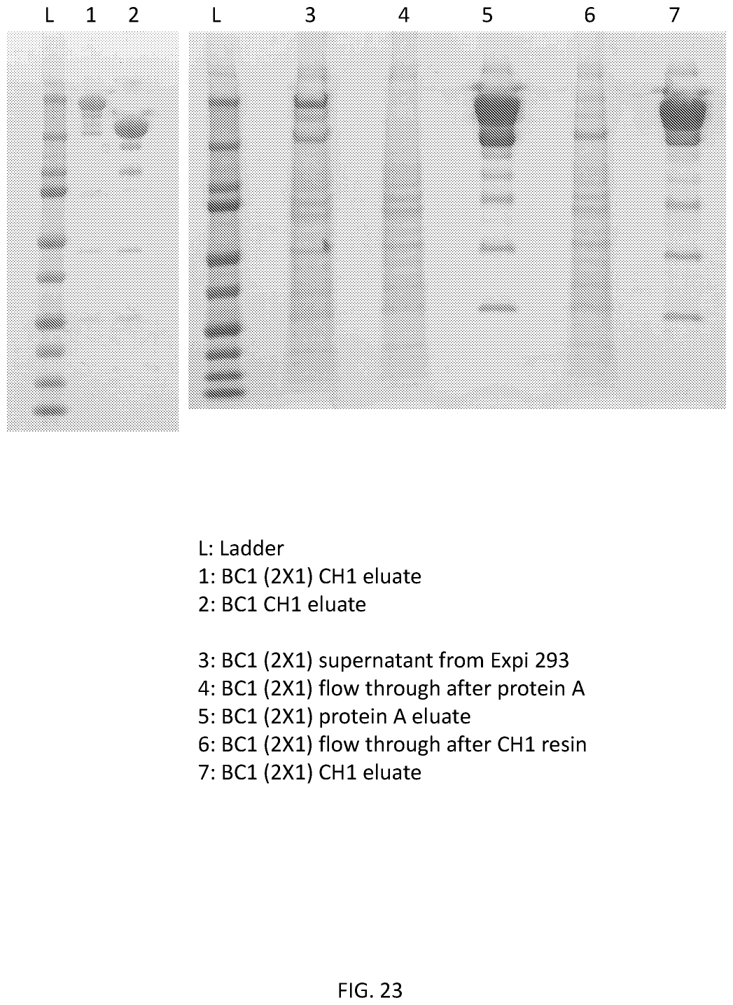

FIG. 23 shows non-reducing SDS-PAGE of protein expressed using the ThermoFisher Expi293 transient transfection system.

Lane 1 shows the eluate of the trivalent 2×1 “BC1-2×1” protein following one-step purification using the CaptureSelect™ CH1 affinity resin. Lane 2 shows the lower molecular weight, faster migrating, bivalent “BC1” protein following one-step purification using the CaptureSelect™ CH1 affinity resin. Lanes 3-5 demonstrate purification of “BC1-2×1” using protein A. Lanes 6 and 7 show purification of “BC1-2×1” using CH1 affinity resin.

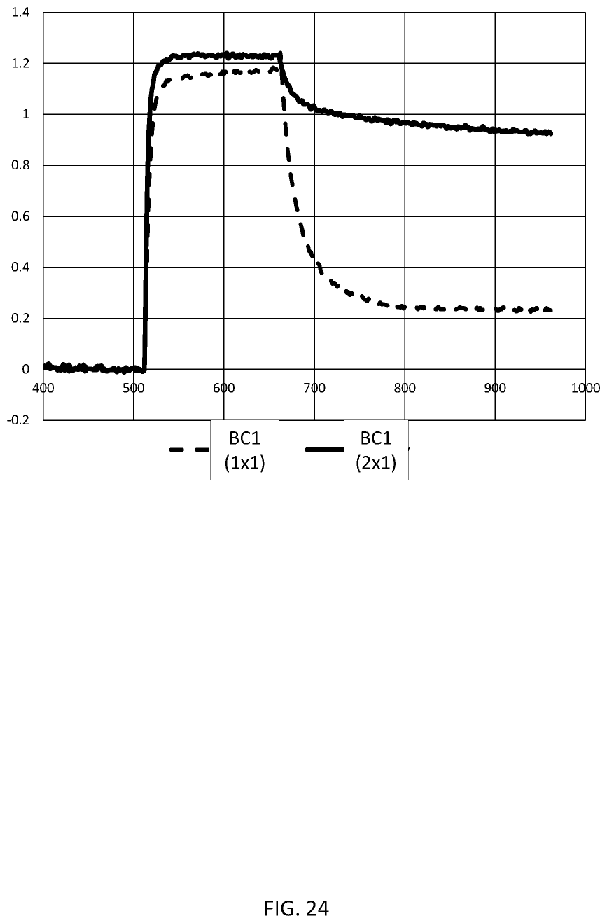

FIG. 24 compares the avidity of the bivalent “BC1” construct to the avidity of the trivalent 2×1 “BC1-2×1” construct using an Octet (Pall ForteBio) analysis. Biotinylated antigen “A” is immobilized on the surface, and the antibody constructs are passed over the surface for binding analysis.

### 6.14.9. Example 8: Trivalent 2×1 Trispecific B-Body Construct (“TB111”)

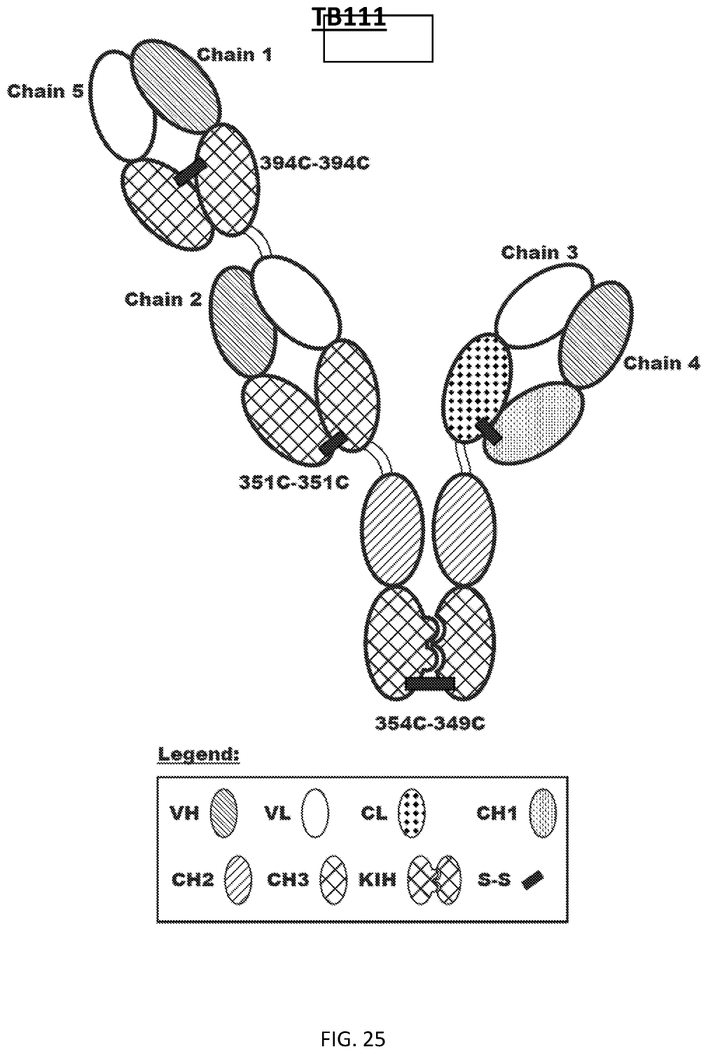

We designed a trivalent 2×1 trispecific molecule, “TB111”, having the architecture schematized in FIG. 25. With reference to the domain naming conventions set forth in FIG. 21, TB111 has the following architecture (“Ada” indicates a V region from adalimumab):
polypeptide chain 1
   Domain N: VH (“Ada”)
   Domain O: CH3 (T366K, 394C)
   Domain A: VL (“Antigen A”)
   Domain B: CH3 (T366K, 349C)
   Domain D: CH2
   Domain E: CH3 (Knob, 354C)
polypeptide chain 5
   Domain P: VL (“Ada”)
   Domain Q: CH3 (L351D, 394C)
polypeptide chain 2
   Domain F: VH (“Antigen A”)
   Domain G: CH3 (L351D, 351C)
polypeptide chain 3
   Domain H: VL (“Nivo”)
   Domain I: CL (kappa)
   Domain J: CH2
   Domain K: CH3 (Hole, 349C)
polypeptide chain 4 (=“BC1” chain 4)
   Domain L: VH (“Nivo”)
   Domain M: CH1

This construct did not express.

### 6.14.10. Example 9: Trivalent 1×2 Bispecific Construct (“BC28-1×2”)

We constructed a trivalent 1×2 bispecific B-Body having the following domain structure:
1^{st} polypeptide chain (=“BC28” chain 1) (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:37)
   Domain R=VL (Antigen “A”)
   Domain S=CH3 (Y349C; 445P, 446G, 447K insertion)
   Linker=GSGSGS (SEQ ID NO: 40)
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11):
   Domain L=VH (“Nivo”)
   Domain M=CH1.
6^{th} polypeptide chain (=“BC28” chain 2) (SEQ ID NO:25)
   Domain T=VH (Antigen “A”)
   Domain U=CH3 (S354C; 445P, 446G, 447K insertion)

The A:F antigen binding site is specific for “Antigen A”, as is the H:L binding antigen binding site. The R:T antigen binding site is specific for PD. The specificity of this construct is thus Antigen “A” x(PD1-Antigen “A”).

### 6.14.11. Example 10: Trivalent 1×2 Bispecific Construct (“CTLA4-4 x Nivo x CTLA4-4”)

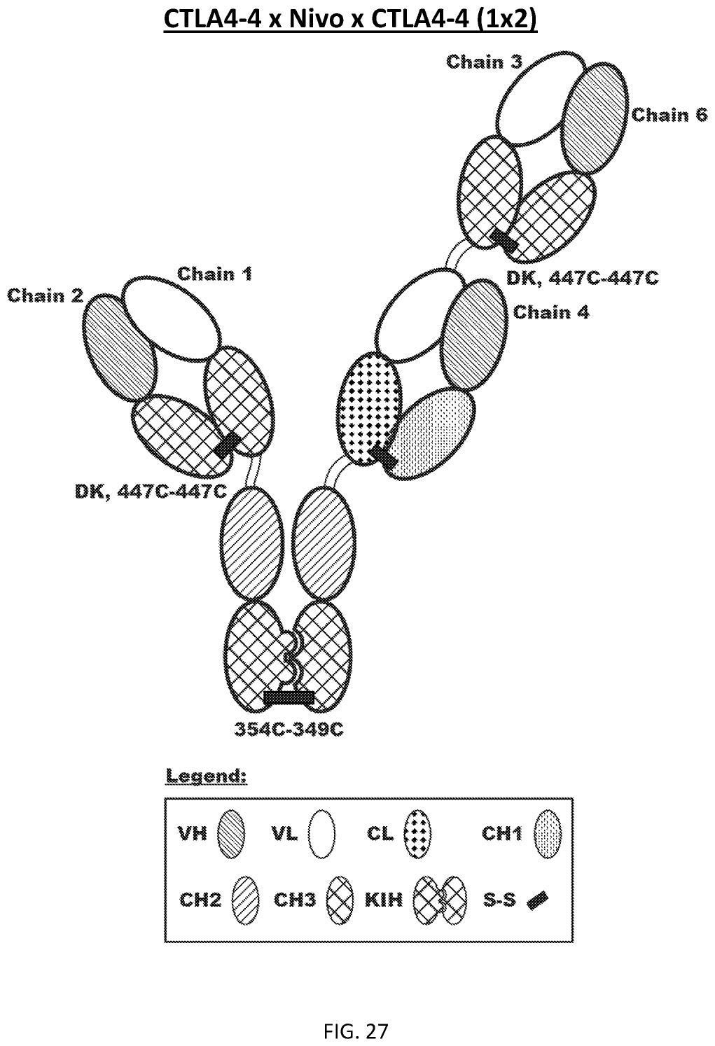

We constructed a trivalent 1×2 bispecific molecule having the general structure schematized in FIG. 27 (“CTLA4-4 x Nivo x CTLA4-4”). Domain nomenclature is set forth in FIG. 26.

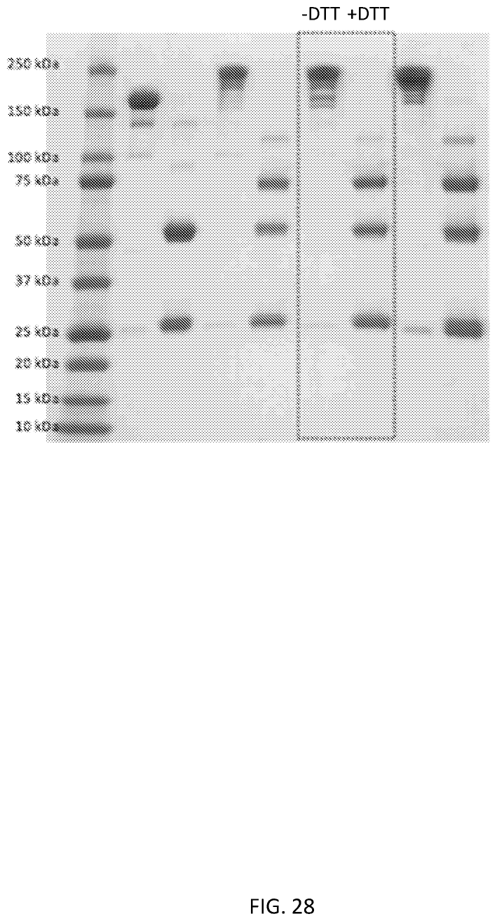

FIG. 28 is a SDS-PAGE gel in which the lanes showing the “CTLA4-4 x Nivo x CTLA4-4” construct under non-reducing and reducing conditions have been boxed.

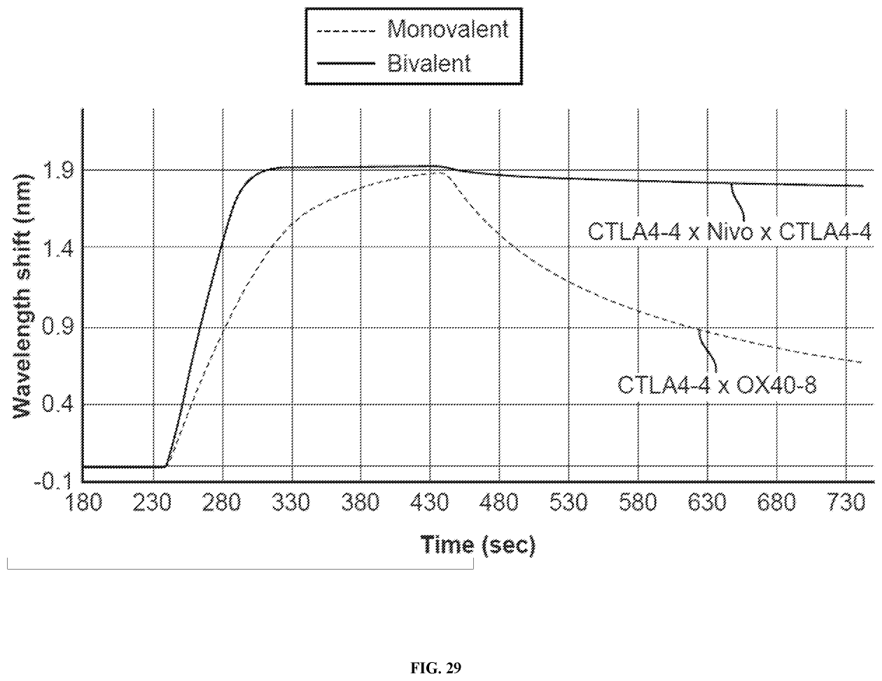

FIG. 29 compares antigen binding of two antibodies: “CTLA4-4 x OX40-8” and “CTLA4-4 x Nivo x CTLA4-4”. “CTLA4-4 x OX40-8” binds to CTLA4 monovalently; while “CTLA4-4 x Nivo x CTLA4-4” bind to CTLA4 bivalently.

### 6.14.12. Example 11: Trivalent 1×2 Trispecific Construct “BC28-1×1×1a”

We constructed a trivalent 1×2 trispecific molecule having the general structure schematized in FIG. 30. With reference to the domain nomenclature set forth in FIG. 26,
1^{st} polypeptide chain (=“BC28” chain1) [SEQ ID NO:24]
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:45)
   Domain R=VL (CTLA4-4)
   Domain S=CH3 (T366K; 445K, 446S, 447C insertion)
   Linker=GSGSGS (SEQ ID NO: 40)
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11)
   Domain L=VH (“Nivo”)
   Domain M=CH1.
6^{th} polypeptide chain (=hCTLA4-4 chain2) (SEQ ID NO:53)
   Domain T=VH (CTLA4)
   Domain U=CH3 (L351D, 445G, 446E, 447C insertion)

The antigen binding sites of this trispecific construct were:

Antigen binding site A:F was specific for “Antigen A”

Antigen binding site H:L was specific for PD1 (nivolumab sequence)

Antigen binding site R:T was specific for CTLA4.

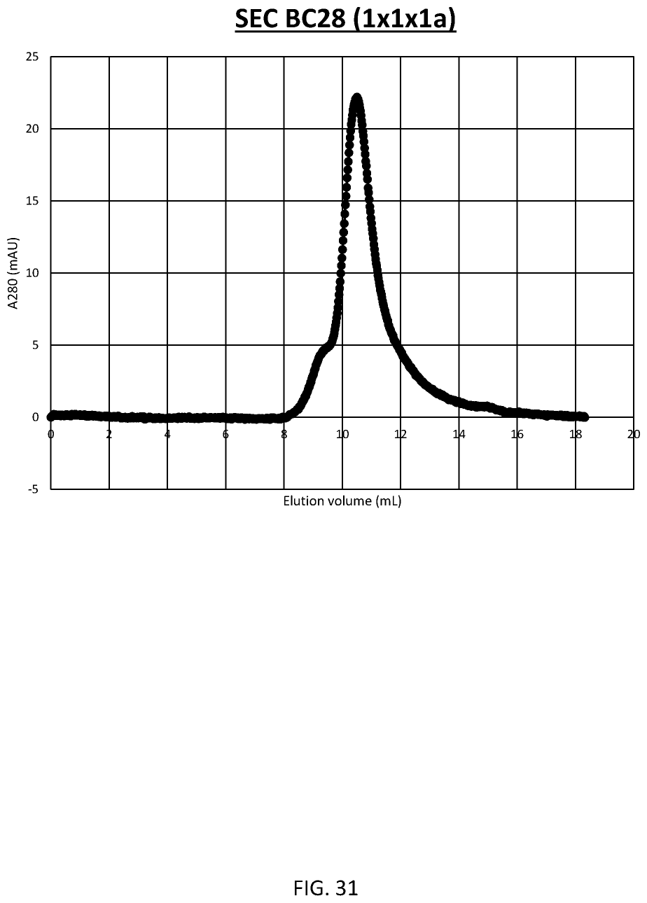

FIG. 31 shows size exclusion chromatography with “BC28-1×1×1a” following transient expression and one-step purification using the CaptureSelect™ CH1 affinity resin, demonstrating a single well-defined peak.

### 6.14.13. Example 12: SDS-PAGE Analysis of Bivalent and Trivalent Constructs

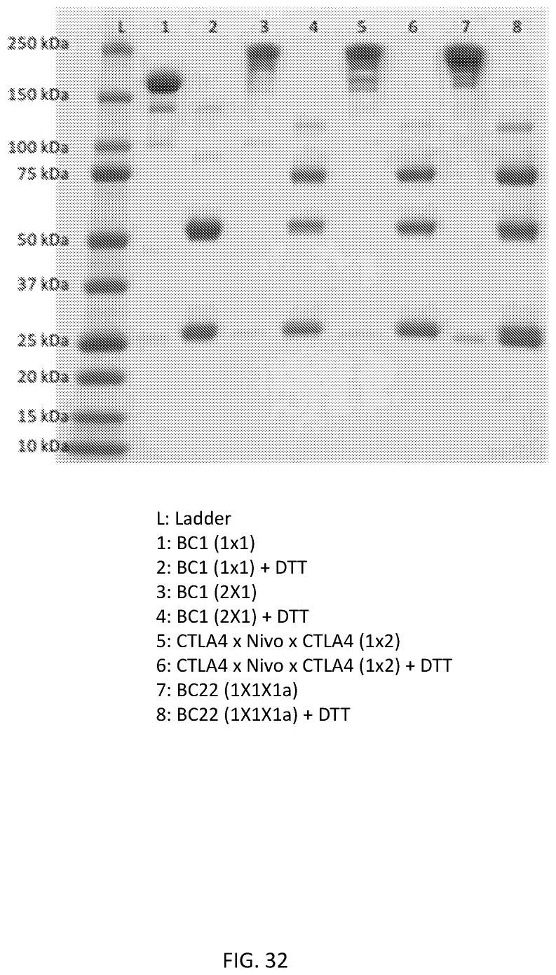

FIG. 32 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the trivalent bispecific 2×1 construct “BC1-2×1” (see Example 7). Lanes 5 (nonreducing) and 6 (reducing) are the trivalent 1×2 bispecific construct “CTLA4-4 x Nivo x CTLA4-4” (see Example 10). Lanes 7 (nonreducing) and 8 (reducing) are the trivalent 1×2 trispecific “BC28-1×1×1a” construct described in Example 11.

The SDS-PAGE gel demonstrates the complete assembly of each construct, with the predominant band in the non-reducing gel appearing at the expected molecular weight for each construct.

### 6.14.14. Example 13: Binding Analysis

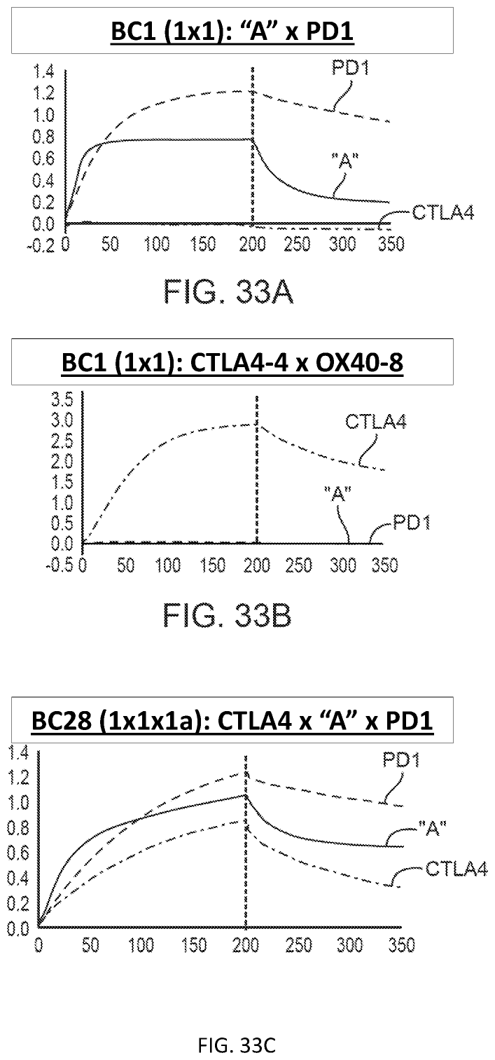

FIG. 33 shows Octet binding analyses to 3 antigens: PD1, Antigen “A”, and CTLA-4. In each instance, the antigen is immobilized and the B-Body is the analyte. For reference, 1×1 bispecifics “BC1” and “CTLA4-4x OX40-8” were also compared to demonstrate 1×1 B-Bodies bind specifically only to antigens for which the antigen binding sites were selected.

FIG. 33A shows binding of “BC1” to PD1 and to Antigen “A”, but not CTLA4. FIG. 33B shows binding of a bivalent bispecific 1×1 construct “CTLA4-4x OX40-8” to CTLA4, but not to Antigen “A” or PD1. FIG. 33C shows the binding of the trivalent trispecific 1×2 construct, “BC28-1×1×1a” to PD1, Antigen “A”, and CTLA4.

### 6.14.15. Example 14: Tetravalent Constructs

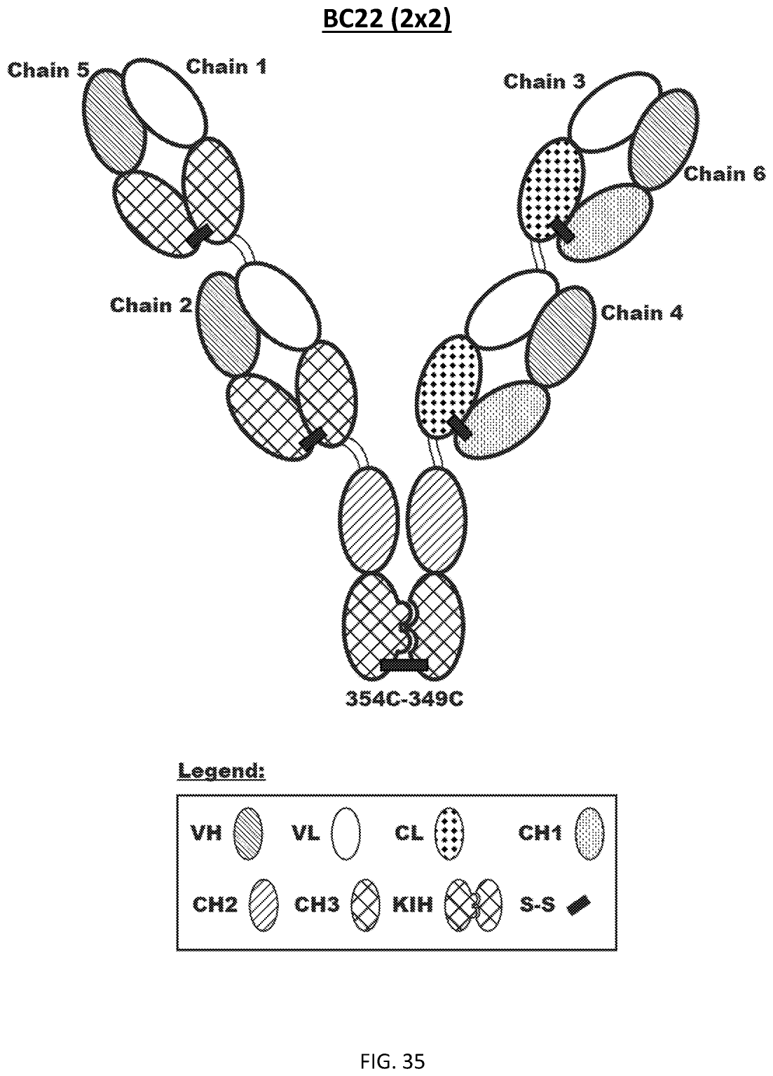

FIG. 35 shows the overall architecture of a 2×2 tetravalent bispecific construct “BC22-2×2”. The 2×2 tetravalent bispecific was constructed with “BC1” scaffold by duplicating each variable domain-constant domain segment. Domain nomenclature is schematized in FIG. 34.

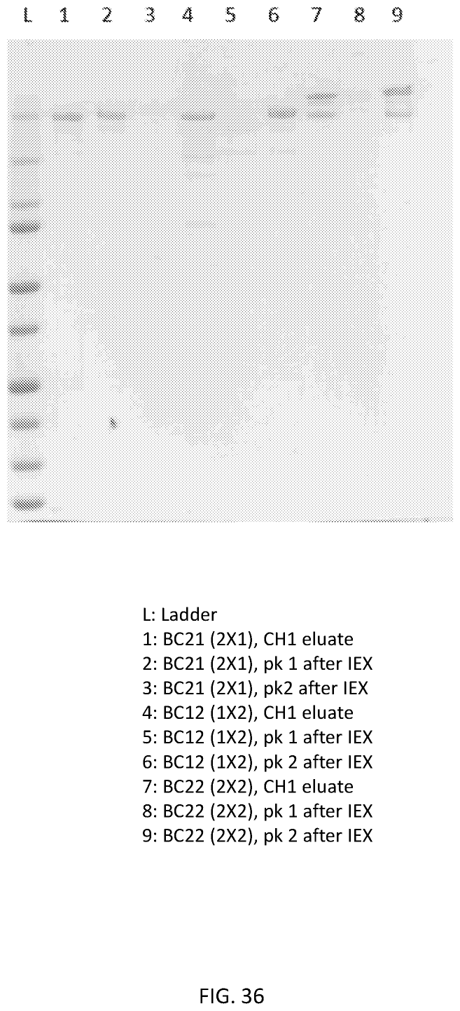

FIG. 36 is a SDS-PAGE gel. Lanes 7-9 show the “BC22-2×2” tetravalent construct respectively following one-step purification using the CaptureSelect™ CH1 affinity resin (“CH1 eluate”), and after an additional ion exchange chromatography purification (lane 8, “pk 1 after IEX”; lane 9, “pk 2 after IEX”). Lanes 1-3 are the trivalent 2×1 construct “BC21-2×1” after CH1 affinity purification (lane 1) and, in lanes 2 and 3, subsequent ion exchange chromatography. Lanes 4-6 are the 1×2 trivalent construct “BC12-1×2”.

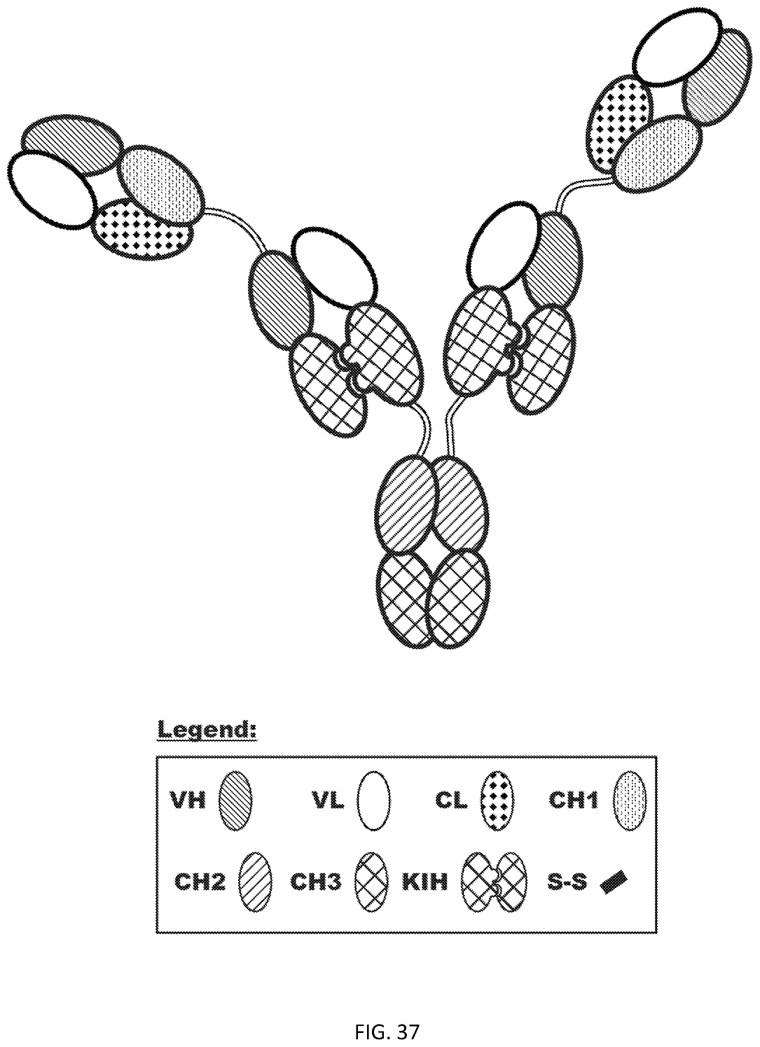

FIG. 37 shows the overall architecture of a 2×2 tetravalent construct.

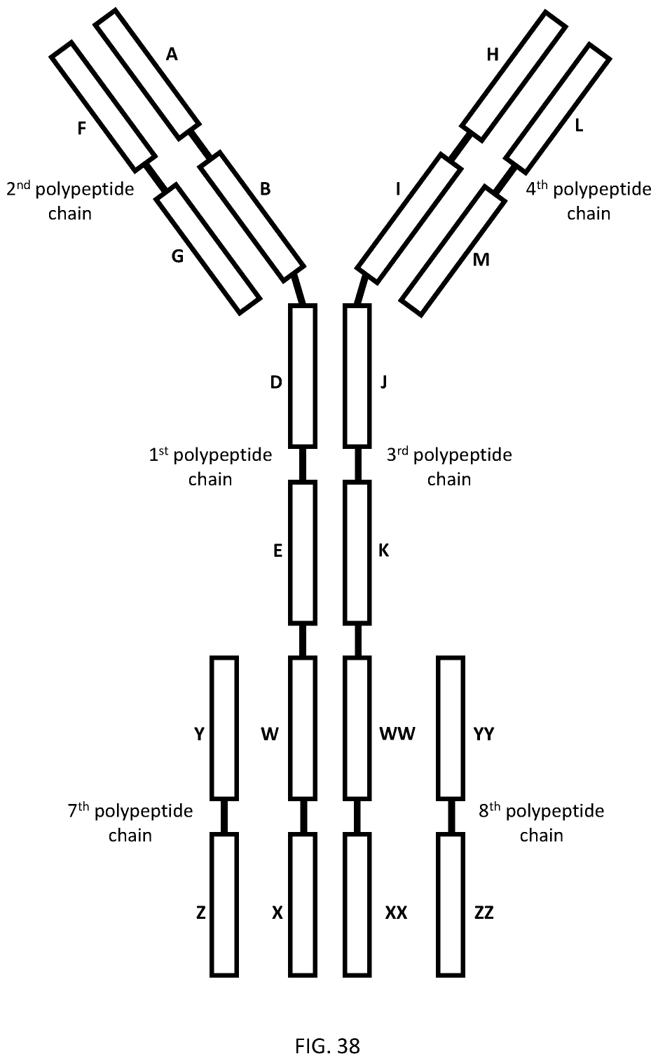

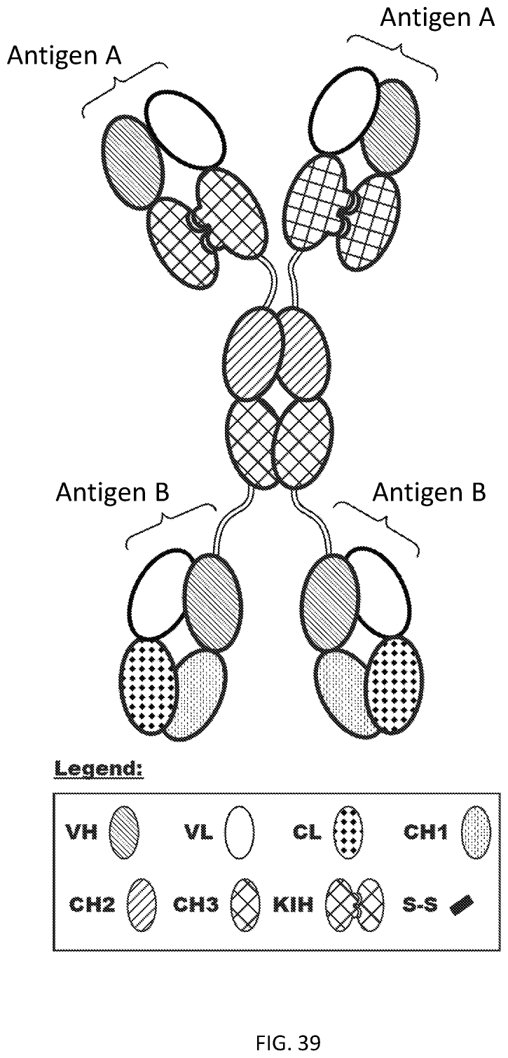

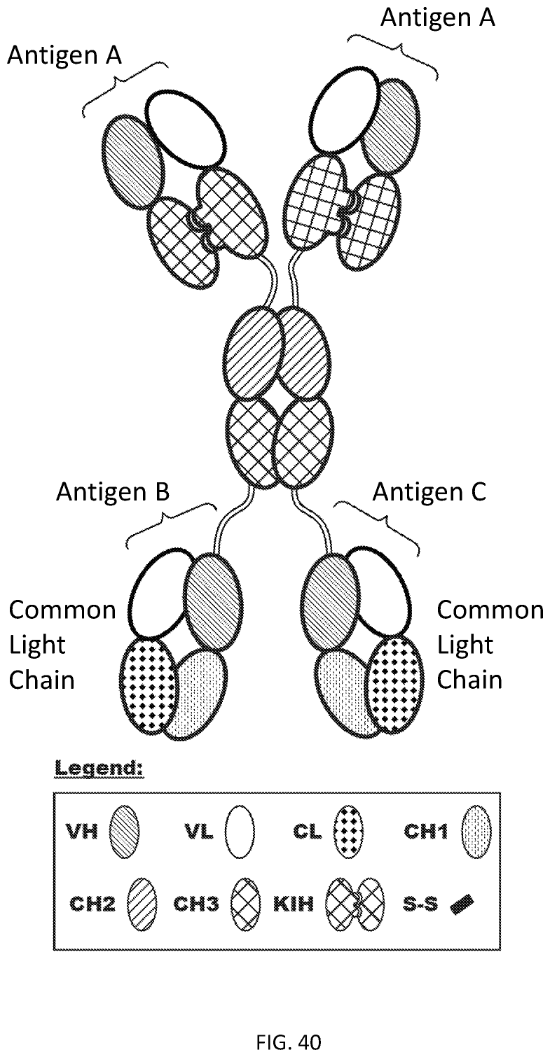

FIGS. 39 and 40 schematize tetravalent constructs having alternative architectures. Domain nomenclature is presented in FIG. 38.

### 6.14.16. Example 15: Bispecific Antigen Engagement by B-Body

A tetravalent bispecific 2×2 B-Body “B-Body-IgG 2×2” was constructed. In greater detail, using the domain and polypeptide chain references summarized in FIG. 38,
1^{st} polypeptide chain
   Domain A=VL (Certolizumab)
   Domain B=CH3 (IgG1, knob)
   Domain D=CH2 (IgG1)
   Domain E=CH3 (IgG1)
   Domain W=VH (Antigen “A”)
   Domain X=CH1 (IgG1)
3^{rd} polypeptide chain (identical to first polypeptide chain)
   Domain H=VL (Certolizumab)
   Domain I=CH3 (IgG1, knob)
   Domain J=CH2 (IgG1)
   Domain K=CH3 (IgG1)
   Domain WW=VH (Antigen “A”)
   Domain XX=CH1 (IgG1)
2^{nd} polypeptide chain
   Domain F=VH (Certolizumab)
   Domain G=CH3 (IgG1, hole)
4^{th} polypeptide chain (identical to third polypeptide chain)
   Domain F=VH (Certolizumab)
   Domain G=CH3 (IgG1, hole)
7^{th} polypeptide chain
   Domain Y=VH (“Antigen A”)
   Domain Z=CL Kappa
8^{th} polypeptide chain (identical to seventh polypeptide chain)
   Domain YY=VH (“Antigen A”)
   Domain ZZ=CL Kappa.

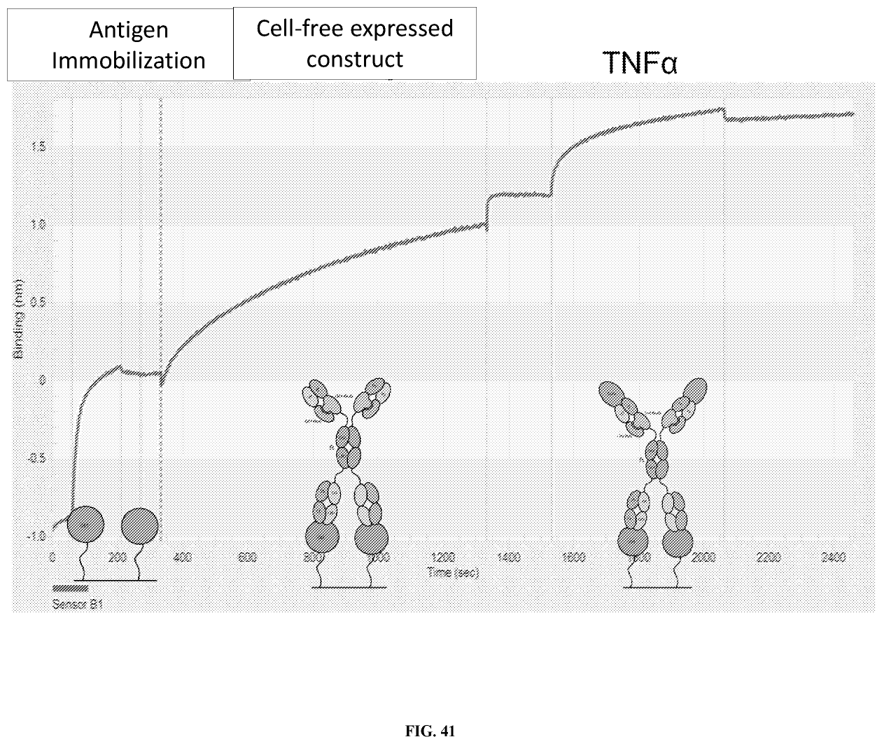

This was cloned and expressed as described in Example 1. Here, the BLI experiment consisted of immobilization of biotinylated antigen “A” on a streptavidin sensor, followed by establishing baseline with 10× kinetic buffer. The sensor was then dipped in cell-free expressed “B-Body-IgG 2×2” followed by establishment of a new baseline. Finally, the sensor was dipped in 100 nM TNFα where a second binding event was observed, confirming the bispecific binding of both antigens by a single “B-Body-IgG 2×2” construct. Results are shown in FIG. 41.

### 6.14.17. Example 16: Antigen-Specific Cell Binding of “BB-IgG 2×2”

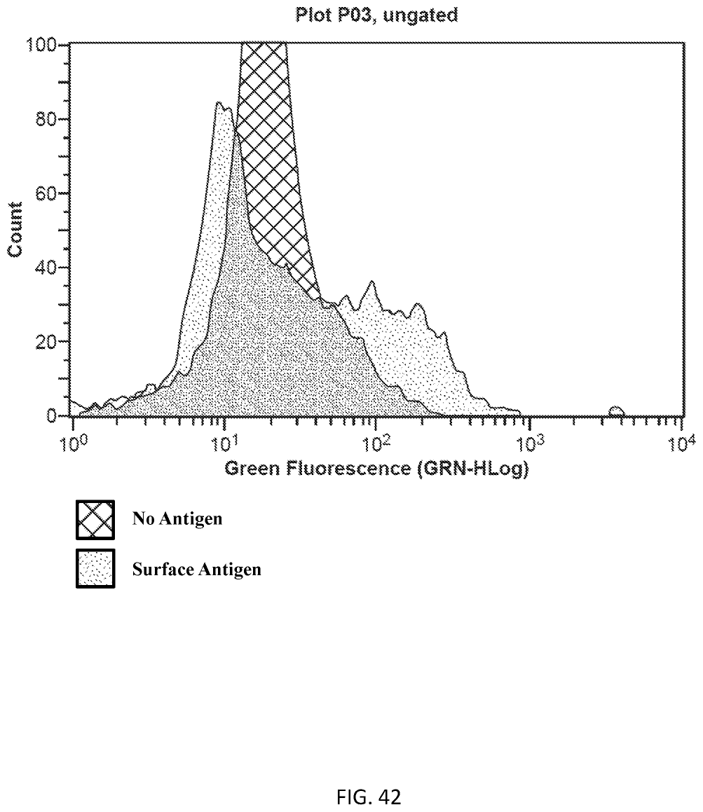

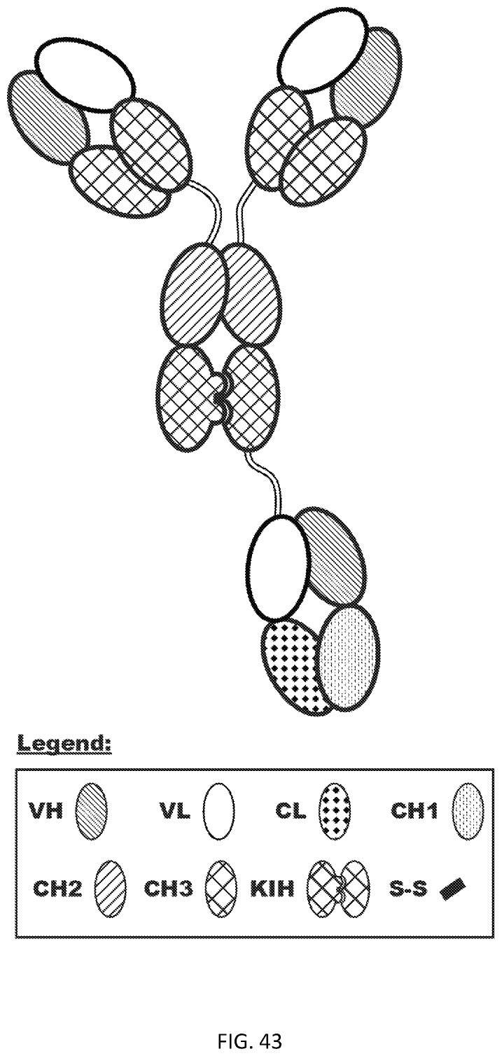

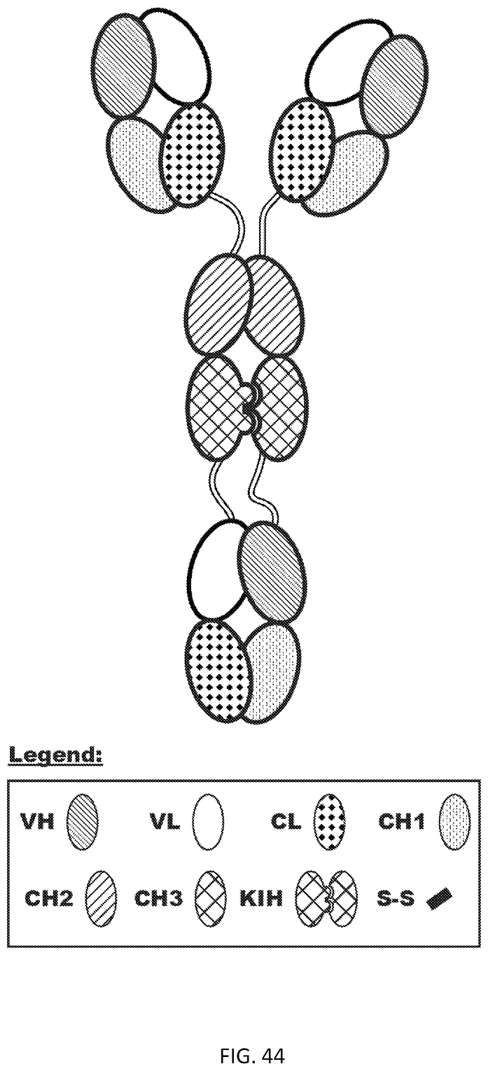

Expi-293 cells were either mock transfected or transiently transfected with Antigen “B” using the Expi-293 Transfection Kit (Life Technologies). Forty-eight hours after transfection, the Expi-293 cells were harvested and fixed in 4% paraformaldehyde for 15 minutes at room temperature. The cells were washed twice in PBS. 200,000 Antigen B or Mock transfected Expi-293 cells were placed in a V-bottom 96 well plate in 100 μL of PBS. The cells were incubated with the “B-Body-IgG 2×2” at a concentration of 3 μg/mL for 1.5 hours at room temperature. The cells were centrifuged at 300×G for 7 minutes, washed in PBS, and incubated with 100 μL of FITC labeled goat-anti human secondary antibody at a concentration of 8 μg/mL for 1 hour at room temperature. The cells were centrifuged at 300×G for 7 minutes, washed in PBS, and cell binding was confirmed by flow cytometry using a Guava easyCyte. Results are shown in FIG. 42.

### 6.14.18. Example 17: SDS-PAGE Analysis of Bivalent and Trivalent Constructs

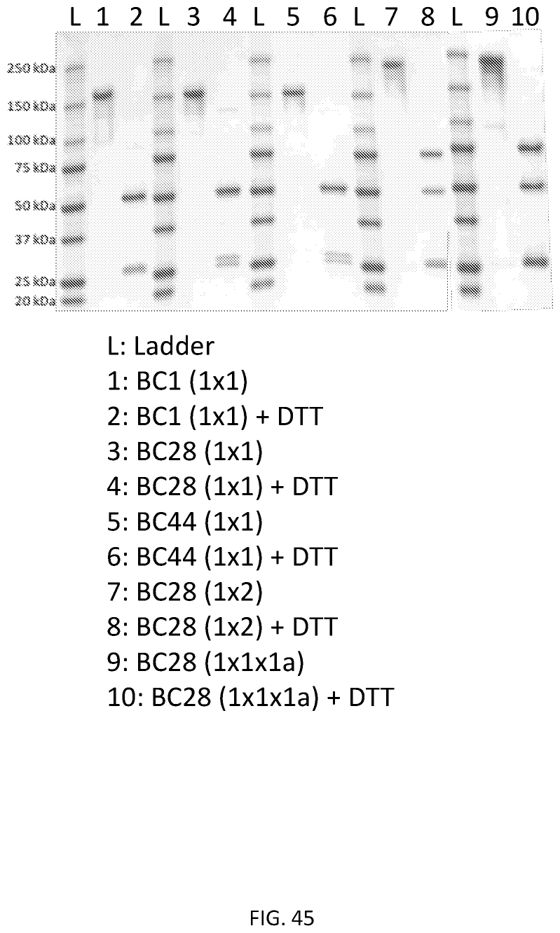

FIG. 45 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the bivalent 1×1 bispecific construct “BC28” (see Example 4). Lanes 5 (nonreducing) and 6 (reducing) are the bivalent 1×1 bispecific construct “BC44” (see Example 5). Lanes 7 (nonreducing) and 8 (reducing) are the trivalent 1×2 bispecific “BC28-1×2” construct (see Example 9). Lanes 9 (nonreducing) and 10 (reducing) are the trivalent 1×2 trispecific “BC28-1×1×1a” construct described in Example 11.

The SDS-PAGE gel demonstrates the complete assembly of each construct, with the predominant band in the non-reducing gel appearing at the expected molecular weight for each construct.

### 6.14.19. Example 18: Stability Analysis of Variable-CH3 Junction Engineering

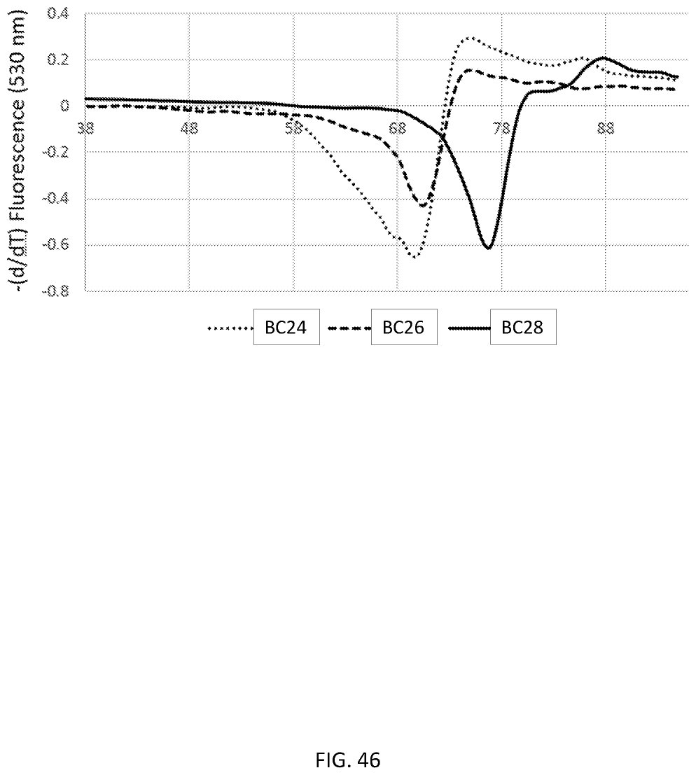

Pairing stability between various junctional variant combinations was assessed. Differential scanning fluorimetry was performed to determine the melting temperature of various junctional variant pairings between VL-CH3 polypeptides from Chain 1 (domains A and B) and VH-CH3 polypeptides from 2 (domains F and G). Junctional variants “BC6jv”, “BC28jv”, “BC30jv”, “BC44jv”, and “BC45jv”, each having the corresponding junctional sequences of “BC6”, “BC28”, “BC30”, “BC44”, and “BC45” found in Table 2 and Table 3 above, demonstrate increased pairing stability with Tm's in the 76-77 degree range (see Table 4). FIG. 46 shows differences in the thermal transitions for “BC24jv”, “BC26jv”, and “BC28jv”, with “BC28jv” demonstrating the greatest stability of the three. The x-axis of the figure is temperature and the y-axis is the change in fluorescence divided by the change in temperature (−dFluor/dTemp). Experiments were performed as described in Niesen et al. (Nature Protocols, (2007) 2, 2212-2221), which is hereby incorporated by reference for all it teaches.

TABLE 4
Melting Temperatures of Junctional Variant Pairs

| JUNCTIONAL | MELTING TEMP | MELTING TEMP |
|---|---|---|
| VARIANT PAIR | #1 (° C.) | #2 (° C.) |
| BC1jv | 69.7 | 55.6 |
| BC5jv | 71.6 | |
| BC6jv | 77 | |
| BC15jv | 68.2 | 54 |
| BC16jv | 65.9 | |
| BC17jv | 68 | |
| BC24jv | 69.7 | |
| BC26jv | 70.3 | |
| BC28jv | 76.7 | |
| BC30jv | 76.8 | |
| BC44jv | 76.2 | |
| BC45jv | 76 | |

### 6.14.20. Example 19: CD3 Candidate Binding Molecules

Various CD3 antibodies were constructed and tested as described below.

### 6.14.21. Example 21: Single Step Purification of Bispecific Antibodies Having CH1 in Both Arms

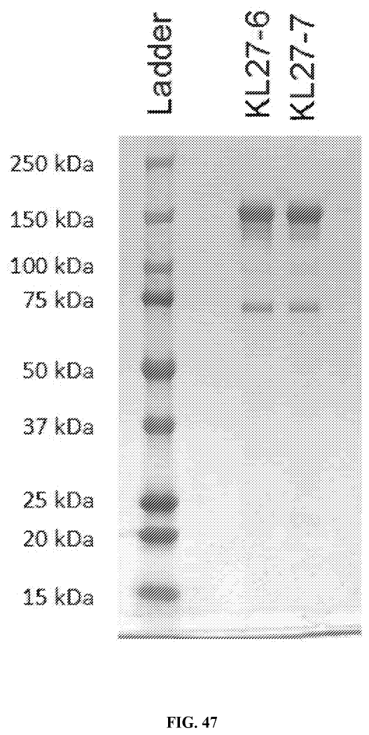

Anti-CH1 purification efficiency of bispecific antibodies was also tested for binding molecules having only standard knob-hole orthogonal mutations introduced into CH3 domains found in their native positions within the Fc portion of the bispecific antibody with no other domain modifications. Therefore, the two antibodies tested, KL27-6 and KL27-7, each contained two CH1 domains, one on each arm of the antibody. As described in more detail herein, each bispecific antibody was expressed, purified from undesired protein products on an anti-CH1 column, and run on an SDS-PAGE gel. As shown in FIG. 47, a significant band at 75 kDa representing an incomplete bispecific antibody was present, interpreted as a complex containing only (i) a first and second or (ii) third and fourth polypeptide chains with reference to FIG. 3. Thus, methods using anti-CH1 to purify complete bispecific molecules that have a CH1 domain in each arm resulted in background contamination due to incomplete antibody complexes.

### 6.14.22. Example 22: Fc Mutations Reducing Effector Function

A series of engineered Fc variants were generated in the monoclonal IgG1 antibody trastuzumab (Herceptin, “WT-IgG1”) with mutations at positions L234, L235, and P329 of the CH2 domain. The specific mutations for the variants tested are described in Table 5 below and include sFc1 (PALALA), sFc7 (PGLALA), and sFc10 (PKLALA). All variants were produced by Expi293 expression as described herein.

TABLE 5
Fc variants

| Variant | L234 | L235 | P329 |
|---|---|---|---|
| sFc1 | A | A | P |
| sFc2 | G | G | P |
| sFc3 | L | L | A |
| sFc4 | A | A | A |
| sFc5 | G | G | A |
| sFc6 | L | L | G |
| sFc7 | A | A | G |
| sFc8 | G | G | G |
| sFc9 | L | L | K |
| sFc10 | A | A | K |
| sFc11 | G | G | K |
| wt IgG1 | L | L | P |

Stability Analysis

The protein melting temperature was determined using the Protein Thermal Shift Dye Kit (Thermo Fisher). Briefly, proteins of interest were brought to a concentration of 1 mg/ml. Thermal shift dye mix (water, Thermal shift buffer, and Thermal Shift Dye) was added to the protein of interest. The protein/thermal dye mix was added to glass capillary tubes and analyzed using a thermal gradient on a Roche Light Cycler. Proteins were incubated at 37° C. for 2 minutes before initiating a thermal gradient from 37° C. to 99° C. with a temperature increase rate of 0.1° C./sec. Fluorescence increase was measured over time and used to calculate the thermal melting temperature.

Table 6 depicts results from the Protein Thermal Shift experiment above. All variants showed comparable stability as the wild-type IgG.

TABLE 6
melting temperature analysis

| Variant | L234 | L235 | P329 | TM1 (° C.) | TM2 (° C.) |
|---|---|---|---|---|---|
| sFc1 | A | A | P | 68.1 | 81.8 |
| sFc2 | G | G | P | 69.7 | 81.7 |
| sFc3 | L | L | A | 65.6 | 81.7 |
| sFc4 | A | A | A | 66.4 | 81.9 |
| sFc5 | G | G | A | 67.5 | 81.5 |
| sFc6 | L | L | G | 64.6 | 81.4 |
| sFc7 | A | A | G | 65.7 | 81.8 |
| sFc8 | G | G | G | 66.2 | 81.8 |
| sFc9 | L | L | K | 64.5 | 81.2 |
| sFc10 | A | A | K | 65.3 | 81.1 |
| sFc11 | G | G | K | 66.2 | 81.5 |
| wt IgG1 | L | L | P | 66.2 | 81.1 |

Interaction of the trastuzumab Fc variants with CD64 was assessed using bio-layer interferometry (Octet/FORTEBIO®). Briefly, Her2 antigen or anti-CH1 antibody was immobilized onto the biosensor tip surface. Antibody solutions comprising the Fc variants listed in Table 5 were flowed over the biosensor, followed by one wash for baseline equilibration followed by an analyte solution containing 200 nM CD64 Response profiles were generated in real time.

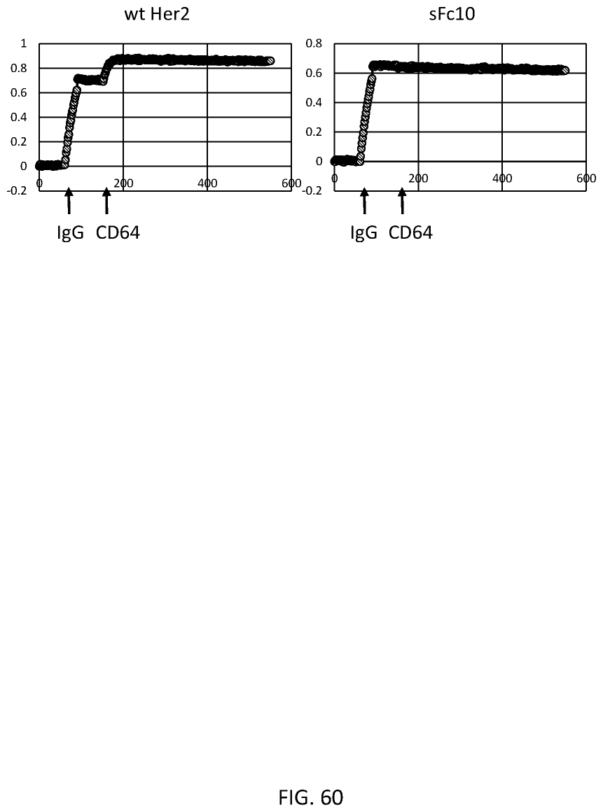

FIG. 60 depicts an exemplary Octet binding analysis. The native trastuzumab antibody (WT Her2) condition revealed a first shift when the antibody solution was flowed over the biosensor, and a second shift when CD64 was added. By contrast, the sFc10 variant condition, while revealing a first shift when the antibody solution was flowed over the biosensor, revealed no shift when CD64 was added.

TABLE 7
summary of Octet analysis

| Variant | L234 | L235 | P329 | Her2 Binding | CD64 Binding? |
|---|---|---|---|---|---|
| sFc1 | A | A | P | Yes (strong) | Yes (weak) |
| sFc2 | G | G | P | Yes (strong) | No |
| sFc3 | L | L | A | Yes (strong) | Yes (strong) |
| sFc4 | A | A | A | Yes (strong) | No |
| sFc5 | G | G | A | Yes (strong) | No |
| sFc6 | L | L | G | Yes (strong) | Yes (strong) |
| sFc7 | A | A | G | Yes (strong) | No |
| sFc8 | G | G | G | Yes (strong) | No |
| sFc9 | L | L | K | Yes (strong) | Yes (weak) |
| sFc10 | A | A | K | Yes (strong) | No |
| sFc11 | G | G | K | Yes (strong) | No |
| wt IgG1 | L | L | P | Yes (strong) | Yes (strong) |

Antibody-Dependent Cell-Mediated Cytotoxicity (ADCC) Assay

The impact of selected Fc mutations on FCγRIIIa effector function was assessed using the ADCC Bioreporter Assay Kit (Promega). Briefly, a serial dilution of each variant was incubated with SKBR3 cells. The reactions were then incubated at 37° C. in a humidified CO2 incubator with the ADCC Bioassay effector cells according to the manufacturer's protocol and incubated for 6 to 24 hrs. After incubation, the Bio-Glo™ Luciferase Assay Reagent was added to each sample and the luminescent signal was measured with a plate reader with glow-type luminescence read capabilities.

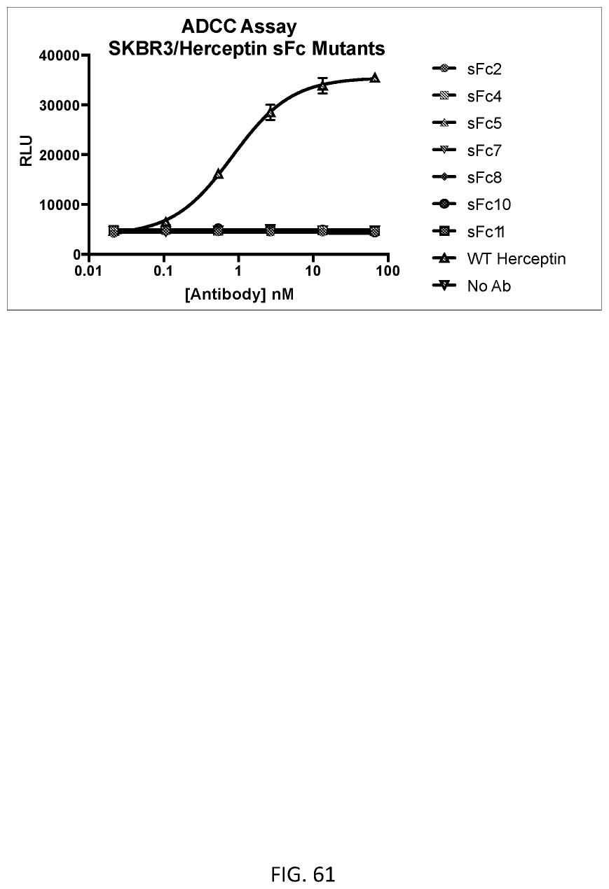

FIG. 61 depicts results from the above ADCC assay. The native trastuzumab antibody (WT Herceptin) exhibited robust ADCC activity. By contrast, the Fc variants exhibited no ADCC activity.

Clq Binding Profiles

The impact of Fc mutations on Clq binding was assessed using an ELISA assay. Up to 128 μg/ml IgG was immobilized for each of the variants. Here the ELISA was performed with 12 μg/ml Clq, 1/400 dilution of the Clq-HRP secondary antibody. Washes and samples were diluted in PBST-BSA (1%).

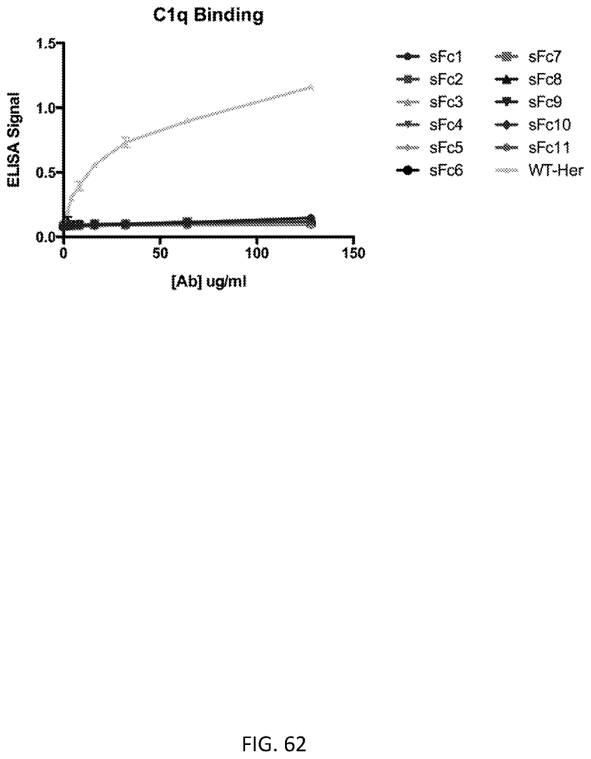

FIG. 62 depicts results from the above Clq ELISA assay. The native trastuzumab antibody exhibited robust binding to Clq. By contrast, all of the Fc variants exhibited virtually no detectable binding to Clq.

### 6.14.23. Example 23: Construction of a 1×1×1 Trispecific Antibody with Orthogonal CH1/CL Modifications

Antibody Construction

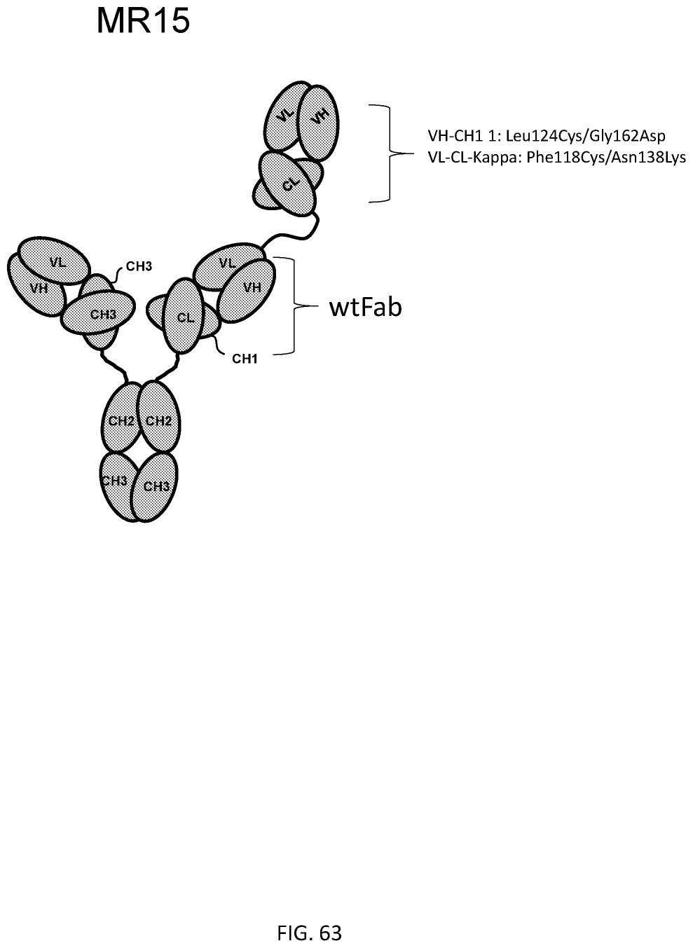

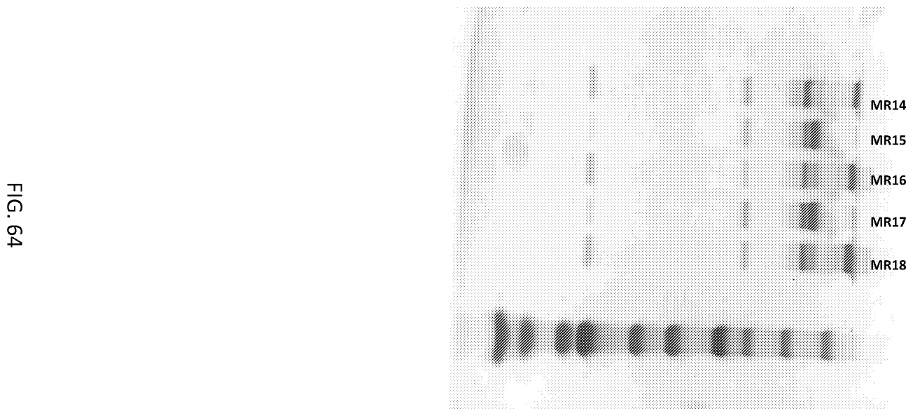

We constructed binding molecule “MR-15” having the architecture depicted in FIG. 63. MR-15 has a first, second, third, fourth, and fifth polypeptide chain, wherein (a) the first polypeptide chain comprises, from N-terminus to C-terminus, a first VL amino acid sequence, a human IgG1 CH3 amino acid sequence with a Y349C mutation and a C-terminal extension incorporating a PGK tripeptide sequence that is followed by the DKTHT motif (SEQ ID NO: 185) of an IgG1 hinge region, a human IgG1 CH2 amino acid sequence, and a human IgG1 CH3 amino acid with a S354C and a T366W mutation; (b) the second polypeptide comprises, from N-terminus to C-terminus, a first VH amino acid sequence and a human IgG1 CH3 amino acid sequence with a S354C mutation and a C-terminal extension incorporating a PGK tripeptide sequence; (c) the third polypeptide comprises, from N-terminus to C-terminus, a second VL amino acid sequence which is a wtFab sequence, a first CL kappa amino acid sequence comprising Phe118Cys and Asn138Lys mutations, a third VL amino acid sequence which is a wtFab sequence, a second CL kappa amino acid sequence which is a wtFab sequence; (d) the fourth polypeptide comprises, from N-terminus to C-terminus, a second VH sequence which is a wtFab sequence, a first CH1 sequence comprising Leu128Phe118Cys and Asn138Lys mutations; and (e) the fifth polypeptide comprises, from N-terminus to C-terminus and a third VH sequence which is a wtFab sequence, and a second CH1 sequence which is a wtFab sequence.

SDS-PAGE Analysis

Purified variants were analyzed by non-reducing SDS-PAGE for the presence of complete product, incomplete product, and overall purity. 2 μg of each sample was added to 15 μL SDS loading buffer. Non-reducing samples were incubated at 95° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. FIG. 64 depicts an image of the destained gel, showing that MR15 largely assembles into the intended full-length construct with minimal incomplete assembly.

Mass Spectrometry Analysis

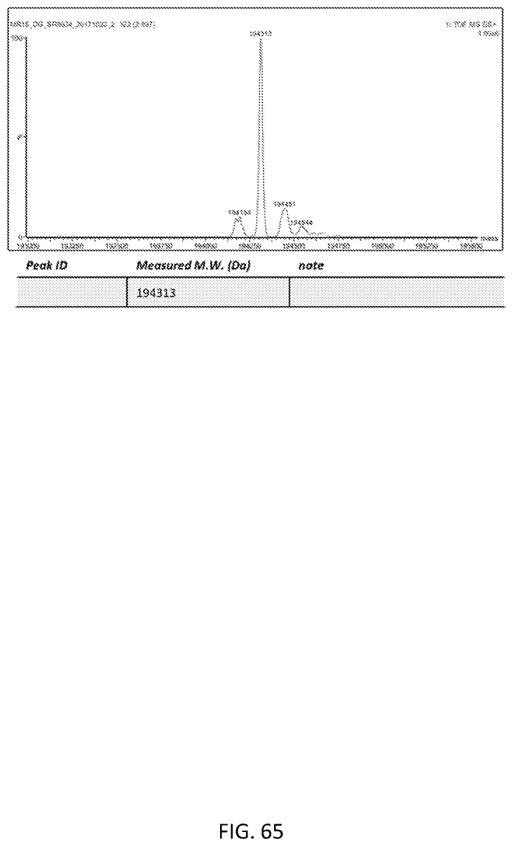

MR variants, including MR15, were purified and analyzed by mass spectrometry to confirm the correct species by molecular weight. Briefly, samples were treated with a cocktail of enzymes to remove glycosylation. Samples were both tested in the reduced format to specifically identify each chain by molecular weight. Samples were also tested under non-reducing conditions to identify the molecular weights of all complexes in the purified samples. FIG. 65 depicts mass spectrogram results, indicating a lack of incompletely assembled variants.

### 6.14.24. Example 24: Bivalent, Bispecific Antibody with Distinct Orthogonal Pair CH1/CL Modifications in Each Arm Improves Full Antibody Assembly

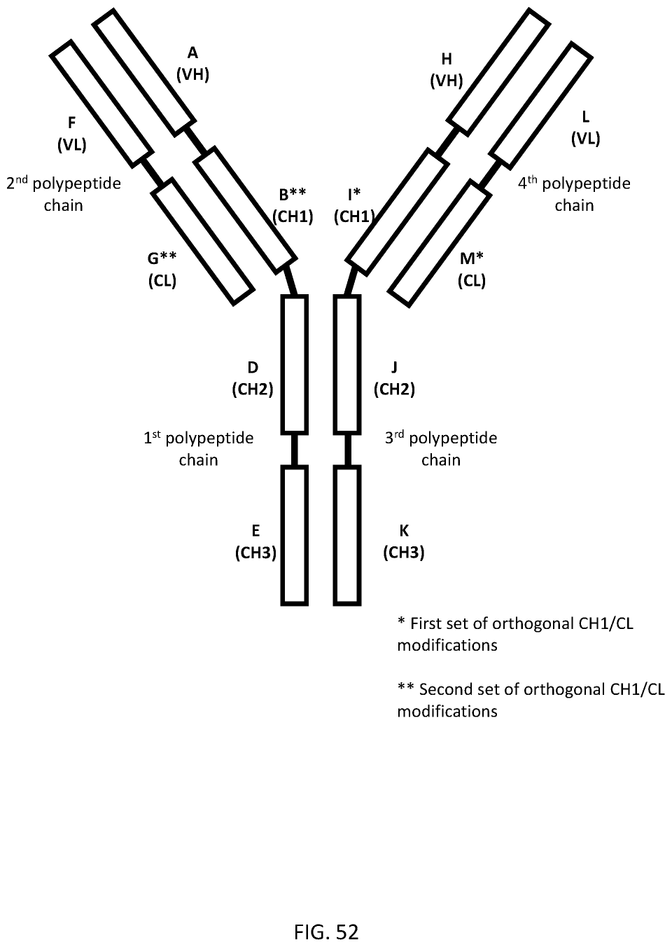

A bivalent, bispecific antibody having distinct orthogonal CH1/CL modifications in each arm, and having a knob-in-hole orthogonal mutation in CH3, was constructed. With reference to FIG. 52, the bivalent, bispecific antibody had the following architecture and amino acid substitutions:
Polypeptide 1: A-B-D-E, wherein
   A=VH
   B=CH1, Leu128Cys/Gly166Asp
   D=CH2
   E=CH3, Ser354Cys/Thr366Trp
Polypeptide 2: F-G, wherein
   F=VL
   G=CL, Phe118Cys/Asn138Lys
Polypeptide 3: H-I-J-K, wherein
   H=VH
   I=CH1, Gly166Lys
   J=CH2
   K=CH3, Tyr349Cys/Thr366Ser/Leu368A1a/Tyr407Val
Polypeptide 4: L-M, wherein
   L=VL
   M=CL, Asn138Asp

Briefly, the four polypeptides were transfected together in Expi293 cells. The supernatants were collected after five days. Antibodies were purified with Protein-A using standard procedures in step 1. In a subsequent step Mono-S ion exchange chromatography was used to isolate the intact, full-length species. Fractions 1, 2, and 3 were collected.

Purified antibodies were analyzed by non-reducing SDS-PAGE for the presence of complete product, incomplete product, and overall purity. 2 μg of each sample was added to 15 μL SDS loading buffer. Non-reducing samples were incubated at 95° C. for 5 minutes without reducing agent. The non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis.

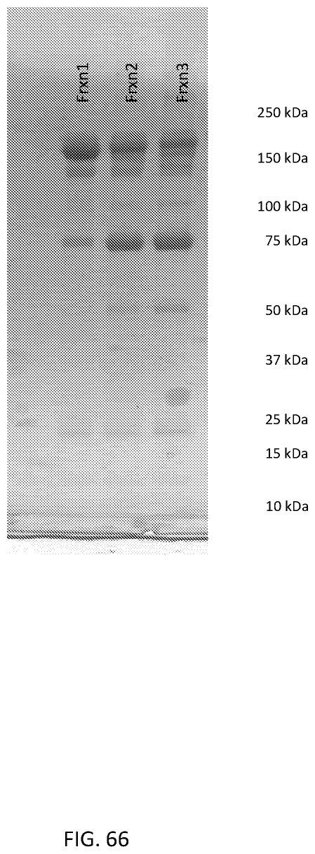

FIG. 66 depicts an image of the destained gel, showing that the fully assembled antibody is largely isolated in the first fraction, and that the second and third fractions generally contain incomplete product.

### 6.14.25. Example 25: Bivalent, Bispecific Antibody with Distinct Orthogonal Pair CH1/CL Modifications in Each Arm Improves Yield, Full Antibody Assembly, and Correct Heavy/Light Chain Pairings in Each Arm

Bivalent, bispecific antibodies having distinct orthogonal CH1/CL modifications in each arm, and having a knob-in-hole orthogonal modification in CH3/CH3, were constructed. With reference to FIG. 52, the bivalent, bispecific antibodies had the following architecture and amino acid substitutions:
Polypeptide 1: A-B-D-E, wherein
   A=VH
   B=CH1, Gly166Asp
   D=CH2
   E=CH3, Ser354Cys/Thr366Trp
Polypeptide 2: F-G, wherein
   F=VL
   G=CL, Asn138Lys
Polypeptide 3: H-I-J-K, wherein
   H=VH
   I=CH1, Gly166Lys
   J=CH2
   K=CH3, Tyr349Cys/Thr366Ser/Leu368A1a/Tyr407Val
Polypeptide 4: L-M, wherein
   L=VL
   M=CL, Asn138Asp

Three different antibodies were expressed by transfection in Expi293 cells. MR30 expressed polypeptides 1, 2, 3, and 4. MR31 expressed polypeptides 1, 3, and 4. MR32 expressed polypeptides 1, 2, and 3. Briefly, the four polypeptides were transfected together in Expi293 cells. The supernatants were collected after five days. Antibodies were purified with Protein-A using standard procedures in step 1.

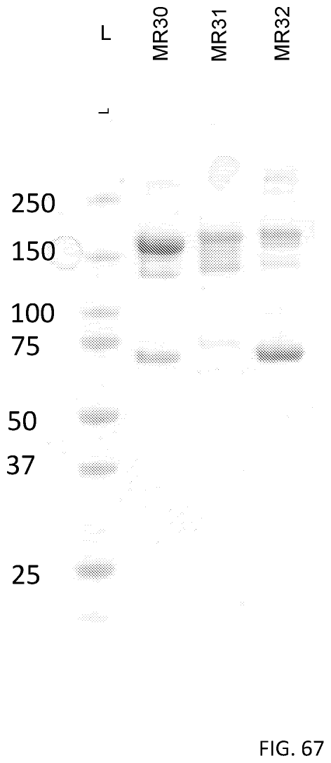

Purified antibodies were analyzed by SDS-PAGE analysis as described herein. Protein concentration was estimated measuring the absorbance at 280 nm which revealed high antibody yields when all four polypeptides were expressed. MR30 yielded 2.5 mg antibody, MR31 yielded 250 μg antibody, and MR32 yielded 2.7 mg antibody. Further, SDS-PAGE analysis, as depicted in FIG. 67, revealed that single-step Protein A purification resulted in overall higher purity of fully-assembled antibody. The MR31 and MR32 lanes revealed that polypeptide 1 interacts poorly with polypeptide 4, and that polypeptide 3 interacts poorly with polypeptide 2. These results indicate that full assembly of the bispecific antibody is driven by the correct pairings of polypeptides 1 and 2, and polypeptides 3 and 4, respectively.

### 6.14.26. Example 26: Bivalent Bispecific B-Body “BA1” with IgA-CH3

### 6.14.26.1. Construction of BA Binding Molecules

We constructed a bivalent bispecific B-Body, termed “BA”, specific for a first antigen PD1 and a second antigen (“Antigen A”). Salient features of the general BA architecture are illustrated in FIG. 3. In greater detail, with domain and polypeptide chain references in accordance with FIG. 3, the architecture of binding molecule “BA” was:
1^{st} polypeptide chain:
   Domain A=VL (“Antigen A”)
   Domain B=CH3 (IgA, first CH3 linker)
   Domain D=CH2
   Domain E=CH3 (Knob T366W, 354C)
2^{nd} polypeptide chain:
   Domain F=VH (“Antigen A”)
   Domain G=CH3 (IgA, second CH3 linker)
3rd polypeptide chain:
   Domain H=VL (“Nivo”)
   Domain I=CL (Kappa)
   Domain J=CH2
   Domain K=CH3 (Hole, 349C)
4^{th} polypeptide chain:
   Domain L=VH (“Nivo”)
   Domain M=CH1.

The A domain (SEQ ID NO: 12) and F domain (SEQ ID NO: 16) form an antigen binding site (A:F) specific for “Antigen A”. Domain H has the VL sequence from nivolumab (“Nivo”), and domain L has the VH sequence from “Nivo”. H and L associate to form an antigen binding site (H:L) specific for human PD1.

Domain B has the human IgA CH3 sequence, (SEQ ID NO:184), with a first CH3 linker sequence which connects the C-terminus of CH3 to the N-terminus of CH2 (domain D).

Domain G has the human IgA CH3 sequence, (SEQ ID NO:184), with a second CH3 linker sequence which forms a disulfide bridge with the first CH3 linker sequence.

Domain D has the sequence of human IgG1 CH2 domain (SEQ ID NO:20), with a CH2 hinge sequence (SEQ ID NO: 56) appended to the N-terminus of the CH2 domain.

Domain E (SEQ ID NO:15) has the sequence of human IgG1 CH3 with the mutations T366W and S354C.

Domain I (SEQ ID NO:19) has the sequence of human C kappa light chain (Cκ)

Domain J [SEQ ID NO:20] has the sequence of human IgG1 CH2 domain.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in Domain E. The 356E and L358M introduce isoallotype amino acids that reduce immunogenicity. The 366S, 368A, and 407V are “hole” mutations.

Domain M [SEQ ID NO: 23] has the sequence of the human IgG1 CH1 region. 6.14.26.2. Construction of first BA variant “BA1”

A first variant of binding molecule BA, “BA1” was constructed as above, wherein the first CH3 linker sequence that connects domain B to domain D is GEC, and wherein the second CH3 linker sequence is GEC.

The four polypeptide chains of BA1 were designed to comprise the following amino acid sequences in Table 8, below.

TABLE 8
BA1 sequences

| | | SED |
|---|---|---|
| | | ID |
| BA Chain # | Sequence | NO: |
| BA1. | DIQMTQSPSSLSASVGDRVTIT | 100 |
| chain 1 | CRASQDVNTAVAWYQQKPGKAP | |
| | KLLIYSASFLYSGVPSRFSGSR | |
| | SGTDFTLTISSLQPEDFATYYC | |
| | QQHYTTPPTFGQGTKVEIKRTT | |
| | FRPEVHLLPPPSEELALNELVTL | |
| | TCLARGFSPKDVLVRWLQGSQEL | |
| | PREKYLTWASRQEPSQGTTTFAV | |
| | TSILRVAAEDWKKGDTFSCMVGH | |
| | EALPLAFTQKTIDRLGECDKTHT | |
| | CPPCPAPELLGGPSVFLFPPKPK | |
| | DTLMISRTPEVTCVVVDVSHEDP | |
| | EVKFNVVYVDGVEVHNAKTKPRE | |
| | EQYNSTYRVVSVLTVLHQDWLNG | |
| | KEYKCKVSNKALPAPIEKTISKA | |
| | KGQPREPQVYTLPPCRDELTKNQ | |
| | VSLWCLVKGFYPSDIAVEWESNG | |
| | QPENNYKTTPPVLDSDGSFFLYS | |
| | KLTVDKSRWQQGNVFSCSVMHEA | |
| | LHNHYTQKSLSLSPGK | |
| BA1. | EVQLVESGGGLVQPGGSLRLSCA | 101 |
| chain 2 | ASGFTFSDYDIHWVRQAPGKGLE | |
| | WVGDITPYDGTTNYADSVKGRFT | |
| | ISADTSKNTAYLQMNSLRAEDTA | |
| | VYYCARLVGEIATGFDYVVGQGT | |
| | LVTVSSASTFRPEVHLLPPPSEE | |
| | LALNELVTLTCLARGFSPKDVLV | |
| | RWLQGSQELPREKYLTWASRQEP | |
| | SQGTTTFAVTSILRVAAEDWKKG | |
| | DTFSCMVGHEALPLAFTQKTIDR | |
| | LGEC | |
| BA(a11). | EIVLTQSPATLSLSPGERATLSC | 102 |
| chain 3 | RASQSVSSYLAWYQQKPGQAPRL | |
| | LIYDASNRATGIPARFSGSGSGT | |
| | PDFTLTISSLEPEDFAVYYCQQSS | |
| | NWPRTFGQGTKVEIKRTVAAPSVF | |
| | IFPSDEQLKSGTASVVCLLNNFYP | |
| | REAKVQWKVDNALQSGNSQESVTE | |
| | QDSKDSTYSLSSTLTLSKADYEKH | |
| | KVYACEVTHQGLSSPVTKSFNRGE | |
| | CDKTHTCPPCPAPELLGGPSVFLF | |
| | PPKPKDTLMISRTPEVTCVVVDVS | |
| | HEDPEVKFNWYVDGVEVHNAKTKP | |
| | REEQYNSTYRVVSVLTVLHQDWLN | |
| | GKEYKCKVSNKALPAPIEKTISKA | |
| | KGQPREPQVCTLPPSREEMTKNQV | |
| | SLSCAVKGFYPSDIAVEWESNGQP | |
| | ENNYKTTPPVLDSDGSFFLVSKLT | |
| | VDKSRWQQGNVFSCSVMHEALHNH | |
| | YTQKSLSLSPGK | |
| BA(a11). | QVQLVESGGGVVQPGRSLRLDCKA | 103 |
| chain 4 | SGITFSNSGMHWVRQAPGKGLEWV | |
| | AVIWYDGSKRYYADSVKGRFTISR | |
| | DNSKNTLFLQMNSLRAEDTAVYYC | |
| | ATNDDYWGQGTLVTVSSASTKGPS | |
| | VFPLAPSSKSTSGGTAALGCLVKD | |
| | YFPEPVTVSWNSGALTSGVHTFPA | |
| | VLQSSGLYSLSSVVTVPSSSLGTQ | |
| | TYICNVNHKPSNTKVDKKVEPPKS | |
| | C | |

Antibodies were produced by Expi293 expression as described herein.

### 6.14.26.3. BA1 Exhibits Specific Binding to Both PD1 and Antigen A

Interaction of BA1 with PD1 and Antigen A was assessed using bio-layer interferometry (Octet/FORTEBIO®). Briefly, biotinylated PD1 or biotinylated Antigen A (magenta) was immobilized on streptavidin sensor. BA1 was then added, followed by a dissociation step. Response profiles were generated in real time.

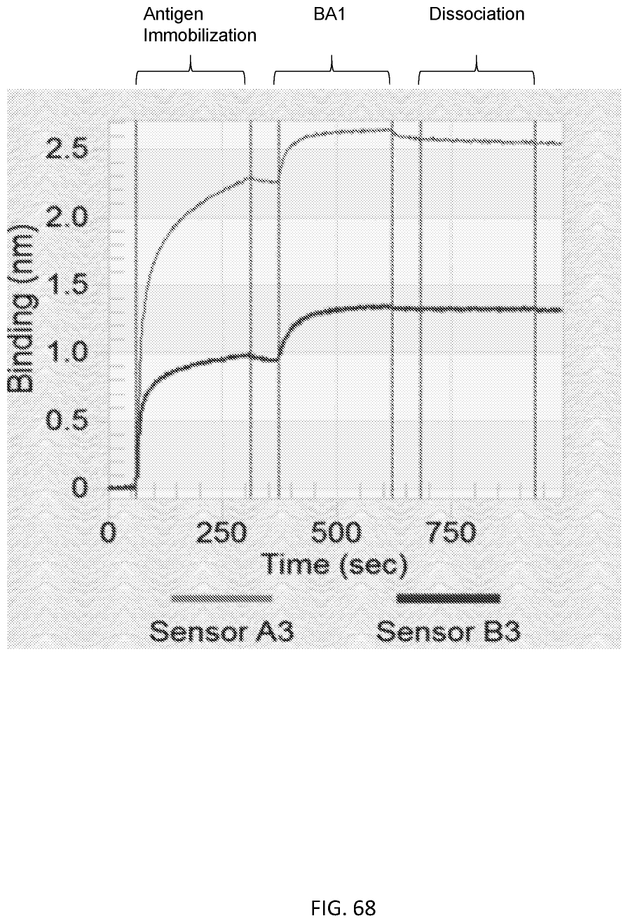

FIG. 68 depicts results from the Octet assay. BA1 contained both binding sites for PD1 and Antigen A.

### 6.14.26.4. Example B2: Optimization of CH3 Linker Sequences

Variants of BA were constructed as described herein, where the first CH3 linker sequence and second CH3 linker sequence were varied according to the following table. For all BA variants, polypeptides 3 (SEQ ID NO:102) and 4 (SEQ ID NO:103) were unchanged.

TABLE 9
provides the amino acid sequences for the first CH3 linker and second
CH3 linkers used in constructing the BA variants.
CH3 linker sequences for BA variants

| BA variant # | First CH3 linker sequence | Second CH3 linker sequence |
|---|---|---|
| BA1 | GEC | GEC |
| BA2 | AGC | AGKGSC (SEQ ID NO: 99) |
| BA3 | AGKGC (SEQ ID NO: 98) | AGC |
| BA4 | AGKGSC (SEQ ID NO: 99) | AGC |
| BA5 | AGKC (SEQ ID NO: 96) | AGC |
| BA9 | AGC | AGC |
| BA10 | AGC | AGKGC (SEQ ID NO: 98) |
| BA11 | AGC | AGKGSC (SEQ ID NO: 99) |
| BA12 | AGC | AGKC (SEQ ID NO: 96) |
| BA13 | AGC | GEC |
| BA14 | AGC | PGKC (SEQ ID NO: 97) |
| BA15 | AGKGC (SEQ ID NO: 98) | AGC |
| BA16 | AGKGC (SEQ ID NO: 98) | AGKGC (SEQ ID NO: 98) |
| BA17 | AGKGC (SEQ ID NO: 98) | AGKGSC (SEQ ID NO: 99) |
| BA18 | AGKGC (SEQ ID NO: 98) | AGKC (SEQ ID NO: 96) |
| BA19 | AGKGC (SEQ ID NO: 98) | GEC |
| BA20 | AGKGC (SEQ ID NO: 98) | PGKC (SEQ ID NO: 97) |
| BA21 | AGKGSC (SEQ ID NO: 99) | AGC |
| BA22 | AGKGSC (SEQ ID NO: 99) | AGKGC (SEQ ID NO: 98) |
| BA23 | AGKGSC (SEQ ID NO: 99) | AGKGSC (SEQ ID NO: 99) |
| BA24 | AGKC (SEQ ID NO: 96) | AGKGSC (SEQ ID NO: 99) |
| BA25 | AGKGSC (SEQ ID NO: 99) | GEC |
| BA26 | AGKGSC (SEQ ID NO: 99) | PGKC (SEQ ID NO: 97) |
| BA27 | AGKC (SEQ ID NO: 96) | AGC |
| BA28 | AGKC (SEQ ID NO: 96) | AGKGC (SEQ ID NO: 98) |
| BA29 | AGKC (SEQ ID NO: 96) | AGKGSC (SEQ ID NO: 99) |
| BA30 | AGKC (SEQ ID NO: 96) | AGKC (SEQ ID NO: 96) |
| BA31 | AGKC (SEQ ID NO: 96) | GEC |
| BA32 | AGKC (SEQ ID NO: 96) | PGKC (SEQ ID NO: 97) |
| BA33 | GEC | AGC |
| BA34 | GEC | AGKGC (SEQ ID NO: 98) |
| BA35 | GEC | AGKGSC (SEQ ID NO: 99) |
| BA36 | GEC | AGKC (SEQ ID NO: 96) |
| BA37 | GEC | GEC |
| BA38 | GEC | PGKC (SEQ ID NO: 97) |
| BA39 | PGKC (SEQ ID NO: 97) | AGC |
| BA40 | PGKC (SEQ ID NO: 97) | AGKGC (SEQ ID NO: 98) |
| BA41 | PGKC (SEQ ID NO: 97) | AGKGSC (SEQ ID NO: 99) |
| BA42 | PGKC (SEQ ID NO: 97) | AGKC (SEQ ID NO: 96) |
| BA43 | PGKC (SEQ ID NO: 97) | GEC |
| BA44 | PGKC (SEQ ID NO: 97) | PGKC (SEQ ID NO: 97) |

Table 10 provides the amino acid sequences for polypeptide 1 of the additional BA variants.

TABLE 10
additional BA variants, chain 1 sequences

| | | SEQ ID |
|---|---|---|
| BA# | Chain 1 Sequence | NO: |
| BA2 | DIQMTQSPSSLSASVGDRVTITCRASQDVNTAVAWYQQKPGKAPKLLIYSASFL | 104 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA3 | DIQMTQSPSSLSASVGDRVTITCRASQDVNTAVAWYQQKPGKAPKLLIYSASFL | 105 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA4 | DIQMTQSPSSLSASVGDRVTITCRASQDVNTAVAWYQQKPGKAPKLLIYSASFL | 106 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA5 | DIQMTQSPSSLSASVGDRVTITCRASQDVNTAVAWYQQKPGKAPKLLIYSASFL | 107 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA9 | DIQMTQSPSSLSASVGDRVTITCRASQDVNTAVAWYQQKPGKAPKLLIYSASFL | 108 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA10 | DIQMTQSPSSLSASVGDRVTITCRASQDVNTAVAWYQQKPGKAPKLLIYSASFL | 109 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA11 | DIQMTQSPSSLSASVGDRVTITCRASQDVNTAVAWYQQKPGKAPKLLIYSASFL | 110 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA12 | DIQMTQSPSSLSASVGDRVTITCRASQDVNTAVAWYQQKPGKAPKLLIYSASFL | 111 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA13 | DIQMTQSPSSLSASVGDRVTITCRASQDVNTAVAWYQQKPGKAPKLLIYSASFL | 112 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA14 | DIQMTQSPSSLSASVGDRVTITCRASQDVNTAVAWYQQKPGKAPKLLIYSASFL | 113 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA15 | DIQMTQSPSSLSASVGDRVTITCRASQDVNTAVAWYQQKPGKAPKLLIYSASFL | 114 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA16 | DIQMTQSPSSLSASVGDRVTITCRASQDVNTAVAWYQQKPGKAPKLLIYSASFL | 115 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA17 | DIQMTQSPSSLSASVGDRVTITCRASQDVNTAVAWYQQKPGKAPKLLIYSASFL | 116 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA18 | DIQMTQSPSSLSASVGDRVTITCRASQDVNTAVAWYQQKPGKAPKLLIYSASFL | 117 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA19 | DIQMTQSPSSLSASVGDRVTITCRASQDVNTAVAWYQQKPGKAPKLLIYSASFL | 118 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA20 | DIQMTQSPSSLSASVGDRVTITCRASQDVNTAVAWYQQKPGKAPKLLIYSASFL | 119 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA21 | DIQMTQSPSSLSASVGDRVTITCRASQDVNTAVAWYQQKPGKAPKLLIYSASFL | 120 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA22 | DIQMTQSPSSLSASVGDRVTITCRASQDVNTAVAWYQQKPGKAPKLLIYSASFL | 121 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA23 | DIQMTQSPSSLSASVGDRVTITCRASQDVNTAVAWYQQKPGKAPKLLIYSASFL | 122 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA24 | DIQMTQSPSSLSASVGDRVTITCRASQDVNTAVAWYQQKPGKAPKLLIYSASFL | 123 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA25 | DIQMTQSPSSLSASVGDRVTITCRASQDVNTAVAWYQQKPGKAPKLLIYSASFL | 124 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA26 | DIQMTQSPSSLSASVGDRVTITCRASQDVNTAVAWYQQKPGKAPKLLIYSASFL | 125 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA27 | DIQMTQSPSSLSASVGDRVTITCRASQDVNTAVAWYQQKPGKAPKLLIYSASFL | 126 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA28 | DIQMTQSPSSLSASVGDRVTITCRASQDVNTAVAWYQQKPGKAPKLLIYSASFL | 127 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA29 | DIQMTQSPSSLSASVGDRVTITCRASQDVNTAVAWYQQKPGKAPKLLIYSASFL | 128 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA30 | DIQMTQSPSSLSASVGDRVTITCRASQDVNTAVAWYQQKPGKAPKLUYSASFL | 129 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA31 | DIQMTQSPSSLSASVGDRVTITCRASQDVNTAVAWYQQKPGKAPKLLIYSASFL | 130 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA32 | DIQMTQSPSSLSASVGDRVTITCRASQDVNTAVAWYQQKPGKAPKLLIYSASFL | 131 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA33 | DIQMTQSPSSLSASVGDRVTITCRASQDVNTAVAWYQQKPGKAPKLLIYSASFL | 132 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA34 | DIQMTQSPSSLSASVGDRVTITCRASQDVNTAVAWYQQKPGKAPKLLIYSASFL | 133 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA35 | DIQMTQSPSSLSASVGDRVTITCRASQDVNTAVAWYQQKPGKAPKLLIYSASFL | 134 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA36 | DIQMTQSPSSLSASVGDRVTITCRASQDVNTAVAWYQQKPGKAPKLLIYSASFL | 135 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA37 | DIQMTQSPSSLSASVGDRVTITCRASQDVNTAVAWYQQKPGKAPKLLIYSASFL | 136 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA38 | DIQMTQSPSSLSASVGDRVTITCRASQDVNTAVAWYQQKPGKAPKLLIYSASFL | 137 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA39 | DIQMTQSPSSLSASVGDRVTITCRASQDVNTAVAWYQQKPGKAPKLLIYSASFL | 138 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA40 | DIQMTQSPSSLSASVGDRVTITCRASQDVNTAVAWYQQKPGKAPKLLIYSASFL | 139 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA41 | DIQMTQSPSSLSASVGDRVTITCRASQDVNTAVAWYQQKPGKAPKLLIYSASFL | 140 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA42 | DIQMTQSPSSLSASVGDRVTITCRASQDVNTAVAWYQQKPGKAPKLLIYSASFL | 141 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA43 | DIQMTQSPSSLSASVGDRVTITCRASQDVNTAVAWYQQKPGKAPKLLIYSASFL | 142 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA44 | DIQMTQSPSSLSASVGDRVTITCRASQDVNTAVAWYQQKPGKAPKLLIYSASFL | 143 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |

Table 11 provides the amino acid sequences for polypeptide 2 of the additional BA variants.

TABLE 11
additional BA variants, chain 2 sequences

| | | SEQ ID |
|---|---|---|
| BA# | Chain 2 Sequence (base + linker) | NO: |
| BA2 | EVQLVESGGGLVQPGGSLRLSCAASGFTFSDYDIHWVRQAPGKGLEWVGDITPY | 144 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA3 | EVQLVESGGGLVQPGGSLRLSCAASGFTFSDYDIHWVRQAPGKGLEWVGDITPY | 145 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA4 | EVQLVESGGGLVQPGGSLRLSCAASGFTFSDYDIHWVRQAPGKGLEWVGDITPY | 146 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA5 | EVQLVESGGGLVQPGGSLRLSCAASGFTFSDYDIHWVRQAPGKGLEWVGDITPY | 147 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA9 | EVQLVESGGGLVQPGGSLRLSCAASGFTFSDYDIHWVRQAPGKGLEWVGDITPY | 148 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA10 | EVQLVESGGGLVQPGGSLRLSCAASGFTFSDYDIHWVRQAPGKGLEWVGDITPY | 149 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA11 | EVQLVESGGGLVQPGGSLRLSCAASGFTFSDYDIHWVRQAPGKGLEWVGDITPY | 150 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA12 | EVQLVESGGGLVQPGGSLRLSCAASGFTFSDYDIHWVRQAPGKGLEWVGDITPY | 151 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA13 | EVQLVESGGGLVQPGGSLRLSCAASGFTFSDYDIHWVRQAPGKGLEWVGDITPY | 152 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA14 | EVQLVESGGGLVQPGGSLRLSCAASGFTFSDYDIHWVRQAPGKGLEWVGDITPY | 153 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA15 | EVQLVESGGGLVQPGGSLRLSCAASGFTFSDYDIHWVRQAPGKGLEWVGDITPY | 154 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA16 | EVQLVESGGGLVQPGGSLRLSCAASGFTFSDYDIHWVRQAPGKGLEWVGDITPY | 155 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA17 | EVQLVESGGGLVQPGGSLRLSCAASGFTFSDYDIHWVRQAPGKGLEWVGDITPY | 156 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA18 | EVQLVESGGGLVQPGGSLRLSCAASGFTFSDYDIHWVRQAPGKGLEWVGDITPY | 157 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA19 | EVQLVESGGGLVQPGGSLRLSCAASGFTFSDYDIHWVRQAPGKGLEWVGDITPY | 158 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA20 | EVQLVESGGGLVQPGGSLRLSCAASGFTFSDYDIHWVRQAPGKGLEWVGDITPY | 159 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA21 | EVQLVESGGGLVQPGGSLRLSCAASGFTFSDYDIHWVRQAPGKGLEWVGDITPY | 160 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA22 | EVQLVESGGGLVQPGGSLRLSCAASGFTFSDYDIHWVRQAPGKGLEWVGDITPY | 161 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA23 | EVQLVESGGGLVQPGGSLRLSCAASGFTFSDYDIHWVRQAPGKGLEWVGDITPY | 162 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA24 | EVQLVESGGGLVQPGGSLRLSCAASGFTFSDYDIHWVRQAPGKGLEWVGDITPY | 163 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA25 | EVQLVESGGGLVQPGGSLRLSCAASGFTFSDYDIHWVRQAPGKGLEWVGDITPY | 164 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA26 | EVQLVESGGGLVQPGGSLRLSCAASGFTFSDYDIHWVRQAPGKGLEWVGDITPY | 165 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA27 | EVQLVESGGGLVQPGGSLRLSCAASGFTFSDYDIHWVRQAPGKGLEWVGDITPY | 166 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA28 | EVQLVESGGGLVQPGGSLRLSCAASGFTFSDYDIHWVRQAPGKGLEWVGDITPY | 167 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA29 | EVQLVESGGGLVQPGGSLRLSCAASGFTFSDYDIHWVRQAPGKGLEWVGDITPY | 168 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA30 | EVQLVESGGGLVQPGGSLRLSCAASGFTFSDYDIHWVRQAPGKGLEWVGDITPY | 169 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA31 | EVQLVESGGGLVQPGGSLRLSCAASGFTFSDYDIHWVRQAPGKGLEWVGDITPY | 170 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA32 | EVQLVESGGGLVQPGGSLRLSCAASGFTFSDYDIHWVRQAPGKGLEWVGDITPY | 171 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA33 | EVQLVESGGGLVQPGGSLRLSCAASGFTFSDYDIHWVRQAPGKGLEWVGDITPY | 172 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA34 | EVQLVESGGGLVQPGGSLRLSCAASGFTFSDYDIHWVRQAPGKGLEWVGDITPY | 173 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA35 | EVQLVESGGGLVQPGGSLRLSCAASGFTFSDYDIHWVRQAPGKGLEWVGDITPY | 174 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA36 | EVQLVESGGGLVQPGGSLRLSCAASGFTFSDYDIHWVRQAPGKGLEWVGDITPY | 175 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA37 | EVQLVESGGGLVQPGGSLRLSCAASGFTFSDYDIHWVRQAPGKGLEWVGDITPY | 176 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA38 | EVQLVESGGGLVQPGGSLRLSCAASGFTFSDYDIHWVRQAPGKGLEWVGDITPY | 177 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA39 | EVQLVESGGGLVQPGGSLRLSCAASGFTFSDYDIHWVRQAPGKGLEWVGDITPY | 178 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA40 | EVQLVESGGGLVQPGGSLRLSCAASGFTFSDYDIHWVRQAPGKGLEWVGDITPY | 179 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA41 | EVQLVESGGGLVQPGGSLRLSCAASGFTFSDYDIHWVRQAPGKGLEWVGDITPY | 180 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA42 | EVQLVESGGGLVQPGGSLRLSCAASGFTFSDYDIHWVRQAPGKGLEWVGDITPY | 181 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA43 | EVQLVESGGGLVQPGGSLRLSCAASGFTFSDYDIHWVRQAPGKGLEWVGDITPY | 182 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA44 | EVQLVESGGGLVQPGGSLRLSCAASGFTFSDYDIHWVRQAPGKGLEWVGDITPY | 183 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |

CH1 Purification and SDS-PAGE Analysis

Protein was expressed by transient expression in Expi 293 cells as described above, and purified by one-step affinity chromatography using CH1 resin.

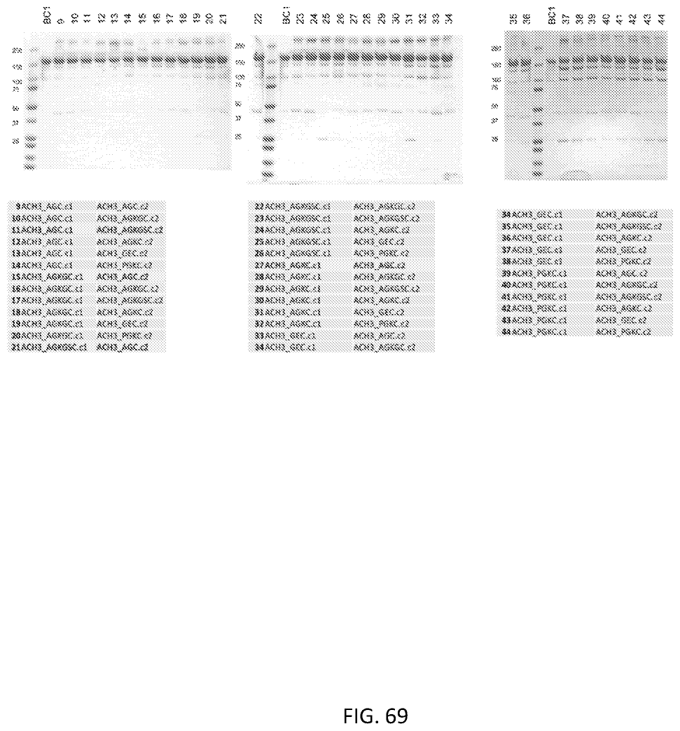

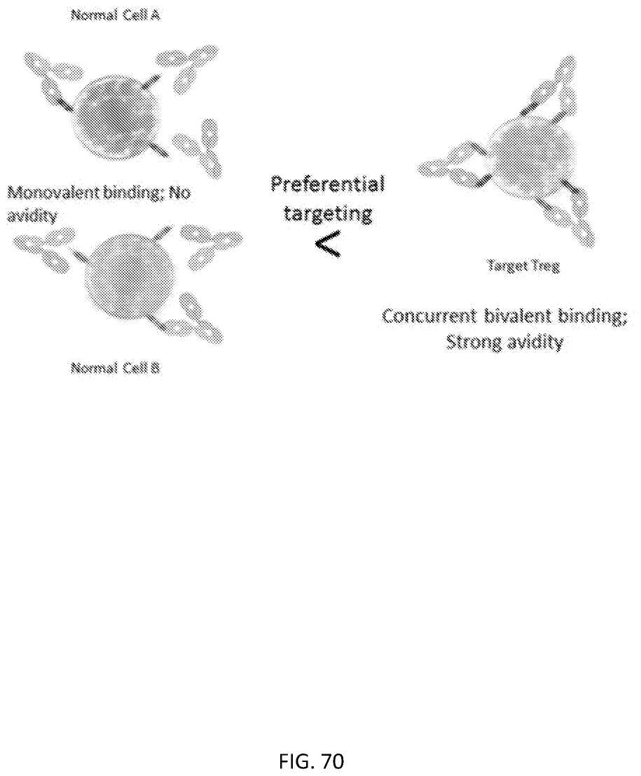

FIG. 69 depicts SDS-PAGE analysis of BC1 and variants BA9-44. Variants exhibiting high yield from transient transfection, low levels of low molecular weight contaminants, and low levels of high molecular weight contaminants, were selected for further optimization. It was observed that use of non-identical CH3 linkers for domains B and G resulted in improved yield and reduced contaminants. Variants BA11, BA15, BA21, and BA27 in particular exhibited high yield and high-fidelity assembly.

### 6.14.27. Example 27: Validation of Treg Cell Surface Antigens for Design of SNIPER Candidates

Target Validation

Antigen combinations useful for specific targeting of tumor-associated Tregs were validated.

Dissociated tumor samples and peripheral blood mononuclear cells (PBMCs) were obtained from two lung cancer patients and analyzed for immune cell content by immunofluorescence staining followed by flow cytometry. Cell subpopulations were identified by flow cytometry according to the parameters in Table 14.

TABLE 14
gating parameters

| Cell Population | Gating parameter | Antibody |
|---|---|---|
| Live cells | Ghost Dye ™ | Ghost Dye ™ Red 780 |
| | Red 780 (low | (Tonbo Bioscience 13-0865- |
| | staining) | T100) |
| Lymphocytes | CD45+ | Mouse anti-human CD45− |
| | | BD Horizon ™ V500 |
| | | (BD Biosciences 560779) |
| All T-cells | CD3+ | Mouse anti-human CD3− |
| | | BD Horizon ™ BV605 |
| | | (BD Biosciences 563217) |
| Killer T cells | CD8+ | Mouse Anti-Human CD8− |
| | | BD Horizon ™ Alexa Fluor |
| | | 700 ThermoFisher 56-0086-41) |
| Teffector and Treg | CD4+ | Anti-Human CD4 PerCP- |
| | | eFluor ® 710 |
| | | (ThermoFisher 46-0047-41) |
| Treg | CD25+ and | Mouse Anti-Human CD25− |
| | CD127 low | BD Horizon ™ BB515 |
| | | (BD Biosciences 564467) |
| | | CD127 Monoclonal |
| | | Antibody-PE-Cyanine7 |
| | | (Thermo Fisher eFluor) |

The matched dissociated tumor samples and PMBCs from the lung cancer patients were also subjected to immunofluorescence staining and flow cytometry for the following targets: OX40, CTLA4, CD25, GITR, and TIGIT. Briefly matched dissociated tumor cells and PBMCs from lung cancer patients were stained with Ghost-dye Red 780, anti-CD45, anti-CD3, anti-CD8, anti-CD4, anti-CD127, anti-CD25, anti-OX40, anti-CTLA4, anti-GITR, and anti-TIGIT antibodies for 45 min at 4° C. Cells were then pelleted by centrifugation and resuspended in flow cytometry buffer (Hank's Balanced Salt Solution+25 mM HEPES+0.1% BSA). Samples were analyzed by flow cytometry. Two-target combinations that were overrepresented in tumor-associated Tregs as compared to other cell subpopulations were selected for further assessment. Exemplary selected target combinations include CD25 and OX40, CD25 and CTLA-4, OX40 and CTLA-4.

TABLE 15
list of antibodies used to detect target antigens

| Target | Antibody |
|---|---|
| OX40 | OX40 monoclonal antibody-PE |
| | (ThermoFisher 12-1347-42) |
| GITR | GITR monoclonal antibody PE-eFluor 610 |
| | (ThermoFisher 61-5875-41) |
| TIGIT | TIGIT monoclonal antibody eFluor450 |
| | (ThermoFisher 48-9500-41) |
| CTLA4 | Mouse anti-human CTLA4 APC |
| | (BD Horizon ™ 560938) |
| CD25 | Mouse Anti-Human CD25− |
| | BD Horizon ™ BB515 |
| | (BD Biosciences 564467) |

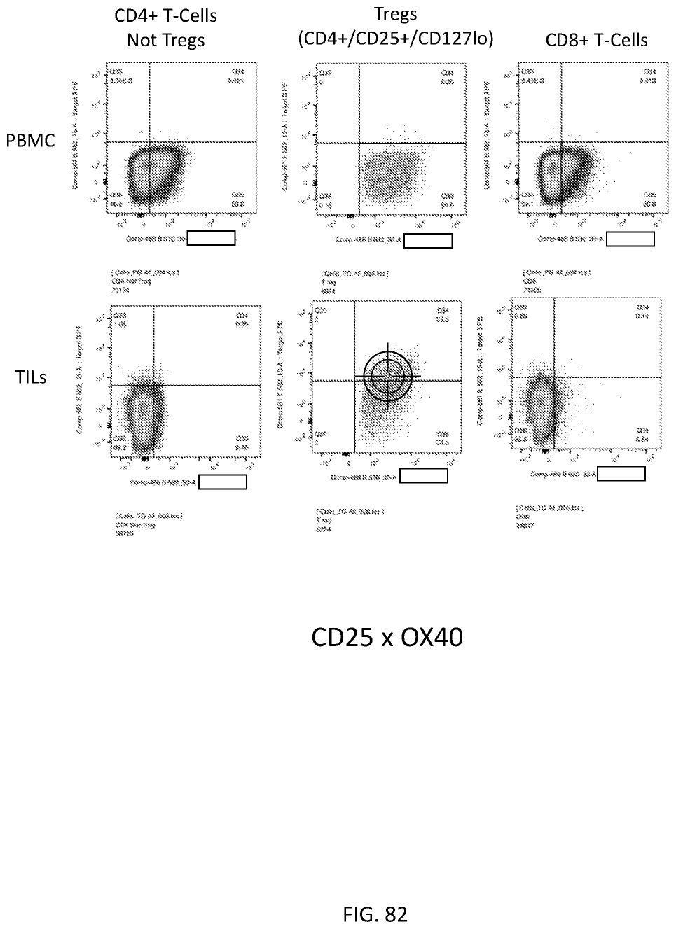

FIG. 82 depicts flow cytometry results from the OX40/CD25 staining experiment. Only tumor-infiltrating Tregs exhibited appreciable double positive staining for OX40 and CD25.

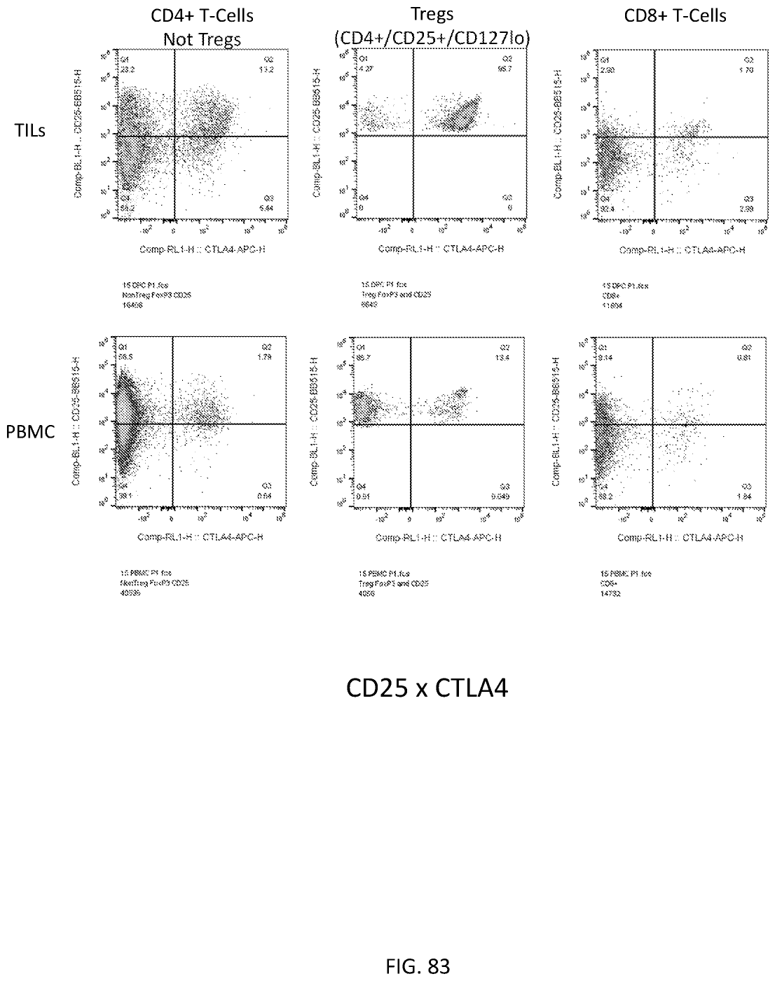

FIG. 83 depicts flow cytometry results from the CTLA4/CD25 staining experiment. A majority of tumor-infiltrating Tregs, but not a majority of other cell types, exhibited double positive staining for CTLA4 and CD25.

### 6.14.28. Example 28: Human SNIPER Antibody Discovery to CTLA4/CD25

Preparation of Phage Library

Phage display of human Fab libraries was carried out using standard protocols. Phage clones were screened for the ability to bind to CTLA4 or CD25 by phage ELISA using standard protocols. Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain are expressed as separate polypeptides, and the light chain and heavy chain-pIII fusion assemble in the bacterial periplasm, where the redox potential enables disulfide bond formation, to form the antibody containing the candidate ABS.

The library was created using sequences derived from a specific human heavy chain variable domain (VH3-23) and a specific human light chain variable domain (Vk-1). Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:74) and light chain (SEQ ID NO:75) scaffolds used in the library are listed below, where a lower case “x” represents CDR amino acids that were varied to create the library, and bold italic represents the CDR sequences that were constant.

Diversity was created through Kunkel mutagenesis using primers to introduce diversity into VL CDR3 and VH CDR1 (H1), CDR2 (H2) and CDR3 (H3) to mimic the diversity found in the natural antibody repertoire, as described in more detail in Kunkel, T A (PNAS Jan. 1, 1985. 82 (2) 488-492), herein incorporated by reference in its entirety. Briefly, single-stranded DNA were prepared from isolated phage using standard procedures and Kunkel mutagenesis carried out. Chemically synthesized DNA was then electroporated into TG1 cells, followed by recovery. Recovered cells were sub-cultured and infected with M13K07 helper phage to produce the phage library.

Phage Panning

Phage panning was performed using standard procedures. Briefly, the first round of phage panning was performed with target (CTLA4 or CD25) immobilized on streptavidin magnetic beads which were subjected to ˜5×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD600-0.6. After 20 minutes, infected cells were sub-cultured in 25 mL 2×YT+ Ampicillin and M13K07 helper phage and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for either CTLA4 or CD25. Target-specific enrichment was confirmed using polyclonal and monoclonal phage ELISA. DNA sequencing was used to determine isolated Fab clones containing a candidate ABS.

Parent Binding Molecules

To measure binding affinity in discovery campaigns, the VL and VH domains were formatted into a bivalent monospecific native human full-length IgG1 architecture and immobilized to a biosensor on an Octet (Pall ForteBio) biolayer interferometer. Soluble antigens CTLA4 or CD25 were then added to the system and binding measured.

Samples containing parent monoclonal antibodies CTLA4-19 and CD25-8 were assessed by TSK-gel size exclusion chromatography as described herein.

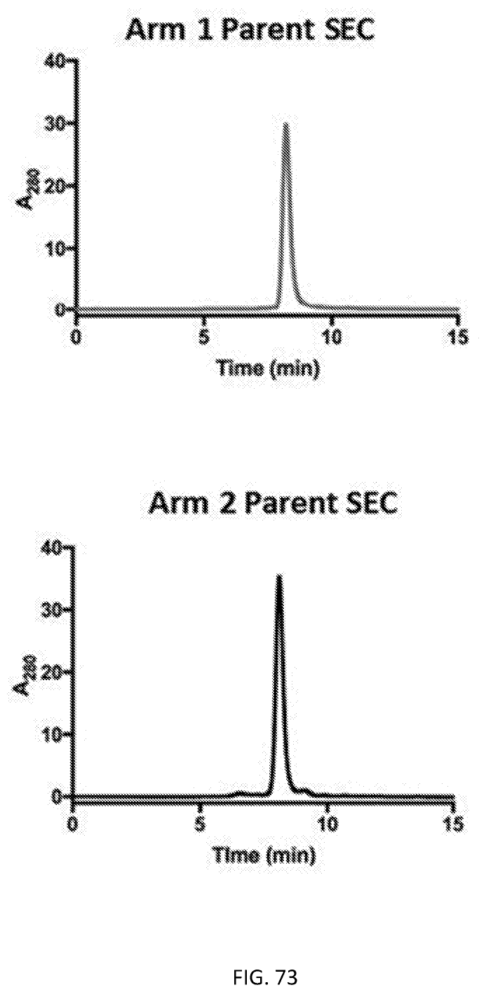

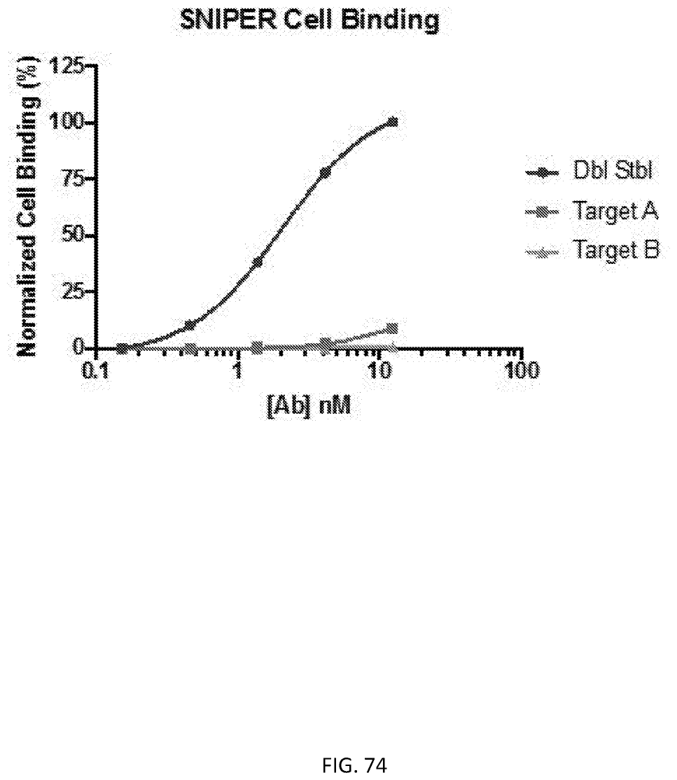

Results are shown in FIG. 73. Each antibody eluted as a single, monodisperse peak after single-step Protein-A purification.

Creation of Candidate SNIPER Binding Molecules to CTLA4 and CD25

Bispecific SNIPER B-Body binding proteins to CTLA4 and CD25 were created by (a) formatting VL variable regions of individual clones into Domain A and/or H, and (b) formatting VH variable regions into Domain F and/or L of a bivalent 1×1 B-Body “BC1” scaffold shown below and with reference to FIG. 3.

“BC1” Scaffold:
1^{st} polypeptide chain (SEQ ID NO:78)
   Domain A=Antigen 1 B-Body Domain A/H Scaffold (SEQ ID NO:76)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:79):
   Domain F=Antigen 1 B-Body Domain F/L Scaffold (SEQ ID NO:77)
   Domain G=CH3 (L351D; 445G, 446E, 447C tripeptide insertion)
3^{rd} polypeptide chain (SEQ ID NO:80):
   Domain H=Antigen 2 B-Body Domain A/H Scaffold (SEQ ID NO:76)
   Domain I=CL (Kappa)
   Domain J=CH2
   Domain K=CH3 (Y349C, D356E, L358M, T366S, L368A, Y407V)
4^{th} polypeptide chain (SEQ ID NO:81):
   Domain L=Antigen 2 B-Body Domain F/L Scaffold (SEQ ID NO:77)
   Domain M=CH1.

Antibodies were generated and harvested as described herein.

Testing of candidate SNIPER binding molecules to CTLA4 and CD25

Pools of HEK293 cells stably expressing either CTLA4 (Target A), CD25 (Target B), or both CTLA4 and CD25 (Dbl Stbl) were tested for cell binding with a dilution series of various bispecific SNIPER™ candidates. Cells were labeled with the SNIPER™ candidates, followed by an AlexaFluor488 labeled goat anti-human Fab, and the mean fluorescence intensity (MFI) determined by flow cytometry. Fluorescence intensity data was normalized to MFI from control monoclonal parent antibodies to the single targets.

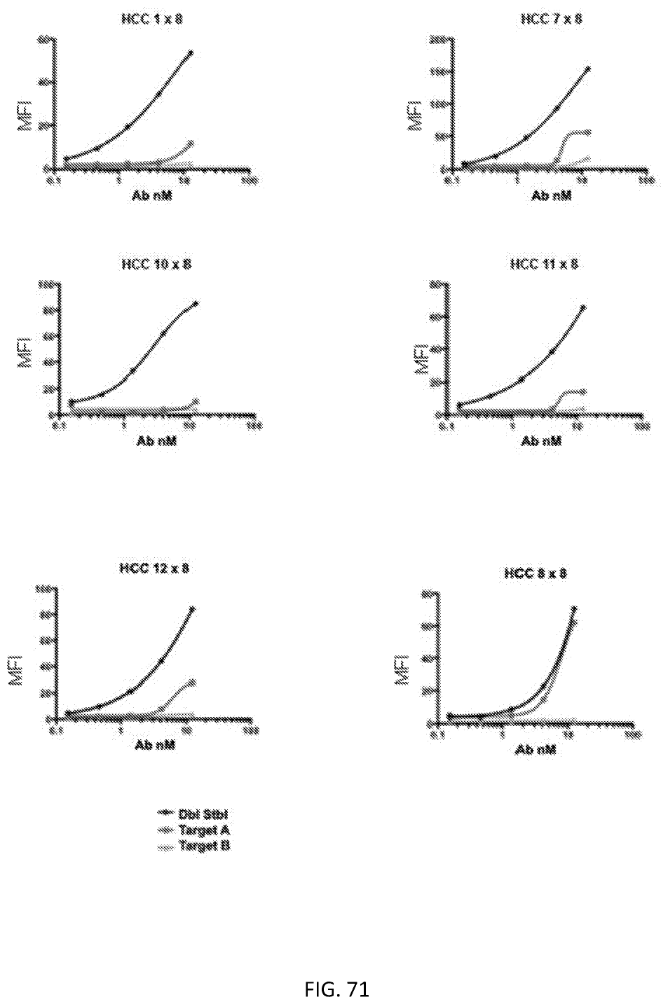

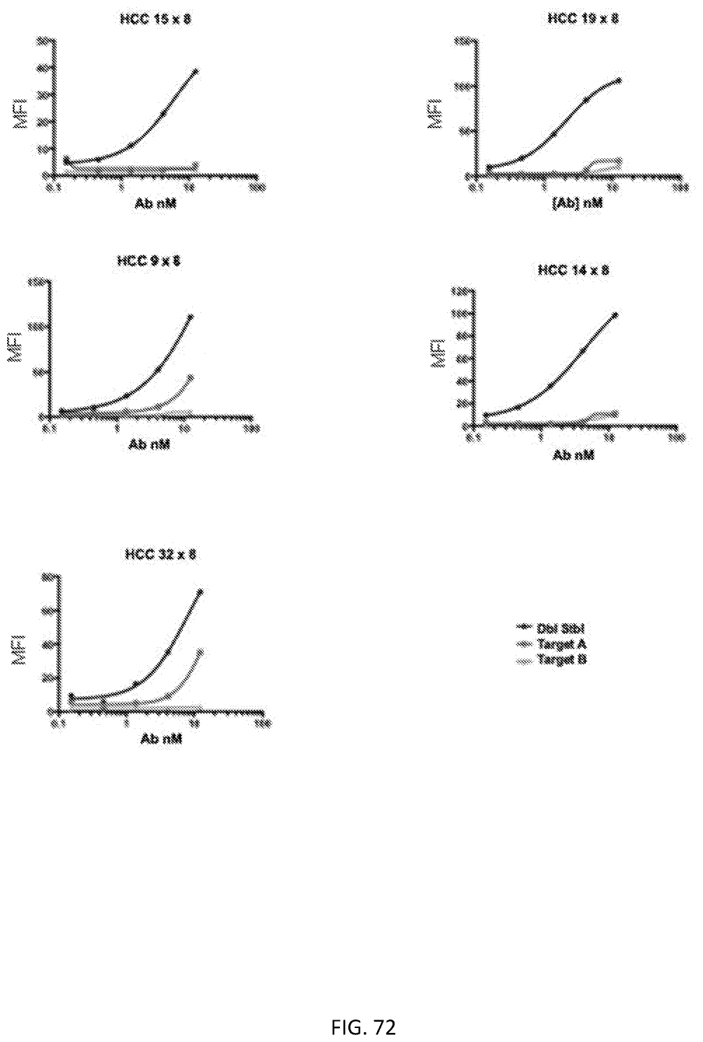

FIGS. 71 and 72 depict MFI results of the above experiment for various candidate SNIPER molecules. Candidate SNIPER molecules exhibiting higher binding avidity to doubly-expressing cells as compared to cells expressing single antigens were selected as desirable candidates.

### 6.14.29. Example 29: Creation of BC1 1×1 B-Body SNIPER 19×8 Binding Molecule

A bivalent, bispecific SNIPER construct, termed “SNIPER 19×8”, with low binding affinity to CTLA4 and CD25, was constructed.

In greater detail, with domain and polypeptide chain references in accordance with FIG. 3 and modifications from native sequences indicated in parentheses, the architecture was:
1^{st} polypeptide chain
   Domain A=VL (“CTLA4-19”)
   Domain B=CH3 (T366K; 445K, 446S, 447C tripeptide insertion)
   Domain D=CH2
   Domain E=CH3 (T366W, S354C)
2nd polypeptide chain
   Domain F=VH (“CTLA4-19”)
   Domain G=CH3 (L351D; 445G, 446E, 447C tripeptide insertion)
3rd polypeptide chain
   Domain H=VL (“CD25-8”)
   Domain I=CL (Kappa)
   Domain J=CH2
   Domain K=CH3 (Y349C, D356E, L358M, T366S, L368A, Y407V)
4th polypeptide chain
   Domain L=VH (“CD25-8”)
   Domain M=CH1.

The sequences for the four polypeptide chains are indicated in Table 16.

TABLE 16
polypeptide chain sequences, SNIPER 19X8

| Polypeptide | |
|---|---|
| Chain # | Sequence |
| 1 | IQMTQSPSSLSASVGDRVTITCRASQSVSSAVAWYQQKPGKA |
| | PKLLIYSASSLYSGVPSRFSGSRSGTDFTLTISSLQPEDFATYY |
| | CQQYYTTPFTFGQGTKVEIKRTPREPQVYTLPPSRDELTKNQ |
| | VSLKCLVKGFYPSDIAVEWESNGQPENNYKTTPPVLDSDGSF |
| | FLYSKLTVDKSRWQQGNVFSCSVMHEALHNHYTQKSLSLSK |
| | SCDKTHTCPPCPAPELLGGPSVFLFPPKPKDTLMISRTPEVTC |
| | VVVDVSHEDPEVKFNWYVDGVEVHNAKTKPREEQYNSTYR |
| | VVSVLTVLHQDWLNGKEYKCKVSNKALPAPIEKTISKAKGQ |
| | PREPQVYTLPPCRDELTKNQVSLWCLVKGFYPSDIAVEWESN |
| | GQPENNYKTTPPVLDSDGSFFLYSKLTVDKSRWQQGNVFSC |
| | SVMHEALHNHYTQKSLSLSPGK (SEQ ID NO: 186) |
| 2 | EVQLVESGGGLVQPGGSLRLSCAASGFTFSGYWIHWVRQAP |
| | GKGLEWVAAITSRDSYTYYADSVKGRFTISADTSKNTAYLQ |
| | MNSLRAEDTAVYYCARGSYLGSAFDYWGQGTLVTVSSASPR |
| | EPQVYTDPPSRDELTKNQVSLTCLVKGFYPSDIAVEWESNGQ |
| | PENNYKTTPPVLDSDGSFFLYSKLTVDKSRWQQGNVFSCSVM |
| | HEALHNHYTQKSLSLSGEC (SEQ ID NO: 187) |
| 3 | SDIQMTQSPSSLSASVGDRVTITCRASQSVSSAVAWYQQKPG |
| | KAPKLLIYSASSLYSGVPSRFSGSRSGTDFTLTISSLQPEDFATY |
| | YCQQYSSQPATFGQGTKVEIKRTVAAPSVFIFPPSDEQLKSGT |
| | ASVVCLLNNFYPREAKVQWKVDNALQSGNSQESVTEQDSKD |
| | STYSLSSTLTLSKADYEKHKVYACEVTHQGLSSPVTKSFNRG |
| | ECDKTHTCPPCPAPELLGGPSVFLFPPKPKDTLMISRTPEVTCV |
| | VVDVSHEDPEVKFNWYVDGVEVHNAKTKPREEQYNSTYRV |
| | VSVLTVLHQDWLNGKEYKCKVSNKALPAPIEKTISKAKGQPR |
| | EPQVCTLPPSREEMTKNQVSLSCAVKGFYPSDIAVEWESNGQ |
| | PENNYKTTPPVLDSDGSFFLVSKLTVDKSRWQQGNVFSCSVM |
| | HEALHNHYTQKSLSLSPGK (SEQ ID NO: 188) |
| 4 | EVQLVESGGGLVQPGGSLRLSCAASGFTFSSYYIHWVRQAPG |
| | KGLEWVAKIQPKGGYTYYADSVKGRFTISADTSKNTAYLQM |
| | NSLRAEDTAVYYCARSYYYRPHAFDYWGQGTLVTVSSASTK |
| | GPSVFPLAPSSKSTSGGTAALGCLVKDYFPEPVTVSWNSGALT |
| | SGVHTFPAVLQSSGLYSLSSVVTVPSSSLGTQTYICNVNHKPS |
| | NTKVDKKVEPPKSC (SEQ ID NO: 189) |

### 6.14.30. Example 30: Antigen Affinities of SNIPER Candidates

Monovalent affinities of the SNIPER candidates to individual antigens CTLA4 and CD25 were assessed by biolayer interferometry as described herein. Equilibrium binding analysis was performed using two-fold serial dilutions of the BC1 1×1 B-Body SNIPER 19×8 binding molecules and their derivatives from 2 μM to 31.25 nM. Briefly, an initial baseline step was performed using an assay buffer. Next, in a loading step, biotinylated antigen (CTLA4 or CD25, respectively) was immobilized onto the streptavidin sensor. After antigen immobilization, the sensor was contacted with a buffer solution to determine loading level of the antigen. Next, the sensor was contacted with a solution containing the various dilutions of the candidate binding molecules to determine association rates. After association, the sensor was contacted with a buffer solution to determine dissociation rates of the multispecific Treg-binding molecules.

The association and dissociation kinetics of the dilution series were used to create steady state analysis plots of maximum binding response by binding molecule concentration.

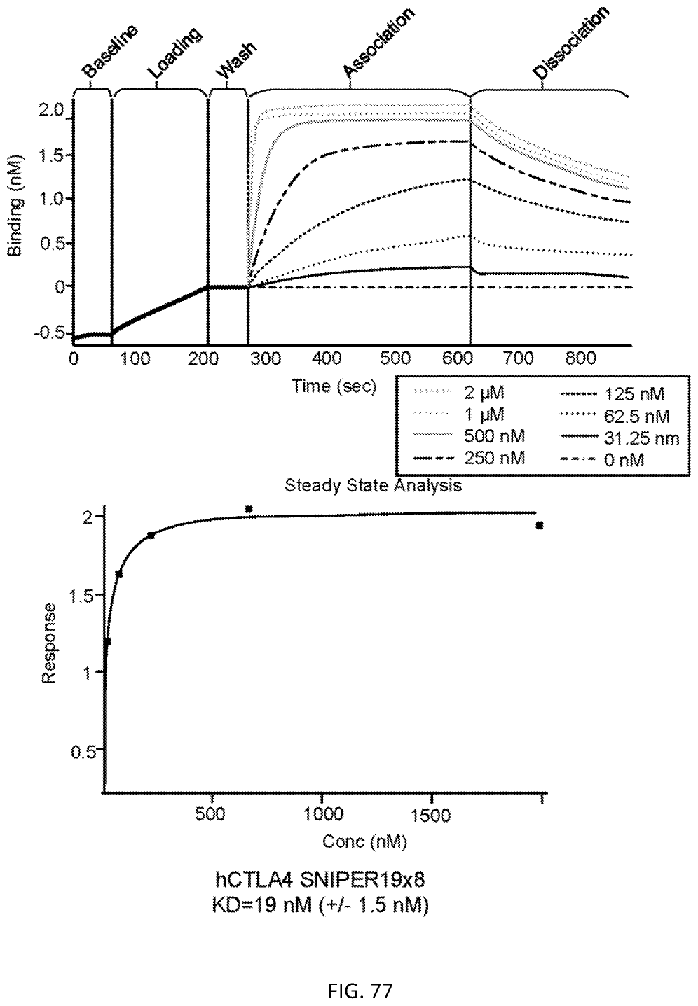

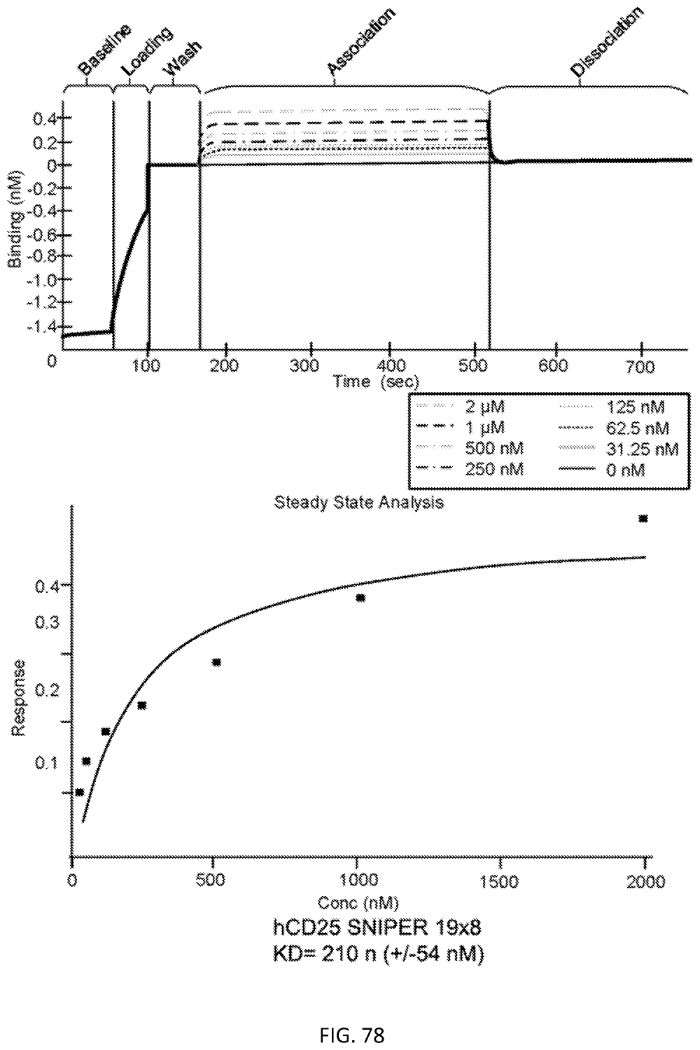

Results from SNIPER 19×8 are depicted in FIGS. 77 and 78, respectively. The Kd of SNIPER 19×8 for hCTLA4 was 19 nM (+/−1.5 nM). See FIG. 77. The Kd of SNIPER 19×8 for CD25 was 210 nM (+/−54 nM). See FIG. 78.

### 6.14.31. Example 31: SNIPER Candidates Having Low Binding Affinity for Treg Cell Surface Antigens CTLA4 and CD25 Selectively Bind Tumor-Associated Tregs Over Circulating Tregs

Matched dissociated tumor samples and PBMCs were obtained from a lung cancer patient and as described herein. Cell subpopulations were analyzed by flow cytometry. Briefly, patient samples were split into two aliquots. Each aliquot was gated according to the following gating parameters.

TABLE 17
Gating parameters for cell subpopulations

| Cell Population | Gating parameter | Antibody |
|---|---|---|
| Live cells | Ghost Dye ™ | Ghost Dye ™ Red 780 |
| | Red 780 (low | (Tonbo Bioscience) |
| | staining) | |
| Lymphocytes | CD45+ | Mouse anti-human CD45− |
| | | BD Horizon ™ V500 |
| | | (BD Biosciences) |
| All T-cells | CD3+ | Mouse anti-human CD3− |
| | | BD Horizon ™ BV605 |
| | | (BD Biosciences) |
| Killer T cells | CD8+ | Mouse Anti-Human CD8− |
| | | BD Horizon ™ BV711 |
| | | (BD Biosciences) |
| Teffector and Treg | CD4+ | Anti-Human CD4 PerCP- |
| | | eFluor ® 710 |
| | | (ThermoFisher) |
| Tregs | FoxP3+ and | FOXP3 PE (R&D Systems) |
| | CD127 low | CD127 Monoclonal |
| | | Antibody-PE-Cyanine7 |
| | | (Thermo Fisher) |

Aliquot 1 used the commercial antibodies to CTLA4 and CD25 to determine the number of double positive CTLA4/CD25 cells there were in the Ghost dye negative/CD45+/CD3+/CD4+/FOXP3+/CD127-gate (the Treg subpopulation). Aliquot 2 was used to determine the number of cells in the same gate (Ghost dye negative/CD45+/CD3+/CD4+/FOXP3+/CD127-) that were labeled with the SNIPER binding molecules.

CD127 Monoclonal Antibody-PE-Cyanine7 (Thermo Fisher eFluor). The sorted cells were then subjected to immunofluorescence staining with the SNIPER 19×8 binding molecule or their derivatives, and assessed by flow cytometry. The concentration of the SNIPER molecules was 10 nM.

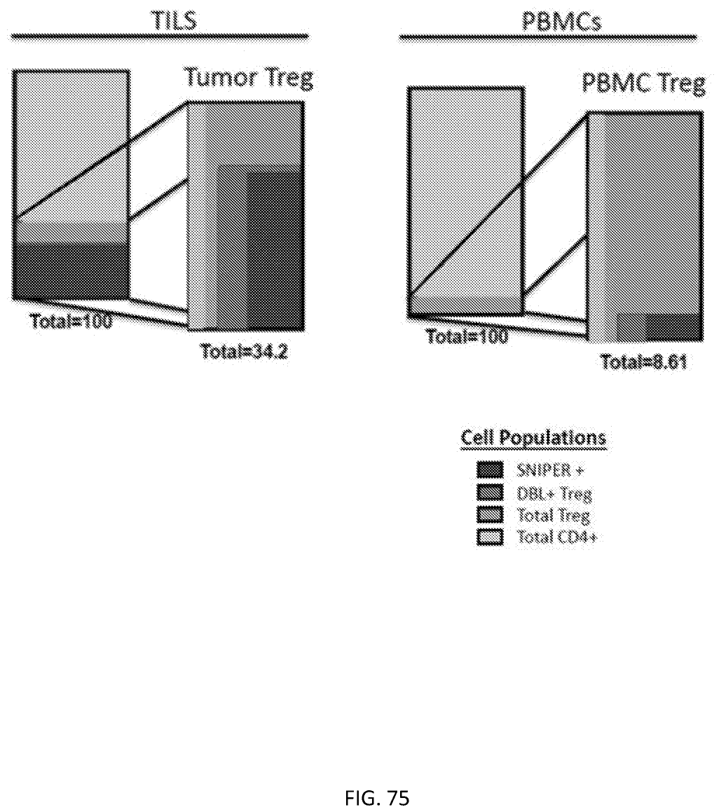

19×8 data from a flow experiment assessing binding of tumor-associated Tregs over circulating Tregs is depicted in FIG. 75. In the dissociated tumor sample, 34.2% of the CD4+ T cells were Treg cells. By contrast, in the PBMCs, just 8.61% of the CD4+ T cells were Treg cells. Of the Treg cells in the tumor, 73% were double positive for expression of CTLA4 and CD25. By contrast, 12% of PBMC Tregs were double positive for CTLA4 and CD25. Without wishing to be bound by theory, these double positive Tregs may have been shed from tumor tissue and are thus desirable targets. The SNIPER 19×8 binding molecule bound to 95% of the double positive cells in the tumor and 98% of the double positive cells in the PBMCs. The SNIPER 19×8 molecule bound 69% of total tumor-infiltrating Tregs. By contrast, the SNIPER 19×8 binding molecule only bound 12% of circulating Tregs.

### 6.14.32. Example 32: SNIPER Candidates Having Low Binding Affinity for Treg Cell Surface Antigens CTLA4 and CD25 Exhibit Very Little Binding to CD8+ Cells

Matched tumor and PBMC cells from a lung cancer patient were processed as described in Example 31. Cell subpopulations were isolated using flow cytometry according to the following gating parameters.

TABLE 18
Gating parameters for cell subpopulations

| Cell Population | Gating parameter | Antibody |
|---|---|---|
| Live cells | Ghost Dye ™ | Ghost Dye ™ Red 780 |
| | Red 780 (low | (Tonbo Bioscience) |
| | stainng) | |
| Lymphocytes | CD45+ | Mouse anti-human CD45− |
| | | BD Horizon ™ V500 |
| | | (BD Biosciences) |
| Killer T cells | CD8+ | Mouse Anti-Human CD8− |
| | | BD Horizon ™ V711 |
| | | (BD Biosciences) |

The sorted cells were then subjected to immunofluorescence staining with the SNIPER 19×8 binding molecule or their derivatives, and assessed by flow cytometry.

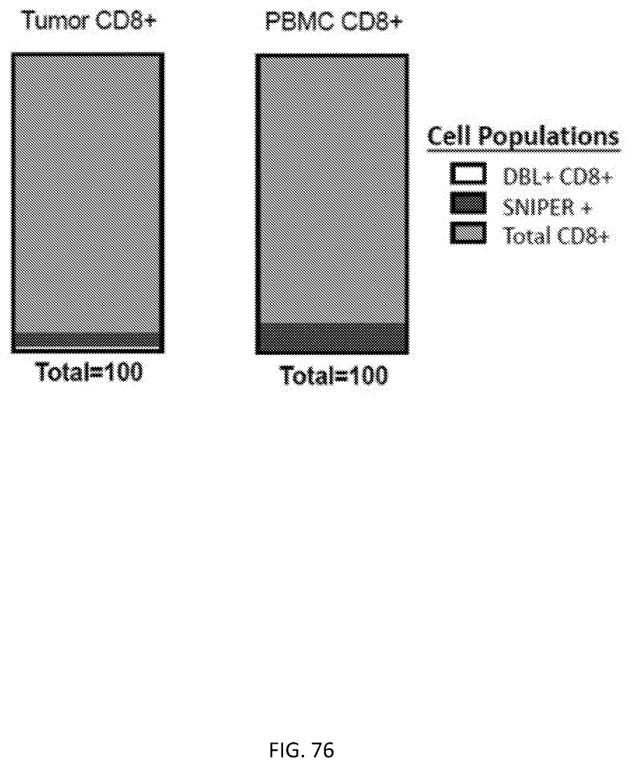

19×8 data from a single flow experiment assessing binding of tumor-infiltrating or circulating CD8+ cells is depicted in FIG. 76. 94% of the CD8+ T-cells were spared in the tumor samples. 90% of the CD8+ T-cells were spared in the PBMCs. These results indicate that SNIPER 19×8 binding molecules spare most effector cells in the tumor, allowing anti-tumor activity from immune cells, and also spare most circulating immune cells, minimizing disruption to the immune system.

A summary of results from the experiments in Examples 31 and 32 is depicted in the table below.

TABLE 19
Percentage of NSCLC Cell Populations Bound by SNIPER

| Sample | Cell Populations | Patient 1 | Patient 2 |
|---|---|---|---|
| TILs | Total CD4+ T-cell | 23% | 7% |
| | Total Tregs | 70% | 51% |
| | Double-Positive Tregs | 95% | 114% |
| | Total CD8+ T-cells | 6% | 4% |
| PBMCs | Total CD4+ T-cells | 1% | 1% |
| | Total Tregs | 12% | 8% |
| | Double-Positive Treg | 98% | 65% |
| | Total CD8+ T-cells | 10% | 12% |

### 6.14.33. Example 33: Optimization of SNIPER 19×8 Variants

A series of SNIPER 19×8 variants were generated by engineering amino acid substitutions at various positions of the CDR sequences in the monovalent CTLA4-19 and CD25-8 arms, respectively. The SNIPER 19×8 variants were expressed and purified as described herein.

The CTLA4-19-67 variant (SNIPER 67) was generated by introducing Y/S mutations in CDR L3. The CTLA4-19-95 variant (SNIPER 95) was generated by introducing Y/S mutations in CDR L3 and a Y/S mutation in CDR H1. These mutations lower the monovalent affinity of this variant for its antigen.

The CDR sequences for the CD25-8 and CTLA4 parent binding arms, as well as for the SNIPER 67 and SNIPER 95 variants, is depicted in the table below. Underlining indicates the introduced Y/S mutations.

TABLE 20
SNIPER binding arm CDR sequences

| | | | | CDR H1 | | |
|---|---|---|---|---|---|---|
| | | | | (SEQ | | |
| | | CDR L2 | CDR L3 | ID NOS | | CDR H3 |
| | CDR L1 (SEQ | (SEQ ID | (SEQ ID | 195-196 | CDR H2 (SEQ | (SEQ ID |
| | ID NOS 190, | NOS 191, | NOS | and | ID NOS 198, | NOS 200, |
| | 190, 190, | 191, 191, | 192-194 | 196- | 199, 199, | 201, 201, and |
| Variant | and 190) | and 191) | and 194) | 197) | 199) | 201) |
| CD25-8 | RASQSVSSAVA | | YSSQPA | FSSYY | AKIQPKGGYTY | SYYYRPHAF |
| CTLA4- | RASQSVSSAVA | SASSLYS | YYTTPF | FSGYW | AAITSRDSYTY | GSYLGSAF |
| 19 | | | | | | |
| CTLA4- | RASQSVSSAVA | SASSLYS | SSTTPF | FSGYW | AAITSRDSYTY | GSYLGSAF |
| 19-67 | | | | | | |
| CTLA4- | RASQSVSSAVA | SASSLYS | SSTTPF | FSGSW | AAITSRDSYTY | GSYLGSAF |
| 19-95 | | | | | | |

19×8 SNIPER variants were assembled as described herein.

In greater detail below, with domain and polypeptide chain references in accordance with FIG. 3 and modifications from native sequences indicated in parentheses, the architecture of SNIPER variant 67 (“SNIPER 67”) was:
1^{st} polypeptide chain
   Domain A=VL (“CTLA4-19, L3 CDR=SSTTPF” (SEQ ID NO: 194))
   Domain B=CH3 (T366K; 445K, 446S, 447C tripeptide insertion)
   Domain D=CH2
   Domain E=CH3 (T366W, S354C)
2nd polypeptide chain
   Domain F=VH (“CTLA4-19”)
   Domain G=CH3 (L351D; 445G, 446E, 447C tripeptide insertion)
3rd polypeptide chain
   Domain H=VL (“CD25-8”)
   Domain I=CL (Kappa)
   Domain J=CH2
   Domain K=CH3 (Y349C, D356E, L358M, T366S, L368A, Y407V)
4th polypeptide chain
   Domain L=VH (“CD25-8”)
   Domain M=CH1.

The sequences for the four polypeptide chains of SNIPER 67 are indicated in Table 21.

TABLE 21
polypeptide chain sequences, SNIPER 67

| Polypeptide | |
|---|---|
| Chain # | Sequence |
| 1 | DIQMTQSPSSLSASVGDRVTITCRASQSVSSAVAWYQQKPGKAPKLLIY |
| | SASSLYSGVPSRFSGSRSGTDFTLTISSLQPEDFATYYCQQSSTTPFTFGQ |
| | GTKVEIKRTPREPQVYTLPPSRDELTKNQVSLKCLVKGFYPSDIAVEWE |
| | SNGQPENNYKTTPPVLDSDGSFFLYSKLTVDKSRWQQGNVFSCSVMHE |
| | ALHNHYTQKSLSLSKSCDKTHTCPPCPAPELLGGPSVFLFPPKPKDTLMI |
| | SRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQV |
| | YTLPPCRDELTKNQVSLWCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 202) |
| 2 | EVQLVESGGGLVQPGGSLRLSCAASGFTFSGYWIHWVRQAPGKGLEW |
| | VAAITSRDSYTYYADSVKGRFTISADTSKNTAYLQMNSLRAEDTAVYY |
| | CARGSYLGSAFDYWGQGTLVTVSSASPREPQVYTDPPSRDELTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSGEC (SEQ ID NO: 203) |
| 3 | SDIQMTQSPSSLSASVGDRVTITCRASQSVSSAVAWYQQKPGKAPKLLI |
| | YSASSLYSGVPSRFSGSRSGTDFTLTISSLQPEDFATYYCQQYSSQPATFG |
| | QGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGECDKTHTCPPCPAPELLGGPSVFLFPPKPKDTLM |
| | ISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQV |
| | CTLPPSREEMTKNQVSLSCAVKGFYPSDIAVEWESNGQPENNYKTTPPV |
| | LDSDGSFFLVSKLTVDKSRWQQGNVFSCSVMHEALHNHYTQKSLSLSP |
| | GK (SEQ ID NO: 204) |
| 4 | EVQLVESGGGLVQPGGSLRLSCAASGFTFSSYYIHWVRQAPGKGLEWV |
| | AKIQPKGGYTYYADSVKGRFTISADTSKNTAYLQMNSLRAEDTAVYYC |
| | ARSYYYRPHAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSS |
| | SLGTQTYICNVNHKPSNTKVDKKVEPPKSC (SEQ ID NO: 205) |

In greater detail below, with domain and polypeptide chain references in accordance with FIG. 3 and modifications from native sequences indicated in parentheses, the architecture of SNIPER variant 95 (“SNIPER 95”) was:
1^{st} polypeptide chain
   Domain A=VL (“CTLA4-19, L3 CDR=SSTTPF” (SEQ ID NO: 194))
   Domain B=CH3 (T366K; 445K, 446S, 447C tripeptide insertion)
   Domain D=CH2
   Domain E=CH3 (T366W, S354C)
2nd polypeptide chain
   Domain F=VH (“CTLA-19, H1 CDR=FSGSW” (SEQ ID NO: 197))
   Domain G=CH3 (L351D; 445G, 446E, 447C tripeptide insertion) 3rd polypeptide chain
   Domain H=VL (“CD25-8”)
   Domain I=CL (Kappa)
   Domain J=CH2
   Domain K=CH3 (Y349C, D356E, L358M, T366S, L368A, Y407V)
4th polypeptide chain
   Domain L=VH (“CD25-8”)
   Domain M=CH1.

The sequences for the four polypeptide chains of SNIPER 95 are indicated in Table 22.

TABLE 22
polypeptide chain sequences, SNIPER 95

| Polypeptide | |
|---|---|
| Chain # | Sequence |
| 1 | DIQMTQSPSSLSASVGDRVTITCRASQSVSSAVAWYQQKPGKAPKLL |
| | IYSASSLYSGVPSRFSGSRSGTDFTLTISSLQPEDFATYYCQQSSTTPFT |
| | FGQGTKVEIKRTPREPQVYTLPPSRDELTKNQVSLKCLVKGFYPSDIA |
| | VEWESNGQPENNYKTTPPVLDSDGSFFLYSKLTVDKSRWQQGNVFS |
| | CSVMHEALHNHYTQKSLSLSKSCDKTHTCPPCPAPELLGGPSVFLFP |
| | PKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKT |
| | ISKAKGQPREPQVYTLPPCRDELTKNQVSLWCLVKGFYPSDIAVEWE |
| | SNGQPENNYKTTPPVLDSDGSFFLYSKLTVDKSRWQQGNVFSCSVM |
| | HEALHNHYTQKSLSLSPGK (SEQ ID NO: 206) |
| 2 | EVQLVESGGGLVQPGGSLRLSCAASGFTFSGSWIHWVRQAPGKGLE |
| | WVAAITSRDSYTYYADSVKGRFTISADTSKNTAYLQMNSLRAEDTA |
| | VYYCARGSYLGSAFDYWGQGTLVTVSSASPREPQVYTDPPSRDELT |
| | KNQVSLTCLVKGFYPSDIAVEWESNGQPENNYKTTPPVLDSDGSFFL |
| | YSKLTVDKSRWQQGNVFSCSVMHEALHNHYTQKSLSLSGEC (SEQ |
| | ID NO: 207) |
| 3 | SDIQMTQSPSSLSASVGDRVTITCRASQSVSSAVAWYQQKPGKAPKL |
| | LIYSASSLYSGVPSRFSGSRSGTDFTLTISSLQPEDFATYYCQQYSSQP |
| | ATFGQGTKVEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGECGGGSGGGGSKTHTCPPCPAPEL |
| | LGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVCTLPPSREEMTKNQVSLSCAVKGFYP |
| | SDIAVEWESNGQPENNYKTTPPVLDSDGSFFLVSKLTVDKSRWQQG |
| | NVFSCSVMHEALHNHYTQKSLSLSPGK (SEQ ID NO: 208) |
| 4 | EVQLVESGGGLVQPGGSLRLSCAASGFTFSSYYIHWVRQAPGKGLE |
| | WVAKIQPKGGYTYYADSVKGRFTISADTSKNTAYLQMNSLRAEDTA |
| | VYYCARSYYYRPHAFDYWGQGTLVTVSSASTKGPSVFPLAPSSKSTS |
| | GGTAALGCLVKDYFPEPVTVSWNSGALTSGVHTFPAVLQSSGLYSL |
| | SSVVTVPSSSLGTQTYICNVNHKPSNTKVDKKVEPPKSC (SEQ ID |
| | NO: 209) |

### 6.14.34. Example 34: Testing of SNIPER 19×8 Variants Binding Molecules to CTLA4 and CD25

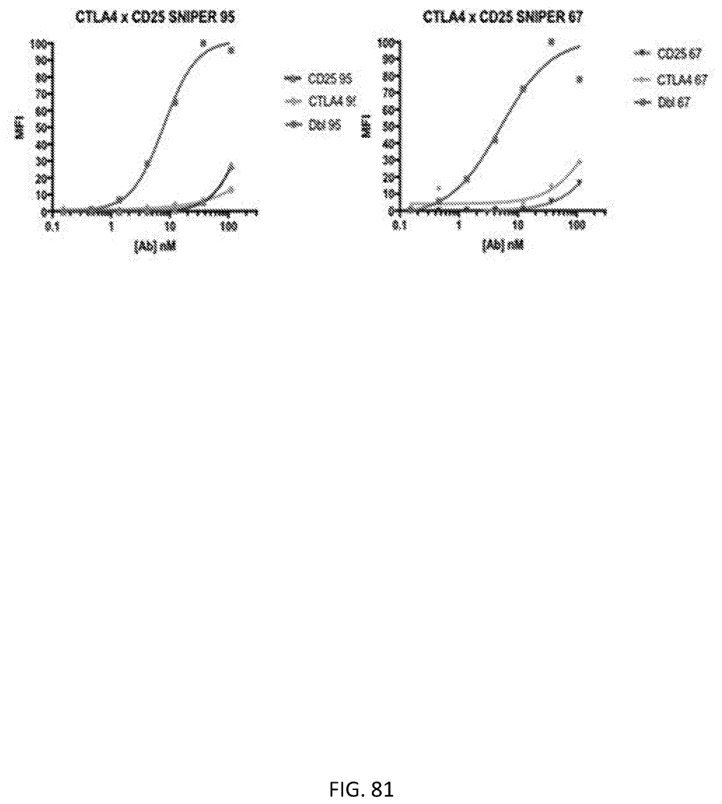

The SNIPER 67 and SNIPER 95 candidates were tested as described herein for binding to HEK293 cells stably expressing single targets CTLA4 (“CTLA4 only”), CD25 (“CD25 only”), or doubly expressing both targets (“double stb”). Binding affinities of the SNIPER candidates to single antigens were extrapolated from binding data from single antigen expressing cells, while binding avidity was assessed using binding data from doubly expressing cells. Results are depicted in FIG. 81, showing dramatically weaker single antigen affinity for singly-expressing HEK293 cells, and significantly stronger avidity to cells expressing both targets. Avidity and affinity results from this assay are depicted below.

TABLE 23
Binding avidity and affinity of SNIPER binding molecules

| | Kd | Kd | Kd |
|---|---|---|---|
| Binding Molecule | (double stb) | (CD25 only) | (CTLA4 only) |
| SNIPER 67 | 4.3 nM | 210 nM | 160 nM |
| SNIPER 95 | 7.9 nM | 210 nM | 801 nM |

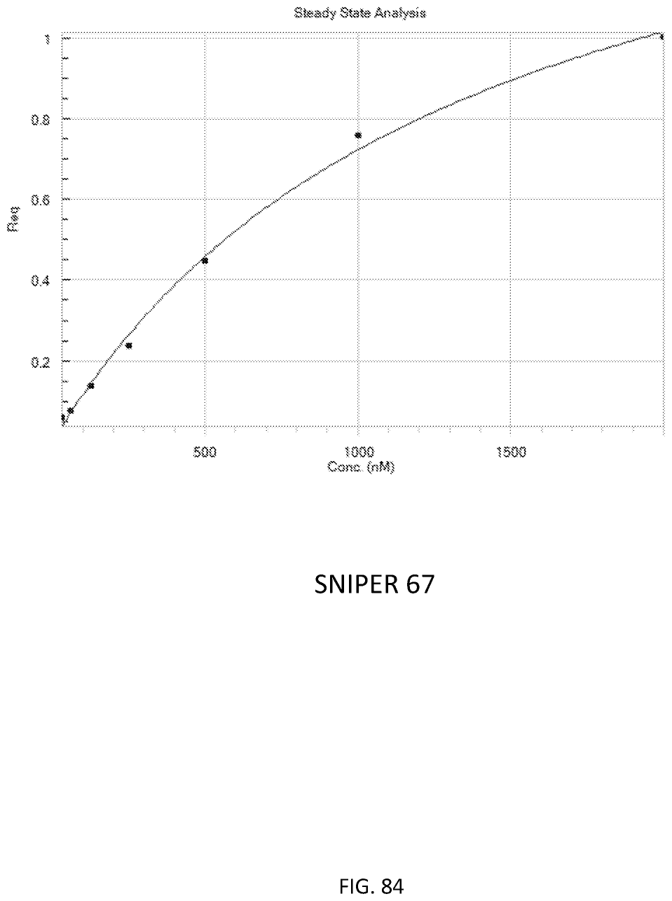

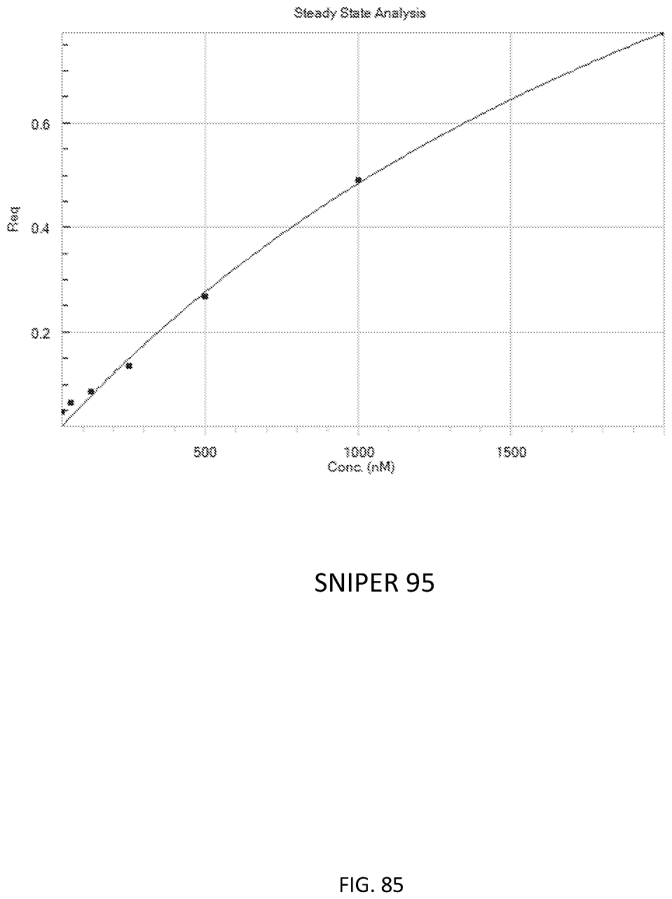

Binding affinity of the SNIPER 67 and SNIPER 95 candidates was also assessed using BLI. BLI experiments were performed as described in Example 30. Results are described in the following table and in FIGS. 84 and 85.

TABLE 24
BLI dose response curve

| Conc. | Response | Response |
|---|---|---|
| (nM) | (SNIPER 67) | (SNIPER 95) |
| 2000 | 1.0576 | 0.8264 |
| 1000 | 0.7723 | 0.506 |
| 500 | 0.4272 | 0.2543 |
| 250 | 0.2018 | 0.126 |
| 125 | 0.1074 | 0.0721 |
| 61.5 | 0.0585 | 0.0588 |
| 31.3 | 0.0447 | 0.0491 |

### 6.14.35. Example 35: Removal of Potential Isomerization Sites in CTLA4-19 Binding Arm

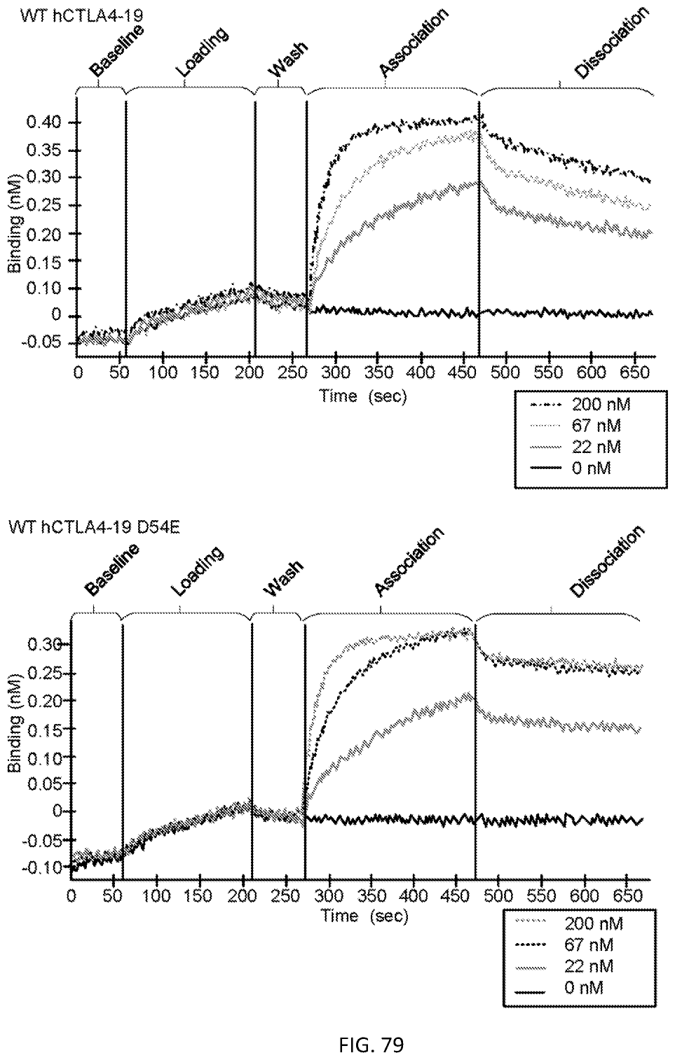

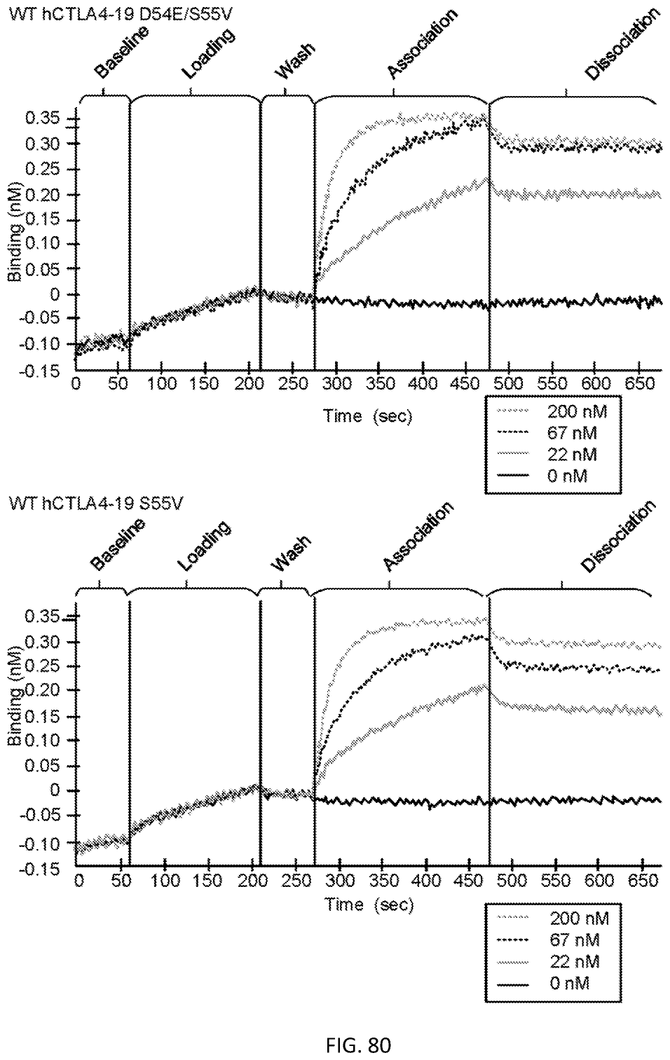

Aspartate isomerization can lead to charge heterogeneity and result in fragmentation and aggregation due to cleavage of the peptide backbone. The risk of immunogenicity may be also increased by aspartate isomerization. A series of SNIPER variants were made to the CTLA4 binding arm of the SNIPER 19×8 binding molecule, to eliminate the potential aspartate isomerization site at position VH-54 of CDR H2. These mutations included D54E, D54E/S55V and S55V. These mutants were tested by BLI as described herein. Briefly, biotinylated human CTLA4 was immobilized to a final response of ˜0.1 nm. Three concentrations of 1×1 bispecific were used for analysis (200, 67 and 22 nM). Results are shown in FIGS. 79 and 80, demonstrating that these mutations do not negatively impact binding.

### 6.14.36. Example 36: ADC Assay

Cells expressing both targets of a SNIPER antibody (i.e. CTLA4 and CD25), one target of a SNIPER antibody (i.e. only CTLA4 or only CD25), or neither target of a SNIPER antibody are added to a 96 well plate. Dose titrations of the SNIPER ADC antibody or an isotype control ADC antibody are added to the wells. Cells are incubated at 37 degrees C. and 5% CO2 for 2-5 days. After 2-5 days, a cell viability assay (i.e. PrestoBlue, AlamarBlue, MTT, MTX, CellTiter Glo, etc.) is performed to determine the percentage of dead cells. Data is normalized to a no antibody control (100% viable) and a detergent killed control (0% viable). Alternatively, a lactate dehydrogenase (LDH) release assay is performed at the end of the incubation period to determine the amount of LDH released into the media by dead/dying cells.

### 6.14.37. Example 37: ADCC (Trivalent, Trispecific Antibody Having Orthogonal CH1/CL Mutations

Antibody Construction

A trispecific binding molecule, comprising a first and second ABS each having low binding affinity for a Treg-specific marker, and a third ABS which specifically binds a cell surface marker for a natural killer T-cell or macrophage (“SNIPER ADCC_CH1/CL”) is constructed. The SNIPER ADCC_CH1/CL binding molecule has the architecture depicted in FIG. A2. Briefly, the SNIPER ADCC_CH1/CL binding molecule has a first, second, third, fourth, and fifth polypeptide chain, wherein (a) the first polypeptide chain comprises, from N-terminus to C-terminus, a first VL amino acid sequence (VL1) specific for a first Treg cell surface marker, a human IgG1 CH3 amino acid sequence with a Y349C mutation and a C-terminal extension incorporating a PGK tripeptide sequence that is followed by the DKTHT motif (SEQ ID NO: 185) of an IgG1 hinge region, a human IgG1 CH2 amino acid sequence, and a human IgG1 CH3 amino acid with a S354C and a T366W mutation; (b) the second polypeptide comprises, from N-terminus to C-terminus, a first VH amino acid sequence (VH1) specific for the first Treg cell surface marker and a human IgG1 CH3 amino acid sequence with a S354C mutation and a C-terminal extension incorporating a PGK tripeptide sequence; (c) the third polypeptide comprises, from N-terminus to C-terminus, a third VL amino acid sequence specific for a cell surface marker for a natural killer T-cell or macrophage, a first CL kappa amino acid sequence comprising Phe118Cys and Asn138Lys mutations, a second VL amino acid sequence specific for a second Treg cell surface marker, a second CL kappa amino acid sequence which is a wtFab sequence; (d) the fourth polypeptide comprises, from N-terminus to C-terminus, a third VH sequence specific for a cell surface marker for a natural killer or macrophage, a first CH1 sequence comprising Leu128Phe118Cys and Asn138Lys mutations; and (e) the fifth polypeptide comprises, from N-terminus to C-terminus and a second VH sequence specific for the second Treg cell surface marker, and a second CH1 sequence which is a wtFab sequence.

Cells expressing both targets of a SNIPER antibody (i.e. CTLA4 and CD25), one target of a SNIPER antibody (i.e. only CTLA4 or only CD25), or neither target of a SNIPER antibody are added to a 96 well plate. Dose titrations of the SNIPER antibody or an isotype control antibody are added to the wells. PBMCs or purified natural killer cells are added to the wells at an E:T ratio between 4:1 and 25:1. Cells are incubated at 37 degrees C. and 5% CO2 for 4-24 hrs. After the incubation, a cell viability/cytotoxicity assay (i.e. PrestoBlue, AlamarBlue, MTT, MTX, CellTiter Glo, Chromium 51 release, LDH release, Calcein release, Propidium Iodide, 7-AAD, etc.) is performed to determine the percentage of dead cells. Data is normalized to a no antibody control (100% viable) and a detergent killed control (0% viable).

### 6.14.38. Example 38: Further Optimization and Characterization of CH3 Linker Sequences

In a further set of experiments, bivalent bispecific binding molecule variants, also termed “BA” specific for a first antigen (“Antigen A”) and second antigen (“Antigen B”) was constructed.

Salient features of the general BA architecture are illustrated in FIG. 3. In greater detail, with domain and polypeptide chain references in accordance with FIG. 3, the architecture of binding molecule “BA” was:
1^{st} polypeptide chain:
   Domain A=V_{L} (“Antigen A”)
   Domain B=C_{H3} (IgA, optional first CH3 linker)
   Domain D=C_{H2}
   Domain E=CH3 (Knob T366W, 354C)
2^{nd} polypeptide chain:
   Domain F=V_{H} (“Antigen A”)
   Domain G=C_{H3} (IgA, optional second CH3 linker)
3^{rd} polypeptide chain:
   Domain H=V_{L} (“Antigen B”)
   Domain I=C_{L} (Kappa)
   Domain J=C_{H2}
   Domain K=IgG1 CH3 (Y349C, D356E, L358M, T366S, L368A, Y407V)
4^{th} polypeptide chain:
   Domain L=V_{H} (“Antigen B”)
   Domain M=CH1.

The A domain (SEQ ID NO: 12) and F domain (SEQ ID NO: 16) form an antigen binding site (A:F) specific for “Antigen A”. Domain H and domain L form an antigen binding site (H:L) specific for “Antigen B”).

Domain B has the human IgA CH3 sequence, (SEQ ID NO:184), with an optional first CH3 linker sequence or modification which connects the C-terminus of CH3 to the N-terminus of CH2 (domain D).

Domain G has the human IgA CH3 sequence, (SEQ ID NO:184), with a second CH3 linker sequence or modification which forms a disulfide bridge with the first CH3 linker sequence.

Domain D has the sequence of human IgG1 CH2 domain, with a CH2 hinge sequence (SEQ ID NO: 56) appended to the N-terminus of the CH2 domain.

Domain E (SEQ ID NO:15) has the sequence of human IgG1 CH3 with the mutations T366W and S354C.

Domain I (SEQ ID NO:19) has the sequence of human C kappa light chain (CIO

Domain J has the sequence of human IgG1 CH2 domain.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in Domain E. The 356E and L358M introduce isoallotype amino acids that reduce immunogenicity. The 366S, 368A, and 407V are “hole” mutations.

Domain M has the sequence of the human IgG1 CH1 region.

The BA variants in this experiment comprise the architecture stated above and comprise different sets of first and second CH3 linkers in the first and second polypeptide chains, respectively (see Table 25 below). The first CH3 linker attaches domain B to domain D. the second CH3 linker comprises an engineered cysteine that forms a disulfide bond with a cysteine in the first CH3 linker.

TABLE 25
Additional BA variants with CH3 linkers

| | Chain 1 (first | Chain 2 (second |
|---|---|---|
| | CH3 linker) | CH3 linker) |
| BA11 (see Table 9) | AGC.c1 | AGKGSC.c2 |
| | | (SEQ ID NO: 99) |
| BA15 | AGKGC.c1 | AGC.c2 |
| BA11 (see Table 9) | (SEQ ID NO: 98) | |
| BA45 | H350C amino | P355C amino |
| | acid substitution | acid substitution |
| BA46 | P355C amino | H350C amino |
| | acid substitution | acid substitution |

For clarity, the residue designated “H350” in the IgA-CH3 domain sequence is the underlined “H” residue in the following endogenous IgA-CH3 sequence:

(SEQ ID NO: 184)
TFRPEVHLLPPPSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

By way of example, an IgA-CH3 amino acid domain sequence with a “H350C” mutation in an otherwise endogenous IgA-CH3 domain has the following sequence:

(SEQ ID NO: 303)
TFRPEVCLLPPPSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

For clarity, the residue designated “P355” in the IgA-CH3 domain sequence is the underlined “P” residue in the following endogenous IgA-CH3 sequence:

(SEQ ID NO: 184)
TFRPEVHLLPPPSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

By way of example, an IgA-CH3 amino acid domain sequence with a “P355C” mutation in an otherwise endogenous IgA-CH3 domain has the following sequence:

(SEQ ID NO: 304)
TFRPEVHLLPP CSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

Protein was expressed by transient expression in Expi 293 cells as described above, and purified by one-step affinity chromatography using CH1 resin.

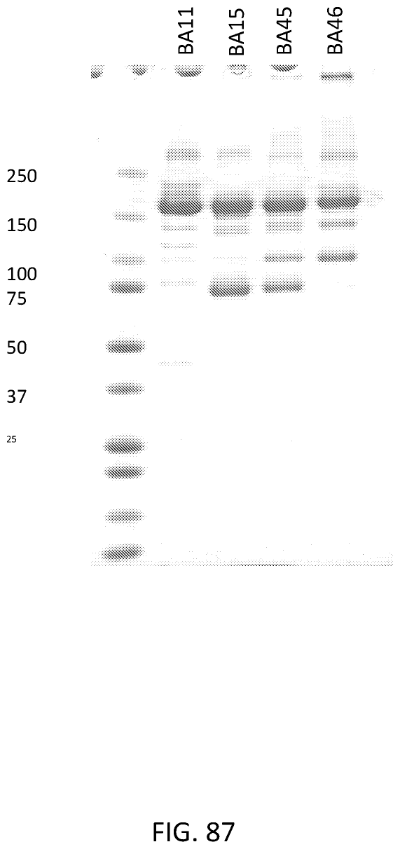

FIG. 87 depicts SDS-PAGE analysis of the BA variants in Table 16. All constructs exhibited full assembly and overall greater yield of fully assembled binding molecules as compared to incomplete products. Of the four variants, BA11 exhibited particularly high-fidelity assembly and high yield.

### 6.14.39. EXAMPLE 39: TRIVALENT CONSTRUCTS WITH IGA-CH3 DOMAIN SWAPS

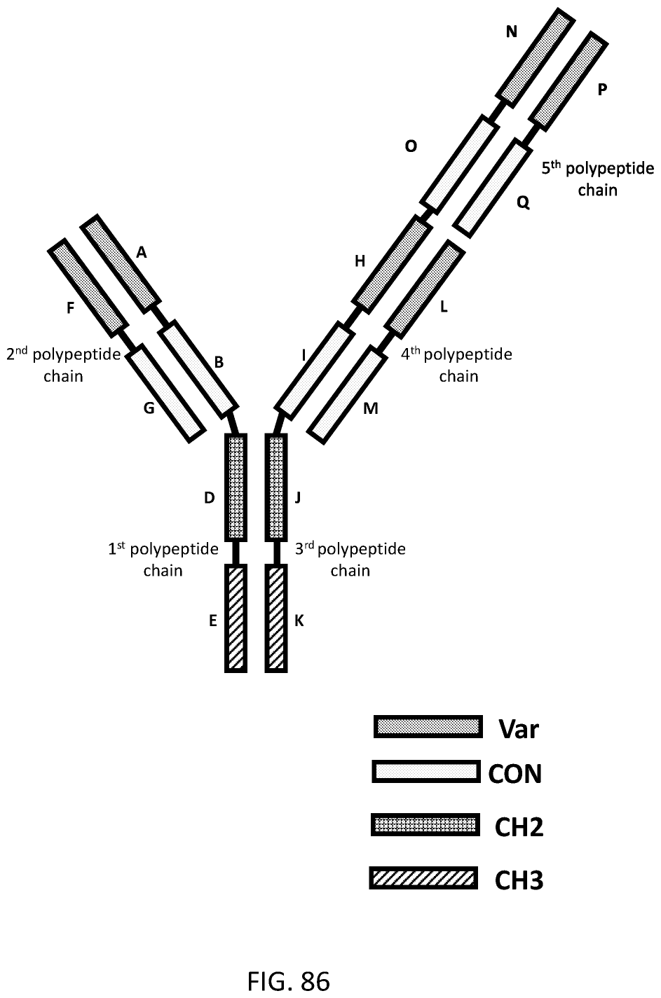

Trivalent binding molecules were constructed according to the general architecture depicted in FIG. 86. The binding molecules comprise a first polypeptide chain, a second polypeptide chain, a third polypeptide chain, a fourth polypeptide chain, and a fifth polypeptide chain.

The first polypeptide chain comprises a domain A, a domain B, a domain D, and a domain E, wherein the domains are arranged, from N-terminus to C-terminus, in an A-B-D-E orientation. Domain A has a variable region amino acid sequence, domain B has a constant region amino acid sequence, domain D has a CH2 sequence, and domain E has a CH3 sequence.

The second polypeptide chain comprises a domain F and a domain G, wherein the domains are arranged, from N-terminus to C-terminus, in a F-G orientation. Domain F has a variable region domain amino acid sequence and domain G has a constant region domain amino acid sequence.

The third polypeptide chain comprises a domain N, a domain O, a domain H, a domain I, a domain J, and a domain K, wherein the domains are arranged, from N-terminus to C-terminus, in a N-O-H-I-J-K orientation. Domain N has a variable region amino acid sequence, domain O has a constant region amino acid sequence, domain H has a variable region domain amino acid sequence, domain I has a constant region amino acid sequence, domain J has a CH2 sequence, and domain K has a CH3 sequence.

The fourth polypeptide chain comprises a domain L and a domain M, wherein the domains are arranged, from N-terminus to C-terminus, in a L-M orientation. Domain L has a variable region domain amino acid sequence and domain M comprises a constant region amino acid sequence.

The fifth polypeptide chain comprises a domain P and a domain Q, wherein the domains are arranged, from N-terminus to C-terminus, in a P-Q orientation. Domain P comprises a variable region amino acid sequence and domain Q comprises a constant region amino acid sequence.

Domains A and F associate to form a first antigen binding site; domains H and L associate to form a second antigen binding site; and domains N and P associate to form a third antigen binding site.

Domains B and G form a first domain pair of associated constant region domains (“first domain pair”), domains I and M form a second domain pair of associated constant region domains (“second domain pair”), and domains Q and O form a third domain pair of associated constant region domains (“third domain pair”). At least one of the first, second, and third domain pairs is a pair of associated IgA-CH3 domains.

The architecture of exemplary constructed trivalent molecules is shown in the Table 17 below. A non-exhaustive list of optional modifications are shown in italics.

TABLE 17
trivalent binding molecules comprising IgA-CH3/IgA-CH3 domain pairs

| Construct | | | | | |
|---|---|---|---|---|---|
| Name | Chain 1 | Chain 2 | Chain 3 | Chain 4 | Chain 5 |
| T26 | A = VL | F = VH | N = VL | L = VH | P = VH |
| | B = IgA-CH3 | G = IgA-CH3 | O = IgA-CH3 | M = CH1 | Q = IgA-CH3 |
| | H350C | P355C | AGKGSC | | AGC |
| | D = CH2 | | linker (SEQ | | linker |
| | L234A, L235A, | | ID NO: 99) | | |
| | P329K | | H = VL | | |
| | E = IgG CH3 | | I = CL | | |
| | Y349C, D356E, | | J = CH2 | | |
| | L358M, T366S, | | L234A, L235A, | | |
| | L368A, Y407V | | P329K | | |
| | | | K = IgG CH3 | | |
| | | | S354C, T366W | | |
| T27 | A = VL | F = VH | N = VL | L = VH | P = VH |
| | B = IgA-CH3 | G = IgA-CH3 | O = IgA-CH3 | M = CH1 | Q = IgA-CH3 |
| | AGC | AGKGSC | H350C | | P355C |
| | linker | linker (SEQ | H = VL | | |
| | D = CH2 | ID NO: 99) | I = CL | | |
| | L234A, L235A, | | J = CH2 | | |
| | P329K | | L234A, L235A, | | |
| | E = IgG CH3 | | P329K | | |
| | Y349C, D356E, | | K = IgG CH3 | | |
| | L358M, T366S, | | S354C, T366W | | |
| | L368A, Y407V | | | | |
| T28 | A = VL | F = VH | N = VL | L = VH | P = VH |
| | B = IgA-CH3 | G = IgA-CH3 | O = IgA-CH3 | M = CH1 | Q = IgA-CH3 |
| | H350C | P355C | AGC | | AGKGSC |
| | D = CH2 | | linker | | linker (SEQ |
| | L234A, L235A, | | H = VL | | ID NO: 99) |
| | P329K | | I = CL | | |
| | E = IgG CH3 | | J = CH2 | | |
| | Y349C, D356E, | | L234A, L235A, | | |
| | L358M, T366S, | | P329K | | |
| | L368A, Y407V | | K = IgG CH3 | | |
| | | | S354C, T366W | | |
| T33 | A = VL | F = VH | N = VL | L = VH | P = VH |
| | B = IgG-CH3 | G = IgG-CH3 | O = IgA-CH3 | M = CH1 | Q = IgA-CH3 |
| | T366K, 447C | L351D, 447C | H350C | | P355C |
| | D = CH2 | | H = VL | | |
| | L234A, L235A, | | I = CL | | |
| | P329K | | J = CH2 | | |
| | E = IgG CH3 | | L234A, L235A, | | |
| | Y349C, D356E, | | P329K | | |
| | L358M, T366S, | | K = IgG CH3 | | |
| | L368A, Y407V | | S354C, T366W | | |
| T34 | A = VL | F = VH | N = VL | L = VH | P = VH |
| | B = IgA-CH3 | G = IgA-CH3 | O = IgG-CH3 | M = CH1 | Q = IgG-CH3 |
| | H350C | P355C | T366K, 447C | | L351D, 447C |
| | D = CH2 | | H = VL | | |
| | L234A, L235A, | | I = CL | | |
| | P329K | | J = CH2 | | |
| | E = IgG CH3 | | L234A, L235A, | | |
| | Y349C, D356E, | | P329K | | |
| | L358M, T366S, | | K = IgG CH3 | | |
| | L368A, Y407V | | S354C, T366W | | |
| T35 | A = VL | F = VH | N = VL | L = VH | P = VH |
| | B = IgG-CH3 | G = IgG-CH3 | O = IgA-CH3 | M = CH1 | Q = IgA-CH3 |
| | T366K, 447C | L351D, 447C | AGC | | AGKGSC |
| | D = CH2 | | linker | | linker (SEQ |
| | L234A, L235A, | | H = VL | | ID NO: 99) |
| | P329K | | I = CL | | |
| | E = IgG CH3 | | J = CH2 | | |
| | Y349C, D356E, | | L234A, L235A, | | |
| | L358M, T366S, | | P329K | | |
| | L368A, Y407V | | K = IgG CH3 | | |
| | | | S354C, T366W | | |
| T36 | A = VL | F = VH | N = VL | L = VH | P = VH |
| | B = IgA-CH3 | G = IgA-CH3 | O = IgG-CH3 | M = CH1 | Q = IgG-CH3 |
| | AGC | AGKGSC | T366K, 447C | | L351D, 447C |
| | linker | linker (SEQ | H = VL | | |
| | D = CH2 | ID NO: 99) | I = CL | | |
| | L234A, L235A, | | J = CH2 | | |
| | P329K | | L234A, L235A, | | |
| | E = IgG CH3 | | P329K | | |
| | Y349C (for | | K = IgG CH3 | | |
| | engineered | | S354C, T366W | | |
| | disulfide bridge), | | | | |
| | D356E, L358M, | | | | |
| | T366S, L368A, | | | | |
| | Y407V | | | | |
| T37 | A = VL | F = VH | N = VL | L = VH | P = VH |
| | B = IgG-CH3 | G = IgG-CH3 | O = IgA-CH3 | M = CH1 | Q = IgA-CH3 |
| | [P343V; Y349C; | (S354C; 445P, | AGC | | AGKGSC |
| | 445P, 446G, | 446G, 447K | linker | | linker (SEQ |
| | 447K | insertion) | H = VL | | ID NO: 99) |
| | insertion] | | I = CL | | |
| | D = CH2 | | J = CH2 | | |
| | L234A, L235A, | | L234A, L235A, | | |
| | P329K | | P329K | | |
| | E = IgG CH3 | | K = IgG CH3 | | |
| | Y349C, D356E, | | S354C, T366W | | |
| | L358M, T366S, | | | | |
| | L368A, Y407V | | | | |
| T38 | A = VH | F = VL | N = VH | L = VL | P = VL |
| | B = IgG-CH3 | G = IgG-CH3 | O = IgA-CH3 | M = C1 | Q = IgA-CH3 |
| | P343V; Y349C; | (S354C; 445P, | AGC | | AGKGSC |
| | 445P, 446G, | 446G, 447K | linker | | linker (SEQ |
| | 447K | insertion) | H = VH | | ID NO: 99) |
| | insertion | | I = CH1 | | |
| | D = CH2 | | J = CH2 | | |
| | L234A, L235A, | | L234A, L235A, | | |
| | P329K | | P329K | | |
| | E = IgG-CH3 | | K = IgG-CH3 | | |
| | Y349C, D356E, | | S354C, T366W | | |
| | L358M, T366S, | | | | |
| | L368A, Y407V | | | | |

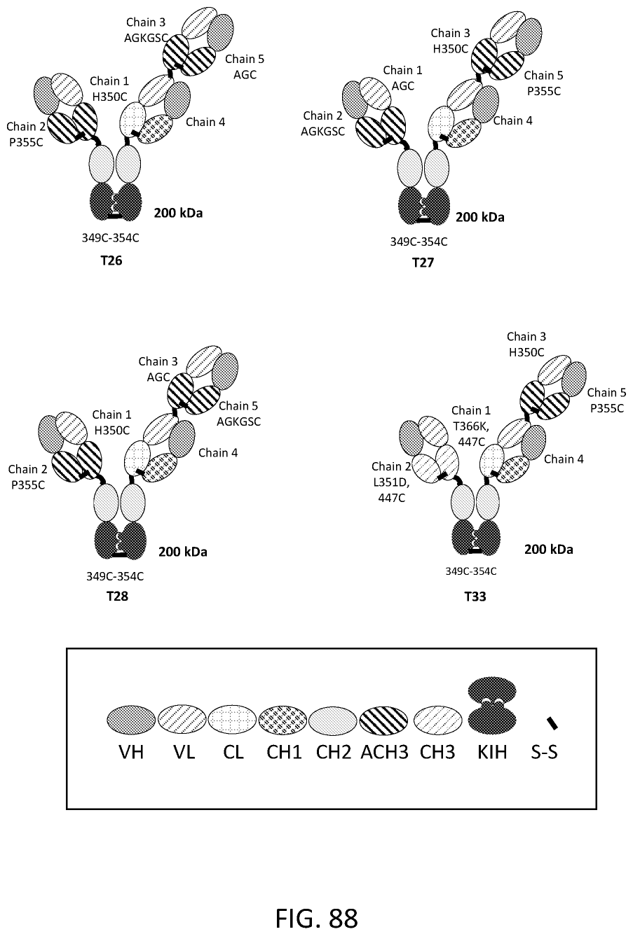

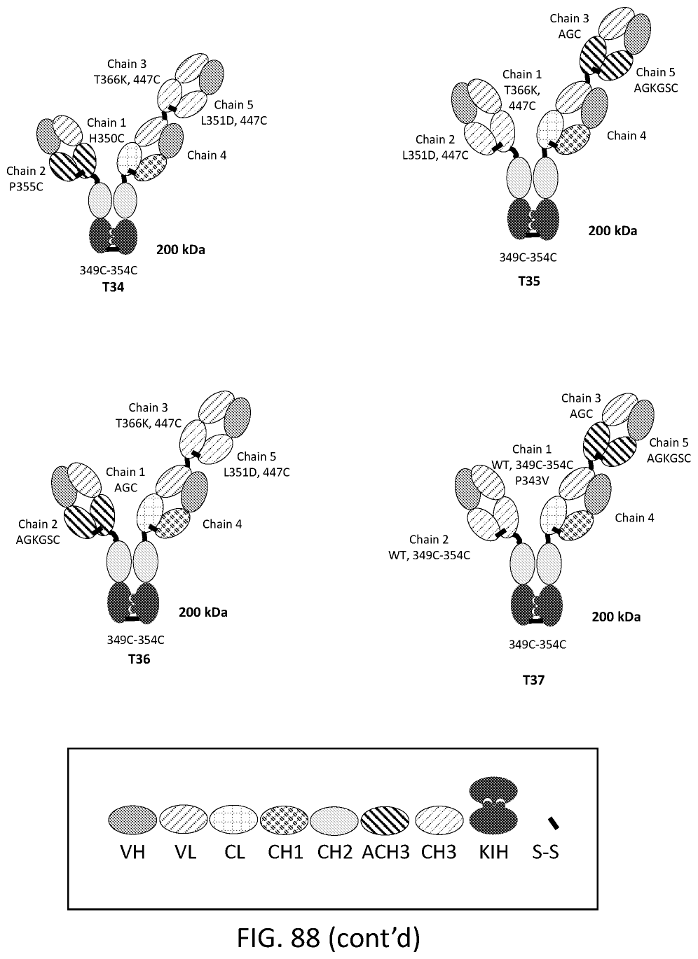

FIG. 88 depicts architectures of the various trivalent molecules (“T26,” “T27,” “T28,” “T33,” “T34,” “T35,” “T36”, “T37,” and “T38”).

Polypeptide chain amino acid sequences of the trivalent molecules T27 and T36 are included in the Sequences section.

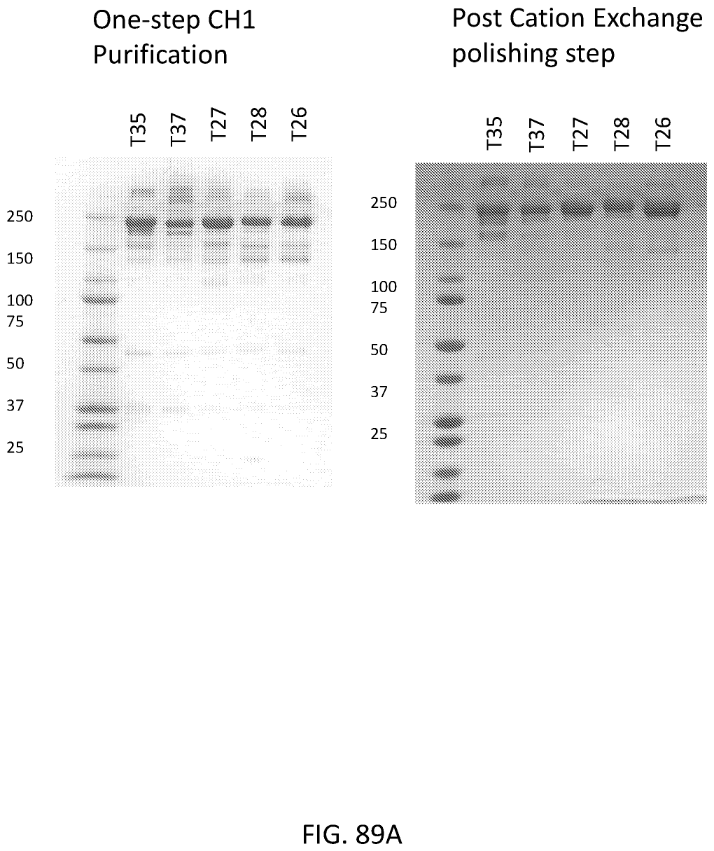

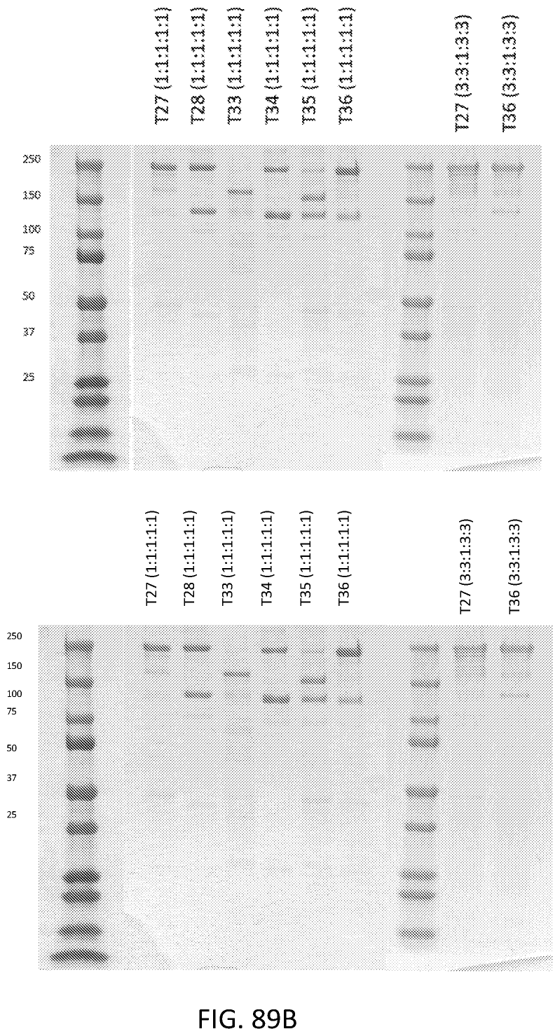

All constructs were expressed using the Expi293 system and isolated using CH1 purification as described herein. In some cases, the resulting products from CH1 purification were subjected to further purification using cation exchange polishing (IEX Chromatography), as described herein in Example 1. The resulting products were subjected to SDS-PAGE analysis. SDS-PAGE gels are shown in FIG. 89A. All constructs exhibited full assembly and overall greater yield of fully assembled binding molecules as compared to incomplete products, both with one-step CH1 purification and two-step purification.

In a further experiment, constructs T27, T28, T33, T34, T35, and T36 were expressed using varying ratios of polypeptide chains (by mass). For clarification, chain ratios are expressed as Chain 1: Chain 2: Chain 3: Chain 4: Chain 5 ratios. By way of example only, a chain ratio of (1:1:1:1:1) describes an experiment in which equal masses of Chain 1: Chain 2: Chain 3: Chain 4: Chain 5 were expressed. Constructs were expressed using the Expi293 system and then purified using one-step CH1 purification as described herein. The resulting SDS-PAGE gel is shown in FIG. 89B. T27 and T37 exhibited particularly high fidelity assembly with a 1:1:1:1:1 chain ratio, and even higher fidelity assembly with a 3:3:1:3:3 chain ratio.

### 6.15. Sequences

>Example 1, bivalent monospecific construct CHAIN 1 [SEQ ID NO: 1]
(VL)~VEIKRTPREPQVYTLPPCRDELTKNQVSLWCLVKGFYPSDIAVEWESNG
QPENNYKTTPPVLDSDGSFFLYSKLTVDKSRWQQGNVFSCSVMHEALHNHY
TQKSLSLSPGKDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIKTISKAKGQPREPQVYTLPPCRDELTKNQVSL
WCLVKGFYPSDIAVEWESNGQPENNYKTTPPVLDSDGSFFLYSKLTVDKSRW
QQGNVFSCSVMHEALHNHYTQKSLSLSPGK
>Example 1, bivalent monospecific construct CHAIN 2 [SEQ ID NO: 2]
(VH)~VTVSSASPREPQVYTLPPCRDELTKNQVSLWCLVKGFYPSDIAVEWES
NGQPENNYKTTPPVLDSDGSFFLYSKLTVDKSRWQQGNVFSCSVMHEALHN
HYTQKSLSLSPGK
>Example 1, bivalent, bispecific construct CHAIN 1 [SEQ ID NO: 3]
(VL)~VEIKRTPREPQVYTLPPCRDELTKNQVSLWCLVKGFYPSDIAVEWESNG
QPENNYKTTPPVLDSDGSFFLYSKLTVDKSRWQQGNVFSCSVMHEALHNHY
TQKSLSLSPGKDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPCRDELTK
NQVSLWCLVKGFYPSDIAVEWESNGQPENNYKTTPPVLDSDGSFFLYSKLTVDKSR
WQQGNVFSCSVMHEALHNHYTQKSLSLSPGK
VL- CH3- Hinge- CH2- CH3(knob)
> Example 1, bivalent, bispecific construct CHAIN 2 [SEQ ID NO: 4]
(VH)~VTVSSASPREPQVYTLPPCRDELTKNQVSLWCLVKGFYPSDIAVEWES
NGQPENNYKTTPPVLDSDGSFFLYSKLTVDKSRWQQGNVFSCSVMHEALHN
HYTQKSLSLSPGK
VH- CH3
>Example 1, bivalent, bispecific construct CHAIN 3_[SEQ ID NO: 5]
(VL)~VEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSP
VTKSFNRGECDKTHTCPPCPAPELLGGPSVFLFPPKPKDTLMISRTPEVTCV
VVDVSHEDPEVKFNWYVDGVEVHNAKTKPREEQYNSTYRVVSVLTVLH
QDWLNGKEYKCKVSNKALPAPIEKTISKAKGQPREPQVCTLPPSREEMTKN
QVSLSCAVKGFYPSDIAVEWESNGQPENNYKTTPPVLDSDGSFFLVSKLTVDKSRW
QQGNVFSCSVMHEALHNHYTQKSLSLSPGK
VL- CL- Hinge- CH2- CH3(hole)
>Example 1, bivalent, bispecific construct CHAIN 4 [SEQ ID NO: 6]
(VH)~VTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKK
VEPPKSC
VH- CH1
>Fc Fragment of Human IgG1 [SEQ ID NO: 7]
GQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLS
P
>BC1 chain 1 [SEQ ID NO: 8]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLS
KSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPCRDELTKNQVSLWCLV
KGFYPSDIAVEWESNGQPENNYKTTPPVLDSDGSFFLYSKLTVDKSRWQQGNVFS
CSVMHEALHNHYTQKSLSLSPGK
Domain arrangement:
A- B- Hinge- D- E
VL- CH3- Hinge- CH2- CH3(knob)
Mutations in first CH3 (Domain B):
T366K; 445K, 446S, 447C insertion
Mutations in second CH3 (Domain E):
S354C, T366W
>BC1 chain 2 [SEQ ID NO: 9]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSASPREPQVYTDPPSRDELTKNQVSLTCLVKGFYPSDI
AVEWESNGQPENNYKTTPPVLDSDGSFFLYSKLTVDKSRWQQGNVFSCSVM
HEALHNHYTQKSLSLSGEC
Domain arrangement:
F- G
VH- CH3
Mutations in CH3 (Domain G):
L351D; 445G, 446E, 447C insertion
>BC1 chain 3 [SEQ ID NO: 10]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GECDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVCTLPPSREEMTKNQVSLSCAV
KGFYPSDIAVEWESNGQPENNYKTTPPVLDSDGSFFLVSKLTVDKSRWQQGNVFS
CSVMHEALHNHYTQKSLSLSPGK
Domain arrangement:
H- I- Hinge- J- K
VL- CL- Hinge- CH2- CH3(hole)
Mutations in CH3 (domain K):
Y349C, D356E, L358M, T366S, L368A, Y407V
>BC1 chain 4 [SEQ ID NO: 11]
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKV
DKKVEPPKSC
Domain arrangement:
L- M
VH- CH1
>BC1, BA1 Domain A [SEQ ID NO: 12]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC1 Domain B [SEQ ID NO: 13]
PREPQVYTLPPSRDELTKNQVSLKCLVKGFYPSDIAVEWESNGQPENNYKTTP
PVLDSDGSFFLYSKLTVDKSRWQQGNVFSCSVMHEALHNHYTQKSLSLSKC
>BC1 Domain D [SEQ ID NO: 14]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC1, BA1 Domain E [SEQ ID NO: 15]
GQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLS
PGK
>BA1, BC1 Domain F, [SEQ ID NO: 16]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSAS
>BC1 Domain G [SEQ ID NO: 17]
PREPQVYTDPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSGEC
>BC1 Domain H [SEQ ID NO: 18]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
>BC1, BA1 Domain I [SEQ ID NO: 19]
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GEC
>BC1 Domain J [SEQ ID NO: 20]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC1, BA1 Domain K [SEQ ID NO: 21]
GQPREPQVCTLPPSREEMTKNQVSLSCAVKGFYPSDIAVEWESNGQPENNYK
TTPPVLDSDGSFFLVSKLTVDKSRWQQGNVFSCSVMHEALHNHYTQKSLSLS
PGK
>BC1 Domain L [SEQ ID NO: 22]
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSS
>BC1, BA1 Domain M [SEQ ID NO: 23]
ASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPPKSC
>BC28 chain 1 [SEQ ID NO: 24]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RTPREPQVCTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSP
GKDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGK
EYKCKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQGNVFSC
SVMHEALHNHYTQKSLSLSPGK
Domain arrangement:
A- B- Hinge- D- E
VL- CH3- Hinge- CH2- CH3(knob)
Mutations in domain B:
Y349C; 445P, 446G, 447K insertion
Mutations in domain E:
S354C, T366W
>BC28 chain 2 [SEQ ID NO: 25]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYVVGQGTLVTVSSASPREPQVYTLPPCRDELTKNQVSLTCLVKGFYPSDI
AVEWESNGQPENNYKTTPPVLDSDGSFFLYSKLTVDKSRWQQGNVFSCSVM
HEALHNHYTQKSLSLSPGK
Domain arrangement:
F- G
VH- CH3
Mutations in domain G:
S354C; 445P, 446G, 447K insertion
>BC28 domain A [SEQ ID NO: 26]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC28 domain B [SEQ ID NO: 27]
PREPQVCTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC28 domain D [SEQ ID NO: 28]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC28 domain E [SEQ ID NO: 29]
GQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLS
PGK
>BC28 domain F [SEQ ID NO: 30]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSAS
>BC28 domain G [SEQ ID NO: 31]
PREPQVYTLPPCRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC44 chain 1 [SEQ ID NO: 32]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVREPQVC
TLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGKDKTHTCPPCPAPELLG
GPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQP
REPQVYTLPPCRDELTKNQVSLWCLVKGFYPSDIAVEWESNGQPENNYKTTPPVLDSDGS
FFLYSKLTVDKSRWQQGNVFSCSVMHEALHNHYTQKSLSLSPGK
Domain arrangement:
A- B- Hinge- D- E
VL- CH3- Hinge- CH2- CH3(knob)
Mutations in domain B:
P343V; Y349C; 445P, 446G, 447K insertion
Mutations in domain E:
S354C, T366W
>BC44 Domain A [SEQ ID NO: 33]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC44 Domain B [SEQ ID NO: 34]
VREPQVCTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC44 Domain D [SEQ ID NO: 35]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC44 Domain E [SEQ ID NO: 36]
GQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLS
PGK
>BC28 bivalent chain 3 equivalent to SEQ ID NO: 10
>BC28 bivalent chain 4 equivalent to SEQ ID NO: 11
>BC28 1x2 chain 3 [SEQ ID NO: 37]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RTPREPQVCTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSP
GKGSGSGS

[symbol]

[symbol]

[symbol]

[symbol]

[symbol]

[symbol]

[symbol]

RTVAAPSVFIFPPSDEOLKSGTASVVCLLNNFYPREAKVQWKV
DNALQSGNSQESVTEQDSKDSTYSLSSTLTLSKADYEKHKVYACEVTHQG
LSSPVTKSFNRGECDKTHTCPPCPAPELLGGPSVFLFPPKPKDTLMISRTPE
VTCVVVDVSHEDPEVKFNWYVDGVEVHNAKTKPREEQYNSTYRVVSVL
TVLHQDWLNGKEYKCKVSNKALPAPIEKTISKAKGQPREPQVCTLPPSREE
MTKNQVSLSCAVKGFYPSDIAVEWESNGQPENNYKTTPPVLDSDGSFFLVSKLTVD
KSRWQQGNVFSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
Y349C; 445P, 446G, 447K insertion
Six amino acids linker insertion: GSGSGS (SEQ ID NO: 40)
Mutations in domain K:
Y349C, D356E, L358M, T3665, L368A, Y407V
>BC28 1x2 domain R [SEQ ID NO: 38]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC28 1x2 domain S [SEQ ID NO: 39]
PREPQVCTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC28 1x2 linker [SEQ ID NO: 40]
GSGGS
>BC28 1x2 domain H [SEQ ID NO: 41]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
>BC28 1x2 domain I [SEQ ID NO: 42]
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GEC
>BC28 1x2 domain J [SEQ ID NO: 43]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC28 1x2 domain K [SEQ ID NO: 44]
GQPREPQVCTLPPSREEMTKNQVSLSCAVKGFYPSDIAVEWESNGQPENNYK
TTPPVLDSDGSFFLVSKLTVDKSRWQQGNVFSCSVMHEALHNHYTQKSLSLS
PGK
>BC28-1x1x1a chain 3 [SEQ ID NO: 45]
DIQMTQSPSSLSASVGDRVTITCRASQSVSSAVAWYQQKPGKAPKLLIYSASS
LYSGVPSRFSGSRSGTDFTLTISSLQPEDFATYYCQQRDSYLWTFGQGTKVEIK
RTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLS
KSCGSGSGS

[symbol]

[symbol]

[symbol]

[symbol]

[symbol]

[symbol]

[symbol]

RTVAAPSVFIFPPSDEOLKSGTASVVCLLNNFYPREAKVQWK
VDNALQSGNSQESVTEQDSKDSTYSLSSTLTLSKADYEKHKVYACEVTHQ
GLSSPVTKSFNRGECDKTHTCPPCPAPELLGGPSVFLFPPKPKDTLMISRTP
EVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRE
EMTKNQVSLSCAVKGFYPSDIAVEWESNGQPENNYKTTPPVLDSDGSFFLVSKLTV
DKSRWQQGNVFSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
T366K; 445K, 446S, 447C insertion
Six amino acids linker insertion: GSGSGS (SEQ ID NO: 40)
Mutations in domain K:
Y349C, D356E, L358M, T3665, L368A, Y407V
>BC28-1x1x1a domain R [SEQ ID NO: 46]
DIQMTQSPSSLSASVGDRVTITCRASQSVSSAVAWYQQKPGKAPKLLIYSASS
LYSGVPSRFSGSRSGTDFTLTISSLQPEDFATYYCQQRDSYLWTFGQGTKVEIK
RT
>BC28-1x1x1a domain S [SEQ ID NO: 47]
PREPQVYTLPPSRDELTKNQVSLKCLVKGFYPSDIAVEWESNGQPENNYKTTP
PVLDSDGSFFLYSKLTVDKSRWQQGNVFSCSVMHEALHNHYTQKSLSLSKSC
>BC28-1x1x1a linker [SEQ ID NO: 48]
GSGSGS
>BC28-1x1x1a domain H [SEQ ID NO: 49]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
>BC28-1x1x1a domain I [SEQ ID NO: 50]
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GEC
>BC28-1x1x1a domain J [SEQ ID NO: 51]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC28-1x1x1a domain K [SEQ ID NO: 52]
GQPREPQVCTLPPSREEMTKNQVSLSCAVKGFYPSDIAVEWESNGQPENNYK
TTPPVLDSDGSFFLVSKLTVDKSRWQQGNVFSCSVMHEALHNHYTQKSLSLS
PGK
>hCTLA4-4.chain 2 [SEQ ID NO: 53]
EVQLVESGGGLVQPGGSLRLSCAASGFTFSTYYIHWVRQAPGKGLEWVAVIY
PYTGFTYYADSVKGRFTISADTSKNTAYLQMNSLRAEDTAVYYCARGEYTV
LDYWGQGTLVTVSSASPREPQVYTDPPSRDELTKNQVSLTCLVKGFYPSDIA
VEWESNGQPENNYKTTPPVLDSDGSFFLYSKLTVDKSRWQQGNVFSCSVMH
EALHNHYTQKSLSLSGEC
Domain arrangement
F- G
VH- CH3
Mutations in domain G
L351D, 445G, 446E, 447C insertion
>hCTLA4-4 domain F [SEQ ID NO: 54]
EVQLVESGGGLVQPGGSLRLSCAASGFTFSTYYIHWVRQAPGKGLEWVAVIY
PYTGFTYYADSVKGRFTISADTSKNTAYLQMNSLRAEDTAVYYCARGEYTV
LDYWGQGTLVTVSSAS
>hCTLA4-4 domain G [SEQ ID NO: 55]
PREPQVYTDPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSGEC
Other Sequences:
>Hinge: DKTHTCPPCP [SEQ ID NO: 56]
>BC1-Polypeptide 1 Domain Junction: IKRTPREP [SEQ ID NO: 57]
>BC15-Polypeptide 1 Domain Junction: IKRTVREP [SEQ ID NO: 58]
>BC16-Polypeptide 1 Domain Junction: IKRTREP [SEQ ID NO: 59]
>BC17-Polypeptide 1 Domain Junction: IKRTVPREP [SEQ ID NO: 60]
>BC26-Polypeptide 1 Domain Junction: IKRTVAEP [SEQ ID NO: 61]
>BC27-Polypeptide 1 Domain Junction: IKRTVAPREP [SEQ ID NO: 62]
>BC1-Polypeptide 2 Domain Junction: SSASPREP [SEQ ID NO: 63]
>BC13-Polypeptide 2 Domain Junction: SSASTREP [SEQ ID NO: 64]
>BC14-Polypeptide 2 Domain Junction: SSASTPREP [SEQ ID NO: 65]
>BC24-Polypeptide 2 Domain Junction: SSASTKGEP [SEQ ID NO: 66]
>BC25-Polypeptide 2 Domain Junction: SSASTKGREP [SEQ ID NO: 67]
>Phage display heavy chain [SEQ ID NO: 74]
EVQLVESGGGLVQPGGSLRLSCAASGFTFxxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSR
WQQGNVFSCSVMHEALHNHYTQKSLSLSPGK
>Phage display light chain [SEQ ID NO: 75]
DIQMTQSPSSLSASVGDRVTITC [symbol] VAWYQQKPGKAPKLLIY [symbol] [symbol]
GVPSRFSGSRSGTDFTLTISSLQPEDFATYYC [symbol] xxxxxx [symbol] GQGTKVEIKRT
VAAPSVFIFPPSDSQLKSGTASVVCLLNNFYPREAKVQWKVDNALQSGNSQE
SVTEQDSKDSTYSLSSTLTLSKADYEKHKVYACEVTHQGLSSPVTKSFNRGEC
>B-Body Domain A/H Scaffold [SEQ ID NO: 76]
DIQMTQSPSSLSASVGDRVTITC [symbol] VAWYQQKPGKAPKLLIY [symbol] [symbol]
GVPSRFSGSRSGTDFTLTISSLQPEDFATYYC [symbol] xxxxxx [symbol] GQGTKVEIKRT
>B-Body Domain F/L Scaffold [SEQ ID NO: 77]
EVQLVESGGGLVQPGGSLRLSCAASGFTFxxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSAS
>BC1 Chain 1 Scaffold [SEQ ID NO: 78]
DIQMTQSPSSLSASVGDRVTITC [symbol] VAWYQQKPGKAPKLLIY [symbol] GV
PSRFSGSRSGTDFTLTISSLQPEDFATYYC [symbol] xxxxxx [symbol] GQGTKVEIKRTPREPQVYTL
PPSRDELTKNQVSLKCLVKGFYPSDIAVEWESNGQPENNYKTTPPVLDSDGSFFLYS
KLTVDKSRWQQGNVFSCSVMHEALHNHYTQKSLSLSKSCDKTHTCPPCPAPELLGG
PSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPRE
PQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGK
“x” represents CDR amino acids that were varied to create the library, and bold italic
represents the CDR sequences that were constant
Domain arrangement:
A- B- Hinge- D- E
VL- CH3- Hinge- CH2- CH3(knob)
Mutations in first CH3 (Domain B):
T366K; 445K, 446S, 447C insertion
Mutations in second CH3 (Domain E):
S354C, T366W
>BC1 Chain 2 Scaffold [SEQ ID NO: 79]
EVQLVESGGGLVQPGGSLRLSCAASGFTFxxxx [symbol] WVRQAPGKGLEWVAxxxx
xxxxxxx [symbol] RFTSADTSKNTAYLQMNSLRAEDTAVYYCARxxxxxxxxxx
xxx [symbol] WGQGTLVTVSSASPREPQVYTDPPSRDELTKNQVSLTCLVKGFYPSDI
AVEWESNGQPENNYKTTPPVLDSDGSFFLYSKLTVDKSRWQQGNVFSCSVM
HEALHNHYTQKSLSLSGEC
“x” represents CDR amino acids that were varied to create the library, and bold italic
represents the CDR sequences that were constant
Domain arrangement:
F- G
VH- CH3
Mutations in CH3 (Domain G):
L351D; 445G, 446E, 447C insertion
>BC1 Chain 3 Scaffold [SEQ ID NO: 80]
DIQMTQSPSSLSASVGDRVTITC [symbol] VAWYQQKPGKAPKLLIY [symbol] [symbol]
GVPSRFSGSRSGTDFTLTISSLQPEDFATYYC [symbol] xxxxxx [symbol] GQGTKVEIKRT
VAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPAPIEKTISKAKGQPREPQVCTLPPSREEMTKNQVSLSCAVKG
FYPSDIAVEWESNGQPENNYKTTPPVLDSDGSFFLVSKLTVDKSRWQQGNVFSCS
VMHEALHNHYTQKSLSLSPGK
“x” represents CDR amino acids that were varied to create the library, and bold italic
represents the CDR sequences that were constant
Domain arrangement:
H- I- Hinge- J- K
VL- CL- Hinge- CH2- CH3(hole)
Mutations in CH3 (domain K):
Y349C, D356E, L358M, T3665, L368A, Y407V
>BC1 Chain 4 Scaffold [SEQ ID NO: 81]
EVQLVESGGGLVQPGGSLRLSCAASGFTFxxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPS
NTKVDKKVEPPKSC
“x” represents CDR amino acids that were varied to create the library, and bold italic
represents the CDR sequences that were constant
Domain arrangement:
L- M
VH- CH1
>BC1 Chain 3 1(A)x2(B-A) SP34-89 Scaffold [SEQ ID NO: 82]
DIQMTQSPSSLSASVGDRVTITC [symbol] VAWYQQKPGKAPKLLIY [symbol] GV
PSRFSGSRSGTDFTLTISSLQPEDFATYYC [symbol] xxxxxx [symbol] GQGTKVEIKRTPREPQVYTL
PPSRDELTKNQVSLKCLVKGFYPSDIAVEWESNGQPENNYKTTPPVLDSDGSFFLYS
KLTVDKSRWQQGNVFSCSVMHEALHNHYTQKSLSLSKSCTASSGGSSSGQAVVTQEP
SLTVSPGGTVTLTCRSSTGAVTTSNYANWVQQKPGQAPRGLIGGTNKRAPWTPARF
SGSLLGGKAALTITGAQAEDEADYYCALWYSNLWVFGGGTKLTVLGRTVAAPSVFI
FPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECDKTHTCPPCPAP
EAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALKAPIEKTISKA
KGQPREPQVCTLPPSREEMTKNQVSLSCAVKGFYPSDIAVEWESNGQPENNYKTTPPVLD
SDGSFFLVSKLTVDKSRWQQGNVFSCSVMHEALHNHYTQKSLSLSPGK
“x” represents CDR amino acids that were varied to create the library, and bold italic
represents the CDR sequences that were constant
Domain arrangement:
R- S- linker- H- I- Hinge- J- K
VL- CH3- linker- SP34- CL- Hinge- CH2- CH3(hole)
Mutations in domain S:
T366K; 445K, 446S, 447C insertion
Ten amino acids linker insertion: TASSGGSSSG (SEQ ID NO: 83)
Mutations in Domain J:
L234A, L235A, and P329K
Mutations in domain K:
Y349C, D356E, L358M, T3665, L368A, Y407V
>BC1 Chain 3 1(A)x2(B-A) SP34-89 S-H Junction [SEQ ID NO: 83]
TASSGGSSSG
>Library Parent Heavy Chain [SEQ ID NO: 84]
EVQLVESGGGLVQPGGSLRLSCAASGFTFxxxx [symbol] WVRQAPGKGLEWVAxxxxxxxxxx
x [symbol] RFTISADTSKNTAYLQMNSLRAEDTAVYYCARxxxxxxxxxxxxx [symbol] WGQG
TLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKP
(SEQ ID NO: 96)
AGKC
(SEQ ID NO: 97)
PGKC
(SEQ ID NO: 98)
AGKGC
(SEQ ID NO: 99)
AGKGSC
>Human IgA CH3 sequence [SEQ ID NO: 184]
TFRPEVHLLPPPSEELALNELVTLTCLARGFSPKDVLVRWLQGSQELPREKYLTWAS
RQEPSQGTTTFAVTSILRVAAEDWKKGDTFSCMVGHEALPLAFTQKTIDRL
>BA1.chain 1 [SEQ ID NO: 210]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGECDKTHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQP
REPQVYTLPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGK
>BA1.chain 2 [SEQ ID NO: 211]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA(a11).chain 3 [SEQ ID NO: 212]
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPPCPAPELLG
GPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVLDSDGSFF
LVSKLTVDKSRWQQGNVFSCSVMHEALHNHYTQKSLSLSPGK
>BA(all) chain 4 [SEQ ID NO: 213]
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPPKSC
>BA2 [SEQ ID NO: 214]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA3 [SEQ ID NO: 215]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA4 [SEQ ID NO: 216]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA5 [SEQ ID NO: 217]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA9 [SEQ ID NO: 218]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA10 [SEQ ID NO: 219]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA11 [SEQ ID NO: 220]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA12 [SEQ ID NO: 221]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA13 [SEQ ID NO: 222]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA14 [SEQ ID NO: 223]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA15 [SEQ ID NO: 224]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA16 [SEQ ID NO: 225]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA17 [SEQ ID NO: 226]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA18 [SEQ ID NO: 227]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA19 [SEQ ID NO: 228]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA20 [SEQ ID NO: 229]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA21 [SEQ ID NO: 230]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA22 [SEQ ID NO: 231]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA23 [SEQ ID NO: 232]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA24 [SEQ ID NO: 233]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA25 [SEQ ID NO: 234]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA26 [SEQ ID NO: 235]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA27 [SEQ ID NO: 236]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA28 [SEQ ID NO: 237]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA29 [SEQ ID NO: 238]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA30 [SEQ ID NO: 239]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA31 [SEQ ID NO: 240]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA32 [SEQ ID NO: 241]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA33 [SEQ ID NO: 242]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA34 [SEQ ID NO: 243]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA35 [SEQ ID NO: 244]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA36 [SEQ ID NO: 245]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA37 [SEQ ID NO: 246]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA38 [SEQ ID NO: 247]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA39 [SEQ ID NO: 248]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA40 [SEQ ID NO: 249]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA41 [SEQ ID NO: 250]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA42 [SEQ ID NO: 251]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA43 [SEQ ID NO: 252]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA44 [SEQ ID NO: 253]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA2 [SEQ ID NO: 254]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA3 [SEQ ID NO: 255]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA4 [SEQ ID NO: 256]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA5 [SEQ ID NO: 257]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA9 [SEQ ID NO: 258]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA10 [SEQ ID NO: 259]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA11 [SEQ ID NO: 260]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA12 [SEQ ID NO: 261]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA13 [SEQ ID NO: 262]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA14 [SEQ ID NO: 263]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA15 [SEQ ID NO: 264]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA16 [SEQ ID NO: 265]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA17 [SEQ ID NO: 266]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA18 [SEQ ID NO: 267]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA19 [SEQ ID NO: 268]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA20 [SEQ ID NO: 269]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA21 [SEQ ID NO: 270]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA22 [SEQ ID NO: 271]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA23 [SEQ ID NO: 272]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA24 [SEQ ID NO: 273]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA25 [SEQ ID NO: 274]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA26 [SEQ ID NO: 275]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA27 [SEQ ID NO: 276]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA28 [SEQ ID NO: 277]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA29 [SEQ ID NO: 278]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA30 [SEQ ID NO: 279]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA31 [SEQ ID NO: 280]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA32 [SEQ ID NO: 281]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA33 [SEQ ID NO: 282]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA34 [SEQ ID NO: 283]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA35 [SEQ ID NO: 284]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA36 [SEQ ID NO: 285]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA37 [SEQ ID NO: 286]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA38 [SEQ ID NO: 287]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA39 [SEQ ID NO: 288]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA40 [SEQ ID NO: 289]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA41 [SEQ ID NO: 290]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA42 [SEQ ID NO: 291]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA43 [SEQ ID NO: 292]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA44 [SEQ ID NO: 293]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>Human IgA CH3 sequence [SEQ ID NO: 294]
TFRPEVHLLPPPSEELALNELVTLTCLARGFSPKDVLVRWLQGSQELPREKYLTWAS
RQEPSQGTTTFAVTSILRVAAEDWKKGDTFSCMVGHEALPLAFTQKTIDRL
>T27, T36 chain 1 [SEQ ID NO: 295]
ELVMTQSPSSLTVTAGEKVTMSCKSSQSLLNSGNQKNYLTWYQQKPGQPPKLLIYW
ASTRESGVPDRFTGSGSGTDFTLTISSVQAEDLAVYYCQNDYSYPLTFGAGTKLEIKR
TTFRPEVHLLPPPSEELALNELVTLTCLARGFSPKDVLVRWLQGSQELPREKYLTWA
SRQEPSQGTTTFAVTSILRVAAEDWKKGDTFSCMVGHEALPLAFTQKTIDRLAGCDK
THTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALK
APIEKTISKAKGQPREPQVCTLPPSREEMTKNQVSLSCAVKGFYPSDIAVEWESNGQ
PENNYKTTPPVLDSDGSFFLVSKLTVDKSRWQQGNVFSCSVMHEALHNHYTQKSLS
LSPGK
Domain arrangement
A B Hinge D E
VL CH3 Hinge CH2 CH3
Mutations in first CH3 (Domain B)
Substitution of IgG-CH3 with IgA-CH3, C-terminus AGC linker addition
Mutations in CH2 (Domain D)
L234A, L235A, P329K
Mutations in second CH3 (Domain E)
Y349C, D356E, L358M, T3665, L368A, Y407V
>T27, T36 chain 2 [SEQ ID NO: 296]
EVQLLEQSGAELVRPGTSVKISCKASGYAFTNYWLGWVKQRPGHGLEWIGDIFPGS
GNIHYNEKFKGKATLTADKSSSTAYMQLSSLTFEDSAVYFCARLRNWDEPMDYWG
QGTTVTVSSASTFRPEVHLLPPPSEELALNELVTLTCLARGFSPKDVLVRWLQGSQEL
PREKYLTWASRQEPSQGTTTFAVTSILRVAAEDWKKGDTFSCMVGHEALPLAFTQK
TIDRLAGKGSC
Domain arrangement
F G
VH CH3
Mutations in CH3 (Domain G)
Substitution of IgG-CH3 with IgA-CH3, C-terminus AGKGSC linker (SEQ
ID NO: 99) addition
>T27 chain 3 [SEQ ID NO: 297]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVC
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLTASSGGSSSGQAVV
TQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLG
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGECDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALKAPIEKTISKAK
GQPREPQVYTLPPCRDELTKNQVSLWCLVKGFYPSDIAVEWESNGQPENNYKT
TPPVLDSDGSFFLYSKLTVDKSRWQQGNVFSCSVMHEALHNHYTQKSLSLSPGK
Domain arrangement
N O Hinge H I Hinge 2 J K
VL CH3 Hinge VL CL Hinge 2 CH2 CH3
Mutations in first CH3 (Domain O)
Substitution of IgG-CH3 with IgA-CH3, H350C
Mutations in CH2 (Domain J)
L234A, L235A, P329K
Mutations in second CH3 (Domain K)
S354C, T366W
>T27 chain 4 [SEQ ID NO: 298]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement
L M
VH CH1
>T27 chain 5 [SEQ ID NO: 299]
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSTFRPEVHLLPPCSEELALNELVTLTCLARGFSPKDVLVRWLQGSQELP
REKYLTWASRQEPSQGTTTFAVTSILRVAAEDWKKGDTFSCMVGHEALPLAFTQKTI
DRL
Domain arrangement
P Q
VH CH3
Mutations in CH3 (Domain Q)
Substitution of IgG-CH3 with IgA-CH3, P355C
>T36 chain 3 [SEQ ID NO: 300]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCTASSGGSSSGQAVVTQ
EPSLTVSPGGTVTLTCRSSTGAVTTSNYANWVQQKPGQAPRGLIGGTNKRAPW
TPARFSGSLLGGKAALTITGAQAEDEADYYCALWYSNLWVFGGGTKLTVLGRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPC
PAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALKAPIEKTISKAKG
QPREPQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSLSPGK
Domain arrangement
N O Hinge H I Hinge 2 J K
VL CH3 Hinge VL CL Hinge 2 CH2 CH3
Mutations in first CH3 (Domain O)
T366K, 445K, 446S, 447C tripeptide insertion
Mutations in CH2 (Domain J)
L234A, L235A, P329K
Mutations in second CH3 (Domain K)
S354C, T366W
>T36 chain 4 [SEQ ID NO: 301]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement
L M
VH CH1
>T36 chain 5 [SEQ ID NO: 302]
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSPREPQVYTDPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSG
EC

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

The application incorporates by reference International Application PCT/US2019/027991, which is incorporated by reference in its entirety. In the event of inconsistent usages between this document and those documents so incorporated by reference, the usage in the incorporated reference(s) should be considered supplementary to that of this document; for irreconcilable inconsistencies, the usage in this document controls.

While various specific embodiments have been illustrated and described, the above specification is not restrictive. It will be appreciated that various changes can be made without departing from the spirit and scope of the invention(s). Many variations will become apparent to those skilled in the art upon review of this specification.

1. A multispecific Treg-binding molecule, comprising
a first antigen binding site (ABS) specific for a first Treg cell surface antigen; and
a second antigen binding site (ABS) specific for a second Treg cell surface antigen;
wherein the first ABS binds the first Treg cell surface antigen with a Kd that is greater than 10 nM,
wherein the second ABS binds the second Treg cell surface antigen with a Kd that is greater than 10 nm, wherein the second Treg cell surface antigen is not the first Treg cell surface antigen, and wherein the multispecific Treg-binding molecule binds to a target Treg with a Kd that is less than 100 nM.

2. The multispecific Treg-binding molecule of claim 1, wherein the target Treg is a tumor-associated Treg.

3. The multispecific Treg-binding molecule of claim 1, wherein the target Treg expresses the first and second Treg cell surface antigens.

4. The multispecific Treg-binding molecule of claim 3, wherein the target Treg overexpresses the first and second Treg cell surface antigens as compared to a non-target cell.

5. The multispecific Treg-binding molecule of claim 1, wherein the first and second Treg cell surface antigens are CTLA4 and CD25.

6. The multispecific Treg-binding molecule of claim 1, wherein the first ABS binds to the first Treg cell surface antigen with a Kd that is greater than 100 nM, the second ABS binds to the second Treg cell surface antigen with a Kd that is greater than 100 nM, and the multispecific Treg-binding molecule binds to a target Treg with a Kd that is less than 10 nM.

7. The multispecific Treg-binding molecule of claim 1, wherein the multispecific Treg-binding molecule comprises
a first, second, third, and fourth polypeptide chain, wherein:
(a) the first polypeptide chain comprises a domain A, a domain B, a domain D, and a domain E,
wherein the domains are arranged, from N-terminus to C-terminus, in a A-B-D-E orientation, and
domain A has a VL amino acid sequence, domain B has a CH3 amino acid sequence, domain D has a CH2 amino acid sequence, and domain E has a constant region domain amino acid sequence;
(b) the second polypeptide chain comprises a domain F and a domain G,
wherein the domains are arranged, from N-terminus to C-terminus, in a F-G orientation, and
wherein domain F has a VH amino acid sequence and domain G has a CH3 amino acid sequence;
(c) the third polypeptide chain comprises a domain H, a domain I, a domain J, and a domain K,
wherein the domains are arranged, from N-terminus to C-terminus, in a H-I-J-K orientation, and
wherein domain H has a variable region domain amino acid sequence, domain I has a constant region domain amino acid sequence, domain J has a CH2 amino acid sequence, and K has a constant region domain amino acid sequence;
(d) the fourth polypeptide chain comprises a domain L and a domain M,
wherein the domains are arranged, from N-terminus to C-terminus, in a L-M orientation, and
wherein domain L has a variable region domain amino acid sequence and domain M has a constant region domain amino acid sequence;
(e) the first and the second polypeptides are associated through an interaction between the A and the F domains and an interaction between the B and the G domains;
(f) the third and the fourth polypeptides are associated through an interaction between the H and the L domains and an interaction between the I and the M domains; and
(g) the first and the third polypeptides are associated through an interaction between the D and the J domains and an interaction between the E and the K domains to form the multispecific Treg-binding molecule,
wherein the interaction between the A domain and the F domain form the first ABS, and wherein the interaction between the H domain and the L domain form the second ABS.

8. The multispecific Treg-binding molecule of claim 1, wherein the first and second Treg cell surface antigens are each independently selected from CTLA4, CD25, OX40, GITR, TNFRII, NRP1, CD30, CD27, ICOS, TIGIT, 4-1BB, LAG-3, and PDL-2.

9. The multispecific Treg-binding molecule of claim 8, wherein the first and second Treg cell surface antigens are each independently selected from CTLA4, CD25, OX40, and NRP1.

10. The multispecific Treg-binding molecule of claim 1, wherein the first Treg cell surface antigen is CTLA4 and the second Treg cell surface antigen is CD25.

11. The multispecific Treg-binding molecule of claim 1, wherein the first Treg cell surface antigen is CTLA4 and the second Treg cell surface antigen is OX40.

12. The multispecific Treg-binding molecule of claim 1, wherein the first ABS comprises a first VL CDR1 amino acid sequence, a first VL CDR2 amino acid sequence, and a first VL CDR3 amino acid sequence of a light chain variable region (VL), wherein the first VL CDR3 sequences are selected from the VL CDR3 sequences from Table 20.

13. The multispecific Treg-binding molecule of claim 12, wherein the first ABS further comprises a first VH CDR1 amino acid sequence, a first VH CDR2 amino acid sequence, and a first VH CDR3 amino acid sequence of a heavy chain variable region (VH), wherein the first VH CDR1, CDR2, and CDR3 sequences are selected from the VH CDR1, CDR2, and CDR3 sequences from Table 20.

14. The multispecific Treg-binding molecule of claim 13, wherein the second ABS comprises a second VL CDR1 amino acid sequence, a second VL CDR2 amino acid sequence, and a second VL CDR3 amino acid sequence of a light chain variable region (VL), wherein the second VL CDR1, CDR2, and CDR3 sequences are selected from Table 20.

15. The multispecific Treg-binding molecule of claim 14, wherein the second ABS further comprises a second VH CDR1 amino acid sequence, a second VH CDR2 amino acid sequence, and a second VH CDR3 amino acid sequence of a heavy chain variable region (VH), wherein the second VH CDR1, CDR2, and CDR3 sequences are selected from Table 20.

16. The multispecific Treg-binding molecule of claim 1, wherein the multispecific Treg-binding molecule is conjugated to a therapeutic agent.

17. The multispecific Treg-binding molecule of claim 1, further comprising a third ABS specific for a cytotoxic lymphocyte.

18. The multispecific Treg-binding molecule of claim 17, wherein the cytotoxic lymphocyte is a natural killer (NK) cell.

19. The multispecific Treg-binding molecule of claim 1, wherein the multispecific Treg-binding molecule binds to the target Treg with 10-fold higher avidity than a T killer cell, T helper cell, memory T cell, or peripheral non-tumor associated Treg.

20. The multispecific Treg-binding molecule of claim 1, wherein the target Treg is a primate Treg.

21. The multispecific Treg-binding molecule of claim 20, wherein the primate Treg is a human Treg or cyno Treg.

22. A pharmaceutical composition comprising an effective amount of a multispecific Treg-binding molecule of claim 1 and a pharmaceutically acceptable excipient.

23. A method of treating a proliferative disease in a human subject, comprising administering to the human subject a pharmaceutical composition of claim 22.

24. The method of claim 23, wherein the proliferative disease is cancer.

25. A method of suppressing activity or number of tumor-associated Tregs in a subject, comprising administering to the subject a pharmaceutical composition of claim 22.

26. A method of screening a set of candidate multispecific Treg-binding molecules for a multispecific Treg-binding molecule that selectively binds a tumor-associated Treg, comprising
assessing binding avidity of a candidate to
a first population of cells comprising the first Treg cell surface antigen but not the second Treg cell surface antigen,
a second population of cells comprising the second Treg cell surface antigen but not the first Treg cell surface antigen, and
a third population of cells comprising the first and second Treg cell surface antigens; and
selecting the candidate as a Treg-binding molecule if the binding avidity to the third population of cells is at least two-fold greater than avidity to the first or second cell.

27. The method of claim 26, comprising selecting the candidate as a Treg-binding molecule if the binding avidity to the third population of cells is at least ten-fold greater than avidity to the first or second population of cells.

28. The method of claim 26, wherein the assessing comprises contacting the first, second, and third populations of cells with a dilution series of library member concentrations.

29. The method of claim 28, wherein the dilution series comprises library member concentrations ranging from 1-2000 nM.

30. The method of claim 26, comprising selecting the library member as a Treg-binding molecule if the library member exhibits less than 15% binding to the first and second populations of cells at 100 nM, but more than 50% binding to the third population of cells at 100 nM.

31. The method of claim 26, comprising selecting the library member as a Treg-binding molecule if the library member exhibits less than 10% binding to the first and second populations of cells at 500 nM, but more than 90% binding to the third population of cells at 500 nM.

32. An isolated polynucleotide encoding an amino acid sequence that is at least 97% identical to any one of the sequences in Tables 16, 21, or 22.

33. A vector comprising the isolated polynucleotide of claim 32.

34. A host cell comprising the vector of claim 33.


SEQUENCE LISTING (304 sequences)

SEQ ID NO: 1 - protein, 338 aa, Artificial Sequence
  Description of Artificial Sequence: Synthetic polypeptide
VEIKRTPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 112 aa, Artificial Sequence
  Description of Artificial Sequence: Synthetic polypeptide
VTVSSASPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 338 aa, Artificial Sequence; identical to SEQ ID NO: 1
  Description of Artificial Sequence: Synthetic polypeptide

SEQ ID NO: 4 - protein, 112 aa, Artificial Sequence; identical to SEQ ID NO: 2
  Description of Artificial Sequence: Synthetic polypeptide

SEQ ID NO: 5 - protein, 338 aa, Artificial Sequence
  Description of Artificial Sequence: Synthetic polypeptide
VEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 109 aa, Artificial Sequence
  Description of Artificial Sequence: Synthetic polypeptide
VTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 7 - protein, 105 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 441 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 227 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 10 - protein, 441 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 217 aa, Artificial Sequence
  Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 12 - protein, 109 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 13 - protein, 105 aa, Artificial Sequence
  Description of Artificial Sequence: Synthetic polypeptide
PREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSC

SEQ ID NO: 14 - protein, 110 aa, Artificial Sequence
  Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 107 aa, Artificial Sequence
  Description of Artificial Sequence: Synthetic polypeptide
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 122 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSAS

SEQ ID NO: 17 - protein, 105 aa, Artificial Sequence
  Description of Artificial Sequence: Synthetic polypeptide
PREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 18 - protein, 107 aa, Artificial Sequence [sequence omitted]

SEQ ID NO: 19 - protein, 107 aa, Artificial Sequence [sequence omitted]

SEQ ID NO: 20 - protein, 110 aa, Artificial Sequence; identical to SEQ ID NO: 14
  Description of Artificial Sequence: Synthetic polypeptide

SEQ ID NO: 21 - protein, 107 aa, Artificial Sequence
  Description of Artificial Sequence: Synthetic polypeptide
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 113 aa, Artificial Sequence [sequence omitted]

SEQ ID NO: 23 - protein, 104 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 24 - protein, 441 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 227 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASPREPQVYTLPPCRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 109 aa, Artificial Sequence; identical to SEQ ID NO: 12
  Description of Artificial Sequence: Synthetic polypeptide

SEQ ID NO: 27 - protein, 105 aa, Artificial Sequence
  Description of Artificial Sequence: Synthetic polypeptide
PREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 110 aa, Artificial Sequence; identical to SEQ ID NO: 14
  Description of Artificial Sequence: Synthetic polypeptide

SEQ ID NO: 29 - protein, 107 aa, Artificial Sequence; identical to SEQ ID NO: 15
  Description of Artificial Sequence: Synthetic polypeptide

SEQ ID NO: 30 - protein, 122 aa, Artificial Sequence; identical to SEQ ID NO: 16
  Description of Artificial Sequence: Synthetic polypeptide

SEQ ID NO: 31 - protein, 105 aa, Artificial Sequence
  Description of Artificial Sequence: Synthetic polypeptide
PREPQVYTLPPCRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 441 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 109 aa, Artificial Sequence; identical to SEQ ID NO: 12
  Description of Artificial Sequence: Synthetic polypeptide

SEQ ID NO: 34 - protein, 105 aa, Artificial Sequence
  Description of Artificial Sequence: Synthetic polypeptide
VREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 110 aa, Artificial Sequence; identical to SEQ ID NO: 14
  Description of Artificial Sequence: Synthetic polypeptide

SEQ ID NO: 36 - protein, 107 aa, Artificial Sequence; identical to SEQ ID NO: 15
  Description of Artificial Sequence: Synthetic polypeptide

SEQ ID NO: 37 - protein, 661 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 109 aa, Artificial Sequence; identical to SEQ ID NO: 12
  Description of Artificial Sequence: Synthetic polypeptide

SEQ ID NO: 39 - protein, 105 aa, Artificial Sequence; identical to SEQ ID NO: 27
  Description of Artificial Sequence: Synthetic polypeptide

SEQ ID NO: 40 - protein, 6 aa, Artificial Sequence
  Description of Artificial Sequence: Synthetic peptide
GSGSGS

SEQ ID NO: 41 - protein, 107 aa, Artificial Sequence [sequence omitted]

SEQ ID NO: 42 - protein, 107 aa, Artificial Sequence [sequence omitted]

SEQ ID NO: 43 - protein, 110 aa, Artificial Sequence; identical to SEQ ID NO: 14
  Description of Artificial Sequence: Synthetic polypeptide

SEQ ID NO: 44 - protein, 107 aa, Artificial Sequence; identical to SEQ ID NO: 21
  Description of Artificial Sequence: Synthetic polypeptide

SEQ ID NO: 45 - protein, 661 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRDSYLWTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCGSGSGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 109 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRDSYLWTFGQGTKVEIKRT

SEQ ID NO: 47 - protein, 105 aa, Artificial Sequence; identical to SEQ ID NO: 13
  Description of Artificial Sequence: Synthetic polypeptide

SEQ ID NO: 48 - protein, 6 aa, Artificial Sequence
  Description of Artificial Sequence: Synthetic peptide
GSGSGS

SEQ ID NO: 49 - protein, 107 aa, Artificial Sequence [sequence omitted]

SEQ ID NO: 50 - protein, 107 aa, Artificial Sequence [sequence omitted]

SEQ ID NO: 51 - protein, 110 aa, Artificial Sequence; identical to SEQ ID NO: 14
  Description of Artificial Sequence: Synthetic polypeptide

SEQ ID NO: 52 - protein, 107 aa, Artificial Sequence; identical to SEQ ID NO: 21
  Description of Artificial Sequence: Synthetic polypeptide

SEQ ID NO: 53 - protein, 224 aa, Artificial Sequence
  Description of Artificial Sequence: Synthetic polypeptide
EVQLVESGGGLVQPGGSLRLSCAASGFTFSTYYIHWVRQAPGKGLEWVAVIYPYTGFTYYADSVKGRFTISADTSKNTAYLQMNSLRAEDTAVYYCARGEYTVLDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 54 - protein, 119 aa, Artificial Sequence
  Description of Artificial Sequence: Synthetic polypeptide
EVQLVESGGGLVQPGGSLRLSCAASGFTFSTYYIHWVRQAPGKGLEWVAVIYPYTGFTYYADSVKGRFTISADTSKNTAYLQMNSLRAEDTAVYYCARGEYTVLDYWGQGTLVTVSSAS

SEQ ID NO: 55 - protein, 105 aa, Artificial Sequence; identical to SEQ ID NO: 17
  Description of Artificial Sequence: Synthetic polypeptide

SEQ ID NO: 56 - protein, 10 aa, Artificial Sequence
  Description of Artificial Sequence: Synthetic peptide
DKTHTCPPCP

SEQ ID NO: 57 - protein, 8 aa, Artificial Sequence
  Description of Artificial Sequence: Synthetic peptide
IKRTPREP

SEQ ID NO: 58 - protein, 8 aa, Artificial Sequence
  Description of Artificial Sequence: Synthetic peptide
IKRTVREP

SEQ ID NO: 59 - protein, 7 aa, Artificial Sequence
  Description of Artificial Sequence: Synthetic peptide
IKRTREP

SEQ ID NO: 60 - protein, 9 aa, Artificial Sequence
  Description of Artificial Sequence: Synthetic peptide
IKRTVPREP

SEQ ID NO: 61 - protein, 8 aa, Artificial Sequence
  Description of Artificial Sequence: Synthetic peptide
IKRTVAEP

SEQ ID NO: 62 - protein, 10 aa, Artificial Sequence
  Description of Artificial Sequence: Synthetic peptide
IKRTVAPREP

SEQ ID NO: 63 - protein, 8 aa, Artificial Sequence
  Description of Artificial Sequence: Synthetic peptide
SSASPREP

SEQ ID NO: 64 - protein, 8 aa, Artificial Sequence
  Description of Artificial Sequence: Synthetic peptide
SSASTREP

SEQ ID NO: 65 - protein, 9 aa, Artificial Sequence
  Description of Artificial Sequence: Synthetic peptide
SSASTPREP

SEQ ID NO: 66 - protein, 9 aa, Artificial Sequence
  Description of Artificial Sequence: Synthetic peptide
SSASTKGEP

SEQ ID NO: 67 - protein, 10 aa, Artificial Sequence
  Description of Artificial Sequence: Synthetic peptide
SSASTKGREP

SEQ ID NO: 68-73 - skipped (6 entries)

SEQ ID NO: 74 - protein, 455 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 109 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRT

SEQ ID NO: 77 - protein, 127 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSAS

SEQ ID NO: 78 - protein, 441 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 232 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 80 - protein, 441 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 81 - protein, 229 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 82 - protein, 668 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 10 aa, Artificial Sequence
  Description of Artificial Sequence: Synthetic peptide
TASSGGSSSG

SEQ ID NO: 84 - protein, 255 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKP

SEQ ID NO: 85-95 - skipped (11 entries)

SEQ ID NO: 96 - protein, 4 aa, Artificial Sequence
  Description of Artificial Sequence: Synthetic peptide
AGKC

SEQ ID NO: 97 - protein, 4 aa, Artificial Sequence
  Description of Artificial Sequence: Synthetic peptide
PGKC

SEQ ID NO: 98 - protein, 5 aa, Artificial Sequence
  Description of Artificial Sequence: Synthetic peptide
AGKGC

SEQ ID NO: 99 - protein, 6 aa, Artificial Sequence
  Description of Artificial Sequence: Synthetic peptide
AGKGSC

SEQ ID NO: 100 - protein, 447 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 233 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLGEC

SEQ ID NO: 102 - protein, 441 aa, Artificial Sequence; identical to SEQ ID NO: 10
  Description of Artificial Sequence: Synthetic polypeptide

SEQ ID NO: 103 - protein, 217 aa, Artificial Sequence; identical to SEQ ID NO: 11
  Description of Artificial Sequence: Synthetic polypeptide

SEQ ID NO: 104 - protein, 220 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGC

SEQ ID NO: 105 - protein, 222 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKGC

SEQ ID NO: 106 - protein, 223 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKGSC

SEQ ID NO: 107 - protein, 221 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKC

SEQ ID NO: 108 - protein, 220 aa, Artificial Sequence; identical to SEQ ID NO: 104
  Description of Artificial Sequence: Synthetic polypeptide

SEQ ID NO: 109 - protein, 220 aa, Artificial Sequence; identical to SEQ ID NO: 104
  Description of Artificial Sequence: Synthetic polypeptide

SEQ ID NO: 110 - protein, 220 aa, Artificial Sequence; identical to SEQ ID NO: 104
  Description of Artificial Sequence: Synthetic polypeptide

SEQ ID NO: 111 - protein, 220 aa, Artificial Sequence; identical to SEQ ID NO: 104
  Description of Artificial Sequence: Synthetic polypeptide

SEQ ID NO: 112 - protein, 220 aa, Artificial Sequence; identical to SEQ ID NO: 104
  Description of Artificial Sequence: Synthetic polypeptide

SEQ ID NO: 113 - protein, 220 aa, Artificial Sequence; identical to SEQ ID NO: 104
  Description of Artificial Sequence: Synthetic polypeptide

SEQ ID NO: 114 - protein, 222 aa, Artificial Sequence; identical to SEQ ID NO: 105
  Description of Artificial Sequence: Synthetic polypeptide

SEQ ID NO: 115 - protein, 222 aa, Artificial Sequence; identical to SEQ ID NO: 105
  Description of Artificial Sequence: Synthetic polypeptide

SEQ ID NO: 116 - protein, 222 aa, Artificial Sequence; identical to SEQ ID NO: 105
  Description of Artificial Sequence: Synthetic polypeptide

SEQ ID NO: 117 - protein, 222 aa, Artificial Sequence; identical to SEQ ID NO: 105
  Description of Artificial Sequence: Synthetic polypeptide

SEQ ID NO: 118 - protein, 222 aa, Artificial Sequence; identical to SEQ ID NO: 105
  Description of Artificial Sequence: Synthetic polypeptide

SEQ ID NO: 119 - protein, 222 aa, Artificial Sequence; identical to SEQ ID NO: 105
  Description of Artificial Sequence: Synthetic polypeptide

SEQ ID NO: 120 - protein, 223 aa, Artificial Sequence; identical to SEQ ID NO: 106
  Description of Artificial Sequence: Synthetic polypeptide

SEQ ID NO: 121 - protein, 223 aa, Artificial Sequence; identical to SEQ ID NO: 106
  Description of Artificial Sequence: Synthetic polypeptide

SEQ ID NO: 122 - protein, 223 aa, Artificial Sequence; identical to SEQ ID NO: 106
  Description of Artificial Sequence: Synthetic polypeptide

SEQ ID NO: 123 - protein, 223 aa, Artificial Sequence; identical to SEQ ID NO: 106
  Description of Artificial Sequence: Synthetic polypeptide

SEQ ID NO: 124 - protein, 223 aa, Artificial Sequence; identical to SEQ ID NO: 106
  Description of Artificial Sequence: Synthetic polypeptide

SEQ ID NO: 125 - protein, 223 aa, Artificial Sequence; identical to SEQ ID NO: 106
  Description of Artificial Sequence: Synthetic polypeptide

SEQ ID NO: 126 - protein, 221 aa, Artificial Sequence; identical to SEQ ID NO: 107
  Description of Artificial Sequence: Synthetic polypeptide

SEQ ID NO: 127 - protein, 221 aa, Artificial Sequence; identical to SEQ ID NO: 107
  Description of Artificial Sequence: Synthetic polypeptide

SEQ ID NO: 128 - protein, 221 aa, Artificial Sequence; identical to SEQ ID NO: 107
  Description of Artificial Sequence: Synthetic polypeptide

SEQ ID NO: 129 - protein, 221 aa, Artificial Sequence; identical to SEQ ID NO: 107
  Description of Artificial Sequence: Synthetic polypeptide

SEQ ID NO: 130 - protein, 221 aa, Artificial Sequence; identical to SEQ ID NO: 107
  Description of Artificial Sequence: Synthetic polypeptide

SEQ ID NO: 131 - protein, 221 aa, Artificial Sequence; identical to SEQ ID NO: 107
  Description of Artificial Sequence: Synthetic polypeptide

SEQ ID NO: 132 - protein, 220 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLGEC

SEQ ID NO: 133 - protein, 220 aa, Artificial Sequence; identical to SEQ ID NO: 132
  Description of Artificial Sequence: Synthetic polypeptide

SEQ ID NO: 134 - protein, 220 aa, Artificial Sequence; identical to SEQ ID NO: 132
  Description of Artificial Sequence: Synthetic polypeptide

SEQ ID NO: 135 - protein, 220 aa, Artificial Sequence; identical to SEQ ID NO: 132
  Description of Artificial Sequence: Synthetic polypeptide

SEQ ID NO: 136 - protein, 220 aa, Artificial Sequence; identical to SEQ ID NO: 132
  Description of Artificial Sequence: Synthetic polypeptide

SEQ ID NO: 137 - protein, 220 aa, Artificial Sequence; identical to SEQ ID NO: 132
  Description of Artificial Sequence: Synthetic polypeptide

SEQ ID NO: 138 - protein, 221 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLPGKC

SEQ ID NO: 139 - protein, 221 aa, Artificial Sequence; identical to SEQ ID NO: 138
  Description of Artificial Sequence: Synthetic polypeptide

SEQ ID NO: 140 - protein, 221 aa, Artificial Sequence; identical to SEQ ID NO: 138
  Description of Artificial Sequence: Synthetic polypeptide

SEQ ID NO: 141 - protein, 221 aa, Artificial Sequence; identical to SEQ ID NO: 138
  Description of Artificial Sequence: Synthetic polypeptide

SEQ ID NO: 142 - protein, 221 aa, Artificial Sequence; identical to SEQ ID NO: 138
  Description of Artificial Sequence: Synthetic polypeptide

SEQ ID NO: 143 - protein, 221 aa, Artificial Sequence; identical to SEQ ID NO: 138
  Description of Artificial Sequence: Synthetic polypeptide

SEQ ID NO: 144 - protein, 236 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKGSC

SEQ ID NO: 145 - protein, 233 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGC

SEQ ID NO: 146 - protein, 233 aa, Artificial Sequence; identical to SEQ ID NO: 145
  Description of Artificial Sequence: Synthetic polypeptide

SEQ ID NO: 147 - protein, 233 aa, Artificial Sequence; identical to SEQ ID NO: 145
  Description of Artificial Sequence: Synthetic polypeptide

SEQ ID NO: 148 - protein, 233 aa, Artificial Sequence; identical to SEQ ID NO: 145
  Description of Artificial Sequence: Synthetic polypeptide

SEQ ID NO: 149 - protein, 235 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKGC

SEQ ID NO: 150 - protein, 236 aa, Artificial Sequence; identical to SEQ ID NO: 144
  Description of Artificial Sequence: Synthetic polypeptide

SEQ ID NO: 151 - protein, 234 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKC

SEQ ID NO: 152 - protein, 233 aa, Artificial Sequence; identical to SEQ ID NO: 101
  Description of Artificial Sequence: Synthetic polypeptide

SEQ ID NO: 153 - protein, 234 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLPGKC

SEQ ID NO: 154 - protein, 233 aa, Artificial Sequence; identical to SEQ ID NO: 145
  Description of Artificial Sequence: Synthetic polypeptide

SEQ ID NO: 155 - protein, 235 aa, Artificial Sequence; identical to SEQ ID NO: 149
  Description of Artificial Sequence: Synthetic polypeptide

SEQ ID NO: 156 - protein, 236 aa, Artificial Sequence; identical to SEQ ID NO: 144
  Description of Artificial Sequence: Synthetic polypeptide

SEQ ID NO: 157 - protein, 234 aa, Artificial Sequence; identical to SEQ ID NO: 151
  Description of Artificial Sequence: Synthetic polypeptide

SEQ ID NO: 158 - protein, 233 aa, Artificial Sequence; identical to SEQ ID NO: 101
  Description of Artificial Sequence: Synthetic polypeptide

SEQ ID NO: 159 - protein, 234 aa, Artificial Sequence; identical to SEQ ID NO: 153
  Description of Artificial Sequence: Synthetic polypeptide

SEQ ID NO: 160 - protein, 233 aa, Artificial Sequence; identical to SEQ ID NO: 145
  Description of Artificial Sequence: Synthetic polypeptide

SEQ ID NO: 161 - protein, 235 aa, Artificial Sequence; identical to SEQ ID NO: 149
  Description of Artificial Sequence: Synthetic polypeptide

SEQ ID NO: 162 - protein, 236 aa, Artificial Sequence; identical to SEQ ID NO: 144
  Description of Artificial Sequence: Synthetic polypeptide

SEQ ID NO: 163 - protein, 234 aa, Artificial Sequence; identical to SEQ ID NO: 151
  Description of Artificial Sequence: Synthetic polypeptide

SEQ ID NO: 164 - protein, 233 aa, Artificial Sequence; identical to SEQ ID NO: 101
  Description of Artificial Sequence: Synthetic polypeptide

SEQ ID NO: 165 - protein, 234 aa, Artificial Sequence; identical to SEQ ID NO: 153
  Description of Artificial Sequence: Synthetic polypeptide

SEQ ID NO: 166 - protein, 233 aa, Artificial Sequence; identical to SEQ ID NO: 145
  Description of Artificial Sequence: Synthetic polypeptide

SEQ ID NO: 167 - protein, 235 aa, Artificial Sequence; identical to SEQ ID NO: 149
  Description of Artificial Sequence: Synthetic polypeptide

SEQ ID NO: 168 - protein, 236 aa, Artificial Sequence; identical to SEQ ID NO: 144
  Description of Artificial Sequence: Synthetic polypeptide

SEQ ID NO: 169 - protein, 234 aa, Artificial Sequence; identical to SEQ ID NO: 151
  Description of Artificial Sequence: Synthetic polypeptide

SEQ ID NO: 170 - protein, 233 aa, Artificial Sequence; identical to SEQ ID NO: 101
  Description of Artificial Sequence: Synthetic polypeptide

SEQ ID NO: 171 - protein, 234 aa, Artificial Sequence; identical to SEQ ID NO: 153
  Description of Artificial Sequence: Synthetic polypeptide

SEQ ID NO: 172 - protein, 233 aa, Artificial Sequence; identical to SEQ ID NO: 145
  Description of Artificial Sequence: Synthetic polypeptide

SEQ ID NO: 173 - protein, 235 aa, Artificial Sequence; identical to SEQ ID NO: 149
  Description of Artificial Sequence: Synthetic polypeptide

SEQ ID NO: 174 - protein, 236 aa, Artificial Sequence; identical to SEQ ID NO: 144
  Description of Artificial Sequence: Synthetic polypeptide

SEQ ID NO: 175 - protein, 234 aa, Artificial Sequence; identical to SEQ ID NO: 151
  Description of Artificial Sequence: Synthetic polypeptide

SEQ ID NO: 176 - protein, 233 aa, Artificial Sequence; identical to SEQ ID NO: 101
  Description of Artificial Sequence: Synthetic polypeptide

SEQ ID NO: 177 - protein, 234 aa, Artificial Sequence; identical to SEQ ID NO: 153
  Description of Artificial Sequence: Synthetic polypeptide

SEQ ID NO: 178 - protein, 233 aa, Artificial Sequence; identical to SEQ ID NO: 145
  Description of Artificial Sequence: Synthetic polypeptide

SEQ ID NO: 179 - protein, 235 aa, Artificial Sequence; identical to SEQ ID NO: 149
  Description of Artificial Sequence: Synthetic polypeptide

SEQ ID NO: 180 - protein, 236 aa, Artificial Sequence; identical to SEQ ID NO: 144
  Description of Artificial Sequence: Synthetic polypeptide

SEQ ID NO: 181 - protein, 234 aa, Artificial Sequence; identical to SEQ ID NO: 151
  Description of Artificial Sequence: Synthetic polypeptide

SEQ ID NO: 182 - protein, 233 aa, Artificial Sequence; identical to SEQ ID NO: 101
  Description of Artificial Sequence: Synthetic polypeptide

SEQ ID NO: 183 - protein, 234 aa, Artificial Sequence; identical to SEQ ID NO: 153
  Description of Artificial Sequence: Synthetic polypeptide

SEQ ID NO: 184 - protein, 108 aa, Homo sapiens
TFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRL

SEQ ID NO: 185 - protein, 5 aa, Artificial Sequence
  Description of Artificial Sequence: Synthetic peptide
DKTHT

SEQ ID NO: 186 - protein, 440 aa, Artificial Sequence
  Description of Artificial Sequence: Synthetic polypeptide
IQMTQSPSSLSASVGDRVTITCRASQSVSSAVAWYQQKPGKAPKLLIYSASSLYSGVPSRFSGSRSGTDFTLTISSLQPEDFATYYCQQYYTTPFTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 226 aa, Artificial Sequence
  Description of Artificial Sequence: Synthetic polypeptide
EVQLVESGGGLVQPGGSLRLSCAASGFTFSGYWIHWVRQAPGKGLEWVAAITSRDSYTYYADSVKGRFTISADTSKNTAYLQMNSLRAEDTAVYYCARGSYLGSAFDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 188 - protein, 442 aa, Artificial Sequence
  Description of Artificial Sequence: Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYSSQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 189 - protein, 224 aa, Artificial Sequence
  Description of Artificial Sequence: Synthetic polypeptide
EVQLVESGGGLVQPGGSLRLSCAASGFTFSSYYIHWVRQAPGKGLEWVAKIQPKGGYTYYADSVKGRFTISADTSKNTAYLQMNSLRAEDTAVYYCARSYYYRPHAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 190 - protein, 11 aa, Artificial Sequence
  Description of Artificial Sequence: Synthetic peptide
RASQSVSSAVA

SEQ ID NO: 191 - protein, 7 aa, Artificial Sequence
  Description of Artificial Sequence: Synthetic peptide
SASSLYS

SEQ ID NO: 192 - protein, 6 aa, Artificial Sequence
  Description of Artificial Sequence: Synthetic peptide
YSSQPA

SEQ ID NO: 193 - protein, 6 aa, Artificial Sequence
  Description of Artificial Sequence: Synthetic peptide
YYTTPF

SEQ ID NO: 194 - protein, 6 aa, Artificial Sequence
  Description of Artificial Sequence: Synthetic peptide
SSTTPF

SEQ ID NO: 195 - protein, 5 aa, Artificial Sequence
  Description of Artificial Sequence: Synthetic peptide
FSSYY

SEQ ID NO: 196 - protein, 5 aa, Artificial Sequence
  Description of Artificial Sequence: Synthetic peptide
FSGYW

SEQ ID NO: 197 - protein, 5 aa, Artificial Sequence
  Description of Artificial Sequence: Synthetic peptide
FSGSW

SEQ ID NO: 198 - protein, 11 aa, Artificial Sequence
  Description of Artificial Sequence: Synthetic peptide
AKIQPKGGYTY

SEQ ID NO: 199 - protein, 11 aa, Artificial Sequence
  Description of Artificial Sequence: Synthetic peptide
AAITSRDSYTY

SEQ ID NO: 200 - protein, 9 aa, Artificial Sequence
  Description of Artificial Sequence: Synthetic peptide
SYYYRPHAF

SEQ ID NO: 201 - protein, 8 aa, Artificial Sequence
  Description of Artificial Sequence: Synthetic peptide
GSYLGSAF

SEQ ID NO: 202 - protein, 441 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSTTPFTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 226 aa, Artificial Sequence; identical to SEQ ID NO: 187
  Description of Artificial Sequence: Synthetic polypeptide

SEQ ID NO: 204 - protein, 442 aa, Artificial Sequence; identical to SEQ ID NO: 188
  Description of Artificial Sequence: Synthetic polypeptide

SEQ ID NO: 205 - protein, 224 aa, Artificial Sequence; identical to SEQ ID NO: 189
  Description of Artificial Sequence: Synthetic polypeptide

SEQ ID NO: 206 - protein, 441 aa, Artificial Sequence; identical to SEQ ID NO: 202
  Description of Artificial Sequence: Synthetic polypeptide

SEQ ID NO: 207 - protein, 226 aa, Artificial Sequence
  Description of Artificial Sequence: Synthetic polypeptide
EVQLVESGGGLVQPGGSLRLSCAASGFTFSGSWIHWVRQAPGKGLEWVAAITSRDSYTYYADSVKGRFTISADTSKNTAYLQMNSLRAEDTAVYYCARGSYLGSAFDYWGQGTLVTVSSASPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 208 - protein, 450 aa, Artificial Sequence
  Description of Artificial Sequence: Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYSSQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGGSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 209 - protein, 224 aa, Artificial Sequence; identical to SEQ ID NO: 189
  Description of Artificial Sequence: Synthetic polypeptide

SEQ ID NO: 210 - protein, 447 aa, Artificial Sequence; identical to SEQ ID NO: 100
  Description of Artificial Sequence: Synthetic polypeptide

SEQ ID NO: 211 - protein, 233 aa, Artificial Sequence; identical to SEQ ID NO: 101
  Description of Artificial Sequence: Synthetic polypeptide

SEQ ID NO: 212 - protein, 441 aa, Artificial Sequence; identical to SEQ ID NO: 10
  Description of Artificial Sequence: Synthetic polypeptide

SEQ ID NO: 213 - protein, 217 aa, Artificial Sequence; identical to SEQ ID NO: 11
  Description of Artificial Sequence: Synthetic polypeptide

SEQ ID NO: 214 - protein, 220 aa, Artificial Sequence; identical to SEQ ID NO: 104
  Description of Artificial Sequence: Synthetic polypeptide

SEQ ID NO: 215 - protein, 222 aa, Artificial Sequence; identical to SEQ ID NO: 105
  Description of Artificial Sequence: Synthetic polypeptide

SEQ ID NO: 216 - protein, 223 aa, Artificial Sequence; identical to SEQ ID NO: 106
  Description of Artificial Sequence: Synthetic polypeptide

SEQ ID NO: 217 - protein, 221 aa, Artificial Sequence; identical to SEQ ID NO: 107
  Description of Artificial Sequence: Synthetic polypeptide

SEQ ID NO: 218 - protein, 220 aa, Artificial Sequence; identical to SEQ ID NO: 104
  Description of Artificial Sequence: Synthetic polypeptide

SEQ ID NO: 219 - protein, 220 aa, Artificial Sequence; identical to SEQ ID NO: 104
  Description of Artificial Sequence: Synthetic polypeptide

SEQ ID NO: 220 - protein, 220 aa, Artificial Sequence; identical to SEQ ID NO: 104
  Description of Artificial Sequence: Synthetic polypeptide

SEQ ID NO: 221 - protein, 220 aa, Artificial Sequence; identical to SEQ ID NO: 104
  Description of Artificial Sequence: Synthetic polypeptide

SEQ ID NO: 222 - protein, 220 aa, Artificial Sequence; identical to SEQ ID NO: 104
  Description of Artificial Sequence: Synthetic polypeptide

SEQ ID NO: 223 - protein, 220 aa, Artificial Sequence; identical to SEQ ID NO: 104
  Description of Artificial Sequence: Synthetic polypeptide

SEQ ID NO: 224 - protein, 222 aa, Artificial Sequence; identical to SEQ ID NO: 105
  Description of Artificial Sequence: Synthetic polypeptide

SEQ ID NO: 225 - protein, 222 aa, Artificial Sequence; identical to SEQ ID NO: 105
  Description of Artificial Sequence: Synthetic polypeptide

SEQ ID NO: 226 - protein, 222 aa, Artificial Sequence; identical to SEQ ID NO: 105
  Description of Artificial Sequence: Synthetic polypeptide

SEQ ID NO: 227 - protein, 222 aa, Artificial Sequence; identical to SEQ ID NO: 105
  Description of Artificial Sequence: Synthetic polypeptide

SEQ ID NO: 228 - protein, 222 aa, Artificial Sequence; identical to SEQ ID NO: 105
  Description of Artificial Sequence: Synthetic polypeptide

SEQ ID NO: 229 - protein, 222 aa, Artificial Sequence; identical to SEQ ID NO: 105
  Description of Artificial Sequence: Synthetic polypeptide

SEQ ID NO: 230 - protein, 223 aa, Artificial Sequence; identical to SEQ ID NO: 106
  Description of Artificial Sequence: Synthetic polypeptide

SEQ ID NO: 231 - protein, 223 aa, Artificial Sequence; identical to SEQ ID NO: 106
  Description of Artificial Sequence: Synthetic polypeptide

SEQ ID NO: 232 - protein, 223 aa, Artificial Sequence; identical to SEQ ID NO: 106
  Description of Artificial Sequence: Synthetic polypeptide

SEQ ID NO: 233 - protein, 223 aa, Artificial Sequence; identical to SEQ ID NO: 106
  Description of Artificial Sequence: Synthetic polypeptide

SEQ ID NO: 234 - protein, 223 aa, Artificial Sequence; identical to SEQ ID NO: 106
  Description of Artificial Sequence: Synthetic polypeptide

SEQ ID NO: 235 - protein, 223 aa, Artificial Sequence; identical to SEQ ID NO: 106
  Description of Artificial Sequence: Synthetic polypeptide

SEQ ID NO: 236 - protein, 221 aa, Artificial Sequence; identical to SEQ ID NO: 107
  Description of Artificial Sequence: Synthetic polypeptide

SEQ ID NO: 237 - protein, 221 aa, Artificial Sequence; identical to SEQ ID NO: 107
  Description of Artificial Sequence: Synthetic polypeptide

SEQ ID NO: 238 - protein, 221 aa, Artificial Sequence; identical to SEQ ID NO: 107
  Description of Artificial Sequence: Synthetic polypeptide

SEQ ID NO: 239 - protein, 221 aa, Artificial Sequence; identical to SEQ ID NO: 107
  Description of Artificial Sequence: Synthetic polypeptide

SEQ ID NO: 240 - protein, 221 aa, Artificial Sequence; identical to SEQ ID NO: 107
  Description of Artificial Sequence: Synthetic polypeptide

SEQ ID NO: 241 - protein, 221 aa, Artificial Sequence; identical to SEQ ID NO: 107
  Description of Artificial Sequence: Synthetic polypeptide

SEQ ID NO: 242 - protein, 220 aa, Artificial Sequence; identical to SEQ ID NO: 132
  Description of Artificial Sequence: Synthetic polypeptide

SEQ ID NO: 243 - protein, 220 aa, Artificial Sequence; identical to SEQ ID NO: 132
  Description of Artificial Sequence: Synthetic polypeptide

SEQ ID NO: 244 - protein, 220 aa, Artificial Sequence; identical to SEQ ID NO: 132
  Description of Artificial Sequence: Synthetic polypeptide

SEQ ID NO: 245 - protein, 220 aa, Artificial Sequence; identical to SEQ ID NO: 132
  Description of Artificial Sequence: Synthetic polypeptide

SEQ ID NO: 246 - protein, 220 aa, Artificial Sequence; identical to SEQ ID NO: 132
  Description of Artificial Sequence: Synthetic polypeptide

SEQ ID NO: 247 - protein, 220 aa, Artificial Sequence; identical to SEQ ID NO: 132
  Description of Artificial Sequence: Synthetic polypeptide

SEQ ID NO: 248 - protein, 221 aa, Artificial Sequence; identical to SEQ ID NO: 138
  Description of Artificial Sequence: Synthetic polypeptide

SEQ ID NO: 249 - protein, 221 aa, Artificial Sequence; identical to SEQ ID NO: 138
  Description of Artificial Sequence: Synthetic polypeptide

SEQ ID NO: 250 - protein, 221 aa, Artificial Sequence; identical to SEQ ID NO: 138
  Description of Artificial Sequence: Synthetic polypeptide

SEQ ID NO: 251 - protein, 221 aa, Artificial Sequence; identical to SEQ ID NO: 138
  Description of Artificial Sequence: Synthetic polypeptide

SEQ ID NO: 252 - protein, 221 aa, Artificial Sequence; identical to SEQ ID NO: 138
  Description of Artificial Sequence: Synthetic polypeptide

SEQ ID NO: 253 - protein, 221 aa, Artificial Sequence; identical to SEQ ID NO: 138
  Description of Artificial Sequence: Synthetic polypeptide

SEQ ID NO: 254 - protein, 236 aa, Artificial Sequence; identical to SEQ ID NO: 144
  Description of Artificial Sequence: Synthetic polypeptide

SEQ ID NO: 255 - protein, 233 aa, Artificial Sequence; identical to SEQ ID NO: 145
  Description of Artificial Sequence: Synthetic polypeptide

SEQ ID NO: 256 - protein, 233 aa, Artificial Sequence; identical to SEQ ID NO: 145
  Description of Artificial Sequence: Synthetic polypeptide

SEQ ID NO: 257 - protein, 233 aa, Artificial Sequence; identical to SEQ ID NO: 145
  Description of Artificial Sequence: Synthetic polypeptide

SEQ ID NO: 258 - protein, 233 aa, Artificial Sequence; identical to SEQ ID NO: 145
  Description of Artificial Sequence: Synthetic polypeptide

SEQ ID NO: 259 - protein, 235 aa, Artificial Sequence; identical to SEQ ID NO: 149
  Description of Artificial Sequence: Synthetic polypeptide

SEQ ID NO: 260 - protein, 236 aa, Artificial Sequence; identical to SEQ ID NO: 144
  Description of Artificial Sequence: Synthetic polypeptide

SEQ ID NO: 261 - protein, 234 aa, Artificial Sequence; identical to SEQ ID NO: 151
  Description of Artificial Sequence: Synthetic polypeptide

SEQ ID NO: 262 - protein, 233 aa, Artificial Sequence; identical to SEQ ID NO: 101
  Description of Artificial Sequence: Synthetic polypeptide

SEQ ID NO: 263 - protein, 234 aa, Artificial Sequence; identical to SEQ ID NO: 153
  Description of Artificial Sequence: Synthetic polypeptide

SEQ ID NO: 264 - protein, 233 aa, Artificial Sequence; identical to SEQ ID NO: 145
  Description of Artificial Sequence: Synthetic polypeptide

SEQ ID NO: 265 - protein, 235 aa, Artificial Sequence; identical to SEQ ID NO: 149
  Description of Artificial Sequence: Synthetic polypeptide

SEQ ID NO: 266 - protein, 236 aa, Artificial Sequence; identical to SEQ ID NO: 144
  Description of Artificial Sequence: Synthetic polypeptide

SEQ ID NO: 267 - protein, 234 aa, Artificial Sequence; identical to SEQ ID NO: 151
  Description of Artificial Sequence: Synthetic polypeptide

SEQ ID NO: 268 - protein, 233 aa, Artificial Sequence; identical to SEQ ID NO: 101
  Description of Artificial Sequence: Synthetic polypeptide

SEQ ID NO: 269 - protein, 234 aa, Artificial Sequence; identical to SEQ ID NO: 153
  Description of Artificial Sequence: Synthetic polypeptide

SEQ ID NO: 270 - protein, 233 aa, Artificial Sequence; identical to SEQ ID NO: 145
  Description of Artificial Sequence: Synthetic polypeptide

SEQ ID NO: 271 - protein, 235 aa, Artificial Sequence; identical to SEQ ID NO: 149
  Description of Artificial Sequence: Synthetic polypeptide

SEQ ID NO: 272 - protein, 236 aa, Artificial Sequence; identical to SEQ ID NO: 144
  Description of Artificial Sequence: Synthetic polypeptide

SEQ ID NO: 273 - protein, 234 aa, Artificial Sequence; identical to SEQ ID NO: 151
  Description of Artificial Sequence: Synthetic polypeptide

SEQ ID NO: 274 - protein, 233 aa, Artificial Sequence; identical to SEQ ID NO: 101
  Description of Artificial Sequence: Synthetic polypeptide

SEQ ID NO: 275 - protein, 234 aa, Artificial Sequence; identical to SEQ ID NO: 153
  Description of Artificial Sequence: Synthetic polypeptide

SEQ ID NO: 276 - protein, 233 aa, Artificial Sequence; identical to SEQ ID NO: 145
  Description of Artificial Sequence: Synthetic polypeptide

SEQ ID NO: 277 - protein, 235 aa, Artificial Sequence; identical to SEQ ID NO: 149
  Description of Artificial Sequence: Synthetic polypeptide

SEQ ID NO: 278 - protein, 236 aa, Artificial Sequence; identical to SEQ ID NO: 144
  Description of Artificial Sequence: Synthetic polypeptide

SEQ ID NO: 279 - protein, 234 aa, Artificial Sequence; identical to SEQ ID NO: 151
  Description of Artificial Sequence: Synthetic polypeptide

SEQ ID NO: 280 - protein, 233 aa, Artificial Sequence; identical to SEQ ID NO: 101
  Description of Artificial Sequence: Synthetic polypeptide

SEQ ID NO: 281 - protein, 234 aa, Artificial Sequence; identical to SEQ ID NO: 153
  Description of Artificial Sequence: Synthetic polypeptide

SEQ ID NO: 282 - protein, 233 aa, Artificial Sequence; identical to SEQ ID NO: 145
  Description of Artificial Sequence: Synthetic polypeptide

SEQ ID NO: 283 - protein, 235 aa, Artificial Sequence; identical to SEQ ID NO: 149
  Description of Artificial Sequence: Synthetic polypeptide

SEQ ID NO: 284 - protein, 236 aa, Artificial Sequence; identical to SEQ ID NO: 144
  Description of Artificial Sequence: Synthetic polypeptide

SEQ ID NO: 285 - protein, 234 aa, Artificial Sequence; identical to SEQ ID NO: 151
  Description of Artificial Sequence: Synthetic polypeptide

SEQ ID NO: 286 - protein, 233 aa, Artificial Sequence; identical to SEQ ID NO: 101
  Description of Artificial Sequence: Synthetic polypeptide

SEQ ID NO: 287 - protein, 234 aa, Artificial Sequence; identical to SEQ ID NO: 153
  Description of Artificial Sequence: Synthetic polypeptide

SEQ ID NO: 288 - protein, 233 aa, Artificial Sequence; identical to SEQ ID NO: 145
  Description of Artificial Sequence: Synthetic polypeptide

SEQ ID NO: 289 - protein, 235 aa, Artificial Sequence; identical to SEQ ID NO: 149
  Description of Artificial Sequence: Synthetic polypeptide

SEQ ID NO: 290 - protein, 236 aa, Artificial Sequence; identical to SEQ ID NO: 144
  Description of Artificial Sequence: Synthetic polypeptide

SEQ ID NO: 291 - protein, 234 aa, Artificial Sequence; identical to SEQ ID NO: 151
  Description of Artificial Sequence: Synthetic polypeptide

SEQ ID NO: 292 - protein, 233 aa, Artificial Sequence; identical to SEQ ID NO: 101
  Description of Artificial Sequence: Synthetic polypeptide

SEQ ID NO: 293 - protein, 234 aa, Artificial Sequence; identical to SEQ ID NO: 153
  Description of Artificial Sequence: Synthetic polypeptide

SEQ ID NO: 294 - protein, 108 aa, Artificial Sequence; identical to SEQ ID NO: 184
  Description of Artificial Sequence: Synthetic polypeptide

SEQ ID NO: 295 - protein, 453 aa, Artificial Sequence
  Description of Artificial Sequence: Synthetic polypeptide
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 296 - protein, 236 aa, Artificial Sequence
  Description of Artificial Sequence: Synthetic polypeptide
EVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKGSC

SEQ ID NO: 297 - protein, 671 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTTFRPEVCLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 224 aa, Artificial Sequence
  Description of Artificial Sequence: Synthetic polypeptide
EVQLVESGGGLVQPGGSLRLSCAASGFTFSDYDIHWVRQAPGKGLEWVGDITPYDGTTNYADSVKGRFTISADTSKNTAYLQMNSLRAEDTAVYYCARLVGEIATGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSC

SEQ ID NO: 299 - protein, 228 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTFRPEVHLLPPCSEELALNELVTLTCLARGFSPKDVLVRWLQGSQELPREKYLTWASRQEPSQGTTTFAVTSILRVAAEDWKKGDTFSCMVGHEALPLAFTQKTIDRL

SEQ ID NO: 300 - protein, 668 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPREPQVYTLPPSRDELTKNQVSLKCLVKGFYPSDIAVEWESNGQPENNYKTTPPVLDSDGSFFLYSKLTVDKSRWQQGNVFSCSVMHEALHNHYTQKSLSLSKSCTASSGGSSSGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 224 aa, Artificial Sequence; identical to SEQ ID NO: 298
  Description of Artificial Sequence: Synthetic polypeptide

SEQ ID NO: 302 - protein, 225 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSPREPQVYTDPPSRDELTKNQVSLTCLVKGFYPSDIAVEWESNGQPENNYKTTPPVLDSDGSFFLYSKLTVDKSRWQQGNVFSCSVMHEALHNHYTQKSLSLSGEC

SEQ ID NO: 303 - protein, 108 aa, Artificial Sequence
  Description of Artificial Sequence: Synthetic polypeptide
TFRPEVCLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRL

SEQ ID NO: 304 - protein, 108 aa, Artificial Sequence
  Description of Artificial Sequence: Synthetic polypeptide
TFRPEVHLLPPCSEELALNELVTLTCLARGFSPKDVLVRWLQGSQELPREKYLTWASRQEPSQGTTTFAVTSILRVAAEDWKKGDTFSCMVGHEALPLAFTQKTIDRL